(19) United States
(12) Patent Application Publication
(10) Pub. No.: US 2007/0258949 A1
(43) Pub. Date: Nov. 8, 2007

(54) Human Cdna Clones Comprising Polynucleotides Encoding Polypeptides and Methods of Their Use

(75) Inventors: Ernestine Lee, San Francisco, CA (US); Kevin Hestir, San Francisco, CA (US); Keting Chu, Woodside, CA (US); Lewis Thomas Williams, San Francisco, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: FIVE PRIME THERAPEUTICS, INC., San Francisco, CA (US)

(21) Appl. No.: 10/591,451

(22) PCT Filed: Mar. 1, 2005
(86) PCT No.: PCT/US05/06473

§ 371 Date: Jun. 4, 2007

Related U.S. Application Data
(60) Provisional application No. 60/548,191, filed on Mar. 1, 2004.
(60) Provisional application No. 60/647,013, filed on Jan. 27, 2005.
(60) Provisional application No. 60/654,229, filed on Feb. 18, 2005.

(51) Int. Cl. A61K 38/00; A61K 48/00; C07H 21/04; C07K 14/00; C07K 16/18; C12N 15/00; C12N 15/87; C12N 5/06; C12P 1/04; C12Q 1/68; G01N 33/53

(52) U.S. Cl. 424/93.1; 435/320.1; 435/325; 435/455; 435/6; 435/69.1; 435/7.1; 514/2; 514/44; 530/350; 530/387.1; 536/22.1

(57) ABSTRACT

The invention provides novel polynucleotides, related polypeptides related nucleic acid and polypeptide compositions corresponding to novel human cDNA clones, and related modulators, such as antibodies and small molecule modulators. The invention also provides methods to make and use these polynucleotides, polypeptides, related compositions, and modulators. These methods include diagnostic, prophylactic and therapeutic applications. The compositions and methods of the invention are useful in treating proliferative disorders, e.g., cancers, and inflammatory, immune, bacterial, and viral disorders.

### PRIORITY CLAIM

This application claims the benefit of U.S. Provisional Applications 60/548,191, filed Mar. 1, 2004; 60/647,013, filed Jan. 27, 2005; and “Fusion Polypeptides of Human Fetuin and Therapeutically Active Polypeptides,” filed Feb. 18, 2005; the disclosures of which are incorporated in their entireties.

### TECHNICAL FIELD

The present invention is related generally to novel human cDNA clones and novel polypeptides encoded therefrom, and their compositions. The present invention also relates to methods of modulating a biological activity through the use of the novel polynucleotides and novel polypeptides of the invention.

### BACKGROUND OF THE INVENTION

Sequencing of the genomes, or portions of the genomes, of numerous biological materials, including humans, animals, microorganisms and various plant varieties, has been, and is being carried out on a large scale. Polynucleotides identified using sequencing techniques may be partial or full-length genes, and may contain open reading frames, or portions of open reading frames, that encode polypeptides. Putative polypeptides may be determined based on polynucleotide sequences. The sequencing data relating to polynucleotides thus represents valuable and useful information.

Polynucleotides may be analyzed for various degrees of novelty by comparing identified sequences to sequences published in various public domain databases, such as EMBL. Newly identified polynucleotides and putative polypeptides may also be compared to polynucleotides and polypeptides contained in public domain information to ascertain homology to known polynucleotides and polypeptides. In this way, the degree of similarity, identity, or homology of polynucleotides and polypeptides of unknown function may be determined relative to polynucleotides and polypeptides having known functions.

Information relating to the sequences of isolated polynucleotides may be used in a variety of ways. Specified polynucleotides having a particular sequence may be isolated or synthesized for use in in vivo or in vitro experimentation as probes or primers. Alternatively, collections of sequences of isolated polynucleotides may be stored using magnetic or optical storage medium, and analyzed or manipulated using computer hardware and software, as well as other types of tools.

### DISCLOSURE OF THE INVENTION

The present invention is related generally to novel polynucleotides and novel polypeptides encoded thereby, their compositions, antibodies directed thereto, and other agonists or antagonists thereto. The polynucleotides and polypeptides are useful in diagnostic, prophylactic, and therapeutic applications for a variety of diseases, disorders, syndromes, and conditions, as well as in discovering new diagnostics, prophylactics, and therapeutics for such diseases, disorders, syndromes, and conditions (hereinafter “disorders”). The present invention also relates to methods of modulating biological activities through the use of the novel polynucleotides and novel polypeptides of the invention and through the use of agonists and antagonists, such as antibodies, thereto.

This application also relates to the field of polypeptides that are associated with regulating cell growth and differentiation, that are over-expressed in cancer, and/or that can be associated with proliferation or inhibition of cancer growth, including hematopoietic cancers such as leukemias, lymphomas, and solid cancers such as pancreatic cancer, prostate cancer, tracheal cancer, breast cancer, and lung cancer, for example, adenocarcinomas and/or squamous cell carcinomas. These polypeptides may also be associated with other conditions, such as degenerative, inflammatory, immune, and metabolic disorders, as well as microbial infections, including viral, bacterial, fungal, and parasitic disorders.

This application further relates to modulators of biological activity that can specifically bind to these polynucleotides or polypeptides, or otherwise specifically modulate their activity. For example, they can directly or indirectly induce antibody-dependent cellular cytotoxicity (ADCC), complement-dependent cytotoxicity (CDC), endocytosis, apoptosis, or recruitment of other cells to effect cell activation, cell inactivation, cell growth or differentiation or inhibition thereof, and cell killing.

The invention provides an isolated nucleic acid molecule with a first polynucleotide having the nucleotide sequence of SEQ ID NOS.:1-187, 375-484; a polynucleotide encoding a polypeptide comprising an amino acid sequence chosen from SEQ ID NOS.:188-374; a polynucleotide with a nucleotide sequence that is complementary to the first nucleotide sequence; or a biologically active fragment of any of these. This nucleic acid molecule can be a cDNA molecule, a genomic DNA molecule, a cRNA molecule, a siRNA molecule, an RNAi molecule, an mRNA molecule, an anti-sense molecule, or a ribozyme. This nucleic acid molecule may also comprise its complement.

The invention also provides a second nucleic acid molecule with a sequence at least about 80%, or about 90%, or about 95% homologous to the first nucleic acid molecule. The invention further provides a second nucleic acid molecule with a sequence encoding a homologous or heterologous secretory leader.

The invention provides an isolated polypeptide with a first amino acid sequence of SEQ ID NOS.:188-375; a sequence encoded by one of SEQ ID NOS.:1-187, 375-484; or an active fragment of any of these. This polypeptide can be present in a cell culture and/or a cell culture medium. Suitable cell cultures include bacterial cell cultures, mammalian cell cultures, insect cell cultures, and yeast cell cultures. The polypeptide can also be present in a plant or a non-human animal.

The invention also provides polypeptides with a second amino acid sequence operably linked to the first, e.g., a homologous or heterologous secretory leader. The invention provides polypeptides consisting essentially of a secretory leader sequence or consisting essentially of a mature polypeptide sequence. The invention provides a polypeptide with at least six contiguous amino acids chosen from SEQ ID NOS.:188-374 or encoded by SEQ ID NOS.:1-187, 375-484.

The invention provides a vector comprising a promoter that regulates the expression of an isolated nucleic acid molecule with a first polynucleotide having the nucleotide sequence of SEQ ID NOS.:1-187, 375-484; a polynucleotide encoding a polypeptide comprising an amino acid sequence chosen from SEQ ID NOS.:188-374; a complementary polynucleotide with a nucleotide sequence that is complementary to the first nucleotide sequence; or a biologically active fragment of any of these. This vector can be a viral vector or a plasmid, e.g., a pTT vector. The promoter can be naturally contiguous or not naturally contiguous to the nucleic acid molecule. The promoter can be inducible, conditionally active, constitutive, or tissue-specific.

The invention provides a recombinant host cell with an isolated nucleic acid molecule with a first polynucleotide having the nucleotide sequence of SEQ ID NOS.:1-187, 375-484; a polynucleotide encoding a polypeptide comprising an amino acid sequence chosen from SEQ ID NOS.:188-374; a complementary polynucleotide with a nucleotide sequence that is complementary to the first nucleotide sequence; or a biologically active fragment of any of these; an isolated polypeptide with a first amino acid sequence of SEQ ID NOS.:188-374; a sequence encoded by one of SEQ ID NOS.:1-187, 375-484; or an active fragment of any of these; and/or a vector, as described above. This recombinant host cell can be prokaryotic or eukaryotic, e.g., human, non-human mammalian, insect, fish, plant, or fungal.

The invention also provides an animal injected with an isolated nucleic acid molecule with a first polynucleotide having the nucleotide sequence of SEQ ID NOS.:1-187, 375-484; a polynucleotide encoding a polypeptide comprising an amino acid sequence chosen from SEQ ID NOS.:188-374; a complementary polynucleotide with a nucleotide sequence that is complementary to the first nucleotide sequence; or a biologically active fragment of any of these; or an isolated polypeptide with a first amino acid sequence of SEQ ID NOS.:188-374; a sequence encoded by one of SEQ ID NOS.:1-187, 375-484; or an active fragment of any of these. Animals suitable for practicing the invention include, but are not limited to rodents, non-human primates, rabbits, dogs, and pigs.

The invention further provides a pharmaceutical composition with a pharmaceutically acceptable carrier and an isolated polynucleotide with a nucleic acid sequence chosen from SEQ ID NOS.:1-187 and 375-484 and/or an amino acid sequence chosen from SEQ ID NOS.:188-374. Pharmaceutical compositions of the invention include those with pharmaceutically acceptable carriers and one or more vectors described above. Pharmaceutical compositions of the invention also include those with pharmaceutically acceptable carriers and one or more host cells described above. The invention provides host cell compositions made up of any of the host cells described above and a pharmaceutically acceptable carrier.

In another aspect, the invention provides a method of producing a recombinant host cell by providing a vector, as described above and allowing a cell to come into contact with the vector to form a recombinant host cell transfected with a nucleic acid molecule of the vector.

The invention also provides a method of producing a polypeptide by providing one or more isolated nucleic acid molecule with a first polynucleotide having the nucleotide sequence of SEQ ID NOS.:1-187, 375-484; a polynucleotide encoding a polypeptide comprising an amino acid sequence chosen from SEQ ID NOS.:188-374; a complementary polynucleotide with a nucleotide sequence complementary to the first nucleotide sequence; or a biologically active fragment of any of these and expressing the nucleic acid molecule in an expression system to produce the polypeptide. This method can be practiced in a prokaryotic or eukaryotic cellular expression system, for example, systems that utilize human cells, non-human mammalian cells, insect cells, fish cells, plant cells, or fungal cells. The expression system can comprise a recombinant host cell transfected with a nucleic acid molecule of the invention, then cultured to produce the polypeptide. This method can also be practiced in a cell-free expression system, e.g., a wheat germ lysate expression system, a rabbit reticulocyte expression system, a ribosomal display, or an E. coli lysate expression system. The invention further provides a polypeptide made by the methods described herein.

The invention further provides a fusion molecule comprising an isolated polypeptide with a first amino acid sequence of SEQ ID NOS.:188-374; a sequence encoded by one of SEQ ID NOS.:1-187, 375-484; or an active fragment of any of these; and a fusion partner. The invention provides a fusion molecule with a first polypeptide that comprises an amino acid sequence of a therapeutic molecule and a second polypeptide with an amino acid sequence of a fusion partner. The fusion molecule may have a higher plasma stability than the therapeutic molecule absent the fusion partner. Suitable fusion partners include a polymer, a polypeptide, a succinyl group, fetuin, leucine zipper nuclear factor erythroid derivative-2 (NFE2), neuroretinal leucine zipper, tetranectin, an Fc fragment, and/or serum albumin.

The invention yet further provides an antibody or a biologically active fragment thereof specifically recognizing, binding to, and/or modulating the biological activity of at least one molecule chosen from a polypeptide encoded by a nucleic acid molecule or a polypeptide of the invention. This antibody or active fragment may modulate by interfering with the binding of the polypeptide with its receptor. Antibodies of the invention may be provided as compositions with pharmaceutically acceptable carriers. Antibodies of the invention may be polyclonal, monoclonal, single chain, or an active fragment of any of these. For example, the fragment may be an antigen binding fragment, an Fc fragment, a cdr fragment, a V_{H} fragment, a V_{C} fragment, or a framework fragment.

In another aspect, the invention provides diagnostic methods and kits. It provides a method of determining the presence of the nucleic acid molecule of one or more of SEQ ID NOS.:1-187, 375-484, or a complement thereof in a sample by providing a complement to the nucleic acid molecule or providing a complement to the complement of the nucleic acid molecule; allowing the molecule to interact with the sample; and determining whether interaction has occurred. It provides a method of determining the presence of the polypeptide of one or more of SEQ ID NOS.:188-374, or fragment thereof, in a sample, by providing an antibody that specifically binds to or interferes with the activity of the polypeptide; allowing the antibody to interact with the polypeptide in the sample, if any; and determining whether interaction has occurred. The invention provides a kit comprising such an antibody or fragment thereof and instructions for its use. The invention also provides a method of determining the presence of a specific antibody to a polypeptide encoded by a nucleotide of SEQ ID NOS.:1-187, 375-484, or fragment thereof; or the polypeptide of one or more of SEQ ID NOS.:188-374, or fragment thereof in a sample by providing the polypeptide; allowing it to interact with a specific antibody in the sample, if present; and determining whether interaction has occurred.

In a further aspect, the invention provides a method of inhibiting tumor growth by providing a composition comprising an isolated polypeptide with a first amino acid sequence of SEQ ID NOS.:188-374; a sequence encoded by one of SEQ ID NOS.:1-187, 375-484; or an active fragment of any of these; which may or may not be operably linked to a secretory leader; and contacting the tumor with the composition.

The invention also provides a method of killing tumor cells by contacting tumor cells with an isolated polypeptide with a first amino acid sequence of SEQ ID NOS.:188-374; a sequence encoded by one of SEQ ID NOS.:1-187, 375-484; or an active fragment of any of these; which may or may not be operably linked to a secretory leader. This method is suitable for killing human tumor cells, e.g., solid or leukemic human tumor cells. The tumor cells may come from, e.g. a carcinoma, an adenocarcinoma, a sarcoma, or a leukemia. They may be, e.g., prostate tumor cells, pancreatic tumor cells, breast tumor cells, colon tumor cells, lung tumor cells, bladder tumor cells, stomach tumor cells, kidney tumor cells, testicular tumor cells, endocrine tumor cells, or skin tumor cells.

The invention further provides a method for treating a tumor in a subject by providing a composition with a pharmaceutically acceptable carrier and an isolated polypeptide with a first amino acid sequence of SEQ ID NOS.:188-374; a sequence encoded by one of SEQ ID NOS.:1-187, 375-484; or an active fragment of any of these; and, optionally, a fusion partner; and administering the composition to the subject. This method can be used to treat, e.g., prostatic or pancreatic tumors.

The invention provides combination therapies. It provides a pharmaceutical composition with a polypeptide encoded by a nucleotide of SEQ ID NOS.:1-187, 375-484, or fragment thereof; or the polypeptide of one or more of SEQ ID NOS.:188-374, or fragment thereof; an anti-cancer agent; and a pharmaceutically acceptable carrier. The anti-cancer agent may be a chemotherapeutic agent, a radiotherapeutic agent, an anti-angiogenic agent, an apoptosis-inducing agent, or any other agent that represents the standard of anti-cancer treatment. Suitable chemotherapeutic agents include, but are not limited to, a steroid, a cytokine, a cytosine arabinoside, fluorouracil, methotrexate, aminopterin, an anthracycline, mitomycin C, a vinca alkaloid, an antibiotic, demecolcine, etoposide, mithramycin, chlorambucil, and melphalan.

The invention also provides a method of treating a tumor in a subject by providing a first composition comprising a polypeptide encoded by a nucleotide of SEQ ID NOS.:1-187, 375-484, or fragment thereof; or the polypeptide of one or more of SEQ ID NOS.:188-374, or fragment thereof; providing a second composition comprising an anti-cancer agent different from the polypeptide; and administering the first and second compositions to the subject. Suitable second compositions include monoclonal antibody compositions, chemotherapeutic agents, or other polypeptides. This method is effective against, e.g., prostatic and pancreatic tumors.

The invention further includes a method of treating an immune disease in a subject by providing a first composition comprising polypeptide encoded by a nucleotide of SEQ ID NOS.:1-187, 375-484, or fragment thereof; or the polypeptide of one or more of SEQ ID NOS.:188-374, or fragment thereof; providing a second composition comprising an agent effective in treating an immune disease different from the polypeptide; and administering the first and second compositions to the subject. Suitable second compositions include monoclonal antibody compositions, chemotherapeutic agents, or other polypeptides.

The invention further includes a method of treating a metabolic disease in a subject by providing a first composition comprising polypeptide encoded by a nucleotide of SEQ ID NOS:1-187, 375-484, or fragment thereof; or the polypeptide of one or more of SEQ ID NOS:188-374, or fragment thereof; providing a second composition comprising an agent effective in treating a metabolic disease different from the polypeptide; and administering the first and second compositions to the subject. Suitable second compositions include monoclonal antibody compositions, chemotherapeutic agents, or other polypeptides.

The invention yet further includes a method of treating a degenerative disease in a subject by providing a first composition comprising polypeptide encoded by a nucleotide of SEQ ID NOS:1-187, 375-484, or fragment thereof; or the polypeptide of one or more of SEQ ID NOS.:188-374, or fragment thereof; providing a second composition comprising an agent effective in treating a degenerative disease different from the polypeptide; and administering the first and second compositions to the subject. Suitable second compositions include monoclonal antibody compositions, chemotherapeutic agents, or other polypeptides.

### MODES FOR CARRYING OUT THE INVENTION

### Definitions

The terminologies used herein have their ordinary meanings. Further, the present invention can be more readily understood in light of the following particular definitions.

The terms “polynucleotide,” “nucleotide,” “nucleic acid,” “nucleic acid molecule,” “nucleic acid sequence,” “polynucleotide sequence,” and “nucleotide sequence” are used interchangeably herein to refer to polymeric forms of nucleotides of any length. The polynucleotides can contain deoxyribonucleotides, ribonucleotides, and/or their analogs or derivatives. For example, nucleic acids can be naturally occurring DNA or RNA, or can be synthetic analogs, as known in the art. The terms may encompass genomic DNA, genes, gene fragments, exons, introns, regulatory sequences or regulatory elements (such as promoters, enhancers, initiation and termination regions, other control regions, expression regulatory factors, and expression controls), DNA comprising one or more single-nucleotide polymorphisms (SNPs), allelic variants, isolated DNA of any sequence, and cDNA. The terms also encompass mRNA, tRNA, rRNA, ribozymes, splice variants, antisense RNA, antisense conjugates, RNAi, and isolated RNA of any sequence. The terms additionally encompass recombinant polynucleotides, heterologous polynucleotides, branched polynucleotides, labeled polynucleotides, hybrid DNA/RNA, polynucleotide constructs, vectors comprising the subject nucleic acids, nucleic acid probes, primers, and primer pairs. The polynucleotides can comprise modified nucleic acid molecules, with alterations in the backbone, sugars, or heterocyclic bases, such as methylated nucleic acid molecules, peptide nucleic acids, and nucleic acid molecule analogs, which may be suitable as, for example, probes, if they demonstrate superior stability and/or binding affinity under assay conditions. The terms also encompass single-stranded, double-stranded and triple helical molecules that are either DNA, RNA, or hybrid DNA/RNA and that may encode a full-length gene or a biologically active fragment thereof. Biologically active fragments of polynucleotides can comprise regulatory regions that regulate the expression of a gene or can encode the polypeptides herein, as well as anti-sense and RNAi molecules. Thus, the full length polynucleotides herein may be treated with enzymes, such as Dicer, to generate a library of short RNAi fragments which are within the scope of the present invention.

“Nucleic acid composition” as used herein is a composition comprising a nucleic acid molecule, including one having a nucleotide sequence open reading frame that encodes a polypeptide and is capable, under appropriate conditions, of being expressed as a polypeptide. The term includes, for example, vectors, including plasmids, cosmids, viral vectors (e.g., retrovirus vectors such as lentivirus, adenovirus, and the like), human, yeast, bacterial, P1-derived artificial chromosomes (HAC's, YAC's, BAC's, PAC's, etc), and mini-chromosomes, in vitro host cells, in vivo host cells, tissues, organs, allogenic or congenic grafts or transplants, multicellular organisms, and chimeric, genetically modified, or transgenic animals comprising a subject nucleic acid sequence.

A “complement” of a nucleic acid molecule is a one that is comprised of its complementary base pairs. Deoxyribonucleotides with the base adenine are complementary to those with the base thymidine, and deoxyribonucleotides with the base thymidine are complementary to those with the base adenine. Deoxyribonucleotides with the base cytosine are complementary to those with the base guanine, and deoxyribonucleotides with the base guanine are complementary to those with the base cytosine. Ribonucleotides with the base adenine are complementary to those with the base uracil, and deoxyribonucleotides with the base uracil are complementary to those with the base adenine. Ribonucleotides with the base cytosine are complementary to those with the base guanine, and deoxyribonucleotides with the base guanine are complementary to those with the base cytosine.

A “promoter,” as used herein, is a DNA regulatory region capable of binding RNA polymerase in a mammalian cell and initiating transcription of a downstream (3′ direction) coding sequence operably linked thereto. For purposes of the present invention, a promoter sequence includes the minimum number of bases or elements necessary to initiate transcription of a gene of interest at levels detectable above background. Within the promoter sequence is a transcription initiation site, as well as protein binding domains (consensus sequences) responsible for the binding of RNA polymerase. Eucaryotic promoters will often, but not always, contain “TATA” boxes and “CAT” boxes. Promoters include those that are naturally contiguous to a nucleic acid molecule and those that are not naturally contiguous to a nucleic acid molecule. Additionally, a promoter includes inducible promoters, conditionally active promoters, such as a cre-lox promoter, constitutive promoters, and tissue specific promoters.

A “vector” is a plasmid that can be used to transfer DNA sequences from one organism to another. An “expression vector” is a cloning vector that contains regulatory sequences that allow transcription and translation of a cloned gene or genes and thus transcribe and clone DNA. Expression vectors can be used to express the polypeptides of the invention and typically include restriction sites to provide for the insertion of nucleic acid sequences encoding heterologous protein or RNA molecules. Artificially constructed “plasmids,” i.e., small, independently replicating pieces of extrachromosomal cytoplasmic DNA that can be transferred from one organism to another, are commonly used as cloning vectors.

The term “host cell” includes an individual cell, cell line, cell culture, or in vivo cell, which can be or has been a recipient of any polynucleotides or polypeptides of the invention, for example, a recombinant vector, an isolated polynucleotide, antibody or fusion protein. Host cells include progeny of a single host cell, and the progeny may not necessarily be completely identical (in morphology, physiology, or in total DNA, RNA, or polypeptide complement) to the original parent cell due to natural, accidental, or deliberate mutation and/or change. Host cells can be prokaryotic or eukaryotic, including mammalian, insect, amphibian, reptile, crustacean, avian, fish, plant, and fungal cells. A host cell includes cells transformed, transfected, transduced, or infected in vivo or in vitro with a polynucleotide of the invention, for example, a recombinant vector. A host cell which comprises a recombinant vector of the invention may be called a “recombinant host cell.”

“Expression of a nucleic acid molecule” refers to the conversion of the information contained in the molecule, into a gene product. A gene product can be the direct transcriptional product of a gene (e.g., mRNA, tRNA, rRNA, antisense RNA, ribozyme, structural RNA, or any other type of RNA) or a peptide or polypeptide produced by translation of an mRNA. Gene products also include RNAs which are modified by processes such as capping, polyadenylation, methylation, and editing, and proteins modified by, for example, methylation, acetylation, phosphorylation, ubiquitination, ADP-ribosylation, myristilation, and glycosylation.

The term “operably linked” refers to nucleotide sequences that are associated or connected in such a manner that their transcription or translation can be associated or connected, e.g., they can be transcribed or translated together. With respect to polypeptides, “operably linked” refers to amino acid sequences that are associated or connected in structure and/or function, e.g., a fusion partner operably linked to a therapeutic polypeptide to form a fusion protein, or a secretory leader sequence operably linked to a mature polypeptide to form a secreted protein.

The terms “polypeptide,” “peptide,” and “protein,” used interchangeably herein, refer to a polymeric form of amino acids of any length, which can include naturally-occurring amino acids, coded and non-coded amino acids, chemically or biochemically modified, derivatized, or designer amino acids, amino acid analogs, peptidomimetics, and depsipeptides, and polypeptides having modified, cyclic, bicyclic, depsicyclic, or depsibicyclic peptide backbones. The term includes single chain protein as well as multimers. The term also includes conjugated proteins, fusion proteins, including, but not limited to, glutathione S-transferase (GST) fusion proteins, fusion proteins with a heterologous amino acid sequence, fusion proteins with heterologous and homologous leader sequences, fusion proteins with or without N-terminal methionine residues, pegolyated proteins, and immunologically tagged, or his-tagged proteins. Also included in this term are variations of naturally occurring proteins, where such variations are homologous or substantially similar to the naturally occurring protein, as well as corresponding homologs from different species. Variants of polypeptide sequences include insertions, additions, deletions, or substitutions compared with the subject polypeptides. The term also includes peptide aptamers.

By “isolated” is meant, when referring to a polynucleotide or polypeptide of the invention, that the indicated molecule is substantially separated, e.g., from the whole organism in which the molecule is found or from the cell culture in which the antibody is produced, or is present in the substantial absence of other biological macromolecules of the same type. For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

A “secreted protein” is one capable of being directed to the endoplasmic reticulum (ER), secretory vesicles, or the extracellular space as a result of a secretory leader, signal peptide, or leader sequence. A “secreted protein” is also one released into the extracellular space, e.g., by exocytosis or proteolytic cleavage, regardless of whether it comprises a signal sequence. A secreted protein may in some circumstances undergo processing to a “mature” polypeptide.

A “leader sequence” comprises a sequence of amino acid residues, beginning at amino acid residue 1 located at the amino terminus of the polypeptide, and extending to a cleavage site, which, upon proteolytic cleavage, results in formation of a mature protein. Leader sequences are generally hydrophobic and have some positively charged residues. Leader sequences can be natural or synthetic, heterologous, or homologous with the protein to which they are attached. A “secretory leader” is a leader sequence that directs a protein to be secreted from the cell. A secretion signal sequence can be naturally occurring or it can be engineered.

A “mature polypeptide” is a polypeptide that has been acted upon by a protease that cleaves the leader sequence, for example, after secretion from the cell, or after being directed to an appropriate intracellular compartment.

The term “receptor” refers to a polypeptide that binds to a specific extracellular molecule and may initiate a cellular response.

A “fusion molecule” is a molecule, e.g., a polynucleotide or polypeptide, that represents the joining of all or portions of more than one gene. For example, a fusion protein can be the product from splicing strands of recombinant DNA and expressing the hybrid gene. A fusion molecule can be made by genetic engineering, e.g., by removing the stop codon from the DNA sequence of the first protein, then appending the DNA sequence of the second protein in frame. That DNA sequence will then be expressed by a cell as a single protein. Typically this is accomplished by cloning a cDNA into an expression vector in frame with an existing gene.

A “fusion partner” is a polypeptide fused in-frame at the N-terminus and/or C-terminus of a therapeutic or prophylactic polypeptide, or internally to a therapeutic or prophylactic polypeptide.

By “fragment” is intended a polypeptide consisting of only a part of the intact full-length or naturally occurring polypeptide sequence and structure. The fragment can include e.g., a C-terminal deletion, an N-terminal deletion, and/or an internal deletion of a native polypeptide or an extracellular domain of a transmembrane protein. A fragment of a protein will generally include at least about 5-10, 15-25, or 20-50 or more contiguous amino acid residues of the full-length molecule, at least about 15-25 contiguous amino acid residues of the full-length molecule, or any integer between five amino acids and the full-length sequence.

A “biologically active” entity, or an entity having “biological activity,” is one having structural, regulatory, or biochemical functions of a naturally occurring molecule or any function related to or associated with a metabolic or physiological process. Biologically active polynucleotide fragments are those exhibiting activity similar, but not necessarily identical, to an activity of a polynucleotide of the present invention. The biological activity can include an improved desired activity, or a decreased undesirable activity. For example, an entity demonstrates biological activity when it participates in a molecular interaction with another molecule, such as hybridization, when it has therapeutic value in alleviating a disease condition, when it has prophylactic value in inducing an immune response, when it has diagnostic value in determining the presence of a molecule, such as a biologically active fragment of a polynucleotide that can, e.g., be detected as unique for the polynucleotide molecule, or when it is used as a primer in PCR. A biologically active polypeptide or fragment thereof includes one that can participate in a biological reaction, for example, one that can serve as an epitope or immunogen to stimulate an immune response, such as production of antibodies, or that can participate in signal transduction by binding to receptors, proteins, or nucleic acids, or activating enzymes or substrates.

The term “antibody” or “immunoglobulin” refers to a protein, e.g., one generated by the immune system, synthetically, or recombinantly, that is capable of recognizing and binding to a specific antigen; antibodies are commonly known in the art. The term includes active fragments, including for example, an antigen binding fragment of an immunoglobulin, a variable and/or constant region of a heavy chain, a variable and/or constant region of a light chain, a complementarity determining region (cdr), and a framework region. The terms include polyclonal and monoclonal antibody preparations, as well as preparations including hybrid antibodies, altered antibodies, chimeric antibodies, hybrid (chimeric) antibody molecules, F(ab′)₂ and F(ab) fragments; Fv molecules (e.g., noncovalent heterodimers), dimeric and trimeric antibody fragment constructs; minibodies, humanized antibody molecules and any functional fragments obtained from such molecules, wherein such fragments retain specific binding.

As used herein, the phrase “pharmaceutically acceptable carrier” is intended to include substances that can be co-administered with the compositions of the invention that allow the composition or active molecule therein to perform its intended function. Examples of such carriers include solutions, solvents, buffers, adjuvants, dispersion media, delay agents, emulsions, and the like. Further, any other conventional carrier, suitable for use with the described compositions, fall within the scope of the instant invention, such as, for example, phosphate buffered saline.

The terms “subject,” “individual,” “host,” and “patient” are used interchangeably herein to refer to a living animal, including a human and a non-human animal. The subject may, e.g., be an organism possessing immune cells capable of responding to antigenic stimulation, and stimulatory and inhibitory signaling transduction through cell surface receptor binding. The subject may be a mammal, such as a human or non-human mammal, for example, dogs, cats, pigs, cows, sheep, goats, horses, rats, and mice. The term “subject” does not preclude individuals that are entirely normal with respect to a disease, or normal in all respects.

“Treatment,” as used herein, covers any treatment of a disorder in a mammal, including a human, and includes preventing the condition or disease from occurring or recurring in a subject who may be predisposed to the disorder but has not yet been diagnosed; inhibiting the disorder, i.e., arresting its development; relieving the disorder, i.e., causing its regression; restoring or repairing a lost, missing, or defective function; or stimulating an inefficient process. In the context of cancer, the term “treating” includes preventing growth of tumor cells or cancer cells, preventing replication of tumor cells or cancer cells, lessening the overall tumor burden, and ameliorating one or more symptoms associated with the disease.

A “disease” is a pathological condition, e.g., one that can be identified by symptoms or other identifying factors as diverging from a healthy or a normal state. The term “disease” includes disorders, syndromes, conditions, and injuries. Diseases include, but are not limited to, proliferative, inflammatory, immune, metabolic, infectious, and ischemic diseases.

A “modulator” of the polypeptides or polynucleotides or an “agent” herein is an agonist or antagonist that interferes with the binding or activity of such polypeptides or polynucleotides. Such modulators or agents include, for example, polypeptide variants, whether agonist or antagonist; antibodies, whether agonist or antagonist; soluble receptors, usually antagonists; small molecule drugs, whether agonist or antagonist; RNAi, usually an antagonist; antisense molecules, usually an antagonist; and ribozymes, usually an antagonist. In some embodiments, an agent is a subject polypeptide, where the subject polypeptide itself is administered to an individual. In some embodiments, an agent is an antibody specific for a subject “target” polypeptide. In some embodiments, an agent is a chemical compound such as a small molecule that may be useful as an orally available drug. Such modulation includes the recruitment of other molecules that directly effect the modulation. For example, an antibody that modulates the activity of a subject polypeptide that is a receptor on a cell surface may bind to the receptor and fix complement, activating the complement cascade and resulting in lysis of the cell. An agent which modulates a biological activity of a subject polypeptide or polynucleotide increases or decreases the activity or binding at least about 10%, at least about 15%, at least about 20%, at least about 25%, at least about 50%, at least about 80%, or at least about 2-fold, at least about 5-fold, or at least about 10-fold or more when compared to a suitable control.

Brief Description of the Tables

Table 1 lists the sequences of the invention. Each is identified by its FP ID number, a SEQ.ID.NO. corresponding to the nucleotide coding sequence (SEQ.ID.NO. (N1)), a SEQ.ID.NO. corresponding to the encoded polypeptide sequence (SEQ.ID.NO. (P1)), and a SEQ.ID.NO. corresponding to the entire nucleotide sequence (SEQ.ID.NO. (N0)). Each is also identified by a Clone ID designation that lists each novel clone of the invention.

Table 2 characterizes the polypeptides encoded by the cDNA clones of the invention. In addition to listing the FP ID and Clone ID, it specifies the predicted number of amino acid residues in the polypeptide (Pred Prot Len). Table 2 also specifies an internal parameter predicting the likelihood that the FP ID is secreted (Treevote), with “1” being a high likelihood of the polypeptide being secreted and “0” being a low likelihood of being secreted. Table 2 also specifies the positions of the amino acid residues that comprise a mature protein (Mature Protein Coords). Additionally, Table 2 specifies the coordinates of an alternate form of a mature protein. In instances where the mature protein start residue overlaps the signal peptide end residue, some of the amino acid residues may be cleaved off such that the mature protein does not start at the next amino acid residue from the signal peptides, resulting in the alternative mature protein coordinate (Altern Mature Protein Coords). Table 2 specifies signal peptide coordinates for the polypeptides of the invention (Signal Peptide Coords). Table 2 also specifies the number of transmembrane domains of each of the polypeptides of the invention (TM), the positions of the amino acid residues that comprise the transmembrane domains (TM Coords), and the positions of the amino acids that do not pass through the membrane (Non-TM Coords). Finally, Table 2 specifies protein family (Pfam) classifications for some of these polypeptides.

Table 3 designates the sequences in the public National Center for Information Biotechnology (NCBI) database displaying the greatest degree of similarity to polypeptides encoded by each novel human cDNA clone of the invention. The NCBI protein with the greatest homology to each FP ID/Clone ID is described by its NCBI accession number (Top Hit Accession ID), and by the NCBI's annotation of that sequence (Top Hit Annotation). Table 3 lists the percent identity of the Five Prime protein with its corresponding NCBI protein (Top Hit % ID). Table 3 also describes the characteristics of the human protein in the NCBI database with the greatest degree of similarity to polypeptides encoded by each novel human cDNA clone of the invention. This corresponding human NCBI protein is described by its NCBI accession number (Top Human Hit Accession ID) and by the NCBI's annotation of that sequence (Top Human Hit Annotation). Finally, Table 3 describes the percent identity of the Five Prime protein with this NCBI protein (Top Human Hit % ID).

Table 4 characterizes a subset of the polypeptides encoded by the cDNA clones of the invention. In addition to listing the FP ID and Clone ID, it specifies the tissue source of the clone. Some of these polypeptides are differentially expressed between different cell and tissue types, and are more highly expressed in the tissues designated in Table 4 as the tissue source. Table 4 also specifies the predicted number of amino acid residues in the polypeptide (Pred Prot Len) and the Treevote. Table 4 provides the coordinates of the hydrophobic domains of the signal peptide sequences based on the starting and ending amino acid residue positions of each polypeptide (Signal Peptide Coords). It also specifies the coordinates of the amino acid residues that comprise a mature protein (Mature Protein Coords). Additionally, Table 4 provides alternate predictions of the signal peptide coordinates (Altern Signal Peptide Coords) and the mature protein coordinates (Altern Mature Protein Coords). Table 4 also specifies the number of transmembrane domains of each of the polypeptides of the invention (TM), the positions of the amino acid residues that comprise the transmembrane domains (TM Coords), and the positions of the amino acids that do not pass through the membrane (Non-TM Coords).

Table 5 designates the sequences in the NCBI database displaying the greatest degree of similarity to a subset of polypeptides encoded by novel human cDNA clones of the invention (FP ID, Clone ID). The Pred Prot Len, Treevote, and TM, as described above for Table 2, are provided for each of these secreted and/or transmembrane polypeptides. Table 5 also provides the Top Hit Annotation and Top Hit Len, as described above for Table 2. It provides the length of the match between the FP ID and the Top Hit sequences, in terms of the number of matching amino acid residues (Top Hit Len # AA Mat). Table 5 further shows the percent identity between the matching amino acid residues and the amino acid residues of the novel sequence (% ID Mat (QL)). For example, the length of FP ID 1014905 is 82 amino acid residues. The number of amino acid matches with the Top Hit is 42 amino acid residues. The % ID over the Query Length is 42/82×100%=51%. Table 5 also provides the percent identity between the-matching amino acid residues and the amino acid residues of the public sequence (% ID Mat (HL)). For example, 42 of the amino acid residues are identical to the 161 amino acid residues of the Top Hit. The % ID over Hit Length is 42/161×100%=26%. Table 5 also provides this information with respect to the Top Human Hit. Finally, Table 5 provides the plasmids used to produce subclones of some of the clones of the invention (Subclone Type), and identification numbers for these subclones (Subclone ID).

Nucleic Acids and Polypeptide Compositions

Nucleic Acids

The present invention provides novel nucleic acid molecules, novel genes encoding proteins, the encoded proteins, and fragments, complements, and homologs thereof having nucleotide sequences such as any one of those shown in the tables and Sequence Listing, for example, any one of SEQ ID NOS:1-187, as well as fusion molecules containing such. Non-limiting embodiments of nucleic acid molecules include genes or gene fragments, exons, introns, mRNA, tRNA, rRNA, siRNA, ribozymes, antisense cDNA, recombinant polynucleotides, branched polynucleotides, plasmids, vectors, isolated DNA of any sequence, isolated RNA of any sequence, nucleic acid probes, and primers. Nucleic acid molecules include splice variants of an mRNA. Nucleic acids can be naturally occurring, e.g. DNA or RNA, or can be synthetic analogs, as known in the art. Such analogs are suitable as probes because they demonstrate stability under assay conditions. A nucleic acid molecule can also comprise modified nucleic acid molecules, such as methylated nucleic acid molecules and nucleic acid molecule analogs. Analogs of purines and pyrimidines are known in the art.

Nucleic acid compositions can comprise a sequence of DNA or RNA, including one having an open reading frame that encodes a polypeptide and is capable, under appropriate conditions, of being expressed as a polypeptide. The nucleic acid compositions also can comprise fragments of DNA or RNA. The term encompasses genomic DNA, cDNA, mRNA, splice variants, antisense RNA, RNAi, siRNA, DNA comprising one or more single-nucleotide polymorphisms (SNP), and vectors comprising nucleic acid sequences of interest.

The nucleic acids of the subject invention can encode all or a part of the subject proteins. Double or single stranded fragments can be obtained from the DNA sequence by chemically synthesizing oligonucleotides in accordance with conventional methods, for example by restriction enzyme digestion or polymerase chain reaction (PCR) amplification. The use of the polymerase chain reaction has been described (Saiki et al., 1985) and current techniques have been reviewed (Sambrook et al., 1989; McPherson et al. 2000; Dieffenbach and Dveksler, 1995). For the most part, DNA fragments will be of at least about 5 nucleotides, at least about 8 nucleotides, at least about 10 nucleotides, at least about 15 nucleotides, at least about 18 nucleotides, at least about 20 nucleotides, at least about 25 nucleotides, at least about 30 nucleotides, or at least about 50 nucleotides, at least about 75 nucleotides, or at least about 100 nucleotides. Nucleic acid compositions that encode at least six contiguous amino acids (i.e., fragments of 18 nucleotides or more), for example, nucleic acid compositions encoding at least 8 contiguous amino acids (i.e., fragments of 24 nucleotides or more), are useful in directing the expression or the synthesis of peptides that can be used as immunogens (Lerner, 1982; Shinnick et al., 1983; Sutcliffe et al., 1983).

Nucleic acid molecules of the invention can comprise heterologous nucleic acid sequences, i.e., nucleic acid sequences of any length other than those specified in the Sequence Listing. For example, the subject nucleic acid molecules can be flanked on the 5′ and/or 3′ ends by heterologous nucleic acid molecules of from about 1 nucleotide to about 10 nucleotides, from about 10 nucleotides to about 20 nucleotides, from about 20 nucleotides to about 50 nucleotides, from about 50 nucleotides to about 100 nucleotides, from about 100 nucleotides to about 250 nucleotides, from about 250 nucleotides to about 500 nucleotides, or from about 500 nucleotides to about 1000 nucleotides, or more in length.

Heterologous sequences of the invention can comprise nucleotides present between the initiation codon and the stop codon, including some or all of the introns that are normally present in a native chromosome. They can further include the 3′ and 5′ untranslated regions found in the mature mRNA. They can further include specific transcriptional and translational regulatory sequences, such as promoters, enhancers, etc., including about 1 kb, about 2 kb, and possibly more, of flanking genomic DNA at either the 5′ or 3′ end of the transcribed region. Genomic DNA can be isolated as a fragment of 100 kbp or smaller; and substantially free of flanking chromosomal sequence. This genomic DNA flanking the coding region, either 3′ or 5′, or internal regulatory sequences as sometimes found in introns, may contain sequences required for proper tissue and stage-specific expression.

The sequence of the 5′ flanking region can be utilized as promoter elements, including enhancer binding sites that provide for tissue-specific expression and developmental regulation in tissues where the subject genes are expressed, providing promoters that mimic the native pattern of expression. Naturally occurring polymorphisms in the promoter region are useful for determining natural variations in expression, particularly those that may be associated with disease. Promoters or enhancers that regulate the transcription of the polynucleotides of the present invention are obtainable by use of PCR techniques using human tissues, and one or more of the present primers.

Regulatory sequences can be used to identify cis acting sequences required for transcriptional or translational regulation of expression, especially in different tissues or stages of development, and to identify cis acting sequences and trans-acting factors that regulate or mediate expression. Such transcription or translational control regions can be operably linked to a gene in order to promote expression of wild type genes or of proteins of interest in cultured cells, embryonic, fetal or adult tissues, and for gene therapy (Hooper, 1993).

The invention provides variants resulting from random or site-directed mutagenesis. Techniques for in vitro mutagenesis of cloned genes are known. Examples of protocols for site specific mutagenesis may be found in Gustin et al., 1993; Barany 1985; Colicelli et al., 1985; Prentki et al., 1984. Methods for site specific mutagenesis can be found in Sambrook et al., 1989 (pp. 15.3-15.108); Weiner et al., 1993; Sayers et al. 1992; Jones and Winistorfer; Barton et al., 1990; Marotti and Tomich 1989; and Zhu, 1989. Such mutated genes can be used to study structure-function relationships of the subject proteins, or to alter properties of the protein that affect its function or regulation. Other modifications of interest include epitope tagging, e.g., with hemagglutinin (HA), FLAG, or c-myc. For studies of subcellular localization, fluorescent fusion proteins can be used.

The invention also provides variants resulting from chemical or other modifications. Modifications in the native structure of nucleic acids, including alterations in the backbone, sugars or heterocyclic bases, have been shown to increase intracellular stability and binding affinity. Among useful changes in the backbone chemistry are phosphorothioates; phosphorodithioates, where both of the non-bridging oxygens are substituted with sulfur; phosphoroamidites; alkyl phosphotriesters, and boranophosphates. Achiral phosphate derivatives include 3′-O′-5′-S-phosphorothioate, 3′-S-5′-O— phosphorothioate, 3′-CH₂-5′-O-phosphonate and 3′-NH-5′-O-phosphoroamidate. Peptide nucleic acids have modifications that replace the entire ribose phosphodiester backbone with a peptide linkage.

Sugar modifications are also used to enhance stability and affinity. The α-anomer of deoxyribose can be used, where the base is inverted with respect to the natural β-anomer. The 2′-OH of the ribose sugar can be altered to form 2′-O-methyl or 2′-O-allyl sugars, which provides resistance to degradation without comprising affinity.

Modification of the heterocyclic bases must maintain proper base pairing. Some useful substitutions include deoxyuridine for deoxythymidine; 5-methyl-2′-deoxycytidine, and 5-bromo-2′-deoxycytidine for deoxycytidine. 5 propynyl-2′-deoxyuridine and 5-propynyl-2′-deoxycytidine have been shown to increase affinity and biological activity when substituted for deoxythymidine and deoxycytidine, respectively.

Mutations can be introduced into the promoter region to determine the effect of altering expression in experimentally defined systems. Methods for the identification of specific DNA motifs involved in the binding of transcriptional factors are known in the art, for example sequence similarity to known binding motifs, and gel retardation studies (Blackwell et al., 1995; Mortlock et al., 1996; Joulin and Richard-Foy, 1995).

In some embodiments, the invention provides isolated nucleic acids that, when used as primers in a polymerase chain reaction, amplify a subject polynucleotide, or a polynucleotide containing a subject polynucleotide. The amplified polynucleotide is from about 20 to about 50, from about 50 to about 75, from about 75 to about 100, from about 100 to about 125, from about 125 to about 150, from about 150 to about 175, from about 175 to about 200, from about 200 to about 250, from about 250 to about 300, from about 300 to about 350, from about 350 to about 400, from about 400 to about 500, from about 500 to about 600, from about 600 to about 700, from about 700 to about 800, from about 800 to about 900, from about 900 to about 1000, from about 1000 to about 2000, from about 2000 to about 3000, from about 3000 to about 4000, from about 4000 to about 5000, or from about 5000 to about 6000 nucleotides or more in length.

The isolated nucleic acids themselves are from about 10 to about 100, from about 100 to about 500, from about 500 to about 1000, from about 1000 to about 2000, from about 2000 to about 3000, or from about 3000 to about 4000 nucleotides in length. Generally, the nucleic acids are used in pairs in a polymerase chain reaction, where they are referred to as “forward” and “reverse” primers.

The subject nucleic acid compositions find use in a variety of different investigative applications. Applications of interest include identifying genomic DNA sequence using molecules of the invention, identifying homologs of molecules of the invention, creating a source of novel promoter elements, identifying expression regulatory factors, creating a source of probes and primers for hybridization applications, identifying expression patterns in biological specimens; preparing cell or animal models to investigate the function of the molecules of the invention, and preparing in vitro models to investigate the function of the molecules of the invention.

The isolated nucleic acids of the invention can be used as probes to detect and characterize gross alteration in a genomic locus, such as deletions, insertions, translocations, and duplications, e.g., by applying fluorescence in situ hybridization (FISH) techniques to examine chromosome spreads (Andreeff et al., 1999). These nucleic acids are also useful for detecting smaller genomic alterations, such as deletions, insertions, additions, translocations, and substitutions (e.g., SNPs).

When used as probes to detect nucleic acid molecules capable of hybridizing with nucleic acids described in the Sequence Listing, the nucleic acid molecules can be flanked by heterologous sequences of any length. When used as probes, a subject nucleic acid can include nucleotide analogs that incorporate labels that are directly detectable, such as radiolabels or fluorescent labels, or nucleotide analogs that incorporate labels that can be visualized in a subsequent reaction.

Polypeptides

The invention provides novel polypeptides and related polypeptide compositions. Generally, a polypeptide of the invention refers to a polypeptide which has the amino acid sequence set forth in the Sequence Listing. The novel polypeptides of the invention include fragments thereof, and variants, as discussed in more detail below.

In an embodiment, the invention provides an isolated polypeptide comprising an amino acid sequence, wherein the amino acid sequence is chosen from the Sequence Listing or the tables, or a biologically active fragment thereof, or is encoded by a polynucleotide sequence chosen from Sequence Listing or the tables, or a biologically active fragment thereof, such as, for example, any one of SEQ ID NOS:188-374.

The proteins of the subject invention have been separated from their naturally occurring environment and are present in a non-naturally occurring environment. In certain embodiments, the proteins are present in a composition where they are more concentrated than in their naturally occurring environment.

The invention provides isolated polypeptides which are substantially free of the materials with which it is associated in nature or other polypeptide sequences that do not include a sequence or fragment of the subject polypeptides. By substantially free is meant that less than about 90%, less than about 80%, less than about 70%, less than about 60%, or less than about 50% of the composition is made up of materials other than the isolated polypeptide. For example, the isolated polypeptide is at least about 50%, at least about 60%, at least about 70%, at least about 80%, at least about 90%, at least about 95%, at least about 97%, or at least about 99% free of the materials with which it is associated in nature. For example, an isolated polypeptide may be present in a composition wherein at least about 50%, at least about 60%, at least about 70%, at least about 80%, at least about 90%, at least about 95%, at least about 97%, or at least about 99% of the total macromolecules (for example, polypeptides, fragments thereof, polynucleotides, fragments thereof, lipids, polysaccharides, and oligosaccharides) in the composition is the isolated polypeptide. Where at least about 99% of the total macromolecules is the isolated polypeptide, the polypeptide is at least about 99% pure, and the composition comprises less than about 1% contaminant.

Polypeptides of the invention include conjugated proteins, fusion proteins, including, but not limited to, GST fusion proteins, fusion proteins with a heterologous amino acid sequences, fusion proteins with heterologous and homologous leader sequences, fusion proteins with or without N-terminal methionine residues, pegolyated proteins, and immunologically tagged proteins. Also included are variations of naturally occurring proteins, where such variations are homologous or substantially similar to the naturally occurring protein, as well as corresponding homologs from different species.

Alterations of the native amino acid sequence may be accomplished by any of a number of known techniques. Mutations can be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered gene having particular codons altered according to the substitution, deletion, or insertion required (Walder and Walder, 1986; Bauer et al., 1985; Craik, 1985; and U.S. Pat. Nos. 4,518,584 and 4,737,462).

In some embodiments, a subject polypeptide is present as an oligomer, including homodimers, homotrimers, homotetramers, and multimers that include more than four monomeric units. Oligomers also include heteromultimers, e.g., heterodimers, heterotrimers, heterotetramers, etc. where the subject polypeptide is present in a complex with proteins other than the subject polypeptide. Where the multimer is a heteromultimer, the subject polypeptide can be present in a 1:1 ratio, a 1:2 ratio, a 2:1 ratio, or other ratio, with the other protein(s).

Oligomers may be formed by disulfide bonds between cysteine residues on different polypeptides, or by non-covalent interactions between polypeptide chains, for example. In other embodiments, oligomers comprise from two to four polypeptides joined via covalent or non-covalent interactions between peptide moieties fused to the polypeptides. Such peptides may be peptide linkers (spacers), or peptides that have the property of promoting oligomerization. Leucine zippers and certain polypeptides derived from antibodies are among the peptides that can promote oligomerization of polypeptides attached thereto, as described in more detail below and in WO 94/10308.

Polypeptides of the invention can be obtained from naturally occurring sources or produced synthetically. The sources of naturally occurring polypeptides will generally depend on the species from which the protein is to be derived, i.e., the proteins will be derived from biological sources that express the proteins. The subject proteins can also be derived from synthetic means, e.g., by expressing a recombinant gene encoding a protein of interest in a suitable system or host or enhancing endogenous expression, as described in more detail below. Further, small peptides can be synthesized in the laboratory by techniques well known in the art.

Protein expression systems known in the art can produce fusion proteins that incorporate the polypeptides of the invention. The invention provides an isolated amino acid molecule with a first polypeptide comprising a molecule chosen from the Sequence Listing, or one or more of its biologically active fragments or variants, and a second molecule. This second molecule can facilitate production, secretion, and/or purification. It can confer a longer half-life to the first polypeptide when administered to an animal. Second molecules suitable for use in the invention include, for example, polyethylene glycol (PEG), human serum albumin, Fc, and/or one or more of their fragments. The invention can also provide a nucleic acid molecule with a second nucleotide sequence that encodes a fusion partner. This second nucleotide sequence can be operably linked to the first nucleotide sequence.

Thus, the invention provides polypeptide fusion partners. They may be part of a fusion molecule, e.g., a polynucleotide or polypeptide, which represents the joining of all of or portions of more than one gene. For example, a fusion protein can be the product obtained by splicing strands of recombinant DNA and expressing the hybrid gene. A fusion molecule can be made by genetic engineering, e.g., by removing the stop codon from the DNA sequence of a first protein, then appending the DNA sequence of a second protein in frame. The DNA sequence will then be expressed by a cell as a single protein. Typically this is accomplished by cloning a cDNA into an expression vector in frame with an existing gene. The invention provides fusion proteins with heterologous and homologous leader sequences, fusion proteins with a heterologous amino acid sequence, and fusion proteins with or without N-terminal methionine residues. The fusion partners of the invention can be either N-terminal fusion partners or C-terminal fusion partners.

As noted above, suitable fusion partners include, but are not limited to, albumin and F_{c}. These fusion partners can include any variant of or any fragment of such. Such modified polypeptides can show, e.g., enhanced activity or increased stability. In addition, they may be purified in higher yields and show better solubility than the corresponding natural polypeptide, at least under certain purification and storage conditions.

Fusion polypeptides can be secreted from the cell by the incorporation of leader sequences that direct the protein to the membrane for secretion. These leader sequences can be specific to the host cell, and are known to skilled artisans; they are also cited in the references. The invention includes appropriate restriction enzyme sites for vector cloning. In addition to facilitating the secretion of these fusion proteins, the invention provides for facilitating their production. This can be accomplished in a number of ways, including producing multiple copies, employing strong promoters, and increasing their intracellular stability, e.g., by fusion with beta-galactosidase.

The invention also provides for facilitating the purification of these fusion proteins. Fusion with a selectable marker can facilitate purification by affinity chromatography. For example, fusion with the selectable marker glutathione S-transferase (GST) produces polypeptides that can be detected with antibodies directed against GST, and isolated by affinity chromatography on glutathione-sepharose; the GST marker can then be removed by thrombin cleavage. Polypeptides that provide for binding to metal ions are also suitable for affinity purification. For example, a fusion protein that incorporates Hisₙ, where n is between three and ten, inclusive (SEQ ID NO.:486), e.g., a 6×His-tag (SEQ ID NO.:485) can be used to isolate a protein by affinity chromatography using a nickel ligand.

The fusion partners of the invention can also include linkers, i.e., fragments of synthetic DNA containing a restriction endonuclease recognition site that can be used for splicing genes. These can include polylinkers, which contain several restriction enzyme recognition sites. A linker may be part of a cloning vector. It may be located either upstream or downstream of the therapeutic protein, and it may be located either upstream or downstream of the fusion partner.

Gene manipulation techniques have enabled the development and use of recombinant therapeutic proteins with fusion partners that impart desirable pharmacokinetic properties. Recombinant human serum albumin fused with synthetic heme protein has been reported to reversibly carry oxygen (Chuang et al., 2002). The long half-life and stability of human serum albumin (HSA) makes it an attractive candidate for fusion to short-lived therapeutic proteins (U.S. Pat. No. 6,686,179).

For example, the short plasma half-life of unmodified interferon alpha makes frequent dosing necessary over an extended period of time, in order to treat viral and proliferative disorders. Interferon alpha fused with HSA has a longer half life and requires less frequent dosing than unmodified interferon alpha; the half-life was 18-fold longer and the clearance rate was approximately 140 times slower (Osborn et al., 2002). Interferon beta fused with HSA also has favorable pharmacokinetic properties; its half life was reported to be 36-40 hours, compared to 8 hours for unmodified interferon beta (Sung et al., 2003). A HSA-interleukin-2 fusion protein has been reported to have both a longer half-life and favorable biodistribution compared to unmodified interleukin-2. This fusion protein was observed to target tissues where lymphocytes reside to a greater extent than unmodified interleukin 2, suggesting that it exerts greater efficacy (Yao et al., 2004).

The Fc receptor of human immunoglobulin G subclass 1 has also been used as a fusion partner for a therapeutic molecule. It has been recombinantly linked to two soluble p75 tumor necrosis factor (TNF) receptor molecules. This fusion protein has been reported to have a longer circulating half-life than monomeric soluble receptors, and to inhibit TNFα-induced proinflammatory activity in the joints of patients with rheumatoid arthritis (Goldenberg, 1999). This fusion protein has been used clinically to treat rheumatoid arthritis, juvenile rheumatoid arthritis, psoriatic arthritis, and ankylosing spondylitis (Nanda and Bathon, 2004).

The peptides of the invention, including the fusion proteins, can be modified with or covalently coupled to one or more of a variety of hydrophilic polymers to increase their solubility and circulation half-life. Suitable nonproteinaceous hydrophilic polymers for coupling to a peptide include, but are not limited to, polyalkylethers as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran and dextran derivatives, etc. Generally, such hydrophilic polymers have an average molecular weight ranging from about 500 to about 100,000 daltons, from about 2,000 to about 40,000 daltons, or from about 5,000 to about 20,000 daltons. The peptide can be derivatized with or coupled to such polymers using any of the methods set forth in Zallipsky 1995; Monfardini et al., 1995; U.S. Pat. Nos. 4,791,192; 4,670,417; 4,640,835; 4,496,689; 4,301,144; 4,179,337 and WO 95/34326.

Conjugating biomolecules with polyethylene glycol (PEG), a process known as pegylation, increases the circulating half-life of therapeutic proteins (Molineux, 2002). Polyethylene glycols are nontoxic water-soluble polymers that, owing to their large hydrodynamic volume, create a shield around the pegylated drug, thus protecting it from renal clearance, enzymatic degradation, and recognition by cells of the immune system.

Pegylated agents have improved pharmacokinetics that permit dosing schedules that are more convenient and more acceptable to patients. This improved pharmacokinetic profile may decrease adverse effects caused by the large variations in peak-to-trough plasma drug concentrations associated with frequent administration and by the immunogenicity of unmodified proteins (Harris et al., 2001). In addition, pegylated proteins may have reduced immunogenicity because PEG-induced steric hindrance can prevent immune recognition (Harris et al., 2001).

Polypeptides of the invention can be isolated by any appropriate means known in the art. For example, convenient protein purification procedures can be employed (e.g., Deuthscher et al., 1990). In general, a lysate can be prepared from the original source, (e.g., a cell expressing endogenous polypeptide, or a cell comprising the expression vector expressing the polypeptide(s)), and purified using HPLC, exclusion chromatography, gel electrophoresis, or affinity chromatography, and the like.

In another aspect, the invention provides a method of making a polypeptide of the invention by providing a nucleic acid molecule that comprises a polynucleotide sequence encoding a polypeptide of the invention, introducing the nucleic acid molecule into an expression system, and allowing the polypeptide to be produced. Briefly, the methods generally involve introducing a nucleic acid construct into a host cell in vitro and culturing the host cell under conditions suitable for expression, then harvesting the polypeptide, either from the culture medium or from the host cell, (e.g., by disrupting the host cell), or both, as described in detail above. The invention also provides methods of producing a polypeptide using cell-free in vitro transcription/translation methods, which are well known in the art, also as provided above.

Antibodies

The invention provides an antibody directed to a polypeptide of the Sequence Listing or encoded by a nucleic acid molecule of the Sequence Listing. The invention also provides an antibody specifically binding to and/or interfering with the biological activity of a polypeptide of the Sequence Listing or encoded by a nucleic acid molecule of the Sequence Listing.

This antibody may be a monoclonal antibody, a polyclonal antibody, a single chain antibody, an Fab fragment, an antibody comprising a backbone of a molecule with an Ig domain, a V_{H} fragment, a V_{L} fragment, a cdr fragment, and/or a framework fragment. It may also be a cytotoxic antibody, targeting antibody, an antibody agonist, an antibody antagonist, an antibody that promotes endocytosis of a target antigen, an antibody that mediates antibody dependent cell cytoxicity (ADCC), and/or an antibody that mediates cell-dependent cytotoxicity (CDC).

An antibody of the invention can be a human antibody, a non-human primate antibody, a non-primate animal antibody, a rabbit antibody, a mouse antibody, a rat antibody, a sheep antibody, a goat antibody, a horse antibody, a porcine antibody, a cow antibody, a chicken antibody, a humanized antibody, a primatized antibody, and a chimeric antibody. These antibodies can comprise a cytotoxic antibody with one or more cytotoxic component chosen from a radioisotope, a microbial toxin, a plant toxin, and a chemical compound. The chemical compound can be chosen from doxorubicin and cisplatin.

In another aspect, the invention provides antibody targets. The polynucleotides and polypeptides of the invention comprise nucleic acid and amino acid sequences that can be recognized by antibodies. A target sequence can be any polynucleotide or amino acid sequence of approximately eighteen or more contiguous nucleotides or six or more amino acids. A variety of comparing means can be used to accomplish comparison of sequence information from a sample (e.g., to analyze target sequences, target motifs, or relative expression levels) with the data storage means. A skilled artisan can readily recognize that any one of the publicly available homology search programs can be used as the search means for the computer based systems of the present invention to accomplish comparison of target sequences and motifs. Computer programs to analyze expression levels in a sample and in controls are also known in the art. A target sequence includes an antibody target sequence, which refers to an amino acid sequence that can be used as an immunogen for injection into animals for production of antibodies or for screening against a phage display or antibody library for identification of binding partners.

The invention provides target structural motifs, or target motifs, i.e., any rationally selected sequences or combination of sequences in which the sequence(s) are chosen based on a three-dimensional configuration formed upon the folding of the target motif, or on consensus sequences of regulatory or active sites. There are a variety of target motifs known in the art. Protein target motifs include, but are not limited to, enzyme active sites and signal sequences. Nucleic acid target motifs include, but are not limited to, hairpin structures, promoter sequences, and other expression elements, such as binding sites for transcription factors.

Antibodies of the invention bind specifically to their targets. The term binds specifically, in the context of antibody binding, refers to high avidity and/or high affinity binding of an antibody to a specific polypeptide, or more accurately, to an epitope of a specific polypeptide. Antibody binding to such epitope on a polypeptide can be stronger than binding of the same antibody to any other epitopes, particularly other epitopes that can be present in molecules in association with, or in the same sample as the polypeptide of interest. For example, when an antibody binds more strongly to one epitope than to another, adjusting the binding conditions can result in antibody binding almost exclusively to the specific epitope and not to any other epitopes on the same polypeptide, and not to any other polypeptide, which does not comprise the epitope. Antibodies that bind specifically to a subject polypeptide may be capable of binding other polypeptides at a weak, yet detectable, level (e.g., 10% or less of the binding shown to the polypeptide of interest). Such weak binding, or background binding, is readily discernible from the specific antibody binding to a subject polypeptide, e.g., by use of appropriate controls. In general, antibodies of the invention bind to a specific polypeptide with a binding affinity of 10⁷ M⁻¹ or greater (e.g., 10⁸ M⁻¹, 10⁹ M⁻¹, 10¹⁰ M⁻¹, 10¹¹ M⁻¹, etc.).

The invention provides antibodies that can distinguish the variant sequences of the invention from currently known sequences. These antibodies can distinguish polypeptides that differ by no more than one amino acid (U.S. Pat. No. 6,656,467). They have high affinity constants, i.e., in the range of approximately 10¹⁰ M⁻¹, and are produced, for example, by genetically engineering appropriate antibody gene sequences, according to the method described by Young et al., in U.S. Pat. No. 6,656,467.

Antibodies of the invention can be provided as matrices, i.e., as geometric networks of antibody molecules and their antigens, as found in immunoprecipitation and flocculation reactions. An antibody matrix can exist in solution or on a solid phase support.

Antibodies of the invention can be provided as a library of antibodies or fragments thereof, wherein at least one antibody or fragment thereof specifically binds to at least a portion of a polypeptide comprising an amino acid sequence according to any one of SEQ ID NOS.:188-374, and/or wherein at least one antibody or fragment thereof interferes with at least one activity of such polypeptide or fragment thereof. In certain embodiments, the antibody library comprises at least one antibody or fragment thereof that specifically inhibits binding of a subject polypeptide to its ligand or substrate, or that specifically inhibits binding of a subject polypeptide as a substrate to another molecule. The present invention also features corresponding polynucleotide libraries comprising at least one polynucleotide sequence that encodes an antibody or antibody fragment of the invention. In specific embodiments, the library is provided on a nucleic acid array or in computer-readable format.

The invention provides a method of making an antibody by introducing an antigen chosen from an isolated nucleic-acid molecule comprising at least one polynucleotide sequence chosen from the Sequence Listing; sequences that hybridize to these sequences under high stringency conditions; sequences having at least 80% sequence identity to these sequences, or sequences that hybridize to them under high stringency conditions; complements of any of these sequences; or biologically active fragments of any of the above-listed sequences or an isolated polypeptide comprising an amino acid sequence, wherein the amino acid sequence is chosen from the Sequence Listing, or a biologically active fragment thereof, or is encoded by a polynucleotide sequence chosen from the Sequence Listing, or a biologically active fragment thereof into an animal in an amount sufficient to elicit generation of antibodies specific to the antigen, and recovering the antibodies therefrom.

Generally, the invention features a method of making an antibody by immunizing a host animal (Coligan, 2002). In this method, a polypeptide or a fragment thereof, a polynucleotide encoding a polypeptide, or a polynucleotide encoding a fragment thereof, is introduced into an animal in a sufficient amount to elicit the generation of antibodies specific to the polypeptide or fragment thereof, and the resulting antibodies are recovered from the animal. Initial immunizations can be performed using either polynucleotides or polypeptides. Subsequent booster immunizations can also be performed with either polynucleotides or polypeptides. Initial immunization with a polynucleotide can be followed with either polynucleotide or polypeptide immunizations, and an initial immunization with a polypeptide can be followed with either polynucleotide or polypeptide immunizations.

The host animal will generally be a different species than the immunogen, e.g., a human protein used to immunize mice. Methods of antibody production are well known in the art (Coligan, 2002; Howard and Bethell, 2000; Harlow et al., 1998; Harlow and Lane, 1988). The invention thus also provides a non-human animal comprising an antibody of the invention. The animal can be a non-human primate, (e.g., a monkey), a rodent (e.g., a rat, a mouse, a hamster, a guinea pig), a chicken, cattle (e.g., a sheep, a goat, a horse, a pig, a cow), a rabbit, a cat, or a dog.

The present invention also features a method of making an antibody by isolating a spleen from an animal injected with a polypeptide or a fragment thereof, a polynucleotide encoding a polypeptide, or a polynucleotide encoding a fragment thereof, and recovering antibodies from the spleen cells. Hybridomas can be made from the spleen cells, and hybridomas secreting specific antibodies can be selected.

The present invention further features a method of making a polynucleotide library from spleen cells, and selecting a cDNA clone that produces specific antibodies, or fragments thereof. The cDNA clone or a fragment thereof can be expressed in an expression system that allows production of the antibody or a fragment thereof, as provided herein.

The immunogen can comprise a nucleic acid, a complete protein, or fragments and derivatives thereof, or proteins expressed on cell surfaces. Proteins domains, e.g., extracellular, cytoplasmic, or luminal domains can be used as immunogens. Immunogens comprise all or a part of one of the subject proteins, where these amino acids contain post-translational modifications, such as glycosylation, found on the native target protein. Immunogens comprising protein extracellular domains are produced in a variety of ways known in the art, e.g., expression of cloned genes using conventional recombinant methods, or isolation from tumor cell culture supernatants, etc. The immunogen can also be expressed in vivo from a polynucleotide encoding the immunogenic peptide introduced into the host animal.

Polyclonal antibodies are prepared by conventional techniques. These include immunizing the host animal in vivo with the target protein (or immunogen) in substantially pure form, for example, comprising less than about 1% contaminant. The immunogen can comprise the complete target protein, fragments, or derivatives thereof. To increase the immune response of the host animal, the target protein can be combined with an adjuvant; suitable adjuvants include alum, dextran, sulfate, large polymeric anions, and oil and water emulsions, e.g., Freund's adjuvant (complete or incomplete). The target protein can also be conjugated to synthetic carrier proteins or synthetic antigens. The target protein is administered to the host, usually intradermally, with an initial dosage followed by one or more, usually at least two, additional booster dosages. Following immunization, blood from the host is collected, followed by separation of the serum from blood cells. The immunoglobulin present in the resultant antiserum can be further fractionated using known methods, such as ammonium salt fractionation, or DEAE chromatography and the like.

Cytokines can also be used to help stimulate immune response. Cytokines act as chemical messengers, recruiting immune cells that help the killer T-cells to the site of attack. An example of a cytokine is granulocyte-macrophage colony-stimulating factor (GM-CSF), which stimulates the proliferation of antigen-presenting cells, thus boosting an organism's response to a cancer vaccine. As with adjuvants, cytokines can be used in conjunction with the antibodies and vaccines disclosed herein. For example, they can be incorporated into the antigen-encoding plasmid or introduced via a separate plasmid, and in some embodiments, a viral vector can be engineered to display cytokines on its surface.

The method of producing polyclonal antibodies can be varied in some embodiments of the present invention. For example, instead of using a single substantially isolated polypeptide as an immunogen, one may inject a number of different immunogens into one animal for simultaneous production of a variety of antibodies. In addition to protein immunogens, the immunogens can be nucleic acids (e.g., in the form of plasmids or vectors) that encode the proteins, with facilitating agents, such as liposomes, microspheres, etc, or without such agents, such as “naked” DNA.

The invention provides a bacteriophage comprising an antibody specifically binding to and/or interfering with the biological activity of an isolated nucleic acid molecule comprising at least one polynucleotide sequence of the Sequence Listing; sequences that hybridize to these sequences under high stringency conditions; sequences having at least 80% sequence identity to the Sequence Listing or sequences that hybridize to them under high stringency conditions; complements of any of these sequences; or biologically active fragments of any of the above-listed sequences; or an isolated polypeptide comprising an amino acid sequence, wherein the amino acid sequence is chosen from the Sequence Listing, or a biologically active fragment thereof, or is encoded by a polynucleotide sequence chosen from Sequence Listing, or a biologically active fragment thereof; or a fragment of such an antibody. The invention further provides a bacterial cell comprising such a bacteriophage. It provides a recombinant host cell that produces such an antibody or a fragment of such an antibody.

In an embodiment, polyclonal antibodies can be prepared using phage display libraries, which are conventional in the art. In this method, a collection of bacteriophages displaying antibody properties on their surfaces are made to contact subject polypeptides, or fragments thereof. Bacteriophages displaying antibody properties that specifically recognize the subject polypeptides are selected, amplified, for example, in E. coli, and harvested. Such a method typically produces single chain antibodies, which are further described below.

Phage display technology can be used to produce Fab antibody fragments, which can be then screened to select those with strong and/or specific binding to the protein targets. The screening can be performed using methods that are known to those of skill in the art, for example, ELISA, immunoblotting, immunohistochemistry, or immunoprecipitation. Fab fragments identified in this manner can be assembled with an Fc portion of an antibody molecule to form a complete immunoglobulin molecule.

Monoclonal antibodies are also produced by conventional techniques, such as fusing an antibody-producing plasma cell with an immortal cell to produce hybridomas. Suitable animals will be used, e.g., to raise antibodies against a mouse polypeptide of the invention, the host animal will generally be a hamster, guinea pig, goat, chicken, or rabbit, and the like. Generally, the spleen and/or lymph nodes of an immunized host animal provide the source of plasma cells, which are immortalized by fusion with myeloma cells to produce hybridoma cells. Culture supernatants from individual hybridomas are screened using standard techniques to identify clones producing antibodies with the desired specificity. The antibody can be purified from the hybridoma cell supernatants or from ascites fluid present in the host by conventional techniques, e.g., affinity chromatography using antigen, e.g., the subject protein, bound to an insoluble support, e.g., protein A sepharose, etc.

The antibody can be produced as a single chain, instead of the normal multimeric structure of the immunoglobulin molecule. Single chain antibodies have been previously described (i.e., Jost et al., 1994). DNA sequences encoding parts of the immunoglobulin, for example, the variable region of the heavy chain and the variable region of the light chain are ligated to a spacer, such as one encoding at least about four small neutral amino acids, i.e., glycine or serine. The protein encoded by this fusion allows the assembly of a functional variable region that retains the specificity and affinity of the original antibody.

The invention also provides intrabodies that are intracellularly expressed single-chain antibody molecules designed to specifically bind and inactivate target molecules inside cells. Intrabodies have been used in cell assays and in whole organisms (Chen et al., 1994; Hassanzadeh et al., 1998). Inducible expression vectors can be constructed with intrabodies that react specifically with a protein of the invention. These vectors can be introduced into host cells and model organisms.

The invention also provides “artificial” antibodies, e.g., antibodies and antibody fragments produced and selected in vitro. In some embodiments, these antibodies are displayed on the surface of a bacteriophage or other viral particle, as described above. In other embodiments, artificial antibodies are present as fusion proteins with a viral or bacteriophage structural protein, including, but not limited to, M13 gene III protein. Methods of producing such artificial antibodies are well known in the art (U.S. Pat. Nos. 5,516,637; 5,223,409; 5,658,727; 5,667,988; 5,498,538; 5,403,484; 5,571,698; and 5,625,033). The artificial antibodies, selected, for example, on the basis of phage binding to selected antigens, can be fused to a Fc fragment of an immunoglobulin for use as a therapeutic, as described, for example, in U.S. Pat. No. 5,116,964 or WO 99/61630. Antibodies of the invention can be used to modulate biological activity of cells, either directly or indirectly. A subject antibody can modulate the activity of a target cell, with which it has primary interaction, or it can modulate the activity of other cells by exerting secondary effects, i.e., when the primary targets interact or communicate with other cells. The antibodies of the invention can be administered to mammals, and the present invention includes such administration, particularly for therapeutic and/or diagnostic purposes in humans.

The antibodies can be partially human or fully human antibodies. For example, xenogenic antibodies, which are produced in animals that are transgenic for human antibody genes, can be employed to make a fully human antibody. By xenogenic human antibodies is meant antibodies that are fully human antibodies, with the exception that they are produced in a non-human host that has been genetically engineered to express human antibodies (e.g., WO 98/50433; WO 98/24893 and WO 99/53049).

Chimeric immunoglobulin genes constructed with immunoglobulin cDNA are known in the art (Liu et al. 1987a; Liu et al. 1987b). Messenger RNA is isolated from a hybridoma or other cell producing the antibody and used to produce cDNA. The cDNA of interest can be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant (C) regions genes are known in the art (Kabat et al., 1991). Human C region genes are readily available from known clones. The choice of isotype will be guided by the desired effector functions, such as complement fixation, or antibody-dependent cellular cytotoxicity. IgG1, IgG3, and IgG4 isotypes, and either of the kappa or lambda human light chain constant regions can be used. The chimeric, humanized antibody is then expressed by conventional methods.

Consensus sequences of heavy (H) and light (L) J regions can be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

A convenient expression vector for producing antibodies is one that encodes a functionally complete human CH or CL immunoglobulin sequence, with appropriate restriction sites engineered so that any VH or VL sequence can be easily inserted and expressed, such as plasmids, retroviruses, YACs, or EBV derived episomes, and the like. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody can be joined to any strong promoter, including retroviral LTRs, e.g., SV-40 early promoter, (Okayama, et al. 1983), Rous sarcoma virus LTR (Gorman et al. 1982), and Moloney murine leukemia virus LTR (Grosschedl et al. 1985), or native immunoglobulin promoters.

Antibody fragments, such as Fv, F(ab′)2, and Fab can be prepared by cleavage of the intact protein, e.g., by protease or chemical cleavage. These fragments can include heavy and light chain variable regions. Alternatively, a truncated gene can be designed, e.g., a chimeric gene encoding a portion of the F(ab′)₂ fragment that includes DNA sequences encoding the CH1 domain and hinge region of the H chain, followed by a translational stop codon.

Antibodies may be administered by injection systemically, such as by intravenous injection; or by injection or application to the relevant site, such as by direct injection into a tumor, or direct application to the site when the site is exposed in surgery; or by topical application, such as if the disorder is on the skin, for example.

For in vivo use, particularly for injection into humans, in some embodiments it is desirable to decrease the antigenicity of the antibody. An immune response of a recipient against the antibody may potentially decrease the period of time that the therapy is effective. Methods of humanizing antibodies are known in the art. The humanized antibody can be the product of an animal having transgenic human immunoglobulin genes, e.g., constant region genes (e.g., Grosveld and Kolias, 1992; Murphy and Carter, 1993; Pinkert, 1994; and International Patent Applications WO 90/10077 and WO 90/04036). Alternatively, the antibody of interest can be engineered by recombinant DNA techniques to substitute the CH1, CH2, CH3, hinge domains, and/or the framework domain with the corresponding human sequence (see, e.g., WO 92/02190). Humanized antibodies can also be produced by immunizing mice that make human antibodies, such as Abgenix xenomice, Medarex's mice, or Kirin's mice, and can be made using the technology of Protein Design Labs, Inc. (Fremont, Calif.) (Coligan, 2002). Both polyclonal and monoclonal antibodies made in non-human animals may be humanized before administration to human subjects.

The antibodies of the present invention may be administered alone or in combination with other molecules for use as a therapeutic, for example, by linking the antibody to cytotoxic agent or radioactive molecule. Radioactive antibodies that are specific to a cancer cell, disease cell, or virus-infected cell may be able to deliver a sufficient dose of radioactivity to kill such cancer cell, disease cell, or virus-infected cell. The antibodies of the present invention can also be used in assays for detection of the subject polypeptides. In some embodiments, the assay is a binding assay that detects binding of a polypeptide with an antibody specific for the polypeptide; the subject polypeptide or antibody can be immobilized, while the subject polypeptide and/or antibody can be detectably labeled. For example, the antibody can be directly labeled or detected with a labeled secondary antibody. That is, suitable, detectable labels for antibodies include direct labels, which label the antibody to the protein of interest, and indirect labels, which label an antibody that recognizes the antibody to the protein of interest.

These labels include radioisotopes, including, but not limited to ⁶⁴Cu, ⁶⁷Cu, ⁹⁰Y, ¹²⁴I, ¹²⁵I, ¹³¹I, ¹³⁷Cs, ¹⁸⁶Re, ²¹¹At, ²¹²Bi, ²¹³Bi, ²²³Ra, ²⁴¹Am, and ²⁴⁴CM; enzymes having detectable products (e.g., luciferase, β-galactosidase, and the like); fluorescers and fluorescent labels, e.g., as provided herein; fluorescence emitting metals, e.g., ¹⁵²Eu, or others of the lanthanide series, attached to the antibody through metal chelating groups such as EDTA; chemiluminescent compounds, e.g., luminol, isoluminol, or acridinium salts; and bioluminescent compounds, e.g., luciferin, or aequorin (green fluorescent protein), specific binding molecules, e.g., magnetic particles, microspheres, nanospheres, and the like.

Alternatively, specific-binding pairs may be used, involving, e.g., a second stage antibody or reagent that is detectably labeled and that can amplify the signal. For example, a primary antibody can be conjugated to biotin, and horseradish peroxidase-conjugated strepavidin added as a second stage reagent. Digoxin and antidigoxin provide another such pair. In other embodiments, the secondary antibody can be conjugated to an enzyme such as peroxidase in combination with a substrate that undergoes a color change in the presence of the peroxidase. The absence or presence of antibody binding can be determined by various methods, including flow cytometry of dissociated cells, microscopy, radiography, or scintillation counting. Such reagents and their methods of use are well known in the art.

Nucleic acid, polypeptides, and antibodies of the invention can be provided in the form of arrays, i.e., collections of plural biological molecules such as nucleic acids, polypeptides, or antibodies, having locatable addresses that may be separately detectable. Generally, a microarray encompasses use of submicrogram quantities of biological molecules. The biological molecules may be affixed to a substrate or may be in solution or suspension. The substrate can be porous or solid, planar or non-planar, unitary or distributed, such as a glass slide, a 96 well plate, with or without the use of microbeads or nanobeads. As such, the term “microarray” includes all of the devices referred to as microarrays in Schena, 1999; Bassett et al., 1999; Bowtell, 1999; Brown and Botstein, 1999; Chakravarti, 1999; Cheung et al., 1999; Cole et al., 1999; Collins, 1999; Debouck and Goodfellow, 1999; Duggan et al., 1999; Hacia, 1999; Lander, 1999; Lipshutz et al., 1999; Southern, et al., 1999; Schena, 2000; Brenner et al, 2000; Lander, 2001; Steinhaur et al., 2002; and Espejo et al, 2002. Nucleic acid microarrays include both oligonucleotide arrays (DNA chips) containing expressed sequence tags (ESTs) and arrays of larger DNA sequences representing a plurality of genes bound to the substrate, either one of which can be used for hybridization studies. Protein and antibody microarrays include arrays of polypeptides or proteins, including but not limited to, polypeptides or proteins obtained by purification, fusion proteins, and antibodies, and can be used for specific binding studies (Zhu and Snyder, 2003; Houseman et al., 2002; Schaeferling et al., 2002; Weng et al., 2002; Winssinger et al., 2002; Zhu et al., 2001; Zhu et al. 2001; and MacBeath and Schreiber, 2000).

All of the immunogenic methods of the invention can be used alone or in combination with other conventional or unconventional therapies. For example, immunogenic molecules can be combined with other molecules that have a variety of antiproliferative effects, or with additional substances that help stimulate the immune response, i.e., adjuvants or cytokines.

Protein Families

The sequences of the invention encompass a variety of different types of nucleic acids and polypeptides with different structures and functions. They can encode or comprise polypeptides belonging to different protein families (Pfam). The “Pfam” system is an organization of protein sequence classification and analysis, based on conserved protein domains; it can be publicly accessed in a number of ways, for example, at http://pfam.wustl.edu. Protein domains are portions of proteins that have a tertiary structure and sometimes have enzymatic or binding activities; multiple domains can be connected by flexible polypeptide regions within a protein. Pfam domains can comprise the N-terminus or the C-terminus of a protein, or can be situated at any point in between. The Pfam system identifies protein families based on these domains and provides an annotated, searchable database that classifies proteins into families (Bateman et al., 2002).

Sequences of the invention can encode or be comprised of more than one Pfam. Sequences encompassed by the invention include, but are not limited to, the polypeptide and polynucleotide sequences of the molecules shown in the Sequence Listing and corresponding molecular sequences found at all developmental stages of an organism. Sequences of the invention can comprise genes or gene segments designated in the Sequence Listing, and their gene products, i.e., RNA and polypeptides. They also include variants of those presented in the Sequence Listing that are present in the normal physiological state, e.g., variant alleles such as SNPs, splice variants, as well as variants that are affected in pathological states, such as disease-related mutations or sequences with alterations that lead to pathology, and variants with conservative amino acid changes. Some sequences of the invention are categorized below with respect to one or more protein family. Any given sequence can belong to one or more than one category.

Screening and Diagnostic Methods

Identifying Biological Molecules that Interact with a Polypeptide

Formation of a binding complex between a subject polypeptide and an interacting polypeptide or other macromolecule (e.g., DNA, RNA, lipids, polysaccharides, and the like) can be detected using any known method. Suitable methods include: a yeast two-hybrid system (Zhu et al., 1997; Fields and Song, 1989; U.S. Pat. No. 5,283,173; Chien et al. 1991); a mammalian cell two-hybrid method; a fluorescence resonance energy transfer (FRET) assay; a bioluminescence resonance energy transfer (BRET) assay; a fluorescence quenching assay; a fluorescence anisotropy assay (Jameson and Sawyer, 1995); an immunological assay; and an assay involving binding of a detectably labeled protein to an immobilized protein.

Detecting mRNA Levels and Monitoring Gene Expression

The present invention provides methods for detecting the presence of sFPR-3 mRNA in a biological sample. The methods can be used, for example, to assess whether a test compound affects sFPR-3 gene expression, either directly or indirectly. The present invention provides diagnostic methods to compare the abundance of an sFPR-3 nucleic acid with that of a control value, either qualitatively or quantitatively, and to relate the value to a normal or abnormal expression pattern.

Methods of measuring mRNA levels are known in the art, as described in for example, WO 97/27317. These methods generally comprise contacting a sample with a polynucleotide of the invention under conditions that allow hybridization and detecting hybridization, if any, as an indication of the presence of the polynucleotide of interest. Detection can be accomplished by any known method, including, but not limited to, in situ hybridization, PCR, RT-PCR, and “Northern” or RNA blotting, or combinations of such techniques, using a suitably labeled subject polynucleotide. A common method employed is use of microarrays which can be purchased or customized, for example, through conventional vendors such as Affymetrix.

Detecting and Monitoring Polypeptide Presence and Biological Activity

The present invention provides methods for detecting the presence and/or biological activity of a subject polypeptide in a biological sample. The assay used will be appropriate to the biological activity of the particular polypeptide. Thus, e.g., where the biological activity is binding to a second macromolecule, the assay detects protein-protein binding, protein-DNA binding, protein-carbohydrate binding, or protein-lipid binding, as appropriate, using well known assays. Where the biological activity is signal transduction (e.g., transmission of a signal from outside the cell to inside the cell) or transport, an appropriate assay is used, such as measurement of intracellular calcium ion concentration, measurement of membrane conductance changes, or measurement of intracellular potassium ion concentration.

The present invention also provides methods for detecting the presence or measuring the level of a normal or abnormal polypeptide in a biological sample using a specific antibody. The methods generally comprise contacting the sample with a specific antibody and detecting binding between the antibody and molecules of the sample. Specific antibody binding, when compared to a suitable control, is an indication that a polypeptide of interest is present in the sample.

A variety of methods to detect specific antibody-antigen interactions are known in the art, e.g., standard immunohistological methods, immunoprecipitation, enzyme immunoassay, and radioimmunoassay. Briefly, antibodies are added to a cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, specific-binding pairs may be used, involving, e.g., a second stage antibody or reagent that is detectably-labeled, as described above. Such reagents and their methods of use are well known in the art

Modulating mRNA and Peptides in Biological Samples

The present invention provides screening methods for identifying agents that modulate the level of a mRNA molecule of the invention, agents that modulate the level of a polypeptide of the invention, and agents that modulate the biological activity of a polypeptide of the invention. In some embodiments, the assay is cell-free; in others, it is cell-based. Where the screening assay is a binding assay, one or more of the molecules can be joined to a label, where the label can directly or indirectly provide a detectable signal.

In these embodiments, the candidate agent is combined with a cell possessing a polynucleotide transcriptional regulatory element operably linked to a polypeptide-coding sequence of interest, e.g., a subject cDNA or its genomic component; and determining the agent's effect on polynucleotide expression, as measured, for example by the level of mRNA, polypeptide, or fusion polypeptide

In other embodiments, for example, a recombinant vector can comprise an isolated polynucleotide transcriptional regulatory sequence, such as a promoter sequence, operably linked to a reporter gene (e.g., β-galactosidase, CAT, luciferase, or other gene that can be easily assayed for expression). In these embodiments, the method for identifying an agent that modulates a level of expression of a polynucleotide in a cell comprises combining a candidate agent with a cell comprising a transcriptional regulatory element operably linked to a reporter gene; and determining the effect of said agent on reporter gene expression.

Known methods of measuring mRNA levels can be used to identify agents that modulate mRNA levels, including, but not limited to, PCR with detectably-labeled primers. Similarly, agents that modulate polypeptide levels can be identified using standard methods for determining polypeptide levels, including, but not limited to an immunoassay such as ELISA with detectably-labeled antibodies.

A wide variety of cell-based assays can also be used to identify agents that modulate eukaryotic or prokaryotic mRNA and/or polypeptide levels. Examples include transformed cells that over-express a cDNA construct and cells transformed with a polynucleotide of interest associated with an endogenously-associated promoter operably linked to a reporter gene. Expression levels are measured and compared in the test and control samples.

The present invention further provides methods of identifying agents that modulate a biological activity of the polypeptides of the invention. The method generally comprises contacting a test agent with a sample containing the subject polypeptide and assaying a biological activity of the subject polypeptide in the presence of the test agent. An increase or a decrease in the assayed biological activity in comparison to the activity in a suitable control (e.g., a sample comprising a subject polypeptide in the absence of the test agent) is an indication that the substance modulates a biological activity of the subject polypeptide. The mixture of components is added in any order that provides for the requisite interaction.

Accordingly, the present invention provides a method for identifying an agent, particularly a biologically active agent that modulates the level of expression of a nucleic acid in a cell, the method comprising: combining a candidate agent to be tested with a cell comprising a nucleic acid that encodes the polypeptide, and determining the agent's effect on polypeptide expression.

Agents that decrease a biological activity can find use in treating disorders associated with the biological activity of the molecule. Alternatively, some embodiments will detect agents that increase a biological activity. Agents that increase a biological activity of a molecule of the invention can find use in treating disorders associated with a deficiency in the biological activity.

A variety of different candidate agents can be screened by the above methods. Candidate agents encompass numerous chemical classes, as described herein. Candidate agents are obtained from a wide variety of sources including libraries of synthetic or natural compounds. Numerous means are available for random and directed synthesis of a wide variety of organic compounds and biomolecules, including expression of randomized oligonucleotides and oligopeptides. For example, random peptide libraries obtained by yeast two-hybrid screens (Xu et al., 1997), phage libraries (Hoogenboom et al., 1998), or chemically generated libraries. Alternatively, libraries of natural compounds in the form of bacterial, fungal, plant and animal extracts are available or readily produced, including antibodies produced upon immunization of an animal with subject polypeptides, or fragments thereof, or with the encoding polynucleotides. Additionally, natural or synthetically produced libraries and compounds are readily modified through conventional chemical, physical and biochemical means, and can be used to produce combinatorial libraries. Further, known pharmacological agents can be subjected to directed or random chemical modifications, such as acylation, alkylation, esterification, and amidification, etc, to produce structural analogs.

Kits

The present invention provides methods for diagnosing disease states based on the detected presence and/or level of polynucleotides, polypeptides, or antibodies in a biological sample, and/or the detected presence and/or level of biological activity of the polynucleotide or polypeptide. These detection methods can be provided as part of a kit. Thus, the invention further provides kits for detecting the presence and/or a level of a polynucleotide, polypeptide, or antibody of interest in a biological sample.

Where the kit provides for polypeptide detection, it can include one or more specific antibodies. In some embodiments, the antibody specific to the polypeptide of interest is detectably labeled. In other embodiments, the antibody specific to the polypeptide is not labeled; instead, a second, detectably labeled antibody is provided that binds to the specific antibody. The kit may further include blocking reagents, buffers, and reagents for developing and/or detecting the detectable marker. The kit may further include instructions for use, controls, and interpretive information.

The present invention provides for kits with unit doses of an active agent. In some embodiments, the agent is provided in oral or injectable doses. Such kits will comprise containers containing the unit doses and an informational package insert describing the use and attendant benefits of the drugs in treating a condition of interest.

Therapeutic Compositions

The invention further provides agents identified using a screening assay of the invention, and compositions comprising the agents, subject polypeptides, subject polynucleotides, modulators thereof including antibodies, recombinant vectors, and/or host cells, including pharmaceutical compositions containing such in a pharmaceutically acceptable carrier or excipient for therapeutic administration. The subject compositions can be formulated using well-known reagents and methods. These compositions can include a buffer, which is selected according to the desired use of the agent, polypeptide, polynucleotide, recombinant vector, or host cell, and can also include other substances appropriate to the intended use. Those skilled in the art can readily select an appropriate buffer, a wide variety of which are known in the art, suitable for an intended use.

Excipients and Formulations

In some embodiments, compositions are provided in formulation with pharmaceutically acceptable excipients, a wide variety of which are known in the art (Gennaro, 2000; Ansel et al., 2004; Kibbe et al., 2000). Pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In pharmaceutical dosage forms, the compositions of the invention can be administered in the form of their pharmaceutically acceptable salts, or they can also be used alone or in appropriate association, as well as in combination, with other pharmaceutically active compounds. The subject compositions are formulated in accordance to the mode of potential administration. Administration of the agents can be achieved in various ways, including oral, buccal, nasal, rectal, parenteral, intraperitoneal, intradermal, transdermal, subcutaneous, intravenous, intra-arterial, intracardiac, intraventricular, intracranial, intratracheal, and intrathecal administration, etc., or otherwise by implantation or inhalation. Thus, the subject compositions can be formulated into preparations in solid, semi-solid, liquid or gaseous forms, such as tablets, capsules, powders, granules, ointments, solutions, suppositories, injections, inhalants and aerosols. The following methods and excipients are merely exemplary and are in no way limiting.

Compositions for oral administration can form solutions, suspensions, tablets, pills, granules, capsules, sustained release formulations, oral rinses, or powders. For oral preparations, the agents, polynucleotides, and polypeptides can be used alone or in combination with appropriate additives, for example, with conventional additives, such as lactose, mannitol, corn starch, or potato starch; with binders, such as crystalline cellulose, cellulose derivatives, acacia, corn starch, or gelatins; with disintegrators, such as corn starch, potato starch, or sodium carboxymethylcellulose; with lubricants, such as talc or magnesium stearate; and if desired, with diluents, buffering agents, moistening agents, preservatives, and flavoring agents.

Suitable excipient vehicles are, for example, water, saline, dextrose, glycerol, ethanol, or the like, and combinations thereof. In addition, if desired, the vehicle can contain minor amounts of auxiliary substances such as wetting or emulsifying agents or pH buffering agents. Actual methods of preparing such dosage forms are known, or will be apparent, to those skilled in the art (Gennaro, 2003). The composition or formulation to be administered will, in any event, contain a quantity of the agent adequate to achieve the desired state in the subject being treated.

The agents, polynucleotides, and polypeptides can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Other formulations for oral or parenteral delivery can also be used, as conventional in the art

The antibodies, agents, polynucleotides, and polypeptides can be utilized in aerosol formulation to be administered via inhalation. The compounds of the present invention can be formulated into pressurized acceptable propellants such as dichlorodifluoromethane, propane, nitrogen, and the like. Further, the agent, polynucleotides, or polypeptide composition may be converted to powder form for administration intranasally or by inhalation, as conventional in the art.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

A polynucleotide, polypeptide, or other modulator, can also be introduced into tissues or host cells by other routes, such as viral infection, microinjection, or vesicle fusion. For example, expression vectors can be used to introduce nucleic acid compositions into a cell as described above. Further, jet injection can be used for intramuscular administration (Furth et al., 1992). The DNA can be coated onto gold microparticles, and delivered intradermally by a particle bombardment device, or “gene gun” as described in the literature (Tang et al., 1992), where gold microprojectiles are coated with the DNA, then bombarded into skin cells.

Unit dosage forms for oral or rectal administration such as syrups, elixirs, and suspensions can be provided wherein each dosage unit, for example, teaspoonful, tablespoonful, tablet, or suppository, contains a predetermined amount of the composition containing one or more agents. Similarly, unit dosage forms for injection or intravenous administration can comprise the agent(s) in a composition as a solution in sterile water, normal saline or another pharmaceutically acceptable carrier.

Active Agents (or Modulators)

The nucleic acid, polypeptide, and modulator compositions of the subject invention find use as therapeutic agents in situations where one wishes to modulate an activity of a subject polypeptide in a host, particularly the activity of the subject polypeptides, or to provide or inhibit the activity at a particular anatomical site. Thus, the compositions are useful in treating disorders associated with an activity of a subject polypeptide. The following provides further details of active agents of the present invention.

Antisense Oligonucleotides

In certain embodiments of the invention, the active agent is an agent that modulates, and generally decreases or down regulates, polypeptide expression in a host, i.e., antisense molecules. Anti-sense reagents include antisense oligonucleotides (ODN), i.e., synthetic ODN having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g., by reducing the amount of mRNA available for translation, through activation of RNase H, or steric hindrance. One or a combination of antisense molecules can be administered, where a combination can comprise multiple different sequences.

Antisense molecules can be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides can be chemically synthesized by methods known in the art (Wagner et al., 1993; Milligan et al., 1993). Antisense oligonucleotides will generally be at least about 7, at least about 12, or at least about 20 nucleotides in length, and not more than about 500, not more than about 50, or not more than about 35 nucleotides in length, where the length is governed by efficiency of inhibition, and specificity, including absence of cross-reactivity, and the like. Short oligonucleotides, of from about 7 to about 8 bases in length, can be strong and selective inhibitors of gene expression (Wagner et al., 1996).

As an alternative to anti-sense inhibitors, catalytic nucleic acid compounds, e.g., ribozymes, or anti-sense conjugates can be used to inhibit gene expression. Ribozymes can be synthesized ill vitro and administered to the patient, or can be encoded in an expression vector, from which the ribozyme is synthesized in the targeted cell (WO 9523225; Beigelman et al., 1995). Examples of oligonucleotides with catalytic activity are described in WO 9506764. Conjugates of anti-sense ODN with a metal complex, e.g., terpyridyl Cu(II), capable of mediating mRNA hydrolysis are described in Bashkin et al., 1995.

Interfering RNA

In some embodiments, the active agent is an interfering RNA (RNAi), including dsRNAi. RNA interference provides a method of silencing eukaryotic genes. Use of RNAi to reduce a level of a particular mRNA and/or protein is based on the interfering properties of double-stranded RNA derived from the coding regions of a gene. The technique is an efficient high-throughput method for disrupting gene function (O'Neil, 2001). RNAi can also help identify the biochemical mode of action of a drug and to identify other genes encoding products that can respond or interact with specific compounds.

In one embodiment of the invention, complementary sense and antisense RNAs derived from a substantial portion of the subject polynucleotide are synthesized in vitro. The resulting sense and antisense RNAs are annealed in an injection buffer, and the double-stranded RNA injected or otherwise introduced into the subject, i.e., in food or by immersion in buffer containing the RNA (Gaudilliere et al., 2002; O'Neil et al., 2001; WO99/32619). In another embodiment, dsRNA derived from a gene of the present invention is generated in vivo by simultaneously expressing both sense and antisense RNA from appropriately positioned promoters operably linked to coding sequences in both sense and antisense orientations.

Peptides and Modified Peptides

In some embodiments of the present invention, the active agent is a peptide. Suitable peptides include peptides of from about 5 amino acids to about 50, from about 6 to about 30, or from about 10 to about 20 amino acids in length. In some embodiments, a peptide has a sequence of from about 7 amino acids to about 45, from about 9 to about 35, or from about 12 to about 25 amino acids of corresponding naturally-occurring protein. In some embodiments, a peptide exhibits one or more of the following activities: inhibits binding of a subject polypeptide to an interacting protein or other molecule; inhibits subject polypeptide binding to a second polypeptide molecule; inhibits a signal transduction activity of a subject polypeptide; inhibits an enzymatic activity of a subject polypeptide; or inhibits a DNA binding activity of a subject polypeptide.

Peptides can include naturally-occurring and non-naturally occurring amino acids. Peptides can comprise D-amino acids, a combination of D- and L-amino acids, and various “designer” amino acids (e.g., α-methyl amino acids, Cα-methyl amino acids, and Nα-methyl amino acids, etc.) to convey special properties. Additionally, peptides can be cyclic. Peptides can include non-classical amino acids in order to introduce particular conformational motifs. Any known non-classical amino acid can be used. Non-classical amino acids include, but are not limited to, 1,2,3,4-tetrahydroisoquinoline-3-carboxylate; (2S,3S)-methylphenylalanine, (2S,3R)-methyl-phenylalanine, (2R,3S)-methyl-phenylalanine and (2R,3R)-methyl-phenylalanine; 2-aminotetrahydronaphthalene-2-carboxylic acid; hydroxy-1,2,3,4-tetrahydroisoquinoline-3-carboxylate; β-carboline (D and L); HIC (histidine isoquinoline carboxylic acid); and HIC (histidine cyclic urea). Amino acid analogs and peptidomimetics can be incorporated into a peptide to induce or favor specific secondary structures, including, but not limited to, LL-Acp (LL-3-amino-2-propenidone-6-carboxylic acid), a β-turn inducing dipeptide analog; β-sheet inducing analogs; β-turn inducing analogs; α-helix inducing analogs; γ-turn inducing analogs; Gly-Ala turn analogs; amide bond isostere; or tretrazol, and the like.

In addition to the foregoing N-terminal and C-terminal modifications, a peptide or peptidomimetic can be modified with or covalently coupled to one or more of a variety of hydrophilic polymers to increase solubility and circulation half-life of the peptide. Suitable nonproteinaceous hydrophilic polymers for coupling to a peptide include, but are not limited to, polyalkylethers as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran, and dextran derivatives. Generally, such hydrophilic polymers have an average molecular weight ranging from about 500 to about 100,000 daltons, from about 2,000 to about 40,000 daltons, or from about 5,000 to about 20,000 daltons. The peptide can be derivatized with or coupled to such polymers using any of the methods set forth in Zallipsky, (1995); Monfardini et al., (1995); U.S. Pat. Nos. 4,640,835; 4,496,689; 4,301,144; 4,670,417; 4,791,192; 4,179,337, or WO 95/34326.

Peptide Aptamers

Another suitable agent for modulating an activity of a subject polypeptide is a peptide aptamer. Peptide aptamers are peptides or small polypeptides that act as dominant inhibitors of protein function. Peptide aptamers specifically bind to target proteins, blocking their functional ability (Kolonin and Finley, 1998). Due to the highly selective nature of peptide aptamers, they can be used not only to target a specific protein, but also to target specific functions of a given protein (e.g., a signaling function). Further, peptide aptamers can be expressed in a controlled fashion by use of promoters which regulate expression in a temporal, spatial or inducible manner. Peptide aptamers act dominantly, therefore, they can be used to analyze proteins for which loss-of-function mutants are not available.

Peptide aptamers that bind with high affinity and specificity to a target protein can be isolated by a variety of techniques known in the art. Peptide aptamers can be isolated from random peptide libraries by yeast two-hybrid screens (Xu et al., 1997). They can also be isolated from phage libraries (Hoogenboom et al., 1998) or chemically generated peptides/libraries.

Therapeutic Applications: Methods of Use

The instant invention provides various therapeutic methods. In some embodiments, methods of modulating, including increasing and inhibiting, a biological activity of a subject protein are provided. In other embodiments, methods of modulating a signal transduction activity of a subject protein are provided. In further embodiments, methods of modulating interaction of a subject protein with another, interacting protein or other macromolecule (e.g., DNA, carbohydrate, lipid), are provided.

Thus, in an embodiment, the therapeutic compositions herein are administered to subjects for treatment of a proliferative disease such as a tumor or psoriasis. In another embodiment, the therapeutic compositions herein are administered to subjects for modulation of immune related diseases or infections. In further embodiments, the therapeutic compositions herein are administered to subjects for modulation of apoptosis-related diseases, metabolic diseases, infectious diseases, and/or degenerative diseases. Such compositions are administered either locally or systemically, for example, intranasally or by inhalation, by intravenous, intramusclular, subcutaneous, intrathecal, intraventricular, or intraperitoneal administration.

As mentioned above, an effective amount of the active agent (e.g., small molecule, antibody specific for a subject polypeptide, a subject polypeptide, or a subject polynucleotide) is administered to the host, where “effective amount” means a dosage sufficient to produce a desired effect or result. In some embodiments, the desired result is at least a reduction in a given biological activity of a subject polypeptide as compared to a control, for example, a decreased level of expression or activity of the subject protein in the individual, or in a localized anatomical site in the individual. In further embodiments, the desired result is at least an increase in a biological activity of a subject polypeptide as compared to a control.

The agents can be provided in unit dosage forms, i.e., physically discrete units suitable as unitary dosages for human and animal subjects, each unit containing a predetermined quantity of compounds of the present invention calculated in an amount sufficient to produce the desired effect in association with a pharmaceutically acceptable diluent, carrier, or vehicle. The specifications for the novel unit dosage forms of the present invention depend on the particular compound employed and the effect to be achieved, and the pharmacodynamics associated with each compound in the host.

An effective amount of the active is administered to the host at a dosage sufficient to produce a desired result. In some embodiments, the desired result is at least a reduction in a given biological activity of a subject polypeptide as compared to a control. In other embodiments, the desired result is an increase in the level of the active subject polypeptide (in the individual, or in a localized anatomical site in the individual), as compared to a control. In some embodiments, the desired result is at least a reduction in enzymatic activity of a subject polypeptide as compared to a control. In other embodiments, the desired result is an increase in the level of enzymatically active subject polypeptide (in the individual, or in a localized anatomical site in the individual), as compared to a control. In still other embodiments, the desired result is a decrease in ischemic cardiac injury as compared to a control. A decrease in ischemic cardiac injury may be indicated by a variety of indicia known in the art or described herein.

Typically, the compositions of the instant invention will contain from less than 1% to about 95% of the active ingredient, in some embodiments, about 10% to about 50%. Generally, between about 100 mg and 500 mg of the compositions will be administered to a child and between about 500 mg and 5 grams will be administered to an adult. Administration is generally by injection and often by injection to a localized area. The frequency of administration will be determined by the care given based on patient responsiveness. Other effective dosages can be readily determined by one of ordinary skill in the art through trials establishing dose response curves.

In order to calculate the amount of therapeutic agent to be administered, those skilled in the art could use readily available information with respect to the amount of agent necessary to have the desired effect. The amount of an agent necessary to increase a level of active subject polypeptide can be calculated from in vitro experimentation. The amount of agent will, of course, vary depending upon the particular agent used.

Typically, the compositions of the instant invention will contain from less than about 1% to about 99% of the active ingredient, about 10% to about 90%, or 20% to about 80%, or 30% to about 70%, or 40% to about 60%, or about 50%. Generally, between about 100 mg and about 500 mg will be administered to a child and between about 500 mg and about 5 grams will be administered to an adult.

Other effective dosages can be readily determined by one of ordinary skill in the art through routine trials establishing dose response curves, for example, the amount of agent necessary to increase a level of active subject polypeptide can be calculated from in vitro experimentation. Those of skill will readily appreciate that dose levels can vary as a function of the specific compound, the severity of the symptoms, and the susceptibility of the subject to side effects, and preferred dosages for a given compound are readily determinable by those of skill in the art by a variety of means. For example, in order to calculate the polypeptide, polynucleotide, or modulator dose, those skilled in the art can use readily available information with respect to the amount necessary to have the desired effect, depending upon the particular agent used.

The active agent(s) can be administered to the host via any convenient means capable of resulting in the desired result. Administration is generally by injection and often by injection to a localized area. The frequency of administration will be determined by the care given based on patient responsiveness. For example, the agents may be administered daily, weekly, or as conventionally determined appropriate.

A variety of hosts are treatable according to the subject methods. The host, or patient, may be from any animal species, and will generally be mammalian, e.g., primate sp., e.g., monkeys, chimpanzees, and particularly humans; rodents, including mice, rats and hamsters, guinea pig; rabbits; cattle, including equines, bovines, pig, sheep, goat, canines; felines; etc. Animal models are of interest for experimental investigations, providing a model for treatment of human disease.

Proliferative Conditions

In some embodiments, a protein of the present invention is involved in the control of cell proliferation, and an agent of the invention inhibits undesirable cell proliferation. Such agents are useful for treating disorders that involve abnormal cell proliferation, including, but not limited to, cancer, psoriasis, and scleroderma. Whether a particular agent and/or therapeutic regimen of the invention is effective in reducing unwanted cellular proliferation, e.g., in the context of treating cancer, can be determined using standard methods.

The therapeutic compositions and methods of the invention can be used in the treatment of cancer, i.e., an abnormal malignant cell or tissue growth, e.g., a tumor. In an embodiment, the compositions and methods of the invention kill tumor cells. In an embodiment, they inhibit tumor development. Cancer is characterized by the proliferation of abnormal cells that tend to invade the surrounding tissue and metastasize to new body sites. The growth of cancer cells exceeds that of and is uncoordinated with the normal cells and tissues. In an embodiment, the compositions and methods of the invention inhibit the progression of premalignant lesions to malignant tumors.

Cancer encompasses carcinomas, which are cancers of epithelial cells, and are the most common forms of human cancer; carcinomas include squamous cell carcinoma, adenocarcinoma, melanomas, and hepatomas. Cancer also encompasses sarcomas, which are tumors of mesenchymal origin, and includes osteogenic sarcomas, leukemias, and lymphomas. Cancers can have one or more than one neoplastic cell type. Some characteristics that can, in some instances, apply to cancer cells are that they are morphologically different from normal cells, and may appear anaplastic; they have a decreased sensitivity to contact inhibition, and may be less likely than normal cells to stop moving when surrounded by other cells; and they have lost their dependence on anchorage for cell growth, and may continue to divide in liquid or semisolid surroundings, whereas normal cells must be attached to a solid surface to grow.

Treatment herein refers to obtaining a desired pharmacologic and/or physiologic effect, covering any treatment of a pathological condition or disorder in a mammal, including a human. The effect may be prophylactic in terms of completely or partially preventing a disorder or symptom thereof and/or may be therapeutic in terms of a partial or complete cure for a disorder and/or adverse affect attributable to the disorder. That is, “treatment” includes (1) preventing the disorder from occurring or recurring in a subject who may be predisposed to the disorder but has not yet been diagnosed as having it, (2) inhibiting the disorder, such as arresting its development, (3) stopping or terminating the disorder or at least symptoms associated therewith, so that the host no longer suffers from the disorder or its symptoms, such as causing regression of the disorder or its symptoms, for example, by restoring or repairing a lost, missing or defective function, or stimulating an inefficient process, or (4) relieving, alleviating, or ameliorating the disorder, or symptoms associated therewith, where ameliorating is used in a broad sense to refer to at least a reduction in the magnitude of a parameter, such as inflammation, pain, and/or tumor size.

The polynucleotides, polypeptides, and antibodies described above can be used to treat cancer. In an embodiment, a fusion protein or conjugate can additionally comprise a tumor-targeting moiety. Suitable moieties include those that enhance delivery of an therapeutic molecule to a tumor. For example, compounds that selectively bind to cancer cells compared to normal cells, selectively bind to tumor vasculature, selectively bind to the tumor type undergoing treatment, or enhance penetration into a solid tumor are included in the invention. Tumor targeting moieties of the invention can be peptides. Nucleic acid and amino acid molecules of the invention can be used alone or as an adjunct to cancer treatment. For example, a nucleic acid or amino acid molecules of the invention may be added to a standard chemotherapy regimen. It may be combined with one or more of the wide variety of drugs that have been employed in cancer treatment, including, but are not limited to, cisplatin, taxol, etoposide, Novantrone (mitoxantrone), actinomycin D, camptohecin (or water soluble derivatives thereof), methotrexate, mitomycins (e.g., mitomycin C), dacarbazine (DTIC), and anti-neoplastic antibiotics such as doxorubicin and daunomycin.

Drugs employed in cancer therapy may have a cytotoxic or cytostatic effect on cancer cells, or may reduce proliferation of the malignant cells. Drugs employed in cancer treatment can also be peptides. A nucleic acid or amino acid molecules of the invention can be combined with radiation therapy. A nucleic acid or amino acid molecules of the invention may be used adjunctively with therapeutic approaches described in De Vita et al., 2001. For those combinations in which a nucleic acid or amino acid molecule of the invention and a second anti-cancer agent exert a synergistic effect against cancer cells, the dosage of the second agent may be reduced, compared to the standard dosage of the second agent when administered alone. A method for increasing the sensitivity of cancer cells comprises co-administering a nucleic acid or amino acid molecule of the invention with an amount of a chemotherapeutic anti-cancer drug that is effective in enhancing sensitivity of cancer cells. Co-administration may be simultaneous or non-simultaneous administration. A nucleic acid or amino acid molecule of the invention may be administered along with other therapeutic agents, during the course of a treatment regimen. In one embodiment, administration of a nucleic acid or amino acid molecule of the invention and other therapeutic agents is sequential. An appropriate time course may be chosen by the physician, according to such factors as the nature of a patients illness, and the patient's condition.

The invention also provides a method for prophylactic or therapeutic treatment of a subject needing or desiring such treatment by providing a vaccine that can be administered to the subject. The vaccine may comprise one or more of a polynucleotide, polypeptide, or modulator of the invention, for example an antibody vaccine composition, a polypeptide vaccine composition, or a polynucleotide vaccine composition, useful for treating cancer, proliferative, inflammatory, immune, metabolic, bacterial, or viral disorders.

For example, the vaccine can be a cancer vaccine, and the polypeptide can concomitantly be a cancer antigen. The vaccine may be an anti-inflammatory vaccine, and the polypeptide can concomitantly be an inflammation-related antigen. The vaccine may be a viral vaccine, and the polypeptide can concomitantly be a viral antigen. In some embodiments, the vaccine comprises a polypeptide fragment, comprising at least one extracellular fragment of a polypeptide of the invention, and/or at least one extracellular fragment of a polypeptide of the invention minus the signal peptide, for the treatment, for example, of proliferative disorders, such as cancer. In certain embodiments, the vaccine comprises a polynucleotide encoding one or more such fragments, administered for the treatment, for example, of proliferative disorders, such as cancer. Further, the vaccine can be administered with or without an adjuvant.

Vaccine therapy involves the use of polynucleotides, polypeptides, or agents of the invention as immunogens for tumor antigens (Machiels et al., 2002). For example, peptide-based vaccines of the invention include unmodified subject polypeptides, fragments thereof, and MHC class I and class II-restricted peptide (Knutson et al., 2001), comprising, for example, the disclosed sequences with universal, nonspecific MHC class II-restricted epitopes. Peptide-based vaccines comprising a tumor antigen can be given directly, either alone or in conjunction with other molecules. The vaccines can also be delivered orally by producing the antigens in transgenic plants that can be subsequently ingested (U.S. Pat. No. 6,395,964).

In some embodiments, antibodies themselves can be used as antigens in anti-idiotype vaccines. That is, administering an antibody to a tumor antigen stimulates B cells to make antibodies to that antibody, which in turn recognize the tumor cells

Nucleic acid-based vaccines can deliver tumor antigens as polynucleotide constructs encoding the antigen. Vaccines comprising genetic material, such as DNA or RNA, can be given directly, either alone or in conjunction with other molecules. Administration of a vaccine expressing a molecule of the invention, e.g., as plasmid DNA, leads to persistent expression and release of the therapeutic immunogen over a period of time, helping to control unwanted tumor growth.

In some embodiments, nucleic acid-based vaccines encode subject antibodies. In such embodiments, the vaccines (e.g., DNA vaccines) can include post-transcriptional regulatory elements, such as the post-transcriptional regulatory acting RNA element (WPRE) derived from Woodchuck Hepatitis Virus. These post-transcriptional regulatory elements can be used to target the antibody, or a fusion protein comprising the antibody and a co-stimulatory molecule, to the tumor microenvironment (Pertl et al., 2003).

Besides stimulating anti-tumor immune responses by inducing humoral responses, vaccines of the invention can also induce cellular responses, including stimulating T-cells that recognize and kill tumor cells directly. For example, nucleotide-based vaccines of the invention encoding tumor antigens can be used to activate the CD8⁺ cytotoxic T lymphocyte arm of the immune system.

In some embodiments, the vaccines activate T-cells directly, and in others they enlist antigen-presenting cells to activate T-cells. Killer T-cells are primed, in part, by interacting with antigen-presenting cells, i.e., dendritic cells. In some embodiments, plasmids comprising the nucleic acid molecules of the invention enter antigen-presenting cells, which in turn display the encoded tumor-antigens that contribute to killer T-cell activation. Again, the tumor antigens can be delivered as plasmid DNA constructs, either alone or with other molecules.

In further embodiments, RNA can be used. For example, dendritic cells can be transfected with RNA encoding tumor antigens (Heiser et al., 2002; Mitchell and Nair, 2000). This approach overcomes the limitations of obtaining sufficient quantities of tumor material, extending therapy to patients otherwise excluded from clinical trials. For example, a subject RNA molecule isolated from tumors can be amplified using RT-PCR. In some embodiments, the RNA molecule of the invention is directly isolated from tumors and transfected into dendritic cells with no intervening cloning steps.

In some embodiments the molecules of the invention are altered such that the peptide antigens are more highly antigenic than in their native state. These embodiments address the need in the art to overcome the poor in vivo immunogenicity of most tumor antigens by enhancing tumor antigen immunogenicity via modification of epitope sequences (Yu and Restifo, 2002).

Another recognized problem of cancer vaccines is the presence of preexisting neutralizing antibodies. Some embodiments of the present invention overcome this problem by using viral vectors from non-mammalian natural hosts, e.g., avian pox viruses. Alternative embodiments that also circumvent preexisting neutralizing antibodies include genetically engineered influenza viruses, and the use of “naked” plasmid DNA vaccines that contain DNA with no associated protein. (Yu and Restifo, 2002).

All of the immunogenic methods of the invention can be used alone or in combination with other conventional or unconventional therapies. For example, immunogenic molecules can be combined with other molecules that have a variety of antiproliferative effects, or with additional substances that help stimulate the immune response, i.e., adjuvants or cytokines.

For example, in some embodiments, nucleic acid vaccines encode an alphaviral replicase enzyme, in addition to tumor antigens. This recently discovered approach to vaccine therapy successfully combines therapeutic antigen production with the induction of the apoptotic death of the tumor cell (Yu and Restifo, 2002).

In some embodiments, a protein of the present invention is involved in the control of cell proliferation, and an agent of the invention inhibits undesirable cell proliferation. Such agents are useful for treating disorders that involve abnormal cell proliferation, including, but not limited to, cancer, psoriasis, and scleroderma. Whether a particular agent and/or therapeutic regimen of the invention is effective in reducing unwanted cellular proliferation, e.g., in the context of treating cancer, can be determined using standard methods. For example, the number of cancer cells in a biological sample (e.g., blood, a biopsy sample, and the like), can be determined. The tumor mass can be determined using standard radiological or biochemical methods.

The polynucleotides, polypeptides, and modulators of the present invention find use in immunotherapy of hyperproliferative disorders, including cancer, neoplastic, and paraneoplastic disorders. That is, the subject molecules can correspond to tumor antigens, That is, the subject molecules can correspond to tumor antigens, of which at least 1770 have been identified (Yu and Restifo, 2002). Immunotherapeutic approaches include passive immunotherapy and vaccine therapy and can accomplish both generic and antigen-specific cancer immunotherapy.

Passive immunity approaches involve antibodies of the invention that are directed toward specific tumor-associated antigens. Such antibodies can eradicate systemic tumors at multiple sites, without eradicating normal cells. In some embodiments, the antibodies are combined with radioactive components, as provided above, for example, combining the antibody's ability to specifically target tumors with the added lethality of the radioisotope to the tumor DNA.

Useful antibodies comprise a discrete epitope or a combination of nested epitopes, i.e., a 10-mer epitope and associated peptide multimers incorporating all potential 8-mers and 9-mers, or overlapping epitopes (Dutoit et al., 2002). Thus a single antibody can interact with one or more epitopes. Further, the antibody can be used alone or in combination with different antibodies, that all recognize either a single or multiple epitopes.

Neutralizing antibodies can provide therapy for cancer and proliferative disorders. Neutralizing antibodies that specifically recognize a protein or peptide of the invention can bind to the protein or peptide, e.g., in a bodily fluid or the extracellular space, thereby modulating the biological activity of the protein or peptide. For example, neutralizing antibodies specific for proteins or peptides that play a role in stimulating the growth of cancer cells can be useful in modulating the growth of cancer cells. Similarly, neutralizing antibodies specific for proteins or peptides that play a role in the differentiation of cancer cells can be useful in modulating the differentiation of cancer cells.

Inflammation and Immunity

In other embodiments, e.g., where the subject polypeptide is involved in modulating inflammation or immune function, the invention provides agents for treating such inflammation or immune disorders. For example, neutralizing antibodies can provide immunosuppressive therapy for inflammatory and autoimmune disorders. Neutralizing antibodies can be used to treat disorders such as, for example, multiple sclerosis, rheumatoid arthritis, inflammatory bowel disease, transplant rejection, and psoriasis. Neutralizing antibodies that specifically recognize a protein or peptide of the invention can bind to the protein or peptide, e.g., in a bodily fluid or the extracellular space, thereby modulating the biological activity of the protein or peptide. For example, neutralizing antibodies specific for proteins or peptides that play a role in activating immune cells are useful as immunosuppressants.

Disorders Related to Cell Death

Where a polypeptide of the invention is involved in modulating cell death, an agent of the invention is useful for treating conditions or disorders relating to cell death (e.g., DNA damage, cell death, apoptosis). Cell death-related indications that can be treated using the methods of the invention to reduce cell death in a eukaryotic cell, include, but are not limited to, cell death associated with Alzheimer's disease, Parkinson's disease, rheumatoid arthritis, autoimmune thyroiditis, septic shock, sepsis, stroke, central nervous system inflammation, intestinal inflammation, osteoporosis, ischemia, reperfusion injury, cardiac muscle cell death associated with cardiovascular disease, polycystic kidney disease, cell death of endothelial cells in cardiovascular disease, degenerative liver disease, multiple sclerosis, amyotropic lateral sclerosis, cerebellar degeneration, ischemic injury, cerebral infarction, myocardial infarction, acquired immunodeficiency syndrome (AIDS), myelodysplastic syndromes, aplastic anemia, male pattern baldness, and head injury damage. Also included are conditions in which DNA damage to a cell is induced by external conditions, including but not limited to irradiation, radiomimetic drugs, hypoxic injury, chemical injury, and damage by free radicals. Also included are any hypoxic or anoxic conditions, e.g., conditions relating to or resulting from ischemia, myocardial infarction, cerebral infarction, stroke, bypass heart surgery, organ transplantation, and neuronal damage, etc.

Apoptosis, or programmed cell death, is a regulated process leading to cell death via a series of well-defined morphological changes. Programmed cell death provides a balance for cell growth and multiplication, eliminating unnecessary cells. The default state of the cell is to remain alive. A cell enters the apoptotic pathway when an essential factor is removed from the extracellular environment or when an internal signal is activated. Genes and proteins of the invention that suppress the growth of tumors by activating cell death provide the basis for treatment strategies for hyperproliferative disorders and conditions.

Apoptosis can be assayed using any known method. Assays can be conducted on cell populations or an individual cell, and include morphological assays and biochemical assays. Procedures to detect cell death based on the TUNEL method are available commercially, e.g., from Boehringer Mannheim (Cell Death Kit) and Oncor (Apoptag Plus).

Such stimulatory properties render polypeptides of the invention and modulators thereto useful for the treatment, prevention, and diagnosis of diseases. Polypeptides of the invention and modulators thereof, such as antibodies thereto, may be used as therapeutic proteins or therapeutic targets in the treatment of diseases involved in the malfunction of the immune system, including inflammatory and autoimmune diseases, such as rheumatoid arthritis and osteoarthritis, psoriasis, inflammatory bowel disease, and multiple sclerosis. Antibodies against these proteins or small molecules inhibiting these proteins or their receptors could also be used to treat inflammatory and autoimmune diseases. These proteins may also be used as immunotherapeutic agent for treatment of cancers and infectious diseases, and in vaccines. They may be used as therapeutic protein to treat cancers, such as by inducing apoptosis.

Investigative Applications

The subject nucleic acid compositions find use in a variety of different investigative applications. Applications of interest include identifying genomic DNA sequence using molecules of the invention, identifying homologs of molecules of the invention, creating a source of novel promoter elements, identifying expression regulatory factors, creating a source of probes and primers for hybridization applications, identifying expression patterns in biological specimens; preparing cell or animal models to investigate the function of the molecules of the invention, and preparing in vitro models to investigate the function of the molecules of the invention.

Genomic DNA Sequences

Human genomic polynucleotide sequences corresponding to molecules of the present invention are identified by conventional means, such as, for example, by probing a genomic DNA library with all or a portion of the polynucleotide sequences.

Homologs are identified by any of a number of methods. By using probes, particularly labeled probes of DNA sequences, one can isolate homologous or related genes, as described in detail above. Briefly, a fragment of the provided cDNA can be used as a hybridization probe against a cDNA library from the target organism of interest, under various stringency conditions, e.g., low stringency conditions. The probe can be a large fragment, or one or more short degenerate primers, and is typically labeled. Sequence identity can be determined by hybridization under stringent conditions, as described in detail above. Nucleic acids having a region of substantial identity or sequence similarity to the provided nucleic acid sequences, for example allelic variants, related genes, or genetically altered versions of the gene, bind to the provided sequences under less stringent hybridization conditions.

Promoter Elements and Expression Regulatory Factors

The sequence of the 5′ flanking region can be utilized as promoter elements, including enhancer binding sites that provide for tissue-specific expression and developmental regulation in tissues where the subject genes are expressed, providing promoters that mimic the native pattern of expression. Naturally occurring polymorphisms in the promoter region are useful for determining natural variations in expression, particularly those that may be associated with disease. Promoters or enhancers that regulate the transcription of the polynucleotides of the present invention are obtainable by use of PCR techniques using human tissues, and one or more of the present primers.

Alternatively, mutations can be introduced into the promoter region to determine the effect of altering expression in experimentally defined systems. Methods for the identification of specific DNA motifs involved in the binding of transcriptional factors are known in the art, for example sequence similarity to known binding motifs, and gel retardation studies (Blackwell et al., 1995; Mortlock et al., 1996; Joulin and Richard-Foy, 1995).

The regulatory sequences can be used to identify cis acting sequences required for transcriptional or translational regulation of expression, especially in different tissues or stages of development, and to identify cis acting sequences and trans-acting factors that regulate or mediate expression. Such transcription or translational control regions can be operably linked to a gene in order to promote expression of wild type genes or of proteins of interest in cultured cells, embryonic, fetal or adult tissues, and for gene therapy (Hooper, 1993).

Primers and Probes

Small DNA fragments are useful as primers for reactions that involve nucleic acid hybridization, as described in detail above. Briefly, pairs of primers will be used in amplification reactions, such as PCR. Amplification primers hybridize to complementary strands of DNA, for example, under stringent conditions, and will prime towards each other. In some embodiments a pair of primers will generate an amplification product of at least about 50 nt, or at least about 100 nt. Algorithms for the selection of primer sequences are generally known, and are available in commercial software packages.

The nucleotides can also be used as probes to identify genomic DNA or gene expression in a biological specimen, as described above and as is well established in the art. Briefly, DNA or mRNA is isolated from a cell sample. Detection of mRNA hybridizing to the subject sequence is indicative of gene expression in the sample. The mRNA can be amplified by RT-PCR, using reverse transcriptase to form a complementary DNA strand, followed by polymerase chain reaction amplification using primers specific for the subject DNA sequences. Alternatively, the mRNA sample is separated by gel electrophoresis, transferred to a suitable support, e.g., nitrocellulose, nylon, etc., and then probed with a fragment of the subject nucleotides as a probe. Other techniques, such as oligonucleotide ligation assays, in situ hybridizations, and hybridization to probes arrayed on a solid chip may also find use.

Targeted Mutations for In Vivo and Ili Vitro Models

The sequence of a gene according to the subject invention, including flanking promoter regions and coding regions, can be mutated in various ways known in the art to generate targeted changes, i.e., changes in promoter strength, or sequence of the encoded protein, etc. The DNA sequence or protein product of such a mutation will usually be substantially similar to the sequences provided herein. The sequence changes can be substitutions, insertions, deletions, or a combination thereof. Deletions can further include larger changes, such as deletions of a domain or exon.

Techniques for in vitro mutagenesis of cloned genes are known. Examples of protocols for site specific mutagenesis may be found in Gustin et al., 1993; Barany 1985; Colicelli et al., 1985; Prentki et al., 1984. Methods for site specific mutagenesis can be found in Sambrook et al., 1989,(pp. 15.3-15.108); Weiner et al., 1993; Sayers et al. 1992; Jones and Winistorfer; Barton et al., 1990; Marotti and Tomich 1989; and Zhu, 1989. Such mutated genes can be used to study structure-function relationships of the subject proteins, or to alter properties of the protein that affect its function or regulation. Other modifications of interest include epitope tagging, e.g., with hemagglutinin (HA), FLAG, or c-myc. For studies of subcellular localization, fluorescent fusion proteins can be used.

The subject nucleic acids can be used to generate transgenic, non-human animals and/or site-specific gene modifications in cell lines; suitable methods are known in the art (Grosveld and Kollias, 1992; Hooper, 1993; Murphy and Carter, 1993; Pinkert, 1994). Thus, in some embodiments, the invention provides a non-human transgenic animal comprising, as a transgene integrated into the genome of the animal, a nucleic acid molecule comprising a sequence encoding a subject polypeptide in operable linkage with a promoter, such that the subject polypeptide-encoding nucleic acid molecule is expressed in a cell of the animal. Either a complete or partial sequence of a gene native to the host can be introduced. Alternatively, a complete or partial sequence of a gene exogenous to the host animal, e.g., a human sequence of the subject invention, can be introduced. Transgenic animals can be made through homologous recombination, where the endogenous locus is altered. Thus, DNA constructs for homologous recombination will comprise at least a portion of the human gene or of a gene native to the species of the host animal, wherein the gene has the desired genetic modification(s), and includes regions of homology to the target locus. Methods for generating mammalian cells having targeted gene modifications through homologous recombination are known in the art (Keown et al., 1990).

Alternatively, a nucleic acid construct is randomly integrated into the genome. Vectors for stable integration include plasmids, retroviruses and other animal viruses, and YACs. DNA constructs for random integration need not include regions of homology to mediate recombination.

Conveniently, markers for positive and negative selection are included. A detectable marker, such as lac Z can be introduced into a locus at which up-regulation of expression will result in a detectable change in phenotype.

Transformed ES or embryonic cells can be used to produce transgenic animals. An embryonic stem (ES) cell line can be a source of embryonic stem cells, or they can be newly obtained from a host animal, e.g., a mouse, rat, or guinea pig. The cells are grown on an appropriate fibroblast-feeder layer or in the presence of leukemia inhibiting factor (LIF). Following transformation, the cells are plated for growth onto a feeder layer in an appropriate medium. Cells containing the relevant construct can be detected by employing a selective medium and analyzing them for the occurrence of homologous recombination or integration of the construct. Positive colonies can be used for embryo manipulation and blastocyst injection. Blastocysts are obtained from 4 to 6 week old super-ovulated females. The ES cells are trypsinized, and the modified cells are injected into the blastocoel of the blastocyst. After injection, the blastocysts are returned to each uterine horn of pseudopregnant female animals that proceed to term. The resulting offspring are screened for the construct. By providing for a different phenotype of the blastocyst and the genetically modified cells, chimeric progeny can be readily detected.

The chimeric animals are screened for the presence of the modified gene and males and females having the modification are mated to produce homozygous progeny. If the gene alterations cause lethality at some point in development, tissues or organs can be maintained as allogeneic or congenic grafts or transplants, or in in vitro culture. The transgenic animals can be any non-human mammal.

The modified cells or animals are useful in the study of gene function and regulation. For example, a series of small deletions and/or substitutions can be made in the host's native gene to determine the role of different exons in biological processes such as oncogenesis or signal transduction. Of interest is the use of genes to construct transgenic animal models for cancer, where expression of the subject protein is specifically reduced or absent. Specific constructs of interest include anti-sense constructs, which will block expression, expression of dominant negative mutations, and gene over-expression.

One can also provide for expression of the gene, e.g., a subject gene, or variants thereof, in cells or tissues where it is not normally expressed, at levels not normally present in such cells or tissues, or at abnormal times of development. One can also generate host cells (including host cells in transgenic animals) that comprise a heterologous nucleic acid molecule which encodes a polypeptide which functions to modulate expression of an endogenous promoter or other transcriptional regulatory region, or the biological activity of a subject polypeptide. The transgenic animals can also be used in functional studies, for example drug screening, to determine the effect of a candidate drug on a biological activity.

While the present invention has been described with reference to the specific embodiments thereof, it should be understood by those skilled in the art that various changes may be made and equivalents may be substituted without departing from the true spirit and scope of the invention. In addition, many modifications can be made to adapt a particular situation, material, composition of matter, process, process step or steps, to the objective, spirit and scope of the present invention. All such modifications are intended to be within the scope of the claims appended hereto.

It is to be understood that both the foregoing general description and the following detailed description are exemplary and explanatory only and are not restrictive of the invention, as claimed. Moreover, it must be understood that the invention is not limited to the particular embodiments described, as such may, of course, vary. Further, the terminology used to describe particular embodiments is not intended to be limiting, since the scope of the present invention will be limited only by its claims.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test the invention. Further, all publications mentioned herein are incorporated by reference.

It must be noted that, as used herein and in the appended claims, the singular forms “a,” “or,” and “the” include plural referents unless the context clearly dictates otherwise. Thus, for example, reference to “a subject polypeptide” includes a plurality of such polypeptides and reference to “the agent” includes reference to one or more agents and equivalents thereof known to those skilled in the art, and so forth.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### EXAMPLES

The examples, which are intended to be purely exemplary of the invention and should therefore not be considered to limit the invention in any way, also describe and detail aspects and embodiments of the invention discussed above. The examples are not intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The polypeptides described in the Tables and Sequence Listing were tested in the assays described below. The data presented in Examples 1-13 represent the results of high throughput screening assays.

### Example 1

Glucose to Glycogen Assay (Gu2Gy3T3)

The objective of the Gu2Gy3T3 assay is to select secreted factors that stimulate glucose uptake in adipocytes. The assay is based on the principle that the glucose taken up is metabolized and stored as glycogen. The glucose uptake activity is quantified by measuring labeled glycogen, e.g., radiolabeled glycogen, synthesized after the addition of the labeled glucose (³H-glucose) and conditioned medium with the potentially active factor of interest. This assay may, e.g., be performed in a 96-well plate format, and may, e.g., be performed with 3T3L1 cells. Glucose to glycogen assays can be performed, e.g., as described by Ludvigsen et al. (1979) or Urs\acute{ø} et al. (1999).

When this assay was performed as described above, HG1014930 (CLN00156143) obtained from testis tissue and HG1014958 (CLN00185984) obtained from breast tissue stimulated glucose uptake in adipocytes.

### Example 2

Lipogenesis in Juvenile Rat Adipocytes (1 nM Insulin) (LPGNJRAHI)

The objective of the LPGNJRAHI assay is to select secreted factors that modulate lipogenesis in human adipocytes. The proteins are assayed in buffer containing 1 nM insulin, in order to discover insulin inhibitors. The assay is based on the principle that insulin induces adipocytes to incorporate glucose into fat depots, and labeled glucose, e.g., D-3-³H-glucose, added to the medium will result in label incorporation, e.g., tritium incorporation, into the lipid pool. Lipids are extracted, e.g., using MicroScint-E scintillator, prior to measuring radioactivity. The level of glucose uptake by the adipocytes is measured by quantifying the amount of incorporated label. This assay may, e.g., be performed in a 96-well plate format. Lipogenesis assays can be performed, e.g., as described by Schaffer et al. (2003).

When this assay was performed as described above, HG1014954 (CLN00018656) obtained from bladder, brain, lung, and spleen tissue and HG1014958 (CLN000185984) obtained from breast tissue inhibited the effect of insulin on lipogenesis.

### Example 3

Lipogenesis in Juvenile Rat Adipocytes (100 pM Ins) (LPGNJRALI)

The objective of the LPGNJRALI assay is to select secreted factors that modulate lipogenesis in human adipocytes. The proteins are assayed in buffer containing 100 pM insulin in order to discover insulin potentiators. The assay is based on the principle that insulin induces adipocytes to incorporate glucose into fat depots, and labeled glucose, e.g., D-3-³H-glucose, added to the medium will result in label incorporation, e.g., tritium incorporation, into the lipid pool. Lipids are extracted, e.g., using MicroScint-E scintillator, prior to measuring radioactivity. The level of glucose uptake by the adipocytes is measured by quantifying the amount of incorporated label. This assay may, e.g., be performed in a 96-well plate format. Lipogenesis assays can be performed, e.g., as described by Schaffer et al. (2003).

When this assay was performed as described above, HG1014905 (CLN00082984) obtained from muscle tissue; HG1014958 (CLN00156600) obtained from testis tissue; HG1015022 (CLN00192344) obtained from lung tissue; and HG1014932 (CLN00062536) obtained from kidney tissue potentiated insulin-mediated lipogenesis.

### Example 4

Lipogenesis in Juvenile Rat Adipocytes (Insulin-Free) (LPGNJRANI)

The objective of the LPGNJRANI assay is to select secreted factors that modulate lipogenesis in human adipocytes. The proteins are assayed in insulin-free buffer, in order to discover insulin mimics. The assay is based on the principle that insulin induces adipocytes to incorporate glucose into fat depots, and labeled glucose, e.g., D-3-³H-glucose, added to the medium will result in label incorporation, e.g., tritium incorporation, into the lipid pool. Lipids are extracted, e.g., using MicroScint-E scintillator, prior to measuring radioactivity. The level of glucose uptake by the adipocytes is measured by quantifying the amount of incorporated label. This assay may, e.g., be performed in a 96-well plate format. Lipogenesis assays can be performed, e.g., as described by Schaffer et al. (2003).

When this assay was performed as described above, HG1014930 (CLN00156143) obtained from testis tissue; HG1014958 (CLN00156600) obtained from testis tissue; HG1014986 (CLN00138883) obtained from intestine, pancreas, and stomach tissue; HG1015022 (CLN00192344) obtained from lung tissue; and HG1014954 (CLN00118656) obtained from bladder, brain, lung, and spleen tissue induced adipocytes to incorporate glucose into fat deposits.

### Example 5

PCK1 Gene Expression in Rat H4IIE Hepatoma Cells

(PCK1bDNAH4IIE)

The objective of the PCK1bDNAH4IIE assay is to identify factors that down-regulate dexamethasone (dex)-induced PCK1 mRNA expression. It can be performed, e.g., in rat H4IIE hepatoma cell lines using the Genospectra branched DNA (bDNA) detection method according to the manufacturer's instructions (Wu et al., 2003).

When this assay was performed as described above, HG1014917 (CLN00142812) obtained from colon tissue; HG1015007 (CLN00200943) obtained from prostate tissue; HG1015074 (CLN00202085) obtained from colon tissue; and HG1014974 (CLN00041527) obtained from adrenal gland tissue down-regulated dex-induced PCK1 mRNA expression.

### Example 6

Lipolysis Induction Assay with Isolated Rat Fat Cells (RatLipoInd)

The objective of the RatLipoInd assay is to select secreted factors that induce lipolysis, the process in which triglycerides are hydrolyzed into glycerol and free fatty acids, in adipocytes. Isoproterenol, a non-specific β-adrenergic agonist that can induce lipolysis via β-adrenergic receptors is a suitable positive control. The lipolysis activity can be quantified, e.g., by measuring the free fatty acid level in the adipocyte medium after the addition of the conditioned medium. This assay may, e.g., be performed in a 96-well plate format. Lipolysis assays can be performed, e.g., as described by Kuo et al., 1969; and Jong et al. (1996).

When this assay was performed as described above, HG1014986 (CLN00138883) obtained from intestine, pancreas, and stomach tissue induced lipolysis. When this assay was performed as described above, HG1015022 (CLN00192344) obtained from lung tissue inhibited lipolysis.

### Example 7

Lipolysis Fluorogenic Induction Assay (RatLipoIndFl))

The objective of the RatLipoIndFl assay is also to select secreted factors that induce lipolysis, the process in which triglycerides are hydrolyzed into glycerol and free fatty acids, in adipocytes. Isoproterenol, a non-specific B-adrenergic agonist that can induce lipolysis via 13-adrenergic receptors is a suitable positive control. The lipolysis activity can be quantified, e.g., by measuring the free fatty acid level in the adipocyte medium after the addition of the conditioned medium. This assay may, e.g., be performed in a 96-well plate format. It may be performed using the fluorometric substrate Amplex Red (to provide a sensitive asay with a short assay duration) and isolated rat fat cells. Lipolysis assays can be performed, e.g., as described by Kuo et al., 1969; and Jong et al. (1996); Amplex Red can be obtained from Molecular Probes (Eugene Oreg.) and used as described by Wentworth et al. (2001).

### Example 8

Lipolysis Inhibition Assay (RatLipoInh))

The objective of the RatLipoInh assay is to select secreted factors that inhibit lipolysis, the process in which triglycerides are hydrolyzed into glycerol and free fatty acids, in adipocytes. Isoproterenol, a non-specific 3-adrenergic agonist that can induce lipolysis via β-adrenergic receptors is used to induce lipolysis. Insulin, which is used as a positive control, inhibits this induction. The lipolysis activity is quantified by measuring the free fatty acid level in the adipocyte medium after the addition of isoproterenol and conditioned medium. This assay may, e.g., be performed in a 96-well plate format. Lipolysis inhibition assays can be performed, e.g., as described by Castan et al., 1999.

When this assay was performed as described above, HG1014996 (CLN00042242) obtained from muscle tissue; HG1015037 (CLN00114957) obtained from bladder, brain, and lung tissue; HG1015074 (CLN00202085) obtained from colon tissue; and HG1014986 (CLN00138883) obtained from intestine, pancreas, and stomach tissue inhibited isoproterenol-induced lipolysis. When this assay was performed as described above, HG1014930 (CLN00156143) obtained from testis tissue and HG1015007 (CLN00200943) obtained from prostate tissue decreased the inhibitory action of insulin on isoproterenol-induced lipolysis.

### Example 9

Lipolysis Fluorogenic Inhibition Assay (RatLipoInhFl)

The objective of the RatLipoInhFl assay is to select secreted factors that inhibit lipolysis, the process in which triglycerides are hydrolyzed into glycerol and free fatty acids, in adipocytes. Isoproterenol, a non-specific β-adrenergic agonist that can induce lipolysis via β-adrenergic receptors is used to induce lipolysis. Insulin, which is used as positive control, inhibits this induction. The lipolysis activity is quantified by measuring the free fatty acid level in the adipocyte medium after the addition of isoproterenol and conditioned medium. This assay may, e.g., be performed in a 96-well plate format. Lipolysis inhibition assays can be performed, e.g., as described by Castan et al., 1999. This assay may, e.g., be performed in a 96-well plate format. It may be performed on isolated rat fat cells using the fluorometric substrate Amplex Red (to provide a sensitive asay with a short assay duration). Lipolysis assays can be performed, e.g., as described by Kuo et al., 1969; and Jong et al. (1996); Amplex Red can be obtained from Molecular Probes (Eugene Oreg.) and used as described by Wentworth et al. (2001).

### Example 10

Peripheral Blood NK Cell Proliferation Assay (NKGlo)

The objective of the NKGlo assay is to identify factors affecting the proliferation of peripheral blood NK cells. The assay is based on the principle that ATP levels increase with increased cell number upon cell proliferation. Cell proliferation can be measured, e.g., by measuring ATP bioluminescence (Crouch et al. (1993), further described in Promega's CellTitreGlo Technical Bulletin No. 288). For example, ATP levels can be measured by measuring the intensity of luminescence produced in the presence of luciferase and luciferin. This assay may, e.g., be performed in a 96-well plate format.

When this assay was performed as described above, HG1014917 (CLN00142812) obtained from colon tissue and HG1014954 (CLN00118656) obtained from bladder, brain, lung, and spleen tissue stimulated peripheral blood NK cell proliferation. When this assay was performed as described above, HG1014930 (CLN00156143) obtained from testis tissue and HG1014958 (CLN00156600) obtained from testis tissue inhibited peripheral blood NK cell proliferation.

### Example 11

Activated Primary B Cell Proliferation Assay (aBPro4)

The objective of the aBPro4 assay is to select secreted factors that modulate the proliferation of human activated primary B cells. The assay is based on the principle that ATP levels increase with increased cell number upon cell proliferation. Cell proliferation can be measured, e.g., by measuring ATP bioluminescence (Crouch et al. (1993), further described in Promega's CellTitreGlo Technical Bulletin No. 288). For example, ATP levels can be measured by measuring the intensity of luminescence produced in the presence of luciferase and luciferin. This assay may, e.g., be performed in a 96-well plate format. Fixed CHO-CD40L cells can be used to activate B cells.

### Example 12

Activated Monocytes Proliferation Assay (aMonPro3)

The objective of the aMonPro3 assay is to select secreted factors that modulate proliferation of human activated primary monocytes. The assay is based on the principle that ATP levels increase with increased cell number upon cell proliferation. Cell proliferation can be measured, e.g., by measuring ATP bioluminescence (Crouch et al. (1993), further described in Promega's CellTitreGlo Technical Bulletin No.288). For example, ATP levels can be measured by measuring the intensity of luminescence produced in the presence of luciferase and luciferin. This assay may, e.g., be performed in a 96-well plate format. Mouse IgG2a can be used to activate monocytes.

### Example 13

Primary T Cell Proliferation Assay (aTPro4)

The objective of the aTPro4 assay is to identify factors affecting the proliferation of primary T cells. The assay is based on the principle that ATP levels increase with increased cell number upon cell proliferation. T cell proliferation can be measured, e.g., by measuring ATP bioluminescence (Tamada et al., 2000; Crouch et al., 1993; further described in Promega's CellTitreGlo Technical Bulletin No. 288). For example, ATP levels can be measured by measuring the intensity of luminescence produced in the presence of luciferase and luciferin. This assay may, e.g., be performed in a 96-well plate format.

### REFERENCES

The specification is most thoroughly understood in light of the following references, all of which are hereby incorporated by reference in their entireties. The disclosures of the patents and other references cited above are also hereby incorporated by reference.
1. Agou, F., et al. (1996) Biochemistry 35:15,322-15,331.
2. Agrawal, S., Crooke, S. T. eds. (1998) Antisense Research and Application (Handbook of Experimental Pharmacology, Vol 131), Springer-Verlag New York, Inc.
3. Alberts, B., et al. (1994) Molecular Biology of the Cell 3^{rd} ed. Garland Publishing, Inc.
4. Alexander, D. R. (2000) Semin. Immunol 12:349-359.
5. Allison, A. C. (2000) Immunopharmacology 47:63-83.
6. Altschul, S. F., et al. (1997) Nucleic Acids Res. 25:3389-3402.
7. Altschul, S. F., et al. (1990) J. Mol. Biol. 215:403-410.
8. Amor, J. C., et al. (1994) Nature 372:704-708.
9. Andreeff, M., et al. (1999) Introduction to Fluorescence In Situ Hybridization: Principles and Clinical Applications. John Wiley & Sons.
10. Andres, D. A., et al. (1997) Arch. Biochem. Biophys. 346:113-124.
11. Ansel, H. C., et al., eds. (2004) Pharmaceutical Dosage Forms and Drug Delivery Systems. 8th ed. Lippincott Williams & Wilkins.
12. Attardi, L. D., et al. (1999) Cell Mol. Life Sci. 55:48-63.
13. Aubry, M., et al. (1992) Genomics 13:641-648.
14. Ausubel, F., et al. eds. (1999) Short Protocols in Molecular Biology, 4^{th} ed. Wiley & Sons.
15. Baksh, S., et al. (2000) Semin. Immunol. 12:405-415.
16. Ballance, D. J., et al. (1983) Biochem. Biophys. Res. Commun. 112:284-289.
17. Barany, F. (1985) Gene 37:111-123.
18. Barnes, D., Sato, G. (1980) Anal. Biochem. 102:255-270.
19. Barton, M. C., et al. (1990) Nucleic Acids Res. 18:7349-7355.
20. Bashkin, J. K., et al. (1995) Appl. Biochem. Biotechnol. 54:43-56.
21. Bassett, D. E., et al. (1999) Nature Genetics 21:51-55.
22. Bast, R. C., et al., eds. (2000) Cancer Medicine. 5th ed. B.C. Decker, Inc.
23. Bateman, A., et al. (2000) Nucleic Acids Research 30:276-280.
24. Battini, R., et al. (1987) J. Biol. Chem. 262:4355-4359.
25. Beach, D., et al. (1982) Nature 300:706-709.
26. Beigelman, L., et al. (1995) Nucleic Acids Res. 23:4434-4442.
27. Bennett, J. (2000) Curr. Opin. Mol. Ther. 2:420-425.
28. Berinstein, N. L. (2002) J. Clin. Oncol. 20:2197-2207.
29. Bibikova, M., et al. (2003) Science 300:764.
30. Birney, E., et al. (2000) Genome Res. 10:547-548.
31. Blackwell, J. M., et al. (1995) Mol. Med. 1:194-205.
32. Bodzioch, M., (1999) Nat. Genet. 1999 22:347-351.
33. Bonifaci, N., et al. (1997) Proc. Natl. Acad. Sci. 94:5055-5060.
34. Bono, H., et al. (2002) Nucleic Acids Res. 30:116-118.
35. Boshart, M., et al. (1985) Cell 41:521-530.
36. Bowtell, D. D. L. (1999) Nature Genetics 21:25-32.
37. Brenner, S., et al. (2000) Proc. Natl. Acad. Sci. 97:1665-1670.
38. Brinster, R. L., et al. (1981) Cell 27:223-231.
39. Brock, G. (2000) Drugs Today36:125-134.
40. Brown, J. R., et al. (1985) Mol. Cell Biol. 5:1694-1706.
41. Brown, P. O, et al. (1999) Nature Genetics 21:33-37.
42. Brunelleschi, S., et al. (2002) Curr. Pharm. Des. 8:1959-1972.
43. Brutlag, D. L., et al. (1993). Computers and Chemistry 17:203-207.
44. Capecchi, M. R. (1989) Genet. 5:70-76.
45. Carbonell, L. F., et al. (1988) Gene 73:409-418.
46. Castan et al. (1999) Biochem. J. 339:281-289.
47. Chakravarty, A. (1999) Nature Genetics21:56-60.
48. Chalifour, L. E., et al. (1994) Anal. Biochem. 216: 299-304.
49. Chalut, C., et al. (1995) Gene 161:277-282.
50. Chang, A. C., et al. (1978) Nature 275:617-624.
51. Chang, M. S., et al. (2000) Biochem. Biophys. Res. Commun. 279:732-737.
52. Chen, F. W., et al. (1998) Int. Rev. Immunol. 18:429-448.
53. Chen, S. Y., et al. (1994) Hum. Gene Ther. 5:595-601.
54. Cheng, W. F., et al. (2001) J. Clin. Invest. 108:669-678.
55. Cheung, V. G., et al. (1999) Nature Genetics 21:15-19.
56. Chien, C., et al. (1991) Proc. Natl. Acad. Sci. 88:9578-9581.
57. Christa, L., et al. (1994) Gastroenterology 106:1312-1320.
58. Chuang, V. T., et al. (2002) Pharm. Res. 19:569-577.
59. Clark, C. M., et al. (2003) Ann. Intern. Med. 138:400-410.
60. Clark, H. F., et al. Genome Res. 13:2265-2270; Epublished Sep. 15, 2003.
61. Cole, K. A., et al. (1999) Nature Genetics 21:38-41.
62. Colicelli, J., et al. (1985) Mol. Gen. Genet. 199:537-539.
63. Collins, F. S. (1999) Nature Genetics 21:2.
64. Comuzzie, A. G., et al. (1998) Science 280:1374-1377.
65. Cormand, B., et al. (1997) Hum. Genet. 100:75-79.
66. Cregg, J. M., et al. (1985) Mol. Cell. Biol. 5:3376-3385.
67. Crooke, S. T. (1996) Med. Res. Rev. 16:319-344.
68. Crouch et al. (1993) J. Immunol. Meth. 160:81-88.
69. Crouch, R. J. (1990) New Biol. 2:771-777.
70. Curcio, L. D., et al. (1997) Pharmacol. Ther. 74:317-332.
71. Das, S., et al. (1984) J. Bacteriol. 158:1165-1167.
72. Davidow, L. S., et al. (1987) Curr. Genet. 11:377-383.
73. de Boer, H. A., et al. (1993) Proc. Natl. Acad. Sci. 80:21-25.
74. De Louvencourt, L., et al. (1983) J. Bacteriol. 154:737-742.
75. Deasy, B. M., et al. (2002) Curr. Opin. Mol. Ther. 4:382-389.
76. Delahunty, C., et al. (1996) Am. J. Human Genetics 58:1239-1246.
77. Deutscher, M. P., et al. eds. (1990) Guide to Protein Purification: Methods in Enzymology. (Methods in Enzymology Series, Vol 182). Academic Press.
78. Dieffenbach, C. W., et al. eds. (1995) PCR Primer: A Laboratory Manual. Cold Spring Harbor Laboratory Press.
79. Dijkema, R., et al. (1985) EMBO J. 4:761-767.
80. Doerfler, W., et al. eds. (1987) The Molecular Biology Of Baculoviruses. Springer-Verlag, Inc.
81. Doll, A., et al. (2001) Cytogenet. Cell Genet. 95:20-27.
82. Doolittle, R. F., et al. eds. (1996) Computer Methods for Macromolecular Sequence Analysis. 1st ed. Academic Press.
83. Ducrest, A. L., et al. (2002) Oncogene 21:541-52.
84. Dutoit, V., Taub, et al. (2002) J. Clin. Invest. 110:1813-1822.
85. Egilsson, V., et al. (1986) J. Gen. Microbiol. 132:3309-3313.
86. Ehrhardt, G. R., et al. (2001) Oncogene 20:188-197.
87. Espejo, A., et al. (2002) Biochem. J. 367:697-702.
88. Everett, R. D., et al. (1997) EMBO J. 16:1519-1530.
89. Fanning, A. S., et al. (1999) Curr. Opin. Cell Biol. 11:432-439.
90. Fields, S., Song, O. (1989) Nature 340:245-246.
91. Fiers, W. (1991) FEBS Lett. 285:199-212.
92. Fisch, P., Forster, et al. (1993) Oncogene 8:3271-3276.
93. Fishman, P. S., et al. (2002) Curr. Neurol. Neurosci. Rep. 2:296-302.
94. Forgac, M. (1999) J. Biol. Chem. 274:12,951-12,954.
95. Frank, I. (2002) Clin. Lab. Med. 22:741-757.
96. Frithz, G., et al. (1976) Ups J Med Sci. 81:155-158.
97. Funakoshi, I., et al. (1992) Arch. Biochem. Biophys. 295:180-187.
98. Furth, P. A., et al. (1992) Anal. Biochem. 205:365-368.
99. Gaillardin, C., et al. (1987) Yarrowia lipolytica. Curr. Genet. 11:369-375.
100. Gao, X., et al. (2002) Breast Cancer Res. 4:182-186.
101. Gao, Y., et al. (1994) J. Cell Biol. 125:989-996.
102. Gaudilliere, B., et al. (2002) J. Biol. Chem. 277:46,442-46,446.
103. Gavrieli, Y., et al. (1992) J. Cell Biol. 119:493-501.
104. Geffen D. B., et al. (2002) Isr. Med. Assoc. J 4:1124-31.
105. Gennaro, A. R. (2003) Remington: The Science and Practice of Pharmacy with Facts and Comparisons: DrugfactsPlus. 20th ed. Lippincott Williams & Williams.
106. Ghofrani, H. A., et al. (2003) J. Am. Coll. Cardiol. 42:158-164.
107. Gillingham, A. K., et al. (2002) Mol. Biol. Cell 13:3761-3774.
108. Gingras, M. C., et al. (2002) Mol. Immunol. 38:817-824.
109. Girschick, H. J., et al. (2002) Arthritis. Rheum. 46:1255-1263.
110. Gmeiner, W. H., et al. (2001) Cell Biochem. Biophys. 35:127-140.
111. Goeddel, D. V., et al. (1979) Nature 281:544-548.
112. Goldstein, L. S. B., et al. (2000) Annu. Rev. Neurosci. 23:39-71.
113. Golovkina, T. V., et al. (1992) Cell 69:637-645.
114. Gonnet, G. H., et al. (1992) Science 256:1443-1445.
115. Gordan, J. D., et al. (2002) Cytotherapy 4:317-327.
116. Gorman, C. M., et al. (1982) Proc. Natl. Acad. Sci. 79:6777-6781.
117. Gray, T. A., et al. (2002) Genomics. 66:76-86.
118. Griffiths, A. J. F., et al. (1999) Introduction to Genetic Analysis. 7^{th} ed. W. H. Freeman.
119. Griffiths, M., et al. (1997) Nat. Med. 3:89-93.
120. Grosschedl, R., Baltimore, D. (1985) Cell 41:885-897.
121. Grosveld, F., Kollias, G., eds. (1992) Transgenic Animals. 1^{st} ed. Academic Press.
122. Gustin, K., Burk, R. D. (1993) Biotechniques 14:22-24.
123. Hacia, J. G. (1999) Nat. Genetics 21:42-47.
124. Hadano, S., et al. (2001) Genomics 71:200-213.
125. Hall, M., et al. (1985) Clin. Chem. 31:1689-1691.
126. Ham, R. G., et al. (1979) Methods Enzymnol. 58:44-93.
127. Hanada, T., et al. (2000) J. Biol. Chein. 275:28,774-28,784.
128. Harlow, E., et al. eds. (1988) Antibodies. A Laboratory Manual. Cold Spring Harbor Laboratory.
129. Harlow, E., et al. eds. (1998) Using Antibodies: A Laboratory Manual: Portable Protocol NO. I Cold Spring Harbor Laboratory.
130. Hartmann, G., et al. eds. (1999) Manual of Antisense Methodology (Perspectives in Antisense Science). 1^{st} ed. Kluwer Law International.
131. Hassanzadeh, G. H. G., et al. (1998) FEBS Lett. 437:75-80.
132. Hawes, J. W., et al. (1996) J. Biol. Chem. 271:26,430-26,434.
133. Heath, J. K., et al. (1997) Proc. Natl. Acad. Sci. 94:469-474.
134. Heiser, A., Coleman, et al. (2002) J. Clin. Invest. 109:409-417.
135. Henningson, C. T. Jr., et al. (2003) J. Allergy Clin. Immunol. 111:S745-S753.
136. Hinnen, A., et al. (1978) Proc. Natl. Acad. Sci. 75:1929-1933.
137. Hirsch, D. S., et al. (2001) J. Biol. Chem. 276:875-883.
138. Ho, L. W., et al. (2001) Psychol. Med. 31:3-14.
139. Hollis, G. F., et al. (1989) Proc. Natl. Acad. Sci. 86:5552-5556.
140. Homo-Delarche, F. (2001) Braz. J. Med. Biol. Res. 34:437-447.
141. Hong, G. F. (1982) Biosci. Rep. 2:907-912.
142. Hoogenboom, H. R., et al. (1998) Immunotechnology 4:1-20.
143. Hooper, M. L. (1993) Embryonal Stem Cells: Introducing Planned Changes into the Animal Germline. Gordon & Breach Science Pub.
144. Hoozemans, J. J., et al. (2002) Drugs Today (Barc) 38:429-443.
145. Houseman, B. T., et al. (2002) Nature Biotechnol. 20:270-274.
146. Howard, G. C., et al. (2000) Basic Methods in Antibody Production and Characterization. CRC Press.
147. Huynh, D. P., et al. (2003) Hum. Mol. Genet. 12:1485-1496.
148. Ikeda, A., et al. (2002) J. Cell Sci. 115(Pt 1):9-14.
149. Ito, H., et al. (1978) J. Bacteriol. 153:163-168.
150. Jameson, D. M., et al. (1995) Methods Enzymol. 246:283-300.
151. Janeway, C. A., et al. (2001) Immunobiology. 5^{th} ed. Garland Publishing.
152. Jeffery, P., et al. (2002) Novartis Found. Symp. 248:51-75, 277-82.
153. Jimbo, T., et al. (2002) Nat. Cell Biol. 4:323-327.
154. Joberty, G., et al. (1999) Mol. Cell Biol. 19:6585-6597.
155. Johns, T. G., et al. (1997) Mol. Immunol. 34:33-38.
156. Jolliffe, C. N., et al. (2000) Biochem. J. 351:557-565.
157. Jones, D. H., et al. (1992) Biotechniques 12:528-530.
158. Jones, P., ed. (1998a) Vectors: Cloning Applications: Essential Techniques, John Wiley & Son, Ltd.
159. Jones, P., ed. (1998b) Vectors: Expression Systems: Essential Techniques, John Wiley & Son, Ltd.
160. Jong et al. (1996) Arteriosclerosis, Thrombosis, and Vascular Biol. 16:934-940.
161. Jorgensen, C., et al. (2003) Gene Ther. 10:928-931.
162. Jost, C. R. et al. (1994) J. Biol. Chem. 269:26,267-26,273.
163. Joulin, V., et al. (1995) Eur. J. Biochem. 232:620-626.
164. Jurcic, J. G., et al. (2000) Curr. Opin. Hematol. 7:247-254.
165. Jury, J. A., et al. (1999) Mol. Hum. Reprod. 5:1127-1134.
166. Kabat, E. A., Wu T. T. (1991) J. Immunol. 147:1709-1719.
167. Kamitani, T., et al. (1997) J. Biol. Chem. 272:14,001-14,004.
168. Kantoff, P. W., et al. (2001) J. Clin. Oncol. 9:3025-3028.
169. Kao, P. N., et al. (1994) J. Biol. Chem. 269:20,691-20,699.
170. Karanazanashvili, G., et al. (2003) J. Urol. 169:445-457.
171. Kari, C., et al. (2003) Cancer Res. 63:1-5.
172. Kelly, J. M., (1985) EMBO J. 4:475-479.
173. Kenmochi, N., et al. (1998) Genome Res. 8:509-523.
174. Keown, W.A., et al. (1990) Methods Enzymol. 185:527-537.
175. Kibbe, A. H., ed. (2000) Handbook of Pharmaceutical Excipients, 3^{rd} ed. Pharmaceutical Press.
176. Kirkpatrick, K. L., et al. (2001) Eur. J. Surg. Oncol. 27:754-760.
177. Kirsch, K. H., et al. (1999) Proc. Natl. Acad. Sci. 96:6211-6216.
178. Kiryu-Seo, S., et al. (2000) Proc. Natl. Acad. Sci. 97:4345-4350.
179. Klarman, G. J., et al. (2002) AIDS Rev. 4:183-194.
180. Knutson, K. L., et al. (2001) J. Clin. Invest. 107:477-484.
181. Kobayashi, M., et al. (1999) Proc. Natl. Acad. Sci. 96:4814-4819.
182. Kolonin, M. G., et al. (1998) Proc. Natl. Acad. Sci. 95:14,266-14,271.
183. Korner, C., et al. (1999) EMBO J. 18:6816-6822.
184. Kothapalli, R., et al. (1997) J. Clin. Invest. 99:2342-2350.
185. Kovalenko, O. V., et al. (1997) Nucleic Acids Res. 25:4946-4953.
186. Kratzschmar, J., et al. (1996) J. Biol. Chem. 271:4593-4596.
187. Ku, D. H., et al. (1990) J. Biol. Chem. 265:16,060-16,063.
188. Kuisle, O., et al. (1999) Tetrahedron Lett. 40:1203-1206.
189. Kunze, G. et al. (1985) J. Basic Microbiol. 25:141-144.
190. Kuo et al (1969) J. Biol. Chem. 244:2252-2260.
191. Kurtz, M. B., et al. (1986) Mol. Cell. Biol. 6:142-149.
192. Kyo, S., et al. (2000) Histol. Histopathol. 15:813-824.
193. Lander, E. S. (1999) Nature Genetics 21:3-4.
194. Lander, E. S., et al. (2001) Nature 409:860-921.
195. Lasham, A., et al. (2003) J. Biol. Chem. Epub ahead of print, Jun. 30, 2003.
196. Lashkari, A., et al. (1999) Clin. Pediatr. 38:189-208.
197. Lavedan, C. (1998) Genome Res. 8:871-880.
198. Lebacq-Verheyden, A. M., et al. (1988) Mol. Cell. Biol. 8:3129-3135.
199. Lees-Miller, S. P., et al. (1989) J. Biol. Chem. 264:2431-2437.
200. Lerch, M. M., et al. (2000) Med. Clin. North Amer. 84:549-563.
201. Lerner, R. A. (1982) Nature 299:592-596.
202. Li, E., et al. (1996) Eur. J. Biochem. 238:631-638.
203. Lim, D., et al. (August 2002) J. Virol. 76:8360-8373.
204. Lin, B., et al. (1993) Hum. Mol. Genet. 2:1541-1545.
205. Lin, W. J., et al. (1996) J. Biol. Chem. 271:15,034-15,044.
206. Lin, X., et al. (1999) J. Biol. Chem. 274:36,125-36,131.
207. Linnenbach, A. J., et al. (1993) Mol. Cell Biol. 13:1507-1515.
208. Linstedt, A. D., Hauri, et al. (1993) Mol. Biol. Cell 4:679-693.
209. Lipshutz, R. J., et al. (1999) Nature Genetics 21:20-24.
210. Liu, A. Y., et al. (1987a) Proc. Natl. Acad. Sci. 84:3439-3443.
211. Liu, A. Y., et al. (1987b) J. Immunol. 139:3521-3526.
212. Lodish, H., et al. (1999) Molecular Cell Biology. 4th ed. W H Freeman & Co.
213. Loeffen, J. L., et al. (1998) Biochem. Biophys. Res. Commun. 253:415-422.
214. Los, M., et al. (2003) Drug Discov. Today 15:67-77.
215. Lovering R, et al. (1991) Nucleic Acids Res. 19:2921-2928.
216. Luckow, V., et al. (1988) Bio/Technology 6:47-55.
217. Ludvigsen et al. (1979) J. Biol. Chem. 254:1444-1446.
218. MacBeath, G., et al. (2000) Science 289:1760-1763.
219. Machesky, L. M., et al. (1999) Biochem. J. 328:105-112.
220. Machiels, J. P., et al. (2002) Semin. Oncol. 29:494-502.
221. Mackay, A., et al. (2003) Oncogene 22:2680-2688.
222. Maeda, S., et al. (1985) Nature 315:592-594.
223. Mahajan, M. A., et al. (2002) Mol. Cell Biol. 22:6883-6894.
224. Mahimkar, R. M., et al. (2000) J. Am. Soc. Nephrol., 11:595-603.
225. Mahnensmith, R. L., et al. (1985) J. Biol. Chem. 260:12,586-12,592.
226. Mangi, A. A., et al. (2003) Nat. Med. 9:1195-1201. Epublished Aug. 10, 2003.
227. Manning, G., et al. (2002) Science 298:1912-1934.
228. Marotti, K. R., et al. (1989) Gene Anal. Tech. 6:67-70.
229. Martel-Pelletier, J., et al. (2001) Best Pract. Res. Clin. Rheumatol. 15:805-829.
230. Martin, B. M., et al. (1988) DNA 7:99-106.
231. Massari, M. E., et al. (1998) Mol. Cell Biol. 18:3130-3139.
232. Matz, M. V., et al. (1999) Nat. Biotechnol. 17:969-973.
233. Mayer, B. J. (2001) J. CellSci. 114:1253-1263.
234. Mayer, T. U., et al. (1999) Science 286:971-974.
235. McGraw, R. A. III (1984) Anal. Biochem. 143:298-303.
236. McKusick, V. A. (2003) OMIM: Online Mendelian Inheritance in Man http:www.ncbi.nlm.nih.gov, #104300.
237. McPherson, M. J., et al. (2000) PCR Basics: From Background to Bench. Springer Verlag.
238. Merla, G., et al. (2002) Hum. Genet. 110:429-438.
239. Miki, H., et al. (2001) Proc. Natl. Acad. Sci. 98:7004-7011.
240. Milam, A. H., et al. (2002) Proc. Natl. Acad. Sci. 99:473-478.
241. Milligan, J. F., et al. (1993) J. Med. Chem. 36:1923-1937.
242. Mitch, W. E., et al. (1996). N. Engl. J. Med. 335:1897-1905.
243. Mitchell, D. A., et al. (2000) J. Clin. Invest. 106:1065-1069.
244. Miyajima A. (2002) Kokuritsu Iyakuhin Shokuhin Eisei Kenkyusho Hokoku 120:53-74.
245. Miyajima, A., et al. (1987) Gene 58:273-281.
246. Monfardini, C., et al. (1995) Bioconjugate Chem. 6:62-69.
247. Mori, N. (1997) Nihonz Shinkei Seishin Yakurigaku Zasshi 17:159-167.
248. Mortlock, D. P., et al. (1996) Genome Res. 6:327-335.
249. Murphy, D., et al. eds. (1993) Transgenesis Techniques: Principles and Protocols. Humana Press.
250. Myers, E. W., et al. (1988) Comput. Appl. Biosci. 4:11-7.
251. Nagata, K., et al. (1995) Proc. Natl. Acad. Sci. 92:4279-4283.
252. Nanda, S., et al. (2004) Expert Opin. Pharmacother. 5:1175-1186.
253. Naora, H. (1999) Immunol. Cell Biol. 77:197-205.
254. Needleman, S. B., et al. (1970) J. Mol. Biol. 48:443-453.
255. Nelson, N., et al. (1999) Physiol. Rev. 79:361-385.
256. Nishiyama, H., et al. (1997) Gene 204:115-120.
257. Noma, T., et al. (2001) Biochem. J. 358:225-232.
258. Notredame, C., et al. (2000) J. Molec. Biol. 302:205-217.
259. Okayama, H., et al. (1983) Mol. Cell. Biol. 3:280-289.
260. Okazaki, Y., et al. (2002) Nature 420:563-573.
261. Oksenberg, J. R., et al. (1999) Semin. Neurol. 19:281-288.
262. Oliver, C. J., et al. (1998) Frontiers in Bioscience 3:961-972.
263. O'Neil, N. J., et al. (2001) Am. J. Pharmacogenomics 1:45-53.
264. O'Neill, L. A. (2002) Curr. Top. Microbiol. Immunol. 270:47-61.
265. Osborn, B. L., et al (2002) J. Pharmacol. Exp. Ther. 303:540-548.
266. Page, D. C., et al. (1999) Hum. Reprod. 14:1722-1726.
267. Pan, C. X., et al. (1999) Med. Hypothesis 53:130-135.
268. Pang, T., et al. (2001) J. Biol. Chem 276:17,367-17,372.
269. Pang, T., et al. (2002) J. Biol. Chem. 277:43,771-43,777.
270. Papagerakis, S., et al. (2003) Hum. Pathol. 34:565-572.
271. Pearson, W. R. (2000) Methods Mol. Biol. 132:185-219.
272. Peattie, D. A., et al. (1992) Proc. Natl. Acad. Sci. 89:10,974-10,978.
273. Peelle, B., et al. (2001) J. Protein Chem. 20:507-519.
274. Peng, H., et al. (2003) Curr. Opin. Pharmacol. 3:329-333.
275. Pepin, K., et al. (2001) J. Vet. Med. Sci. 63:115-124.
276. Perez Calvo, J. I., et al. (2000) Med. Clin. (Barc) 115:601-604.
277. Perron, H., et al. (1997) Proc. Natl. Acad. Sci. 94:7583-7588.
278. Perry, A. C., et al. (1995) Biochem. J. 312(Pt 1):239-244.
279. Pertl, U., et al. (2003) Blood 101:649-654.
280. Pfutzer, R. H., et al. (2001) Pancreatology 1:457-460.
281. Phillips, M. I., ed. (1999a) Antisense Technology, Part A. Methods in Enzymology Vol. 313. Academic Press, Inc.
282. Phillips, M. I., ed. (1999b) Antisense Technology, Part B. Methods in Enzymology Vol. 314. Academic Press, Inc.
283. Pietu, G., et al. (1996) Genome Res. 6:492-503.
284. Pinkert, C. A., ed. (1994) Transgenic Animal Technology: A Laboratory Handbook. Academic Press.
285. Pisegna, J. R., et al. (1996) J. Biol. Chem. 271:17,267-17,274.
286. Prentki, P., et al. (1984) Gene 29:303-313.
287. Price, N. T., et al. (1993) Biochim. Biophys. Acta 1216:170-172.
288. Qin, J., et al. (2003) Radiat. Res. 159:139-148.
289. Racevskis, J., et al. (1996) Cell. Growth Differ. 7:271-280.
290. Ramalho-Santos, M. (2002) Science 298:597-600.
291. Raval, P. (1994) J. Pharmacol. Toxicol. Methods 32:125-127.
292. Rebbe, N. F., et al. (1987) Gene 53:235-245.
293. Rechid, R., et al. (1989) Comput. Appl. Biosci. 5:107-113.
294. Rehli, M., et al. (1995) J. Biol. Chem. 270:15644-15649.
295. Remington, J. P. (1985) Remington's Pharmaceutical Sciences. 17th ed. Mack Publishing Co.
296. Ribardo, D. A., et al. (2002) Indian J. Exp. Biol. 40:129-138.
297. Riley, J., et al. (1990) Nuc. Acids Res. 18:2887-2890.
298. Ritter, R. C., et al. (1994) Am. N.Y. Acad. Sci. 713:255-267.
299. Robertson, H. M. (1996) Mol. Gen. Genet. 252:761-766.
300. Robertson, H. M., et al. (1997) Gene 205:203-217.
301. Roepman, R., et al. (2000) Hum. Mol. Genet. 9:2095-2105.
302. Roessler, B. J., et al. (1993) J. Biol. Chem. 268:26476-26481.
303. Roggenkamp, R., et al. (1984) Mol. Gen. Genet. 194:489-493.
304. Rosato, R. R., et al. (2003) Cancer Biol. Ther. 2:30-37.
305. Rosen, R. C., et al. (2002) Ann. Rev. Sex Res. 13:36-88.
306. Rosenblum, M. G., et al. (1989) Crit. Rev. Immunol. 9:21-44.
307. Rowland, J. M. (2002) Pediatr. Clin. North Am. 49:1415-1435.
308. Saha, S., et al. (2001) Science 294:1343-1346.
309. Saiki, R. K, et al. (1988) Science 239:487-491.
310. Sambrook, J., et al. (2001) Molecular Cloning, A Laboratory Manual. 3^{nd} ed. Cold Spring Harbor Laboratory Press.
311. Sanchez, E. R., et al. (1990) Biochemistry 29:5145-5152.
312. Sasaki,T., et al. (2003) Endocr. Pathol. 14:141-144.
313. Sayers, J. R., et al. (1992) Biotechniques 13:592-596.
314. Schaeferling, M., et al. (2002) Electrophoresis 23:3097-3105.
315. Schäffer et al. (2003) Proc. Natl. Acad. Sci. 2003 100: 4435-4439.
316. Schaffer, J. E., et al. (1994) Cell 79:393-395.
317. Schena, M., ed. (1999) DNA Microarrays: A Practical Approach. Oxford Univ. Press.
318. Schena, M., ed. (2000) Microarray Biochip Technology. 1st ed. Eaton Publ. Co.
319. Schlesinger, D. H. (1988a) MacRomolecular Sequencing and Synthesis: Selected Methods and Applications. Wiley-Liss.
320. Schlesinger, D. H., ed. (1988b) Current Methods in Sequence Comparison and Analysis, Macromolecule Sequencing and Synthesis, Selected Methods and Applications, pp. 127-149, Alan R. Liss, Inc.
321. Schonthal, A. H. (2001) Cancer Lett. 170:1-13.
322. Seelig, H. P., et al. (1994) J. Autoimmun. 7:67-91.
323. Selkoe, D. J. (2001) Proc. Natl. Acad. Sci. 98:11,039-11,041.
324. Setlow, J., et al. eds. (1986) Genetic Engineering: Principles and Methods. Plenum Pub. Corp.
325. Shamay, M., et al. (2002) J. Biol. Chem. 277:9982-9988.
326. Shao, H., et al. (2000) J. Biol. Chem. 275:26,914-26,924.
327. Sheppard, P., et al. (2003) Nat. Immunol. 4:63-68.
328. Shinnick, T. M., et al. (1983) Ann. Rev. Microbiol. 37:425-446.
329. Shorter, J., et al. (2002)J. Cell Biol. 157:45-62.
330. Siebenlist, U., et al. (1980) Cell 20:269-281.
331. Siegal, G. J., et al. eds. (1999) Basic Neurochemistry, Molecular, Cellular, and Medical Aspects. 6th ed. Lippencott, Williams & Wilkins.
332. Sladek, R., et al. (1997)Mol. Cell Biol. 17:5400-5409.
333. Slavin, S., et al. (2001) Cancer Chemother. Pharmacol.48:S79-S84.
334. Smit, A. F., et al. (1996) Proc. Natl. Acad. Sci. 93:1443-1448.
335. Smith, G. E., et al. (1985) Proc. Natl. Acad. Sci. 82:8404-8408.
336. Smith, T. F., et al. (1981) Curr. Opin. Biotechnol. 8:542-546.
337. Soejima, H., et al. (2001) Genomics. 74:115-120.
338. Soulier, S., Vilotte, J et al. (1996) Gene 172:285-289.
339. Southern, E., et al. (1999) Nature Genetics 21:5-9.
340. Stein, C. A., et al. eds. (1998) Applied Antisense Oligonucleotide Technology. Wiley-Liss.
341. Steinhaur, C., et al. (2002) Biotechniques, Supp.:38-45.
342. Stetler-Stevenson, et al. (1993) FASEB J. 7:1434-1441.
343. Stewart, Z. A., et al. (2003) Trends Pharmacol. Sci. 24:139-145.
344. Stolz, L. E., et al. (1996) Mol. Biotechnol. 6:225-230.
345. Sturm, A., et al. (2002) Biochim. Biophys. Acta 1582:282-288.
346. Stutz, F., et al. (2000) RNA 6:638-650.
347. Suh, Y. H., et al. (2002) Pharmacol. Rev. 54:469-525.
348. Sung, C., et al. (2003) J. Interferon Cytokine Res. 23:25-36.
349. Sutcliffe, J. G., et al. (1983) Science 219:660-666.
350. Tamada et al. (2000) J. Immunol. 164:4105-4110
351. Tan, J., et al. (1999) Science286:2352-2355.
352. Tang, D. C., et al. (1992) Nature 356:152-154.
353. Tekur, S., et al. (1999) J. Androl. 20:135-144.
354. Terada, R., et al. (2003). Lab. Invest. 83:665-672.
355. Thompson, J. D., et al. (1994) Nucleic Acids Res. 22:4673-80.
356. Tilburn, J., et al. (1983) Gene 26:205-221.
357. Trounson, A. (2002) Reprod. Biomed. Online 4 Suppl. 1:58-63.
358. Tsuda, T., et al. (1993) Biochem. Biophys. Res. Commun. 195:363-373.
359. Tukey, R. H., et al. (1993) J. Biol. Chem. 268:15,260-15,266.
360. Turgeman, G., et al. (2002) Curr. Opin. Mol. Ther. 4:390-394.
361. Urs\acute{ø} et al. (1999) J. Biol. Chem. 274:30,864-30,873.
362. Vainberg, I. E., et al. (1998) Cell 93:863-873.
363. Vale, R. D. (2003) Cell 112:467-480.
364. Vallejo, M., et al. (1993) Proc. Natl. Acad. Sci. 90:4679-4683.
365. Van Damme, A., et al. (2002) Curr. Gene Ther. 2:195-209.
366. van den Berg, J. A., et al. (1990) Bio/Technology 8:135-139.
367. Van den Berghe, L., et al. (2000) Mol. Endocrinol. 14:1709-1724.
368. Van Den Blink, et al. (2002) Ann. N.Y. Acad. Sci. 973:349-58.
369. van der Spoel, et al. (2002) Proc. Natl. Acad. Sci. 99:17,173-17,178.
370. Van Eerdewegh, P., et al. (2002) Nature. 418:426-430.
371. Van Laar, et al. (2003) Cancer Control 10:57-65.
372. Verhey, K. J., et al. (2001) J. Cell Biol. 152:959-970.
373. Vlak, J. M., et al. (1988) J. Gen. Virol. 69:765-776.
374. Voisset, C., et al. (2000) AIDS Res. Hum. Retroviruses 16:731-740.
375. Wagner, R. W., et al. (1993) Science 260:1510-1513.
376. Wagner, R. W., et al. (1996) Nat. Biotechnol. 14:840-844.
377. Wakefield, et al. (2000) Breast Cancer Res. 2:100-106; Epub Feb. 21, 2000.
378. Walker, J. E., et al. (1992) J. Mol. Biol. 226:1051-1072.
379. Walsh, A. C., et al. (2001) Toxicol. Sci. 61:218-223.
380. Wang, J., et al. (2002) J. Biol. Chem. 277:46659-46668.
381. Wang, M. S., et al. (1999) Am. J. Med. Genet. 86:34-43.
382. Wax, S. D., et al. (1994) J. Biol. Chem. 269:13,041-13,047.
383. Wei, S., et al. (1997) Immunogenetics 45:405-412.
384. Weinberg, J. M., et al. (2003) Cutis. 71:25-29.
385. Weiner, H. L., Selkoe, D. J. (2002) Nature 420:879-884.
386. Weiner, M. P., et al. (1993) Gene 126:35-41.
387. Weinstein, M. E., et al. (1988) Cancer Genet Cytogenet. 35:223-229.
388. Weishaar, R. E., et al. (1985) J. Med. Chem. 28:537-545.
389. Weissman, I. L. (2000) Science 287:1442-1446.
390. Weng, S., et al. (2002) Proteomics 2:48-57.
391. Wenger, R. H., et al. (1993) J. Biol. Chem. 268:23,345-23,352.
392. Wentworth et al. (2001) Science 293:1806-
393. Werner, T., et al. (1990) Virology 174:225-238.
394. Wick, G., et al. (1987) Immunol. Lett. 16:249-257.
395. Wieczorek, H., et al. (1999) Bioessays 21:637-648.
396. Wieser, R. (2002) Leuk. Lymphoma 43:59-65.
397. Winssinger, N., et al. (2002) Proc. Natl. Acad. Sci. 99:11,139-11,144.
398. Wojtowicz-Praga, S. (1999) Drugs R. D. 1:117-129.
399. Wu et al. (2003) Anal. Biochem. 312:162-166.
400. Wu, A. M., et al. (1975) CRC Crit. Rev. Biochem. 3:289-347.
401. Xu, C. W., et al. (1997) Proc. Natl. Acad. Sci.94:12,473-12,478.
402. Xu, Y., et al. (1999) Proc. Natl. Acad. Sci. 96:151-156.
403. Yang, N., et al. (1996) J. Biol. Chem. 271:5795-5804.
404. Yao, Z., et al. (2003) Cancer Immunol. Immunother. 53:404-410; Epub Nov. 18, 2003.
405. Yelton, M. M., et al. (1984) Proc. Natl. Acad. Sci. 81:1470-1474.
406. Yoshihama, M., et al. (2002) Genome Res. 12:379-390.
407. Yu, L., et al. (1995) J. Virol. 69:3007-3016.
408. Yu, Z., Restifo, N. P. (2002) J. Clin. Invest. 110:289-294.
409. Zallipsky, S. (1995) Bioconjugate Chem., 6:150-165.
410. Zhang, Q., et al. (2002) Hum. Mol. Genet. 11:993-1003.
411. Zhang, W. M., et al. (2002) Matrix Biol. 21:513-523.
412. Zhao, H., et al. (2002) Cell Mol. Life Sci. 59:694-707.
413. Zhao, N., et al. (1995) Gene 156:207-215.
414. Zhao, Y., et al. (2003) Proc. Natl. Acad. Sci. 100:3965-3970
415. Zhu, D. L. (1989) Anal. Biochem. 177:120-124.
416. Zhu, H., et al. (2000) Nat. Genetics 26:283-289.
417. Zhu, H., et al. (2001) Science 293:2101-2105.
418. Zhu, H., et al. (2003) Curr. Opin. Chem. Biol. 7:55-63.
419. Zhu, J., et al. (1997) Proc. Natl. Acad. Sci. 94:13,063-13,068

### INDUSTRIAL APPLICABILITY

The polynucleotides and polypeptides of the invention are useful in diagnostic and therapeutic applications for a variety of diseases and conditions.

### Sequence Listing

The instant application contains a “lengthy” Sequence Listing which has been submitted via four CD-R in lieu of a printed paper copy, and is hereby incorporated by reference in its entirety. Said CD-R, recorded on Feb. 25, 2005, are labeled “CRF,” “Copy 1,” “Copy 2,” and “Copy 3” respectively, and each contains only one identical 528 Kb file (89403834.APP).

TABLE 1
Identification Numbers

| FP ID | SEQ.ID.NO. (N1) | SEQ.ID.NO. (P1) | SEQ.ID.NO. (N0) | Clone ID |
|---|---|---|---|---|
| HG1014903 | SEQ.ID.NO.: 1 | SEQ.ID.NO.: 188 | SEQ.ID.NO.: 375 | PLT00014330A02.contig.a |
| HG1014904 | SEQ.ID.NO.: 2 | SEQ.ID.NO.: 189 | | PLT00014330A02.contig.b |
| HG1014905 | SEQ.ID.NO.: 3 | SEQ.ID.NO.: 190 | SEQ.ID.NO.: 376 | CLN00736344 |
| HG1014906 | SEQ.ID.NO.: 4 | SEQ.ID.NO.: 191 | | CLN00736344 |
| HG1014907 | SEQ.ID.NO.: 5 | SEQ.ID.NO.: 192 | SEQ.ID.NO.: 377 | PLT00014330A17.contig.a |
| HG1014908 | SEQ.ID.NO.: 6 | SEQ.ID.NO.: 193 | SEQ.ID.NO.: 378 | PLT00014330A20.contig.a |
| HG1014909 | SEQ.ID.NO.: 7 | SEQ.ID.NO.: 194 | SEQ.ID.NO.: 379 | PLT00014330B02.contig.a |
| HG1014910 | SEQ.ID.NO.: 8 | SEQ.ID.NO.: 195 | | PLT00014330B02.contig.b |
| HG1014911 | SEQ.ID.NO.: 9 | SEQ.ID.NO.: 196 | SEQ.ID.NO.: 380 | PLT00014330B04.contig.a |
| HG1014912 | SEQ.ID.NO.: 10 | SEQ.ID.NO.: 197 | | PLT00014330B04.contig.b |
| HG1014913 | SEQ.ID.NO.: 11 | SEQ.ID.NO.: 198 | SEQ.ID.NO.: 381 | PLT00014330B05.contig.a |
| HG1014914 | SEQ.ID.NO.: 12 | SEQ.ID.NO.: 199 | SEQ.ID.NO.: 382 | PLT00014330B11.contig.a |
| HG1014915 | SEQ.ID.NO.: 13 | SEQ.ID.NO.: 200 | SEQ.ID.NO.: 383 | PLT00014330B13.contig.a |
| HG1014916 | SEQ.ID.NO.: 14 | SEQ.ID.NO.: 201 | | PLT00014330B13.contig.b |
| HG1014917 | SEQ.ID.NO.: 15 | SEQ.ID.NO.: 202 | SEQ.ID.NO.: 384 | CLN00736494 |
| HG1014918 | SEQ.ID.NO.: 16 | SEQ.ID.NO.: 203 | | PLT00014330B18.contig.b |
| HG1014919 | SEQ.ID.NO.: 17 | SEQ.ID.NO.: 204 | SEQ.ID.NO.: 385 | PLT00014330C06.contig.a |
| HG1014920 | SEQ.ID.NO.: 18 | SEQ.ID.NO.: 205 | | PLT00014330C06.contig.b |
| HG1014921 | SEQ.ID.NO.: 19 | SEQ.ID.NO.: 206 | SEQ.ID.NO.: 386 | PLT00014330C12.contig.a |
| HG1014922 | SEQ.ID.NO.: 20 | SEQ.ID.NO.: 207 | SEQ.ID.NO.: 387 | PLT00014330C14.contig.a |
| HG1014923 | SEQ.ID.NO.: 21 | SEQ.ID.NO.: 208 | SEQ.ID.NO.: 388 | PLT00014330C18.contig.a |
| HG1014924 | SEQ.ID.NO.: 22 | SEQ.ID.NO.: 209 | | PLT00014330C18.contig.b |
| HG1014925 | SEQ.ID.NO.: 23 | SEQ.ID.NO.: 210 | SEQ.ID.NO.: 389 | CLN00736483 |
| HG1014926 | SEQ.ID.NO.: 24 | SEQ.ID.NO.: 211 | | CLN00736483 |
| HG1014927 | SEQ.ID.NO.: 25 | SEQ.ID.NO.: 212 | SEQ.ID.NO.: 390 | PLT00014330D05.contig.a |
| HG1014928 | SEQ.ID.NO.: 26 | SEQ.ID.NO.: 213 | | PLT00014330D05.contig.b |
| HG1014929 | SEQ.ID.NO.: 27 | SEQ.ID.NO.: 214 | SEQ.ID.NO.: 391 | PLT00014330D07.contig.a |
| HG1014930 | SEQ.ID.NO.: 28 | SEQ.ID.NO.: 215 | SEQ.ID.NO.: 392 | CLN00736320 |
| HG1014931 | SEQ.ID.NO.: 29 | SEQ.ID.NO.: 216 | | CLN00736320 |
| HG1014932 | SEQ.ID.NO.: 30 | SEQ.ID.NO.: 217 | SEQ.ID.NO.: 393 | CLN00736408 |
| HG1014933 | SEQ.ID.NO.: 31 | SEQ.ID.NO.: 218 | | PLT00014330D12.contig.b |
| HG1014934 | SEQ.ID.NO.: 32 | SEQ.ID.NO.: 219 | SEQ.ID.NO.: 394 | PLT00014330D13.contig.a |
| HG1014935 | SEQ.ID.NO.: 33 | SEQ.ID.NO.: 220 | SEQ.ID.NO.: 395 | PLT00014330D15.contig.a |
| HG1014936 | SEQ.ID.NO.: 34 | SEQ.ID.NO.: 221 | | PLT00014330D15.contig.b |
| HG1014937 | SEQ.ID.NO.: 35 | SEQ.ID.NO.: 222 | SEQ.ID.NO.: 396 | PLT00014330D17.contig.a |
| HG1014938 | SEQ.ID.NO.: 36 | SEQ.ID.NO.: 223 | SEQ.ID.NO.: 397 | PLT00014330E04.contig.a |
| HG1014939 | SEQ.ID.NO.: 37 | SEQ.ID.NO.: 224 | SEQ.ID.NO.: 398 | PLT00014330E14.contig.a |
| HG1014940 | SEQ.ID.NO.: 38 | SEQ.ID.NO.: 225 | | PLT00014330E14.contig.b |
| HG1014941 | SEQ.ID.NO.: 39 | SEQ.ID.NO.: 226 | SEQ.ID.NO.: 399 | PLT00014330E24.contig.a |
| HG1014942 | SEQ.ID.NO.: 40 | SEQ.ID.NO.: 227 | | PLT00014330E24.contig.b |
| HG1014943 | SEQ.ID.NO.: 41 | SEQ.ID.NO.: 228 | SEQ.ID.NO.: 400 | PLT00014330F01.contig.a |
| HG1014944 | SEQ.ID.NO.: 42 | SEQ.ID.NO.: 229 | SEQ.ID.NO.: 401 | PLT00014330F03.contig.a |
| HG1014945 | SEQ.ID.NO.: 43 | SEQ.ID.NO.: 230 | | PLT00014330F03.contig.b |
| HG1014946 | SEQ.ID.NO.: 44 | SEQ.ID.NO.: 231 | SEQ.ID.NO.: 402 | CLN00736568 |
| HG1014947 | SEQ.ID.NO.: 45 | SEQ.ID.NO.: 232 | | PLT00014330F04.contig.b |
| HG1014948 | SEQ.ID.NO.: 46 | SEQ.ID.NO.: 233 | SEQ.ID.NO.: 403 | PLT00014330F05.contig.a |
| HG1014949 | SEQ.ID.NO.: 47 | SEQ.ID.NO.: 234 | SEQ.ID.NO.: 404 | PLT00014330F13.contig.a |
| HG1014950 | SEQ.ID.NO.: 48 | SEQ.ID.NO.: 235 | SEQ.ID.NO.: 405 | PLT00014330G21.contig.a |
| HG1014951 | SEQ.ID.NO.: 49 | SEQ.ID.NO.: 236 | | PLT00014330G21.contig.b |
| HG1014952 | SEQ.ID.NO.: 50 | SEQ.ID.NO.: 237 | | PLT00014330H05.contig.b |
| HG1014953 | SEQ.ID.NO.: 51 | SEQ.ID.NO.: 238 | SEQ.ID.NO.: 406 | PLT00014330H06.contig.a |
| HG1014954 | SEQ.ID.NO.: 52 | SEQ.ID.NO.: 239 | SEQ.ID.NO.: 407 | CLN00736486 |
| HG1014955 | SEQ.ID.NO.: 53 | SEQ.ID.NO.: 240 | | PLT00014330H12.contig.b |
| HG1014956 | SEQ.ID.NO.: 54 | SEQ.ID.NO.: 241 | SEQ.ID.NO.: 408 | PLT00014330H14.contig.a |
| HG1014957 | SEQ.ID.NO.: 55 | SEQ.ID.NO.: 242 | | PLT00014330H14.contig.b |
| HG1014958 | SEQ.ID.NO.: 56 | SEQ.ID.NO.: 243 | SEQ.ID.NO.: 409 | CLN00736439 |
| HG1014959 | SEQ.ID.NO.: 57 | SEQ.ID.NO.: 244 | | PLT00014330H18.contig.b |
| HG1014960 | SEQ.ID.NO.: 58 | SEQ.ID.NO.: 245 | SEQ.ID.NO.: 410 | PLT00014330I11.contig.a |
| HG1014961 | SEQ.ID.NO.: 59 | SEQ.ID.NO.: 246 | SEQ.ID.NO.: 411 | PLT00014330I12.contig.a |
| HG1014962 | SEQ.ID.NO.: 60 | SEQ.ID.NO.: 247 | | PLT00014330I12.contig.b |
| HG1014963 | SEQ.ID.NO.: 61 | SEQ.ID.NO.: 248 | SEQ.ID.NO.: 412 | PLT00014330I13.contig.a |
| HG1014964 | SEQ.ID.NO.: 62 | SEQ.ID.NO.: 249 | | PLT00014330I13.contig.b |
| HG1014965 | SEQ.ID.NO.: 63 | SEQ.ID.NO.: 250 | SEQ.ID.NO.: 413 | PLT00014330J10.contig.a |
| HG1014966 | SEQ.ID.NO.: 64 | SEQ.ID.NO.: 251 | | PLT00014330J10.contig.b |
| HG1014967 | SEQ.ID.NO.: 65 | SEQ.ID.NO.: 252 | SEQ.ID.NO.: 414 | PLT00014330J14.contig.a |
| HG1014968 | SEQ.ID.NO.: 66 | SEQ.ID.NO.: 253 | | PLT00014330J14.contig.b |
| HG1014969 | SEQ.ID.NO.: 67 | SEQ.ID.NO.: 254 | SEQ.ID.NO.: 415 | PLT00014330J15.contig.a |
| HG1014970 | SEQ.ID.NO.: 68 | SEQ.ID.NO.: 255 | SEQ.ID.NO.: 416 | PLT00014330J21.contig.a |
| HG1014971 | SEQ.ID.NO.: 69 | SEQ.ID.NO.: 256 | | PLT00014330J21.contig.b |
| HG1014972 | SEQ.ID.NO.: 70 | SEQ.ID.NO.: 257 | SEQ.ID.NO.: 417 | PLT00014330K01.contig.a |
| HG1014973 | SEQ.ID.NO.: 71 | SEQ.ID.NO.: 258 | SEQ.ID.NO.: 418 | PLT00014330K08.contig.a |
| HG1014974 | SEQ.ID.NO.: 72 | SEQ.ID.NO.: 259 | | PLT00014330K08.contig.b |
| HG1014975 | SEQ.ID.NO.: 73 | SEQ.ID.NO.: 260 | SEQ.ID.NO.: 419 | CLN00736375 |
| HG1014976 | SEQ.ID.NO.: 74 | SEQ.ID.NO.: 261 | | PLT00014330K09.contig.b |
| HG1014977 | SEQ.ID.NO.: 75 | SEQ.ID.NO.: 262 | SEQ.ID.NO.: 420 | PLT00014330K15.contig.a |
| HG1014978 | SEQ.ID.NO.: 76 | SEQ.ID.NO.: 263 | | PLT00014330K15.contig.b |
| HG1014979 | SEQ.ID.NO.: 77 | SEQ.ID.NO.: 264 | SEQ.ID.NO.: 421 | PLT00014330K24.contig.a |
| HG1014980 | SEQ.ID.NO.: 78 | SEQ.ID.NO.: 265 | SEQ.ID.NO.: 422 | PLT00014330L01.contig.a |
| HG1015004 | SEQ.ID.NO.: 79 | SEQ.ID.NO.: 266 | SEQ.ID.NO.: 423 | PLT00014330L24.contig.a |
| HG1014981 | SEQ.ID.NO.: 80 | SEQ.ID.NO.: 267 | SEQ.ID.NO.: 424 | PLT00014330M02.contig.a |
| HG1014982 | SEQ.ID.NO.: 81 | SEQ.ID.NO.: 268 | | PLT00014330M02.contig.b |
| HG1014983 | SEQ.ID.NO.: 82 | SEQ.ID.NO.: 269 | SEQ.ID.NO.: 425 | PLT00014330M08.contig.a |
| HG1014984 | SEQ.ID.NO.: 83 | SEQ.ID.NO.: 270 | | PLT00014330M08.contig.b |
| HG1014985 | SEQ.ID.NO.: 84 | SEQ.ID.NO.: 271 | SEQ.ID.NO.: 426 | PLT00014330M15.contig.a |
| HG1014986 | SEQ.ID.NO.: 85 | SEQ.ID.NO.: 272 | SEQ.ID.NO.: 427 | PLT00014330M17.contig.a |
| HG1014987 | SEQ.ID.NO.: 86 | SEQ.ID.NO.: 273 | | CLN00736332 |
| HG1014988 | SEQ.ID.NO.: 87 | SEQ.ID.NO.: 274 | SEQ.ID.NO.: 428 | PLT00014330N10.contig.a |
| HG1014989 | SEQ.ID.NO.: 88 | SEQ.ID.NO.: 275 | | PLT00014330N10.contig.b |
| HG1014990 | SEQ.ID.NO.: 89 | SEQ.ID.NO.: 276 | SEQ.ID.NO.: 429 | PLT00014330N12.contig.a |
| HG1014991 | SEQ.ID.NO.: 90 | SEQ.ID.NO.: 277 | | PLT00014330N12.contig.b |
| HG1014992 | SEQ.ID.NO.: 91 | SEQ.ID.NO.: 278 | SEQ.ID.NO.: 430 | CLN00736512 |
| HG1014993 | SEQ.ID.NO.: 92 | SEQ.ID.NO.: 279 | | CLN00736512 |
| HG1014994 | SEQ.ID.NO.: 93 | SEQ.ID.NO.: 280 | SEQ.ID.NO.: 431 | PLT00014330N22.contig.a |
| HG1014995 | SEQ.ID.NO.: 94 | SEQ.ID.NO.: 281 | | PLT00014330N22.contig.b |
| HG1014996 | SEQ.ID.NO.: 95 | SEQ.ID.NO.: 282 | SEQ.ID.NO.: 432 | CLN00736478 |
| HG1014997 | SEQ.ID.NO.: 96 | SEQ.ID.NO.: 283 | SEQ.ID.NO.: 433 | PLT00014330O07.contig.a |
| HG1014998 | SEQ.ID.NO.: 97 | SEQ.ID.NO.: 284 | | PLT00014330O07.contig.b |
| HG1015005 | SEQ.ID.NO.: 98 | SEQ.ID.NO.: 285 | SEQ.ID.NO.: 434 | PLT00014330O18.contig.a |
| HG1015006 | SEQ.ID.NO.: 99 | SEQ.ID.NO.: 286 | | PLT00014330O18.contig.b |
| HG1014999 | SEQ.ID.NO.: 100 | SEQ.ID.NO.: 287 | SEQ.ID.NO.: 435 | PLT00014330P07.contig.a |
| HG1015000 | SEQ.ID.NO.: 101 | SEQ.ID.NO.: 288 | | PLT00014330P07.contig.b |
| HG1015001 | SEQ.ID.NO.: 102 | SEQ.ID.NO.: 289 | SEQ.ID.NO.: 436 | PLT00014330P09.contig.a |
| HG1015002 | SEQ.ID.NO.: 103 | SEQ.ID.NO.: 290 | | PLT00014330P09.contig.b |
| HG1015003 | SEQ.ID.NO.: 104 | SEQ.ID.NO.: 291 | SEQ.ID.NO.: 437 | PLT00014330P15.contig.a |
| HG1015007 | SEQ.ID.NO.: 105 | SEQ.ID.NO.: 292 | SEQ.ID.NO.: 438 | CLN00736321 |
| HG1015008 | SEQ.ID.NO.: 106 | SEQ.ID.NO.: 293 | | PLT00014333A03.contig.b |
| HG1015009 | SEQ.ID.NO.: 107 | SEQ.ID.NO.: 294 | SEQ.ID.NO.: 439 | PLT00014333A06.contig.a |
| HG1015010 | SEQ.ID.NO.: 108 | SEQ.ID.NO.: 295 | | PLT00014333A06.contig.b |
| HG1015011 | SEQ.ID.NO.: 109 | SEQ.ID.NO.: 296 | SEQ.ID.NO.: 440 | PLT00014333A08.contig.a |
| HG1015012 | SEQ.ID.NO.: 110 | SEQ.ID.NO.: 297 | SEQ.ID.NO.: 441 | PLT00014333A15.contig.a |
| HG1015013 | SEQ.ID.NO.: 111 | SEQ.ID.NO.: 298 | | CLN00736625 |
| HG1015014 | SEQ.ID.NO.: 112 | SEQ.ID.NO.: 299 | SEQ.ID.NO.: 442 | PLT00014333A16.contig.a |
| HG1015015 | SEQ.ID.NO.: 113 | SEQ.ID.NO.: 300 | | PLT00014333A16.contig.b |
| HG1015016 | SEQ.ID.NO.: 114 | SEQ.ID.NO.: 301 | SEQ.ID.NO.: 443 | PLT00014333B03.contig.a |
| HG1015017 | SEQ.ID.NO.: 115 | SEQ.ID.NO.: 302 | | PLT00014333B03.contig.b |
| HG1015018 | SEQ.ID.NO.: 116 | SEQ.ID.NO.: 303 | SEQ.ID.NO.: 444 | PLT00014333B05.contig.a |
| HG1015019 | SEQ.ID.NO.: 117 | SEQ.ID.NO.: 304 | | PLT00014333B05.contig.b |
| HG1015020 | SEQ.ID.NO.: 118 | SEQ.ID.NO.: 305 | SEQ.ID.NO.: 445 | PLT00014333B15.contig.a |
| HG1015021 | SEQ.ID.NO.: 119 | SEQ.ID.NO.: 306 | SEQ.ID.NO.: 446 | PLT00014333B17.contig.a |
| HG1015022 | SEQ.ID.NO.: 120 | SEQ.ID.NO.: 307 | | CLN00736440 |
| HG1015023 | SEQ.ID.NO.: 121 | SEQ.ID.NO.: 308 | SEQ.ID.NO.: 447 | PLT00014333C02.contig.a |
| HG1015024 | SEQ.ID.NO.: 122 | SEQ.ID.NO.: 309 | | PLT00014333C02.contig.b |
| HG1015025 | SEQ.ID.NO.: 123 | SEQ.ID.NO.: 310 | SEQ.ID.NO.: 448 | PLT00014333C10.contig.a |
| HG1015026 | SEQ.ID.NO.: 124 | SEQ.ID.NO.: 311 | | PLT00014333C10.contig.b |
| HG1015027 | SEQ.ID.NO.: 125 | SEQ.ID.NO.: 312 | SEQ.ID.NO.: 449 | PLT00014333C16.contig.a |
| HG1015028 | SEQ.ID.NO.: 126 | SEQ.ID.NO.: 313 | | PLT00014333C16.contig.b |
| HG1015029 | SEQ.ID.NO.: 127 | SEQ.ID.NO.: 314 | SEQ.ID.NO.: 450 | PLT00014333C21.contig.a |
| HG1015030 | SEQ.ID.NO.: 128 | SEQ.ID.NO.: 315 | | PLT00014333C21.contig.b |
| HG1015031 | SEQ.ID.NO.: 129 | SEQ.ID.NO.: 316 | SEQ.ID.NO.: 451 | PLT00014333C24.contig.a |
| HG1015032 | SEQ.ID.NO.: 130 | SEQ.ID.NO.: 317 | | PLT00014333C24.contig.b |
| HG1015033 | SEQ.ID.NO.: 131 | SEQ.ID.NO.: 318 | SEQ.ID.NO.: 452 | PLT00014333D07.contig.a |
| HG1015034 | SEQ.ID.NO.: 132 | SEQ.ID.NO.: 319 | | PLT00014333D07.contig.b |
| HG1015035 | SEQ.ID.NO.: 133 | SEQ.ID.NO.: 320 | SEQ.ID.NO.: 453 | PLT00014333D15.contig.a |
| HG1015036 | SEQ.ID.NO.: 134 | SEQ.ID.NO.: 321 | | CLN00736385 |
| HG1015037 | SEQ.ID.NO.: 135 | SEQ.ID.NO.: 322 | SEQ.ID.NO.: 454 | CLN00736561 |
| HG1015038 | SEQ.ID.NO.: 136 | SEQ.ID.NO.: 323 | | CLN00736561 |
| HG1015039 | SEQ.ID.NO.: 137 | SEQ.ID.NO.: 324 | SEQ.ID.NO.: 455 | PLT00014333E04.contig.a |
| HG1015040 | SEQ.ID.NO.: 138 | SEQ.ID.NO.: 325 | SEQ.ID.NO.: 456 | PLT00014333E05.contig.a |
| HG1015041 | SEQ.ID.NO.: 139 | SEQ.ID.NO.: 326 | | PLT00014333E05.contig.b |
| HG1015042 | SEQ.ID.NO.: 140 | SEQ.ID.NO.: 327 | SEQ.ID.NO.: 457 | PLT00014333E14.contig.a |
| HG1015043 | SEQ.ID.NO.: 141 | SEQ.ID.NO.: 328 | | PLT00014333E14.contig.b |
| HG1015086 | SEQ.ID.NO.: 142 | SEQ.ID.NO.: 329 | SEQ.ID.NO.: 458 | PLT00014333E15.contig.a |
| HG1015087 | SEQ.ID.NO.: 143 | SEQ.ID.NO.: 330 | | PLT00014333E15.contig.b |
| HG1015044 | SEQ.ID.NO.: 144 | SEQ.ID.NO.: 331 | SEQ.ID.NO.: 459 | PLT00014333E24.contig.b |
| HG1015045 | SEQ.ID.NO.: 145 | SEQ.ID.NO.: 332 | SEQ.ID.NO.: 460 | PLT00014333F07.contig.a |
| HG1015046 | SEQ.ID.NO.: 146 | SEQ.ID.NO.: 333 | SEQ.ID.NO.: 461 | PLT00014333G01.contig.a |
| HG1015047 | SEQ.ID.NO.: 147 | SEQ.ID.NO.: 334 | SEQ.ID.NO.: 462 | PLT00014333G02.contig.a |
| HG1015048 | SEQ.ID.NO.: 148 | SEQ.ID.NO.: 335 | | PLT00014333G02.contig.b |
| HG1015088 | SEQ.ID.NO.: 149 | SEQ.ID.NO.: 336 | SEQ.ID.NO.: 463 | PLT00014333G09.contig.a |
| HG1015089 | SEQ.ID.NO.: 150 | SEQ.ID.NO.: 337 | | PLT00014333G09.contig.b |
| HG1015049 | SEQ.ID.NO.: 151 | SEQ.ID.NO.: 338 | SEQ.ID.NO.: 464 | PLT00014333H11.contig.a |
| HG1015050 | SEQ.ID.NO.: 152 | SEQ.ID.NO.: 339 | SEQ.ID.NO.: 465 | PLT00014333H15.contig.a |
| HG1015051 | SEQ.ID.NO.: 153 | SEQ.ID.NO.: 340 | | PLT00014333H15.contig.b |
| HG1015052 | SEQ.ID.NO.: 154 | SEQ.ID.NO.: 341 | SEQ.ID.NO.: 466 | PLT00014333I18.contig.a |
| HG1015053 | SEQ.ID.NO.: 155 | SEQ.ID.NO.: 342 | | PLT00014333I18.contig.b |
| HG1015054 | SEQ.ID.NO.: 156 | SEQ.ID.NO.: 343 | SEQ.ID.NO.: 467 | PLT00014333I22.contig.a |
| HG1015055 | SEQ.ID.NO.: 157 | SEQ.ID.NO.: 344 | | PLT00014333I22.contig.b |
| HG1015056 | SEQ.ID.NO.: 158 | SEQ.ID.NO.: 345 | SEQ.ID.NO.: 468 | PLT00014333J01.contig.a |
| HG1015057 | SEQ.ID.NO.: 159 | SEQ.ID.NO.: 346 | | PLT00014333J01.contig.b |
| HG1015058 | SEQ.ID.NO.: 160 | SEQ.ID.NO.: 347 | SEQ.ID.NO.: 469 | PLT00014333J13.contig.a |
| HG1015059 | SEQ.ID.NO.: 161 | SEQ.ID.NO.: 348 | | PLT00014333J13.contig.b |
| HG1015060 | SEQ.ID.NO.: 162 | SEQ.ID.NO.: 349 | SEQ.ID.NO.: 470 | PLT00014333J15.contig.a |
| HG1015061 | SEQ.ID.NO.: 163 | SEQ.ID.NO.: 350 | | PLT00014333J15.contig.b |
| HG1015062 | SEQ.ID.NO.: 164 | SEQ.ID.NO.: 351 | SEQ.ID.NO.: 471 | PLT00014333J17.contig.a |
| HG1015063 | SEQ.ID.NO.: 165 | SEQ.ID.NO.: 352 | SEQ.ID.NO.: 472 | PLT00014333J23.contig.a |
| HG1015064 | SEQ.ID.NO.: 166 | SEQ.ID.NO.: 353 | | PLT00014333J23.contig.b |
| HG1015065 | SEQ.ID.NO.: 167 | SEQ.ID.NO.: 354 | SEQ.ID.NO.: 473 | PLT00014333K04.contig.a |
| HG1015066 | SEQ.ID.NO.: 168 | SEQ.ID.NO.: 355 | | PLT00014333K04.contig.b |
| HG1015067 | SEQ.ID.NO.: 169 | SEQ.ID.NO.: 356 | SEQ.ID.NO.: 474 | CLN00625950 |
| | | | | CLN00625952 |
| | | | | CLN00625956 |
| | | | | CLN00625984 |
| | | | | CLN00625986 |
| | | | | CLN00626567 |
| | | | | CLN00626569 |
| | | | | CLN00626571 |
| | | | | CLN00626573 |
| HG1015068 | SEQ.ID.NO.: 170 | SEQ.ID.NO.: 357 | | CLN00625950 |
| | | | | CLN00625952 |
| | | | | CLN00625956 |
| | | | | CLN00625984 |
| | | | | CLN00625986 |
| | | | | CLN00626567 |
| | | | | CLN00626569 |
| | | | | CLN00626571 |
| | | | | CLN00626573 |
| HG1015069 | SEQ.ID.NO.: 171 | SEQ.ID.NO.: 358 | SEQ.ID.NO.: 475 | PLT00014333L13.contig.b |
| HG1015070 | SEQ.ID.NO.: 172 | SEQ.ID.NO.: 359 | SEQ.ID.NO.: 476 | PLT00014333M01.contig.a |
| HG1015071 | SEQ.ID.NO.: 173 | SEQ.ID.NO.: 360 | | PLT00014333M01.contig.b |
| HG1015072 | SEQ.ID.NO.: 174 | SEQ.ID.NO.: 361 | SEQ.ID.NO.: 477 | PLT00014333M02.contig.a |
| HG1015073 | SEQ.ID.NO.: 175 | SEQ.ID.NO.: 362 | | PLT00014333M02.contig.b |
| HG1015074 | SEQ.ID.NO.: 176 | SEQ.ID.NO.: 363 | SEQ.ID.NO.: 478 | CLN00736352 |
| HG1015075 | SEQ.ID.NO.: 177 | SEQ.ID.NO.: 364 | | CLN00736352 |
| HG1015076 | SEQ.ID.NO.: 178 | SEQ.ID.NO.: 365 | SEQ.ID.NO.: 479 | PLT00014333M15.contig.a |
| HG1015077 | SEQ.ID.NO.: 179 | SEQ.ID.NO.: 366 | | PLT00014333M15.contig.b |
| HG1015078 | SEQ.ID.NO.: 180 | SEQ.ID.NO.: 367 | SEQ.ID.NO.: 480 | PLT00014333N05.contig.a |
| HG1015079 | SEQ.ID.NO.: 181 | SEQ.ID.NO.: 368 | | PLT00014333N05.contig.b |
| HG1015080 | SEQ.ID.NO.: 182 | SEQ.ID.NO.: 369 | SEQ.ID.NO.: 481 | PLT00014333N11.contig.a |
| HG1015081 | SEQ.ID.NO.: 183 | SEQ.ID.NO.: 370 | | PLT00014333N11.contig.b |
| HG1015082 | SEQ.ID.NO.: 184 | SEQ.ID.NO.: 371 | SEQ.ID.NO.: 482 | PLT00014333O03.contig.a |
| HG1015083 | SEQ.ID.NO.: 185 | SEQ.ID.NO.: 372 | | PLT00014333O03.contig.b |
| HG1015084 | SEQ.ID.NO.: 186 | SEQ.ID.NO.: 373 | SEQ.ID.NO.: 483 | PLT00014333O10.contig.a |
| HG1015085 | SEQ.ID.NO.: 187 | SEQ.ID.NO.: 374 | SEQ.ID.NO.: 484 | PLT00014333O17.contig.a |

TABLE 2
Structural Characteristics

| | | | | | Altern | | | | | |
|---|---|---|---|---|---|---|---|---|---|---|
| | | | | Mature | Mature | Signal | | | | |
| | | Pred Prot | Tree- | Protein | Protein | Peptide | | | Non- | |
| FP ID | Clone ID | Len | vote | Coords | Coords | Coords | TM | TM Coords | TM Coords | Pfam |
| HG1014903 | PLT00014330A02.contig.a | 89 | 0 | (1-89) | | | 0 | | (1-89) | no_pfam |
| HG1014904 | PLT00014330A02.contig.b | 87 | 0 | (1-87) | | | 0 | | (1-87) | no_pfam |
| HG1014905 | PLT00014330A08.contig.a | 82 | 0.55 | (27-82) | | (1-26) | 1 | (15-37) | (1-14)(38-82) | no_pfam |
| HG1014906 | PLT00014330A08.contig.b | 61 | 0.62 | (24-61) | | (6-23) | 2 | (5-27)(31-53) | (1-4) | no_pfam |
| | | | | | | | | | (28-30)(54-61) | |
| HG1014907 | PLT00014330A17.contig.a | 66 | 0.11 | (1-66) | (39-66) | (11-38) | 0 | | (1-66) | no_pfam |
| HG1014908 | PLT00014330A20.contig.a | 54 | 0.25 | (33-54) | | (18-32) | 0 | | (1-54) | no_pfam |
| HG1014909 | PLT00014330B02.contig.a | 84 | 0 | (1-84) | | | 0 | | (1-84) | no_pfam |
| HG1014910 | PLT00014330B02.contig.b | 73 | 0.07 | (22-73) | (41-73) | (16-40) | 0 | | (1-73) | no_pfam |
| HG1014911 | PLT00014330B04.contig.a | 160 | 0 | (1-160) | | | 0 | | (1-160) | no_pfam |
| HG1014912 | PLT00014330B04.contig.b | 108 | 0.05 | (1-108) | (25-108) | (11-24) | 0 | | (1-108) | no_pfam |
| HG1014913 | PLT00014330B05.contig.a | 79 | 0.02 | (1-79) | | | 0 | | (1-79) | no_pfam |
| HG1014914 | PLT00014330B11.contig.a | 68 | 0.23 | (15-68) | (26-68) | (1-25) | 0 | | (1-68) | no_pfam |
| HG1014915 | PLT00014330B13.contig.a | 55 | 0.05 | (1-55) | (38-55) | (8-37) | 0 | | (1-55) | no_pfam |
| HG1014916 | PLT00014330B13.contig.b | 53 | 0.01 | (1-53) | (20-53) | (1-19) | 0 | | (1-53) | no_pfam |
| HG1014917 | PLT00014330B18.contig.a | 74 | 0.7 | (22-74) | | (2-21) | 0 | | (1-74) | no_pfam |
| HG1014918 | PLT00014330B18.contig.b | 53 | 0.24 | (28-53) | (37-53) | (14-36) | 0 | | (1-53) | no_pfam |
| HG1014919 | PLT00014330C06.contig.a | 101 | 0.53 | (20-101) | (44-101) | (19-43) | 0 | | (1-101) | no_pfam |
| HG1014920 | PLT00014330C06.contig.b | 65 | 0.01 | (1-65) | (18-65) | (1-17) | 0 | | (1-65) | no_pfam |
| HG1014921 | PLT00014330C12.contig.a | 68 | 0.01 | (1-68) | (23-68) | (1-22) | 0 | | (1-68) | no_pfam |
| HG1014922 | PLT00014330C14.contig.a | 66 | 0.02 | (1-66) | | | 0 | | (1-66) | no_pfam |
| HG1014923 | PLT00014330C18.contig.a | 64 | 0 | (1-64) | (20-64) | (1-19) | 0 | | (1-64) | no_pfam |
| HG1014924 | PLT00014330C18.contig.b | 63 | 0 | (1-63) | | | 0 | | (1-63) | no_pfam |
| HG1014925 | PLT00014330D03.contig.a | 132 | 0.81 | (20-132) | | (1-19) | 0 | | (1-132) | no_pfam |
| HG1014926 | PLT00014330D03.contig.b | 74 | 0.43 | (37-74) | | (15-36) | 2 | (12-31)(46-68) | (1-11) | no_pfam |
| | | | | | | | | | (32-45)(69-74) | |
| HG1014927 | PLT00014330D05.contig.a | 60 | 0.07 | (1-60) | (32-60) | (16-31) | 0 | | (1-60) | no_pfam |
| HG1014928 | PLT00014330D05.contig.b | 54 | 0.39 | (1-54) | (27-54) | (1-26) | 0 | | (1-54) | no_pfam |
| HG1014929 | PLT00014330D07.contig.a | 85 | 0.03 | (4-85) | (1-85) | | 0 | | (1-85) | no_pfam |
| HG1014930 | PLT00014330D10.contig.a | 79 | 0.61 | (29-79) | (30-79) | (6-29) | 0 | | (1-79) | no_pfam |
| HG1014931 | PLT00014330D10.contig.b | 73 | 0.87 | (22-73) | (20-73) | (1-19) | 0 | | (1-73) | no_pfam |
| HG1014932 | PLT00014330D12.contig.a | 116 | 0.01 | (1-116) | | | 1 | (21-43) | (1-20)(44-116) | no_pfam |
| HG1014933 | PLT00014330D12.contig.b | 54 | 0.24 | (24-54) | | (1-23) | 0 | | (1-54) | no_pfam |
| HG1014934 | PLT00014330D13.contig.a | 60 | 0 | (1-60) | | | 0 | | (1-60) | no_pfam |
| HG1014935 | PLT00014330D15.contig.a | 92 | 0.01 | (1-92) | (21-92) | (6-20) | 0 | | (1-92) | no_pfam |
| HG1014936 | PLT00014330D15.contig.b | 89 | 0.4 | (36-89) | (46-89) | (16-45) | 1 | (12-34) | (1-11)(35-89) | no_pfam |
| HG1014937 | PLT00014330D17.contig.a | 96 | 0.26 | (30-96) | (27-96) | (10-26) | 0 | | (1-96) | no_pfam |
| HG1014938 | PLT00014330E04.contig.a | 54 | 0.02 | (1-54) | | | 0 | | (1-54) | no_pfam |
| HG1014939 | PLT00014330E14.contig.a | 68 | 0.02 | (1-68) | (19-68) | (1-18) | 0 | | (1-68) | no_pfam |
| HG1014940 | PLT00014330E14.contig.b | 61 | 0 | (1-61) | (27-61) | (9-26) | 0 | | (1-61) | no_pfam |
| HG1014941 | PLT00014330E24.contig.a | 112 | 0.01 | (1-112) | | | 0 | | (1-112) | no_pfam |
| HG1014942 | PLT00014330E24.contig.b | 62 | 0.16 | (1-62) | (35-62) | (17-34) | 1 | (15-34) | (1-14)(35-62) | no_pfam |
| HG1014943 | PLT00014330F01.contig.a | 77 | 0 | (1-77) | | | 1 | (28-45) | (1-27)(46-77) | no_pfam |
| HG1014944 | PLT00014330F03.contig.a | 105 | 0 | (1-105) | | | 0 | | (1-105) | no_pfam |
| HG1014945 | PLT00014330F03.contig.b | 71 | 0.01 | (27-71) | (1-71) | | 0 | | (1-71) | no_pfam |
| HG1014946 | PLT00014330F04.contig.a | 117 | 0.9 | (18-117) | (20-117) | (1-19) | 0 | | (1-117) | no_pfam |
| HG1014947 | PLT00014330F04.contig.b | 104 | 0.09 | (25-104) | | (1-24) | 0 | | (1-104) | no_pfam |
| HG1014948 | PLT00014330F05.contig.a | 50 | 0.01 | (1-50) | (16-50) | (1-15) | 0 | | (1-50) | no_pfam |
| HG1014949 | PLT00014330F13.contig.a | 53 | 0.26 | (28-53) | | (1-27) | 0 | | (1-53) | no_pfam |
| HG1014950 | PLT00014330G21.contig.a | 146 | 0.16 | (28-146) | (29-146) | (6-28) | 0 | | (1-146) | no_pfam |
| HG1014951 | PLT00014330G21.contig.b | 53 | 0.05 | (1-53) | | | 1 | (20-42) | (1-19)(43-53) | no_pfam |
| HG1014952 | PLT00014330H05.contig.b | 97 | 0.01 | (1-97) | (25-97) | (1-24) | 0 | | (1-97) | rvt |
| HG1014953 | PLT00014330H06.contig.a | 50 | 0.16 | (1-50) | (32-50) | (16-31) | 0 | | (1-50) | no_pfam |
| HG1014954 | PLT00014330H12.contig.a | 86 | 0.65 | (19-86) | | (1-18) | 0 | | (1-86) | no_pfam |
| HG1014955 | PLT00014330H12.contig.b | 76 | 0.03 | (1-76) | (19-76) | (1-18) | 0 | | (1-76) | no_pfam |
| HG1014956 | PLT00014330H14.contig.a | 68 | 0.2 | (38-68) | (17-68) | (1-16) | 0 | | (1-68) | no_pfam |
| HG1014957 | PLT00014330H14.contig.b | 66 | 0.05 | (29-66) | (1-66) | | 1 | (43-62) | (1-42)(63-66) | no_pfam |
| HG1014958 | PLT00014330H18.contig.a | 95 | 0.94 | (21-95) | (19-95) | (1-18) | 0 | | (1-95) | no_pfam |
| HG1014959 | PLT00014330H18.contig.b | 77 | 0.01 | (38-77) | (1-77) | | 0 | | (1-77) | no_pfam |
| HG1014960 | PLT00014330I11.contig.a | 62 | 0.05 | (1-62) | | | 1 | (31-53) | (1-30)(54-62) | no_pfam |
| HG1014961 | PLT00014330I12.contig.a | 88 | 0.3 | (8-88) | (19-88) | (1-18) | 0 | | (1-88) | no_pfam |
| HG1014962 | PLT00014330I12.contig.b | 66 | 0.51 | (8-66) | (16-66) | (1-15) | 2 | (4-26)(43-65) | (1-3) | no_pfam |
| | | | | | | | | | (27-42)(66-66) | |
| HG1014963 | PLT00014330I13.contig.a | 103 | 0.04 | (1-103) | (41-103) | (17-40) | 0 | | (1-103) | no_pfam |
| HG1014964 | PLT00014330I13.contig.b | 84 | 0.02 | (1-84) | (18-84) | (5-17) | 0 | | (1-84) | no_pfam |
| HG1014965 | PLT00014330J10.contig.a | 130 | 0.05 | (16-130) | (1-130) | | 0 | | (1-130) | no_pfam |
| HG1014966 | PLT00014330J10.contig.b | 103 | 0 | (1-103) | | | 0 | | (1-103) | no_pfam |
| HG1014967 | PLT00014330J14.contig.a | 79 | 0.02 | (32-79) | (1-79) | | 0 | | (1-79) | no_pfam |
| HG1014968 | PLT00014330J14.contig.b | 57 | 0.03 | (1-57) | (23-57) | (1-22) | 0 | | (1-57) | no_pfam |
| HG1014969 | PLT00014330J15.contig.a | 68 | 0.01 | (1-68) | | | 0 | | (1-68) | no_pfam |
| HG1014970 | PLT00014330J21.contig.a | 80 | 0.1 | (1-80) | (25-80) | (10-24) | 0 | | (1-80) | no_pfam |
| HG1014971 | PLT00014330J21.contig.b | 68 | 0.08 | (1-68) | (22-68) | (1-21) | 0 | | (1-68) | no_pfam |
| HG1014972 | PLT00014330K01.contig.a | 73 | 0 | (1-73) | | | 0 | | (1-73) | no_pfam |
| HG1014973 | PLT00014330K08.contig.a | 99 | 0.16 | (1-99) | (26-99) | (1-25) | 1 | (73-95) | (1-72)(96-99) | no_pfam |
| HG1014974 | PLT00014330K08.contig.b | 50 | 0.26 | (1-50) | (18-50) | (1-17) | 2 | (5-27)(32-49) | (1-4) | no_pfam |
| | | | | | | | | | (28-31)(50-50) | |
| HG1014975 | PLT00014330K09.contig.a | 100 | 0.09 | (20-100) | | (2-19) | 0 | | (1-100) | no_pfam |
| HG1014976 | PLT00014330K09.contig.b | 60 | 0 | (1-60) | (23-60) | (11-22) | 0 | | (1-60) | no_pfam |
| HG1014977 | PLT00014330K15.contig.a | 72 | 0.01 | (1-72) | (26-72) | (2-25) | 0 | | (1-72) | no_pfam |
| HG1014978 | PLT00014330K15.contig.b | 61 | 0 | (1-61) | (33-61) | (9-32) | 0 | | (1-61) | no_pfam |
| HG1014979 | PLT00014330K24.contig.a | 51 | 0.17 | (37-51) | (29-51) | (8-28) | 1 | (13-35) | (1-12)(36-51) | no_pfam |
| HG1014980 | PLT00014330L01.contig.a | 112 | 0.13 | (37-112) | (19-112) | (1-18) | 0 | | (1-112) | no_pfam |
| HG1014981 | PLT00014330M02.contig.a | 106 | 0.01 | (1-106) | | | 0 | | (1-106) | no_pfam |
| HG1014982 | PLT00014330M02.contig.b | 88 | 0.27 | (1-88) | (19-88) | (1-18) | 0 | | (1-88) | no_pfam |
| HG1014983 | PLT00014330M08.contig.a | 72 | 0.46 | (32-72) | | (18-31) | 1 | (45-67) | (1-44)(68-72) | no_pfam |
| HG1014984 | PLT00014330M08.contig.b | 52 | 0.29 | (31-52) | | (17-30) | 1 | (20-42) | (1-19)(43-52) | no_pfam |
| HG1014985 | PLT00014330M15.contig.a | 53 | 0.07 | (1-53) | (53-53) | (19-52) | 0 | | (1-53) | no_pfam |
| HG1014986 | PLT00014330M17.contig.a | 110 | 0.13 | (1-110) | (21-110) | (1-20) | 0 | | (1-110) | no_pfam |
| HG1014987 | PLT00014330M17.contig.b | 82 | 0.45 | (29-82) | (30-82) | (16-29) | 0 | | (1-82) | no_pfam |
| HG1014988 | PLT00014330N10.contig.a | 75 | 0.15 | (38-75) | | (18-37) | 1 | (20-42) | (1-19)(43-75) | no_pfam |
| HG1014989 | PLT00014330N10.contig.b | 68 | 0 | (1-68) | (22-68) | (1-21) | 0 | | (1-68) | no_pfam |
| HG1014990 | PLT00014330N12.contig.a | 56 | 0 | (1-56) | (33-56) | (18-32) | 0 | | (1-56) | no_pfam |
| HG1014991 | PLT00014330N12.contig.b | 56 | 0 | (1-56) | (20-56) | (1-19) | 0 | | (1-56) | no_pfam |
| HG1014992 | PLT00014330N13.contig.a | 83 | 0.87 | (23-83) | (20-83) | (1-19) | 1 | (4-26) | (1-3)(27-83) | no_pfam |
| HG1014993 | PLT00014330N13.contig.b | 55 | 0.29 | (28-55) | (29-55) | (14-28) | 1 | (10-32) | (1-9)(33-55) | no_pfam |
| HG1014994 | PLT00014330N22.contig.a | 74 | 0.02 | (1-74) | (33-74) | (19-32) | 0 | | (1-74) | no_pfam |
| HG1014995 | PLT00014330N22.contig.b | 57 | 0.12 | (1-57) | (20-57) | (1-19) | 0 | | (1-57) | no_pfam |
| HG1014996 | PLT00014330O03.contig.a | 70 | 0.32 | (1-70) | (19-70) | (5-18) | 1 | (7-29) | (1-6)(30-70) | no_pfam |
| HG1014997 | PLT00014330O07.contig.a | 78 | 0 | (1-78) | | | 0 | | (1-78) | no_pfam |
| HG1014998 | PLT00014330O07.contig.b | 73 | 0.06 | (1-73) | (33-73) | (19-32) | 0 | | (1-73) | no_pfam |
| HG1014999 | PLT00014330P07.contig.a | 85 | 0.03 | (1-85) | (33-85) | (1-32) | 0 | | (1-85) | no_pfam |
| HG1015000 | PLT00014330P07.contig.b | 61 | 0.05 | (34-61) | (32-61) | (1-31) | 0 | | (1-61) | no_pfam |
| HG1015001 | PLT00014330P09.contig.a | 101 | 0.17 | (1-101) | (33-101) | (13-32) | 0 | | (1-101) | no_pfam |
| HG1015002 | PLT00014330P09.contig.b | 98 | 0.01 | (1-98) | | | 0 | | (1-98) | no_pfam |
| HG1015003 | PLT00014330P15.contig.a | 61 | 0.02 | (1-61) | | | 0 | | (1-61) | no_pfam |
| HG1015004 | PLT00014330L24.contig.a | 50 | 0.17 | (38-50) | (34-50) | (1-33) | 0 | | (1-50) | no_pfam |
| HG1015005 | PLT00014330O18.contig.a | 82 | 0 | (1-82) | | | 0 | | (1-82) | no_pfam |
| HG1015006 | PLT00014330O18.contig.b | 66 | 0 | (1-66) | | | 0 | | (1-66) | no_pfam |
| HG1015007 | PLT00014333A03.contig.a | 83 | 0.08 | (1-83) | (39-83) | (19-38) | 1 | (15-37) | (1-14)(38-83) | no_pfam |
| HG1015008 | PLT00014333A03.contig.b | 64 | 0.1 | (30-64) | (29-64) | (11-28) | 0 | | (1-64) | no_pfam |
| HG1015009 | PLT00014333A06.contig.a | 153 | 0.01 | (1-153) | | | 0 | | (1-153) | no_pfam |
| HG1015010 | PLT00014333A06.contig.b | 66 | 0.13 | (35-66) | (33-66) | (18-32) | 0 | | (1-66) | no_pfam |
| HG1015011 | PLT00014333A08.contig.a | 66 | 0.26 | (1-66) | (22-66) | (1-21) | 0 | | (1-66) | no_pfam |
| HG1015012 | PLT00014333A15.contig.a | 136 | 0.03 | (1-136) | | | 0 | | (1-136) | no_pfam |
| HG1015013 | PLT00014333A15.contig.b | 67 | 0.8 | (38-67) | (35-67) | (17-34) | 0 | | (1-67) | no_pfam |
| HG1015014 | PLT00014333A16.contig.a | 51 | 0.02 | (1-51) | | | 0 | | (1-51) | no_pfam |
| HG1015015 | PLT00014333A16.contig.b | 50 | 0.46 | (25-50) | (41-50) | (16-40) | 0 | | (1-50) | no_pfam |
| HG1015016 | PLT00014333B03.contig.a | 63 | 0.02 | (1-63) | | | 0 | | (1-63) | no_pfam |
| HG1015017 | PLT00014333B03.contig.b | 50 | 0 | (1-50) | (15-50) | (1-14) | 0 | | (1-50) | no_pfam |
| HG1015018 | PLT00014333B05.contig.a | 55 | 0.05 | (1-55) | | | 1 | (29-51) | (1-28)(52-55) | no_pfam |
| HG1015019 | PLT00014333B05.contig.b | 53 | 0.49 | (1-53) | (18-53) | (1-17) | 0 | | (1-53) | no_pfam |
| HG1015020 | PLT00014333B15.contig.a | 53 | 0 | (1-53) | (28-53) | (3-27) | 0 | | (1-53) | no_pfam |
| HG1015021 | PLT00014333B17.contig.a | 76 | 0.35 | (16-76) | | (1-15) | 0 | | (1-76) | no_pfam |
| HG1015022 | PLT00014333B17.contig.b | 65 | 0.01 | (1-65) | | | 1 | (42-64) | (1-41)(65-65) | no_pfam |
| HG1015023 | PLT00014333C02.contig.a | 77 | 0.03 | (1-77) | | | 0 | | (1-77) | no_pfam |
| HG1015024 | PLT00014333C02.contig.b | 51 | 0.77 | (22-51) | | (8-21) | 1 | (12-34) | (1-11)(35-51) | no_pfam |
| HG1015025 | PLT00014333C10.contig.a | 99 | 0.33 | (1-99) | (50-99) | (19-49) | 0 | | (1-99) | no_pfam |
| HG1015026 | PLT00014333C10.contig.b | 92 | 0.21 | (18-92) | (20-92) | (1-19) | 0 | | (1-92) | no_pfam |
| HG1015027 | PLT00014333C16.contig.a | 363 | 0.04 | (1-363) | (15-363) | (1-14) | 0 | | (1-363) | no_pfam |
| HG1015028 | PLT00014333C16.contig.b | 86 | 0.24 | (1-86) | (27-86) | (1-26) | 0 | | (1-86) | no_pfam |
| HG1015029 | PLT00014333C21.contig.a | 82 | 0.49 | (1-82) | (49-82) | (19-48) | 0 | | (1-82) | no_pfam |
| HG1015030 | PLT00014333C21.contig.b | 77 | 0.03 | (1-77) | (28-77) | (9-27) | 0 | | (1-77) | no_pfam |
| HG1015031 | PLT00014333C24.contig.a | 94 | 0.11 | (1-94) | (30-94) | (15-29) | 1 | (10-32) | (1-9)(33-94) | no_pfam |
| HG1015032 | PLT00014333C24.contig.b | 88 | 0 | (1-88) | | | 2 | (34-56)(61-78) | (1-33) | no_pfam |
| | | | | | | | | | (57-60)(79-88) | |
| HG1015033 | PLT00014333D07.contig.a | 73 | 0.02 | (1-73) | (21-73) | (1-20) | 0 | | (1-73) | no_pfam |
| HG1015034 | PLT00014333D07.contig.b | 67 | 0.23 | (1-67) | (32-67) | (1-31) | 0 | | (1-67) | no_pfam |
| HG1015035 | PLT00014333D15.contig.a | 64 | 0.11 | (32-64) | (31-64) | (16-30) | 0 | | (1-64) | no_pfam |
| HG1015036 | PLT00014333D15.contig.b | 62 | 0.29 | (34-62) | (31-62) | (5-30) | 2 | (13-32)(42-61) | (1-12) | no_pfam |
| | | | | | | | | | (33-41)(62-62) | |
| HG1015037 | PLT00014333E01.contig.a | 73 | 0 | (36-73) | (1-73) | | 1 | (26-48) | (1-25)(49-73) | no_pfam |
| HG1015038 | PLT00014333E01.contig.b | 67 | 0.51 | (35-67) | (26-67) | (8-25) | 1 | (10-32) | (1-9)(33-67) | no_pfam |
| HG1015039 | PLT00014333E04.contig.a | 53 | 0.01 | (1-53) | | | 0 | | (1-53) | no_pfam |
| HG1015040 | PLT00014333E05.contig.a | 66 | 0.01 | (1-66) | (25-66) | (8-24) | 0 | | (1-66) | no_pfam |
| HG1015041 | PLT00014333E05.contig.b | 57 | 0.03 | (1-57) | (45-57) | (1-44) | 0 | | (1-57) | no_pfam |
| HG1015042 | PLT00014333E14.contig.a | 108 | 0.01 | (1-108) | | | 0 | | (1-108) | no_pfam |
| HG1015043 | PLT00014333E14.contig.b | 61 | 0.24 | (26-61) | (29-61) | (14-28) | 0 | | (1-61) | no_pfam |
| HG1015044 | PLT00014333E24.contig.b | 91 | 0.01 | (1-91) | (32-91) | (18-31) | 0 | | (1-91) | Transposase |
| | | | | | | | | | | 1 |
| HG1015045 | PLT00014333F07.contig.a | 52 | 0 | (1-52) | (17-52) | (1-16) | 0 | | (1-52) | no_pfam |
| HG1015046 | PLT00014333G01.contig.a | 69 | 0.24 | (1-69) | (33-69) | (14-32) | 0 | | (1-69) | no_pfam |
| HG1015047 | PLT00014333G02.contig.a | 77 | 0.03 | (19-77) | (1-77) | | 0 | | (1-77) | no_pfam |
| HG1015048 | PLT00014333G02.contig.b | 57 | 0 | (1-57) | | | 0 | | (1-57) | no_pfam |
| HG1015049 | PLT00014333H11.contig.a | 95 | 0.03 | (1-95) | (36-95) | (12-35) | 0 | | (1-95) | no_pfam |
| HG1015050 | PLT00014333H15.contig.a | 90 | 0.23 | (35-90) | | (1-34) | 0 | | (1-90) | no_pfam |
| HG1015051 | PLT00014333H15.contig.b | 60 | 0 | (1-60) | | | 0 | | (1-60) | no_pfam |
| HG1015052 | PLT00014333I18.contig.a | 58 | 0.69 | (22-58) | (34-58) | (12-33) | 1 | (7-29) | (1-6)(30-58) | no_pfam |
| HG1015053 | PLT00014333I18.contig.b | 50 | 0.77 | (22-50) | | (1-21) | 0 | | (1-50) | no_pfam |
| HG1015054 | PLT00014333I22.contig.a | 70 | 0.08 | (1-70) | (19-70) | (1-18) | 0 | | (1-70) | no_pfam |
| HG1015055 | PLT00014333I22.contig.b | 54 | 0.96 | (23-54) | (25-54) | (1-24) | 1 | (6-28) | (1-5)(29-54) | no_pfam |
| HG1015056 | PLT00014333J01.contig.a | 84 | 0.03 | (1-84) | (35-84) | (19-34) | 0 | | (1-84) | no_pfam |
| HG1015057 | PLT00014333J01.contig.b | 66 | 0.08 | (32-66) | (33-66) | (1-32) | 0 | | (1-66) | no_pfam |
| HG1015058 | PLT00014333J13.contig.a | 106 | 0.02 | (1-106) | | | 1 | (46-68) | (1.45)(69-106) | no_pfam |
| HG1015059 | PLT00014333J13.contig.b | 93 | 0.06 | (37-93) | (1-93) | | 0 | | (1-93) | no_pfam |
| HG1015060 | PLT00014333J15.contig.a | 63 | 0.12 | (1-63) | (17-63) | (1-16) | 0 | | (1-63) | no_pfam |
| HG1015061 | PLT00014333J15.contig.b | 62 | 0.18 | (1-62) | (22-62) | (7-21) | 1 | (20-42) | (1-19)(43-62) | no_pfam |
| HG1015062 | PLT00014333J17.contig.a | 88 | 0 | (1-88) | (36-88) | (16-35) | 0 | | (1-88) | no_pfam |
| HG1015063 | PLT00014333J23.contig.a | 66 | 0.05 | (1-66) | (16-66) | (1-15) | 0 | | (1-66) | no_pfam |
| HG1015064 | PLT00014333J23.contig.b | 57 | 0.33 | (1-57) | (31-57) | (14-30) | 0 | | (1-57) | no_pfam |
| HG1015065 | PLT00014333K04.contig.a | 131 | 0.01 | (1-131) | | | 0 | | (1-131) | Gag_p24 |
| HG1015066 | PLT00014333K04.contig.b | 125 | 0.14 | (1-125) | (19-125) | (1-18) | 0 | | (1-125) | integrase |
| HG1015067 | PLT00014333K08.contig.a | 69 | 0.19 | (1-69) | (34-69) | (19-33) | 1 | (28-50) | (1-27)(51-69) | no_pfam |
| HG1015068 | PLT00014333K08.contig.b | 63 | 0.17 | (21-63) | | (1.20) | 0 | | (1-63) | no_pfam |
| HG1015069 | PLT00014333L13.contig.b | 52 | 0 | (1-52) | | | 0 | | (1-52) | maseH |
| HG1015070 | PLT00014333M01.contig.a | 110 | 0.29 | (1-110) | (20-110) | (1-19) | 1 | (86-108) | (1-85) | no_pfam |
| | | | | | | | | | (109-110) | |
| HG1015071 | PLT00014333M01.contig.b | 68 | 0.01 | (1-68) | (18-68) | (1-17) | 1 | (41-63) | (1-40)(64-68) | no_pfam |
| HG1015072 | PLT00014333M02.contig.a | 101 | 0.01 | (38-101) | (43-101) | (12-42) | 0 | | (1-101) | no_pfam |
| HG1015073 | PLT00014333M02.contig.b | 50 | 0 | (1-50) | (14-50) | (1-13) | 0 | | (1-50) | no_pfam |
| HG1015074 | PLT00014333M07.contig.a | 70 | 0.26 | (37-70) | (30-70) | (4-29) | 1 | (13-35) | (1-12)(36-70) | no_pfam |
| HG1015075 | PLT00014333M07.contig.b | 58 | 0.62 | (15-58) | (16-58) | (1-15) | 0 | | (1-58) | no_pfam |
| HG1015076 | PLT00014333M15.contig.a | 80 | 0.04 | (1-80) | (42-80) | (18-41) | 0 | | (1-80) | no_pfam |
| HG1015077 | PLT00014333M15.contig.b | 54 | 0.08 | (1-54) | (42-54) | (18-41) | 0 | | (1-54) | no_pfam |
| HG1015078 | PLT00014333N05.contig.a | 73 | 0.1 | (5-73) | (15-73) | (1-14) | 0 | | (1-73) | no_pfam |
| HG1015079 | PLT00014333N05.contig.b | 70 | 0.45 | (35-70) | (39-70) | (5-38) | 0 | | (1-70) | no_pfam |
| HG1015080 | PLT00014333N11.contig.a | 95 | 0.01 | (1-95) | (30-95) | (15-29) | 0 | | (1-95) | no_pfam |
| HG1015081 | PLT00014333N11.contig.b | 69 | 0.03 | (9-69) | (22-69) | (5-21) | 0 | | (1-69) | no_pfam |
| HG1015082 | PLT00014333O03.contig.a | 72 | 0.21 | (3-72) | (28-72) | (14-27) | 0 | | (1-72) | no_pfam |
| HG1015083 | PLT00014333O03.contig.b | 55 | 0.01 | (1-55) | (25-55) | (10-24) | 0 | | (1-55) | no_pfam |
| HG1015084 | PLT00014333O10.contig.a | 55 | 0.06 | (4-55) | (15-55) | (1-14) | 0 | | (1-55) | no_pfam |
| HG1015085 | PLT00014333O17.contig.a | 71 | 0.11 | (1-71) | (20-71) | (1-19) | 0 | | (1-71) | no_pfam |
| HG1015086 | PLT00014333E15.contig.a | 92 | 0.49 | (20-92) | | (1-19) | 1 | (5-27) | (1-4)(28-92) | no_pfam |
| HG1015087 | PLT00014333E15.contig.b | 78 | 0.01 | (1-78) | | | 1 | (52-71) | (1-51)(72-78) | no_pfam |
| HG1015088 | PLT00014333G09.contig.a | 125 | 0 | (1-125) | | | 0 | | (1-125) | no_pfam |
| HG1015089 | PLT00014333G09.contig.b | 63 | 0.11 | (1-63) | (41-63) | (18-40) | 0 | | (1-63) | no_pfam |

TABLE 3
Similarity to Known Sequences

| | | | | | Top Human | | |
|---|---|---|---|---|---|---|---|
| | | Top Hit | | | Hit Accession | Top Human Hit | Top |
| FP ID | Clone ID | Accession ID | Top Hit Annotation | Top Hit % ID | ID | Annotation | Human Hit % ID |
| HG1014903 | PLT00014330A02.contig.a | gi\|34529187\|dbj\| | unnamed protein | 59 | gi\|34529187\|dbj\| | unnamed protein | 59 |
| | | BAC85656.1\| | product [Homo sapiens] | | BAC85656.1\| | product [Homo sapiens] | |
| HG1014910 | PLT00014330B02.contig.b | gi\|7770237\|gb\|AAF69654.1\| | PRO2822 [Homo sapiens] | 76 | gi\|7770237\|gb\|AAF69654.1\| | PRO2822 [Homo sapiens] | 76 |
| HG1014914 | PLT00014330B11.contig.a | gi\|38085361\|ref\| | similar to RIKEN | 80 | | no_human_hit | |
| | | XP_355822.1\| | cDNA 6330419J24 | | | | |
| | | | gene [Mus musculus] | | | | |
| HG1014933 | PLT00014330D12.contig.b | gi\|8923214\|ref\| | signal-transducing | 57 | gi\|8923214\|ref\| | signal-transducing | 57 |
| | | NP_060190.1\| | adaptor protein-2; brk | | NP_060190.1\| | adaptor protein-2; | |
| | | | kinase substrate | | | brk kinase substrate | |
| | | | [Homo sapiens] | | | [Homo sapiens] | |
| | | | gi\|7020193\|dbj\|BAA91028.1\| | | | gi\|7020193\|dbj\|BAA91028.1\| | |
| | | | unnamed | | | unnamed | |
| | | | protein product [Homo | | | protein product | |
| | | | sapiens] | | | [Homo sapiens] | |
| HG1014948 | PLT00014330F05.contig.a | gi\|34534372\|dbj\| | unnamed protein | 56 | gi\|34534372\|dbj\| | unnamed protein | 56 |
| | | BAC86987.1\| | product [Homo | | BAC86987.1\| | product [Homo | |
| | | | sapiens] | | | sapiens] | |
| HG1014952 | PLT00014330H05.contig.b | gi\|2981631\|dbj\| | ORF2 [Canis | 58 | no_human_hit | | |
| | | BAA25253.1\| | familiaris] | | | | |
| HG1014958 | PLT00014330H18.contig.a | gi\|13310191\|gb\| | recombinant envelope | 52 | no_human_hit | | |
| | | AAK18189.1\| | protein [multiple | | | | |
| | | | sclerosis associated | | | | |
| | | | retrovirus element] | | | | |
| HG1014971 | PLT00014330J21.contig.b | gi\|23503335\|ref\| | hypothetical protein | 64 | gi\|23503335\|ref\| | hypothetical protein | 64 |
| | | NP_694983.1\| | FLJ25952 [Homo sapiens] | | NP_694983.1\| | FLJ25952 [Homo sapiens] | |
| | | | gi\|21758947\|dbj\|BAC05422.1\| | | | gi\|21758947\|dbj\|BAC05422.1\| | |
| | | | unnamed | | | unnamed | |
| | | | protein product [Homo | | | protein product | |
| | | | sapiens] | | | [Homo sapiens] | |
| HG1014975 | PLT00014330K09.contig.a | gi\|34528691\|dbj\| | unnamed protein | 56 | gi\|34528691\|dbj\| | unnamed protein | 56 |
| | | BAC85556.1\| | product [Homo | | BAC85556.1\| | product [Homo | |
| | | | sapiens] | | | sapiens] | |
| HG1014977 | PLT00014330K15.contig.a | gi\|34533624\|dbj\| | unnamed protein | 81 | gi\|34533624\|dbj\| | unnamed protein | 81 |
| | | BAC86755.1\| | product [Homo sapiens] | | BAC86755.1\| | product [Homo sapiens] | |
| HG1014983 | PLT00014330M08.contig.a | gi\|21754422\|dbj\| | unnamed protein | 55 | gi\|21754422\|dbj\| | unnamed protein | 55 |
| | | BAC04501.1\| | product [Homo sapiens] | | BAC04501.1\| | product [Homo sapiens] | |
| HG1014992 | PLT00014330N13.contig.a | gi\|37182643\|gb\| | DRDL5813 [Homo | 56 | gi\|37182643\|gb\| | DRDL5813 [Homo | 56 |
| | | AAQ89122.1\| | sapiens] | | AAQ89122.1\| | sapiens] | |
| HG1015030 | PLT00014333C21.contig.b | gi\|18027736\|gb\| | unknown [Homo | 87 | gi\|18027736\|gb\| | unknown [Homo | 87 |
| | | AAL55829.1\| | sapiens] | | AAL55829.1\| | sapiens] | |
| HG1015044 | PLT00014333E24.contig.b | gi\|1698455\|gb\|AAC52011.1\| | mariner transposase | 79 | gi\|1698455\|gb\|AAC52011.1\| | mariner transposase | 79 |
| | | | [Homo sapiens] | | | [Homo sapiens] | |
| HG1015082 | PLT00014333O03.contig.a | gi\|21754422\|dbj\| | unnamed protein | 75 | gi\|21754422\|dbj\| | unnamed protein | 75 |
| | | BAC04501.1\| | product [Homo sapiens] | | BAC04501.1\| | product [Homo sapiens] | |

TABLE 4
Structural Characteristics and Tissue Source

| | | | | | | | Altern | Altern | | | |
|---|---|---|---|---|---|---|---|---|---|---|---|
| | | | Pred | | Signal | Mature | Signal | Mature | | | |
| | | | Prot | Tree- | Peptide | Protein | Peptide | Protein | | TM | Non-TM |
| FP ID | Clone ID | Tissue Source | Len | vote | Coords | Coords | Coords | Coords | TM | Coords | Coords |
| HG1014905 | CLN00082984 | Muscle, Muscle Pool | 82 | 0.55 | | (1-82) | (14-26) | (27-82) | 1 | (15-37) | (1-14)(38-82) |
| HG1014906 | CLN00082984 | Muscle, Muscle Pool | 61 | 0.62 | (6.23) | (24-61) | (11-23) | (24-61) | 2 | (5-27) | (1-4)(28-30) |
| | | | | | | | | | | (31-53) | (54-61) |
| HG1014917 | CLN00142812 | Colon | 74 | 0.7 | (2-21) | (22-74) | (9-21) | (22-74) | 0 | | (1-74) |
| HG1014918 | CLN00142812 | Colon | 53 | 0.24 | | (1-53) | (15-27) | (28-53) | 0 | | (1-53) |
| HG1014919 | CLN00077158 | Intestine, | 101 | 0.53 | (21-45) | (46-101) | | | 0 | | (1-101) |
| | | Pancreas, Pancreas Pool, | | | | | | | | | |
| | | Stomach, Stomach | | | | | | | | | |
| | | pool, Trachea, Trachea | | | | | | | | | |
| | | pool | | | | | | | | | |
| HG1014925 | CLN00059368 | Kidney | 132 | 0.81 | (1-19) | (20-132) | | | 0 | | (1-132) |
| HG1014926 | CLN00059368 | Kidney | 74 | 0.43 | (15-36) | (37-74) | | | 2 | (12-31) | (1-11)(32-45) |
| | | | | | | | | | | (46-68) | (69-74) |
| HG1014930 | CLN00156143 | Testis, Testis Pool | 79 | 0.61 | (6-29) | (30-79) | (16-28) | (29-79) | 0 | | (1-79) |
| HG1014931 | CLN00156143 | Testis, Testis Pool | 73 | 0.87 | (1-19) | (20-73) | (9-21) | (22-73) | 0 | | (1-73) |
| HG1014932 | CLN00062536 | Kidney | 116 | 0.01 | | (1-116) | | | 1 | (21-43) | (1-20)(44-116) |
| HG1014936 | CLN00163455 | Prostate, Prostate Pool | 89 | 0.4 | (22-35) | (36-89) | (9-21) | (22-89) | 1 | (12-34) | (1-11)(35-89) |
| | | | | | | | (23-35) | (36-89) | | | |
| HG1014937 | CLN00139538 | Breast | 96 | 0.26 | (10-26) | (27-96) | (17-29) | (30-96) | 0 | | (1-96) |
| HG1014942 | CLN00051182 | Bladder, Brain, Brain | 62 | 0.16 | | (1-62) | | | 1 | (15-34) | (1-14)(35-62) |
| | | Pool, Lung, Lung Pool, | | | | | | | | | |
| | | Spleen, Spleen Pool, | | | | | | | | | |
| | | Thymus, Thymus pool | | | | | | | | | |
| HG1014943 | CLN00018119 | Intestine, Pancreas, | 77 | 0 | | (1-77) | | | 1 | (28-45) | (1-27)(46-77) |
| | | Pancreas Pool, | | | | | | | | | |
| | | Stomach, Stomach | | | | | | | | | |
| | | pool, Trachea, Trachea | | | | | | | | | |
| | | pool | | | | | | | | | |
| HG1014946 | CLN00156600 | Testis, Testis Pool | 117 | 0.9 | (1-19) | (20-117) | (5-17) | (18-117) | 0 | | (1-117) |
| HG1014949 | CLN00010970 | Bone Marrow, Bone | 53 | 0.26 | (1-27) | (28-53) | (15-27) | (28-53) | 0 | | (1-53) |
| | | Marrow Pool, Liver | | | | | | | | | |
| HG1014951 | CLN00148049 | Cord Blood, Cord | 53 | 0.05 | | (1-53) | | | 1 | (20-42) | (1-19)(43-53) |
| | | Blood Pool, Placenta, | | | | | | | | | |
| | | Placenta Pool | | | | | | | | | |
| HG1014954 | CLN00118656 | Bladder, Brain, Brain | 86 | 0.65 | (1-18) | (19-86) | | | 0 | | (1-86) |
| | | Pool, Lung, Lung Pool, | | | | | | | | | |
| | | Spleen, Spleen Pool, | | | | | | | | | |
| | | Thymus, Thymus pool | | | | | | | | | |
| HG1014957 | CLN00185900 | Breast | 66 | 0.05 | | (1-66) | (16-28) | (29-66) | 1 | (43-62) | (1-42)(63-66) |
| HG1014958 | CLN00185984 | Breast | 95 | 0.94 | (1-18) | (19-95) | (8-20) | (21-95) | 0 | | (1-95) |
| HG1014960 | CLN00020358 | Intestine, Pancreas, | 62 | 0.05 | | (1-62) | | | 1 | (31-53) | (1-30)(54-62) |
| | | Pancreas Pool, | | | | | | | | | |
| | | Stomach, Stomach | | | | | | | | | |
| | | pool, Trachea, Trachea | | | | | | | | | |
| | | pool | | | | | | | | | |
| HG1014962 | CLN00149057 | Breast | 66 | 0.51 | (1-15) | (16-66) | (8-14) | (15-66) | 2 | (4-26) | (1-3)(27-42) |
| | | | | | | | (2-8) | (9-66) | | (43-65) | (66-66) |
| | | | | | | | (1-7) | (8-66) | | | |
| HG1014973 | CLN00051702 | no tissue source found | 99 | 0.16 | | (1-99) | | | 1 | (73-95) | (1-72)(96-99) |
| HG1014974 | CLN00051702 | no tissue source found | 50 | 0.26 | | (1-50) | | | 2 | (5-27) | (1-4)(28-31) |
| | | | | | | | | | | (32-49) | (50-50) |
| HG1014975 | CLN00041527 | Adrenal Gland, Adrenal | 100 | 0.09 | | (1-100) | (7-19) | (20-100) | 0 | | (1-100) |
| | | Gland Pool | | | | | | | | | |
| HG1014979 | CLN00109327 | Liver | 51 | 0.17 | | (1-51) | | | 1 | (13-35) | (1-12)(36-51) |
| HG1014983 | CLN00054904 | Bladder, Brain, Brain | 72 | 0.46 | (18-31) | (32-72) | (21-33) | (34-72) | 1 | (45-67) | (1-44)(68-72) |
| | | Pool, Lung, Lung Pool, | | | | | (19-31) | (32-72) | | | |
| | | Spleen, Spleen Pool, | | | | | | | | | |
| | | Thymus, Thymus pool | | | | | | | | | |
| HG1014984 | CLN00054904 | Bladder, Brain, Brain | 52 | 0.29 | | (1-52) | (18-30) | (31-52) | 1 | (20-42) | (1-19)(43-52) |
| | | Pool, Lung, Lung Pool, | | | | | | | | | |
| | | Spleen, Spleen Pool, | | | | | | | | | |
| | | Thymus, Thymus pool | | | | | | | | | |
| HG1014987 | CLN00138883 | Intestine, Pancreas, | 82 | 0.45 | (16-29) | (30-82) | (16-28) | (29-82) | 0 | | (1-82) |
| | | Pancreas Pool, | | | | | | | | | |
| | | Stomach, Stomach | | | | | | | | | |
| | | pool, Trachea, Trachea | | | | | | | | | |
| | | pool | | | | | | | | | |
| HG1014988 | CLN00113699 | Bladder, Brain, Brain | 75 | 0.15 | (23-40) | (41-75) | | | 1 | (20-42) | (1-19)(43-75) |
| | | Pool, Lung, Lung Pool, | | | | | | | | | |
| | | Spleen, Spleen Pool, | | | | | | | | | |
| | | Thymus, Thymus pool | | | | | | | | | |
| HG1014992 | CLN00155027 | Testis, Testis Pool | 83 | 0.87 | (1-19) | (20-83) | (10-22) | (23-83) | 1 | (4-26) | (1-3)(27-83) |
| HG1014993 | CLN00155027 | Testis, Testis Pool | 55 | 0.29 | | (1-55) | | | 1 | (10-32) | (1-9)(33-55) |
| HG1014996 | CLN00042242 | Muscle, Muscle Pool | 70 | 0.32 | (5-18) | (19-70) | | | 1 | (7-29) | (1-6)(30-70) |
| HG1015004 | CLN00116255 | Bladder, Brain, Brain | 50 | 0.17 | (23-37) | (38-50) | (21-33) | (34-50) | 0 | | (1-50) |
| | | Pool, Lung, Lung Pool, | | | | | (25-37) | (38-50) | | | |
| | | Spleen, Spleen Pool, | | | | | | | | | |
| | | Thymus, Thymus pool | | | | | | | | | |
| HG1015007 | CLN00200943 | Prostate, Prostate Pool | 83 | 0.08 | | (1-83) | | | 1 | (15-37) | (1-14)(38-83) |
| HG1015010 | CLN00123672 | Intestine, Pancreas, | 66 | 0.13 | | (1-66) | (22-34) | (35-66) | 0 | | (1-66) |
| | | Pancreas Pool, | | | | | | | | | |
| | | Stomach, Stomach | | | | | | | | | |
| | | pool, Trachea, Trachea | | | | | | | | | |
| | | pool | | | | | | | | | |
| HG1015013 | CLN00197177 | Prostate Pool, Prostate | 67 | 0.8 | (17-34) | (35-67) | | | 0 | | (1-67) |
| HG1015015 | CLN00195394 | Lung, Lung Pool | 50 | 0.46 | (16-40) | (41-50) | (25-37) | (38-50) | 0 | | (1-50) |
| | | | | | | | (12-24) | (25-50) | | | |
| HG1015018 | CLN00191228 | Lung, Lung Pool | 55 | 0.05 | | (1-55) | | | 1 | (29-51) | (1-28)(52-55) |
| HG1015019 | CLN00191228 | Lung, Lung Pool | 53 | 0.49 | (23-46) | (47-53) | | | 0 | | (1-53) |
| HG1015022 | CLN00192344 | Lung, Lung Pool | 65 | 0.01 | | (1-65) | | | 1 | (42-64) | (1-41)(65-65) |
| HG1015024 | CLN00236321 | Tonsil, Tonsil pool | 51 | 0.77 | | (1-51) | (9-21) | (22-51) | 1 | (12-34) | (1-11)(35-51) |
| HG1015031 | CLN00041415 | Adrenal Gland, Adrenal | 94 | 0.11 | | (1-94) | | | 1 | (10-32) | (1-9)(33-94) |
| | | Gland Pool | | | | | | | | | |
| HG1015032 | CLN00041415 | Adrenal Gland, Adrenal | 88 | 0 | | (1-88) | | | 2 | (34-56) | (1-33)(57-60) |
| | | Gland Pool | | | | | | | | (61-78) | (79-88) |
| HG1015036 | CLN00081508 | Muscle Pool, Muscle | 62 | 0.29 | | (1-62) | | | 2 | (13-32) | (1-12)(33-41) |
| | | | | | | | | | | (42-61) | (62-62) |
| HG1015037 | CLN00114957 | Bladder, Brain, Brain | 73 | 0 | | (1-73) | | | 1 | (26-48) | (1-25)(49-73) |
| | | Pool, Lung, Lung Pool, | | | | | | | | | |
| | | Spleen, Spleen Pool, | | | | | | | | | |
| | | Thymus, Thymus pool | | | | | | | | | |
| HG1015038 | CLN00114957 | Bladder, Brain, Brain | 67 | 0.51 | (8-25) | (26-67) | | | 1 | (10-32) | (1-9)(33-67) |
| | | Pool, Lung, Lung Pool, | | | | | | | | | |
| | | Spleen, Spleen Pool, | | | | | | | | | |
| | | Thymus, Thymus pool | | | | | | | | | |
| HG1015047 | CLN00123946 | Intestine, Pancreas, | 77 | 0.03 | | (1-77) | (6-18) | (19-77) | 0 | | (1-77) |
| | | Pancreas Pool, | | | | | | | | | |
| | | Stomach, Stomach | | | | | | | | | |
| | | pool, Trachea, Trachea | | | | | | | | | |
| | | pool | | | | | | | | | |
| HG1015050 | CLN00024579 | Bone Marrow, Bone | 90 | 0.23 | (1-34) | (35-90) | (24-36) | (37-90) | 0 | | (1-90) |
| | | Marrow Pool, Liver | | | | | (22-34) | (35-90) | | | |
| HG1015052 | CLN00195792 | Lung, Lung Pool | 58 | 0.69 | (12-33) | (34-58) | (5-17) | (18-58) | 1 | (7-29) | (1-6)(30-58) |
| | | | | | | | (14-26) | (27-58) | | | |
| | | | | | | | (9-21) | (22-58) | | | |
| HG1015053 | CLN00195792 | Lung, Lung Pool | 50 | 0.77 | (1-21) | (22-50) | (9-21) | (22-50) | 0 | | (1-50) |
| HG1015055 | CLN00199902 | Prostate, Prostate Pool | 54 | 0.96 | (1-24) | (25-54) | (10-22) | (23-54) | 1 | (6-28) | (1-5)(29-54) |
| HG1015058 | CLN00023292 | Bone Marrow, Bone | 106 | 0.02 | | (1-106) | | | 1 | (46-68) | (1-45)(69-106) |
| | | Marrow Pool, Liver | | | | | | | | | |
| HG1015061 | CLN00168841 | Tonsil, Tonsil pool | 62 | 0.18 | | (1-62) | | | 1 | (20-42) | (1-19)(43-62) |
| HG1015067 | CLN00197776 | Prostate, Prostate Pool | 69 | 0.19 | | (1-69) | | | 1 | (28-50) | (1-27)(51-69) |
| HG1015068 | CLN00197776 | Prostate, Prostate Pool | 63 | 0.17 | | (1-63) | (8-20) | (21-63) | 0 | | (1-63) |
| HG1015070 | CLN00198831 | Prostate, Prostate Pool | 110 | 0.29 | (1-19) | (20-110) | | | 1 | (86-108) | (1-85) |
| | | | | | | | | | | | (109-110) |
| HG1015071 | CLN00198831 | Prostate, Prostate Pool | 68 | 0.01 | | (1-68) | | | 1 | (41-63) | (1-40)(64-68) |
| HG1015074 | CLN00202085 | Colon | 70 | 0.26 | (22-36) | (37-70) | (24-36) | (37-70) | 1 | (13-35) | (1-12)(36-70) |
| HG1015075 | CLN00202085 | Colon | 58 | 0.62 | (1-15) | (16-58) | | | 0 | | (1-58) |
| HG1015079 | CLN00243977 | Tonsil, Tonsil pool | 70 | 0.45 | (5-38) | (39-70) | | | 0 | | (1-70) |
| HG1015086 | CLN00226626 | Skin, Skin Pool | 92 | 0.49 | | (1-92) | | | 1 | (5-27) | (1-4)(28-92) |
| HG1015087 | CLN00226626 | Skin, Skin Pool | 78 | 0.01 | | (1-78) | | | 1 | (52-71) | (1-51)(72-78) |

TABLE 5
Subclone Identification and Similarity to Known Sequences

| | | | | | | | | | | | | Top | | | | |
|---|---|---|---|---|---|---|---|---|---|---|---|---|---|---|---|---|
| | | | | | | | Top | | | | | Hum | | | | |
| | | | | | | | Hit | | | | | Hit | | | | |
| | | | | | | Top | Len # | % ID | % ID | Top Hum | Top | Len # | % ID | | | |
| | Clone | | Tree- | | Top Hit | Hit | AA | Mat | Mat | Hit | Hum | AA | Mat | % ID Mat | Subclone | Subclone |
| FP ID | ID | Pred Prot Len | vote | TM | Annotation | Len | Mat | (QL) | (HL) | Annotation | Hit Len | Mat | (QL) | (HL) | Type | ID |
| HG1014905 | CLN00082984 | 82 | 0.55 | 1 | unnamed | 161 | 42 | 51% | 26% | unnamed | 177 | 38 | 46% | 21% | pTT5 | CLN00736344 |
| | | | | | protein | | | | | protein | | | | | | |
| | | | | | product | | | | | product | | | | | | |
| | | | | | [Mus | | | | | [Homo | | | | | | |
| | | | | | musculus] | | | | | sapiens] | | | | | | |
| HG1014906 | CLN00082984 | 61 | 0.62 | 2 | unnamed | 198 | 23 | 38% | 12% | unnamed | 198 | 23 | 38% | 12% | pTT5 | CLN00736344 |
| | | | | | protein | | | | | protein | | | | | | |
| | | | | | product | | | | | product | | | | | | |
| | | | | | [Homo | | | | | [Homo | | | | | | |
| | | | | | sapiens] | | | | | sapiens] | | | | | | |
| HG1014917 | CLN00142812 | 74 | 0.7 | 0 | | | | | | | | | | | pTT5 | CLN00736494 |
| HG1014919 | CLN00077158 | 101 | 0.53 | 0 | unnamed | 161 | 49 | 49% | 30% | unnamed | 161 | 49 | 49% | 30% | | |
| | | | | | protein | | | | | protein | | | | | | |
| | | | | | product | | | | | product | | | | | | |
| | | | | | [Homo | | | | | [Homo | | | | | | |
| | | | | | sapiens] | | | | | sapiens] | | | | | | |
| HG1014925 | CLN00059368 | 132 | 0.81 | 0 | | | | | | | | | | | pTT5 | CLN00736483 |
| HG1014926 | CLN00059368 | 74 | 0.43 | 2 | Legionella | 633 | 23 | 31% | 4% | | | | | | pTT5 | CLN00736483 |
| | | | | | vir | | | | | | | | | | | |
| | | | | | homologue | | | | | | | | | | | |
| | | | | | protein | | | | | | | | | | | |
| | | | | | [Legionella | | | | | | | | | | | |
| | | | | | pneumophila | | | | | | | | | | | |
| | | | | | str. Lens] | | | | | | | | | | | |
| HG1014930 | CLN00156143 | 79 | 0.61 | 0 | elongation | 535 | 42 | 53% | 8% | elongation | 535 | 42 | 53% | 8% | pTT5 | CLN00736320 |
| | | | | | protein 4 | | | | | protein 4 | | | | | | |
| | | | | | homolog | | | | | homolog | | | | | | |
| | | | | | [Homo | | | | | [Homo | | | | | | |
| | | | | | sapiens] | | | | | sapiens] | | | | | | |
| HG1014931 | CLN00156143 | 73 | 0.87 | 0 | | | | | | | | | | | pTT5 | CLN00736320 |
| HG1014932 | CLN00062536 | 116 | 0.01 | 1 | PRO0898 | 111 | 45 | 39% | 41% | PRO0898 | 111 | 45 | 39% | 41% | pTT5 | CLN00736408 |
| | | | | | [Homo | | | | | [Homo | | | | | | |
| | | | | | sapiens] | | | | | sapiens] | | | | | | |
| HG1014942 | CLN00051182 | 62 | 0.16 | 1 | Unknown | 591 | 24 | 39% | 4% | | | | | | | |
| | | | | | (protein for | | | | | | | | | | | |
| | | | | | IMAGE: 7122468) | | | | | | | | | | | |
| | | | | | [Rattus | | | | | | | | | | | |
| | | | | | norvegicus] | | | | | | | | | | | |
| HG1014946 | CLN00156600 | 117 | 0.9 | 0 | HERV- | 604 | 75 | 64% | 12% | HERV- | 604 | 75 | 64% | 12% | pTT5 | CLN00736568 |
| | | | | | R_7q21.2 | | | | | R_7q21.2 | | | | | | |
| | | | | | provirus | | | | | provirus | | | | | | |
| | | | | | ancestral | | | | | ancestral | | | | | | |
| | | | | | Env | | | | | Env | | | | | | |
| | | | | | polyprotein | | | | | polyprotein | | | | | | |
| | | | | | precursor | | | | | precursor | | | | | | |
| | | | | | (Envelope | | | | | (Envelope | | | | | | |
| | | | | | polyprotein) | | | | | polyprotein) | | | | | | |
| | | | | | (ERV3 | | | | | (ERV3 | | | | | | |
| | | | | | envelope | | | | | envelope | | | | | | |
| | | | | | protein) | | | | | protein) | | | | | | |
| | | | | | (ERV-3 | | | | | (ERV-3 | | | | | | |
| | | | | | envelope | | | | | envelope | | | | | | |
| | | | | | protein) | | | | | protein) | | | | | | |
| | | | | | (HERV-R | | | | | (HERV-R | | | | | | |
| | | | | | envelope | | | | | envelope | | | | | | |
| | | | | | protein) | | | | | protein) | | | | | | |
| | | | | | (ERV-R | | | | | (ERV-R | | | | | | |
| | | | | | envelope | | | | | envelope | | | | | | |
| | | | | | protein) | | | | | protein) | | | | | | |
| | | | | | [Contains: | | | | | [Contains: | | | | | | |
| | | | | | Surface | | | | | Surface | | | | | | |
| | | | | | protein | | | | | protein | | | | | | |
| | | | | | (SU); | | | | | (SU); | | | | | | |
| | | | | | Transmembrane | | | | | Transmembrane | | | | | | |
| | | | | | protein | | | | | protein | | | | | | |
| | | | | | (TM)] | | | | | (TM)] | | | | | | |
| HG1014949 | CLN00010970 | 53 | 0.26 | 0 | KIAA1822 | 533 | 25 | 47% | 5% | KIAA1822 | 533 | 25 | 47% | 5% | | |
| | | | | | protein | | | | | protein | | | | | | |
| | | | | | [Homo | | | | | [Homo | | | | | | |
| | | | | | sapiens] | | | | | sapiens] | | | | | | |
| HG1014951 | CLN00148049 | 53 | 0.05 | 1 | unnamed | 141 | 16 | 30% | 11% | | | | | | | |
| | | | | | protein | | | | | | | | | | | |
| | | | | | product | | | | | | | | | | | |
| | | | | | [Mus | | | | | | | | | | | |
| | | | | | musculus] | | | | | | | | | | | |
| HG1014954 | CLN00118656 | 86 | 0.65 | 0 | OSJNBa0016O02.7 | 484 | 33 | 38% | 7% | | | | | | pTT5 | CLN00736486 |
| | | | | | [Oryza | | | | | | | | | | | |
| | | | | | sativa | | | | | | | | | | | |
| | | | | | (japonica | | | | | | | | | | | |
| | | | | | cultivar- | | | | | | | | | | | |
| | | | | | group)] | | | | | | | | | | | |
| | | | | | gi\|38606520\| | | | | | | | | | | | |
| | | | | | emb\|CAE05997.2\| | | | | | | | | | | | |
| | | | | | OSJNBa0016O02.7 | | | | | | | | | | | |
| | | | | | [Oryza | | | | | | | | | | | |
| | | | | | sativa | | | | | | | | | | | |
| | | | | | (japonica | | | | | | | | | | | |
| | | | | | cultivar- | | | | | | | | | | | |
| | | | | | group)] | | | | | | | | | | | |
| HG1014958 | CLN00185984 | 95 | 0.94 | 0 | recombinant | 542 | 54 | 57% | 10% | | | | | | pTT5 | CLN00736439 |
| | | | | | envelope | | | | | | | | | | | |
| | | | | | protein | | | | | | | | | | | |
| | | | | | [multiple | | | | | | | | | | | |
| | | | | | sclerosis | | | | | | | | | | | |
| | | | | | associated | | | | | | | | | | | |
| | | | | | retrovirus | | | | | | | | | | | |
| | | | | | element] | | | | | | | | | | | |
| HG1014960 | CLN00020358 | 62 | 0.05 | 1 | hypothetical | 118 | 21 | 34% | 18% | | | | | | | |
| | | | | | protein hcl- | | | | | | | | | | | |
| | | | | | mouse | | | | | | | | | | | |
| | | | | | (fragment) | | | | | | | | | | | |
| | | | | | gi\|1333929\|emb\| | | | | | | | | | | | |
| | | | | | CAA46991.1\| | | | | | | | | | | | |
| | | | | | unnamed | | | | | | | | | | | |
| | | | | | protein | | | | | | | | | | | |
| | | | | | product | | | | | | | | | | | |
| | | | | | [Mus | | | | | | | | | | | |
| | | | | | musculus] | | | | | | | | | | | |
| HG1014962 | CLN00149057 | 66 | 0.51 | 2 | PREDICTED: | 1667 | 20 | 30% | 1% | | | | | | | |
| | | | | | similar to | | | | | | | | | | | |
| | | | | | MGC68847 | | | | | | | | | | | |
| | | | | | protein | | | | | | | | | | | |
| | | | | | [Gallus | | | | | | | | | | | |
| | | | | | gallus] | | | | | | | | | | | |
| HG1014973 | CLN00051702 | 99 | 0.16 | 1 | unnamed | 124 | 36 | 36% | 29% | unnamed | 124 | 36 | 36% | 29% | | |
| | | | | | protein | | | | | protein | | | | | | |
| | | | | | product | | | | | product | | | | | | |
| | | | | | [Homo | | | | | [Homo | | | | | | |
| | | | | | sapiens] | | | | | sapiens] | | | | | | |
| | | | | | gi\|34531176\| | | | | | | | | | | | |
| | | | | | dbj\|BAC86070.1\| | | | | | | | | | | | |
| | | | | | unnamed | | | | | | | | | | | |
| | | | | | protein | | | | | | | | | | | |
| | | | | | product | | | | | | | | | | | |
| | | | | | [Homo | | | | | | | | | | | |
| | | | | | sapiens] | | | | | | | | | | | |
| HG1014974 | CLN00051702 | 50 | 0.26 | 2 | ORF | 157 | 19 | 38% | 12% | | | | | | | |
| | | | | | MSV222 | | | | | | | | | | | |
| | | | | | hypothetical | | | | | | | | | | | |
| | | | | | protein | | | | | | | | | | | |
| | | | | | [Melanoplus | | | | | | | | | | | |
| | | | | | sanguinipesentomopoxvirus] | | | | | | | | | | | |
| | | | | | gi\|11362396\| | | | | | | | | | | | |
| | | | | | pir\|\|T28383 | | | | | | | | | | | |
| | | | | | ORF | | | | | | | | | | | |
| | | | | | MSV222 | | | | | | | | | | | |
| | | | | | hypothetical | | | | | | | | | | | |
| | | | | | protein - | | | | | | | | | | | |
| | | | | | Melanoplus | | | | | | | | | | | |
| | | | | | sanguinipesentomopoxvirus | | | | | | | | | | | |
| | | | | | gi\|9631394\|ref\| | | | | | | | | | | | |
| | | | | | NP_048293.1\| | | | | | | | | | | | |
| | | | | | ORF | | | | | | | | | | | |
| | | | | | MSV222 | | | | | | | | | | | |
| | | | | | hypothetical | | | | | | | | | | | |
| | | | | | protein | | | | | | | | | | | |
| | | | | | [Melanoplus | | | | | | | | | | | |
| | | | | | sanguinipesentomopoxvirus] | | | | | | | | | | | |
| HG1014975 | CLN00041527 | 100 | 0.09 | 0 | PREDICTED: | 178 | 56 | 56% | 31% | PREDICTED: | 178 | 56 | 56% | 31% | pTT5 | CLN00736375 |
| | | | | | similar to | | | | | similar to | | | | | | |
| | | | | | FLJ44076 | | | | | FLJ44076 | | | | | | |
| | | | | | protein | | | | | protein | | | | | | |
| | | | | | [Homo | | | | | [Homo | | | | | | |
| | | | | | sapiens] | | | | | sapiens] | | | | | | |
| HG1014979 | CLN00109327 | 51 | 0.17 | 1 | NADH | 306 | 19 | 37% | 6% | | | | | | | |
| | | | | | dehydrogenese | | | | | | | | | | | |
| | | | | | subunit 5 | | | | | | | | | | | |
| | | | | | [Luciola | | | | | | | | | | | |
| | | | | | lateralis] | | | | | | | | | | | |
| HG1014983 | CLN00054904 | 72 | 0.46 | 1 | unnamed | 129 | 41 | 57% | 32% | unnamed | 129 | 41 | 57% | 32% | | |
| | | | | | protein | | | | | protein | | | | | | |
| | | | | | product | | | | | product | | | | | | |
| | | | | | [Homo | | | | | [Homo | | | | | | |
| | | | | | sapiens] | | | | | sapiens] | | | | | | |
| HG1014984 | CLN00054904 | 52 | 0.29 | 1 | hypothetical | 59 | 16 | 31% | 27% | | | | | | | |
| | | | | | protein | | | | | | | | | | | |
| | | | | | MYPE2715 | | | | | | | | | | | |
| | | | | | [Mycoplasma | | | | | | | | | | | |
| | | | | | penetrans | | | | | | | | | | | |
| | | | | | HF-2] | | | | | | | | | | | |
| | | | | | gi\|26453732\| | | | | | | | | | | | |
| | | | | | dbj\|BAC44063.1\| | | | | | | | | | | | |
| | | | | | unknown | | | | | | | | | | | |
| | | | | | [Mycoplasma | | | | | | | | | | | |
| | | | | | penetrans | | | | | | | | | | | |
| | | | | | HF-2] | | | | | | | | | | | |
| HG1014987 | CLN00138883 | 82 | 0.45 | 0 | | | | | | | | | | | pTT5 | CLN00736332 |
| HG1014988 | CLN00113699 | 75 | 0.15 | 1 | KIAA1657 | 127 | 26 | 35% | 20% | KIAA1657 | 127 | 26 | 35% | 20% | | |
| | | | | | protein | | | | | protein | | | | | | |
| | | | | | [Homo | | | | | [Homo | | | | | | |
| | | | | | sapiens] | | | | | sapiens] | | | | | | |
| HG1014992 | CLN00155027 | 83 | 0.87 | 1 | DRDL5813 | 653 | 49 | 59% | 8% | DRDL5813 | 653 | 49 | 59% | 8% | pTT5 | CLN00736512 |
| | | | | | [Homo | | | | | [Homo | | | | | | |
| | | | | | sapiens] | | | | | sapiens] | | | | | | |
| HG1014993 | CLN00155027 | 55 | 0.29 | 1 | PRO2532 | 71 | 18 | 33% | 25% | PRO2532 | 71 | 18 | 33% | 25% | pTT5 | CLN00736512 |
| | | | | | [Homo | | | | | [Homo | | | | | | |
| | | | | | sapiens] | | | | | sapiens] | | | | | | |
| HG1014996 | CLN00042242 | 70 | 0.32 | 1 | protein with | 695 | 23 | 33% | 3% | | | | | | pTT5 | CLN00736478 |
| | | | | | R3H and G- | | | | | | | | | | | |
| | | | | | patch | | | | | | | | | | | |
| | | | | | domain | | | | | | | | | | | |
| | | | | | [Schizosaccharomyces | | | | | | | | | | | |
| | | | | | pombe] | | | | | | | | | | | |
| | | | | | gi\|3417428\|emb\| | | | | | | | | | | | |
| | | | | | CAA20315.1\| | | | | | | | | | | | |
| | | | | | SPBC30B4.02c | | | | | | | | | | | |
| | | | | | [Schizosaccharomyces | | | | | | | | | | | |
| | | | | | pombe] | | | | | | | | | | | |
| | | | | | gi\|7491581\|pir\|\| | | | | | | | | | | | |
| | | | | | T40168 | | | | | | | | | | | |
| | | | | | hypothetical | | | | | | | | | | | |
| | | | | | protein | | | | | | | | | | | |
| | | | | | SPBC30B4.02c - | | | | | | | | | | | |
| | | | | | fission | | | | | | | | | | | |
| | | | | | yeast | | | | | | | | | | | |
| | | | | | (Schizosaccharomyces | | | | | | | | | | | |
| | | | | | pombe) | | | | | | | | | | | |
| HG1015007 | CLN00200943 | 83 | 0.08 | 1 | | | | | | | | | | | pTT5 | CLN00736321 |
| HG1015013 | CLN00197177 | 67 | 0.8 | 0 | 1- | 227 | 21 | 31% | 9% | | | | | | pTT5 | CLN00736625 |
| | | | | | aminocyclopropane- | | | | | | | | | | | |
| | | | | | 1- | | | | | | | | | | | |
| | | | | | carboxylate | | | | | | | | | | | |
| | | | | | synthase | | | | | | | | | | | |
| | | | | | [Lycopersicon | | | | | | | | | | | |
| | | | | | esculentum] | | | | | | | | | | | |
| HG1015018 | CLN00191228 | 55 | 0.05 | 1 | PREDICTED: | 105 | 29 | 53% | 28% | PREDICTED: | 105 | 29 | 53% | 28% | | |
| | | | | | hypothetical | | | | | hypothetical | | | | | | |
| | | | | | protein | | | | | protein | | | | | | |
| | | | | | XP_499005 | | | | | XP_499005 | | | | | | |
| | | | | | [Homo | | | | | [Homo | | | | | | |
| | | | | | sapiens] | | | | | sapiens] | | | | | | |
| HG1015022 | CLN00192344 | 65 | 0.01 | 1 | | | | | | | | | | | pTT5 | CLN00736440 |
| HG1015031 | CLN00041415 | 94 | 0.11 | 1 | unnamed | 291 | 40 | 43% | 14% | unnamed | 291 | 40 | 43% | 14% | | |
| | | | | | protein | | | | | protein | | | | | | |
| | | | | | product | | | | | product | | | | | | |
| | | | | | [Homo | | | | | [Homo | | | | | | |
| | | | | | sapiens] | | | | | sapiens] | | | | | | |
| HG1015032 | CLN00041415 | 88 | 0 | 2 | unknown | 400 | 41 | 47% | 10% | unknown | 400 | 41 | 47% | 10% | | |
| | | | | | [Homo | | | | | [Homo | | | | | | |
| | | | | | sapiens] | | | | | sapiens] | | | | | | |
| HG1015036 | CLN00081508 | 62 | 0.29 | 2 | | | | | | | | | | | pTT5 | CLN00736385 |
| HG1015037 | CLN00114957 | 73 | 0 | 1 | | | | | | | | | | | pTT5 | CLN00736561 |
| HG1015038 | CLN00114957 | 67 | 0.51 | 1 | unnamed | 128 | 34 | 51% | 27% | unnamed | 128 | 34 | 51% | 27% | pTT5 | CLN00736561 |
| | | | | | protein | | | | | protein | | | | | | |
| | | | | | product | | | | | product | | | | | | |
| | | | | | [Homo | | | | | [Homo | | | | | | |
| | | | | | sapiens] | | | | | sapiens] | | | | | | |
| HG1015050 | CLN00024579 | 90 | 0.23 | 0 | COG0531: | 456 | 33 | 37% | 7% | | | | | | | |
| | | | | | Amino acid | | | | | | | | | | | |
| | | | | | transporters | | | | | | | | | | | |
| | | | | | [Methanococcoides | | | | | | | | | | | |
| | | | | | burtonii | | | | | | | | | | | |
| | | | | | DSM 6242] | | | | | | | | | | | |
| HG1015052 | CLN00195792 | 58 | 0.69 | 1 | PREDICTED: | 6126 | 21 | 36% | 0% | | | | | | | |
| | | | | | similar to | | | | | | | | | | | |
| | | | | | SCO- | | | | | | | | | | | |
| | | | | | spondin | | | | | | | | | | | |
| | | | | | [Pantroglodytes] | | | | | | | | | | | |
| HG1015067 | CLN00197776 | 69 | 0.19 | 1 | olfactory | 311 | 22 | 32% | 7% | | | | | | p-Donor | CLN00625950 |
| | | | | | receptor | | | | | | | | | | | CLN00625952 |
| | | | | | Olr1334 | | | | | | | | | | | CLN00625956 |
| | | | | | [Rattus | | | | | | | | | | | CLN00625984 |
| | | | | | norvegicus] | | | | | | | | | | | CLN00625986 |
| | | | | | | | | | | | | | | | | CLN00626567 |
| | | | | | | | | | | | | | | | | CLN00626569 |
| | | | | | | | | | | | | | | | | CLN00626571 |
| | | | | | | | | | | | | | | | | CLN00626573 |
| HG1015068 | CLN00197776 | 63 | 0.17 | 0 | unnamed | 138 | 34 | 54% | 25% | unnamed | 138 | 34 | 54% | 25% | pDonor | CLN00625950 |
| | | | | | protein | | | | | protein | | | | | | CLN00625952 |
| | | | | | product | | | | | product | | | | | | CLN00625956 |
| | | | | | [Homo | | | | | [Homo | | | | | | CLN00625984 |
| | | | | | sapiens] | | | | | sapiens] | | | | | | CLN00625986 |
| | | | | | | | | | | | | | | | | CLN00626567 |
| | | | | | | | | | | | | | | | | CLN00626569 |
| | | | | | | | | | | | | | | | | CLN00626571 |
| | | | | | | | | | | | | | | | | CLN00626573 |
| HG1015071 | CLN00198831 | 68 | 0.01 | 1 | hypothetical | 508 | 25 | 37% | 5% | | | | | | | |
| | | | | | protein | | | | | | | | | | | |
| | | | | | [Plasmodium | | | | | | | | | | | |
| | | | | | yoelii | | | | | | | | | | | |
| | | | | | yoelii] | | | | | | | | | | | |
| HG1015074 | CLN00202085 | 70 | 0.26 | 1 | | | | | | | | | | | pTT5 | CLN00736352 |
| HG1015075 | CLN00202085 | 58 | 0.62 | 0 | | | | | | | | | | | pTT5 | CLN00736352 |
| HG1015079 | CLN00243977 | 70 | 0.45 | 0 | septin-like | 564 | 24 | 34% | 4% | | | | | | | |
| | | | | | protein | | | | | | | | | | | |
| | | | | | [Rattus | | | | | | | | | | | |
| | | | | | norvegicus] | | | | | | | | | | | |
| | | | | | gi\|25486149\| | | | | | | | | | | | |
| | | | | | pir\|\|JC7365 | | | | | | | | | | | |
| | | | | | septin-like | | | | | | | | | | | |
| | | | | | protein-a - | | | | | | | | | | | |
| | | | | | rat | | | | | | | | | | | |
| | | | | | gi\|6090881\|gb\| | | | | | | | | | | | |
| | | | | | AAF03376.1\| | | | | | | | | | | | |
| | | | | | septin- | | | | | | | | | | | |
| | | | | | like protein | | | | | | | | | | | |
| | | | | | [Rattus | | | | | | | | | | | |
| | | | | | norvegicus] | | | | | | | | | | | |
| HG1015086 | CLN00226626 | 92 | 0.49 | 1 | unnamed | 350 | 46 | 50% | 13% | unnamed | 350 | 46 | 50% | 13% | | |
| | | | | | protein | | | | | protein | | | | | | |
| | | | | | product | | | | | product | | | | | | |
| | | | | | [Homo | | | | | [Homo | | | | | | |
| | | | | | sapiens] | | | | | sapiens] | | | | | | |

1. A nucleic acid molecule comprising a first polynucleotide that comprises a nucleotide sequence chosen from:
(a) SEQ ID NOS.:1-187, 375484;
(b) a polynucleotide encoding a polypeptide comprising an amino acid sequence chosen from SEQ ID NOS.:188-374;
(c) a complementary polynucleotide comprising a complementary nucleotide sequence that is complementary to the first nucleotide sequence of (a); and
(d) a biologically active fragment of any of (a)-(c); and,
wherein the nucleic acid molecule is an isolated molecule.

2-4. (canceled)

5. The nucleic acid molecule of claim 1, further comprising a second polynucleotide.

6. The nucleic acid molecule of claim 5, wherein the second polynucleotide comprises a second nucleotide sequence encoding a secretory leader, and the secretory leader is a homologous or heterologous leader.

7. (canceled)

8. A polypeptide comprising a first amino acid sequence chosen from:
(a) SEQ ID NOS.:188-374;
(b) a sequence encoded by one of SEQ ID NOS.:1-187, 375-484; and
(c) a biologically active fragment of (a) or (b);
wherein the polypeptide is an isolated molecule.

9-12. (canceled)

13. The polypeptide of claim 8, further comprising a second amino acid sequence, wherein the second amino acid sequence is a secretory leader, the secretory leader is a homologous leader or a heterologous leader, and the first and second amino acid sequences are operably linked.

14-15. (canceled)

16. The polypeptide of claim 8, wherein the active fragment comprises a mature polypeptide sequence.

17. A polypeptide of claim 8, wherein the fragment comprises at least six contiguous amino acid residues.

18. A vector comprising the nucleic acid molecule of claim 1 and a promoter that regulates the expression of the nucleic acid molecule.

19-22. (canceled)

23. A recombinant host cell comprising a cell and the nucleic acid of claim 1.

24-29. (canceled)

30. A pharmaceutical composition comprising a pharmaceutically acceptable carrier and a nucleic acid molecule of claim 1.

31. A pharmaceutical composition comprising a pharmaceutically acceptable carrier and a polypeptide of claim 8.

32. A pharmaceutical composition comprising a pharmaceutically acceptable carrier and the vector of claim 18.

33. (canceled)

34. A host cell composition comprising:
(a) a recombinant host cell of claim 23; and (b) a pharmaceutically acceptable carrier.

35. A method of producing a recombinant host cell comprising:
(a) providing a vector comprising the nucleic acid molecule of claim 1; and
(b) allowing a cell to come into contact with the vector to form a recombinant host cell transfected with the nucleic acid molecule.

36. A method of producing a polypeptide comprising:
(a) providing the nucleic acid of claim 1; and
(b) expressing the nucleic acid molecule in an expression system to produce the polypeptide.

37-40. (canceled)

41. A polypeptide produced by the method of claim 36.

42-43. (canceled)

44. An antibody or a biologically active fragment thereof specifically recognizing, binding to, and/or modulating the biological activity of a polypeptide of claim 8.

45-48. (canceled)

49. A fusion molecule comprising a first polypeptide that comprises an amino acid sequence of a therapeutic molecule chosen from SEQ ID NOS.:188-374, a polypeptide encoded by a polynucleotide chosen from SEQ ID NOS.:1-187, 375-484, or a fragment of any of these, and a second polypeptide that comprises an amino acid sequence of a fusion partner.

50-51. (canceled)

52. A method of determining the presence of a nucleic acid molecule of claim 1 in a sample comprising:
(a) providing a complement to the nucleic acid molecule;
(b) allowing the molecule to interact with the sample; and
(c) determining whether interaction has occurred.

53. A method of determining the presence of polypeptide of claim 8 in a sample, comprising:
(a) providing an antibody that specifically binds to or interfere with the activity of the polypeptide;
(b) allowing the antibody to interact with the polypeptide in the sample, if any; and
(c) determining whether interaction has occurred.

54. (canceled)

55. A method of determining the presence of a specific antibody to a polypeptide of claim 8 in a sample, comprising:
(a) providing the polypeptide;
(b) allowing the polypeptide to interact with a specific antibody in the sample, if present; and
(c) determining whether interaction has occurred.

56-63. (canceled)

64. A method for treating a tumor in a subject comprising:
(a) providing a pharmaceutical composition of claim 31; and
(b) administering the composition to the subject.

65-69. (canceled)

70. A method of treating a tumor in a subject comprising:
(a) providing a first composition comprising a pharmaceutical composition of claim 31;
(b) providing a second composition comprising a different anti-cancer agent; and
(c) administering the first and second compositions to the subject.

71-73. (canceled)

74. A method of treating an immune disease in a subject comprising:
(a) providing a first composition comprising a pharmaceutical composition of claim 31;
(b) providing a second composition comprising a different agent effective in treating an immune disease; and
(c) administering the first and second compositions to the subject.

75. (canceled)

76. A method of treating a metabolic disease in a subject comprising:
(a) providing a first composition comprising a pharmaceutical composition of claim 31;
(b) providing a second composition comprising a different agent effective in treating a metabolic disease; and
(c) administering the first and second compositions to the subject.

77. (canceled)

78. A method of treating a degenerative disease in a subject comprising:
(a) providing a first composition comprising a pharmaceutical composition of claim 31;
(b) providing a second composition comprising a different agent effective in treating a degenerative disease; and
(c) administering the first and second compositions to the subject.

79. (canceled)

80. A nucleic acid molecule of claim 1, wherein the biologically active fragment comprises a nucleotide sequence encoding a mature protein.


SEQUENCE LISTING (486 sequences)

SEQ ID NO: 1 - DNA, 270 bp, Homo sapiens
atgcatgtgcatgttcattgcagcactattcacaacagcaaagacatagactcaacccaaatgcccatcaatgatgcattggatgaagaaaatatggtatatttacaccatggaatactatgcagccgtaaaagggaacaagataatgttctttgcaaggacgtgggtggagctggaagccattatcctcagcaaactaacacaggaacagaaaaccaaacaccacatgttctcacttataagtgggagcccaacaatgagaacacatag

SEQ ID NO: 2 - DNA, 264 bp, Homo sapiens
atgatgcattggatgaagaaaatatggtatatttacaccatggaatactatgcagccgtaaaagggaacaagataatgttctttgcaaggacgtgggtggagctggaagccattatcctcagcaaactaacacaggaacagaaaaccaaacaccacatgttctcacttataagtgggagcccaacaatgagaacacatagacacagggaggggaacaacacacacttgggtctgtcatgggggtggagggagggagagcattag

SEQ ID NO: 3 - DNA, 249 bp, Homo sapiens
atgaggtttccaatttctctgcatcctcatcaaaacttgttattatctgtcttttttattttagacgttctagaaggtgtggagtggtatctcattgtggtattgatttgcatttccttaaggactaatgcttttgaacattttttcatgtgcttattggccatttgtatatcttctctggagaaatgtttattcaaatcctttgtccattttttaattggattgtctttttattgttgcatggtataa

SEQ ID NO: 4 - DNA, 186 bp, Homo sapiens
atgtttattcaaatcctttgtccattttttaattggattgtctttttattgttgcatggtataagttctctatatattctggacacaagtctcttattagttataccatttgcaaatattttctcccattctgtgcgttgtcttttaacctttctaatgtgtccattgaaacacaaaagtttttaa

SEQ ID NO: 5 - DNA, 201 bp, Homo sapiens
atgggagacagcctcttacttgccaagaaaatgaaaggattggaccgagctggaaaacctcctttaccagatgctgactggcactggtggtttttgctctcgacagtatccacaatagctgacggctgggtgtttcagtttgaaaatattttgttgccttcatcttcactgcaattttgtgtaaatttctcaaagatctga

SEQ ID NO: 6 - DNA, 165 bp, Homo sapiens
atgacttaccttgaaatctttattcaaaggatatactattcaatttttcagtgtgccactgccaccactgcatgtttttggtcagagtgcagtgcaactaacatcgccatcctgctaggaaaaagatctgcaggttgctggatagtggaagttgcacacatctaa

SEQ ID NO: 7 - DNA, 255 bp, Homo sapiens
atgcaagtacattttgagtgtaacaaagcccacaaatcttttcctgattttgtttacatgaccttgaatttattgagtgatgagagctctgtcagaatcagatttcagagctccttggcccaagccaggaagtgggcatgcatgactgaccaagcccagagtggcaggaggccaacctgctgtgtcttatcttgctttcccatagctcaagaggacaagagcagtgttctaggagaagccaaattattttcctaa

SEQ ID NO: 8 - DNA, 222 bp, Homo sapiens
atggagtctcactctgttgcccaggctggagtgcagtggtgtgatcttggctcagcgcaagctctgcctcctgggttcacaccattctcccacctcagcctcctgagtagccgggaccacaggcgcccgccactacacccagctaattttgtttttgtatttttagtggagacgggttttcactgtgttagccaggatggtctcaatctcctggcctcgtga

SEQ ID NO: 9 - DNA, 483 bp, Homo sapiens
atgaggatccctgagttggggcagagaaagcaggcagggagaaaaatggggacctgcggcgctgggggcccagctggtggagatctccagctaggagatccccagcacagcacttgccctgccctctgtcttgttcagggatgtaacctgcctcctcctgcttctccgcggcacctcagggcaccgcctggggagggtcttgtagtgcacactcagcatctctgttccctggagcgcatggggtgtgaaacgcctgatgccagccagctccccagtcttgagaggctggttgaatggcacatctccctaggaggctccctccccagggtcccctctgcccctgctgtccatgcagtcggtcctcacccgtcaggaaaacgtagcctgcttgcctgggtggttttccccatcaggaactgccaggtactgggcctggatgggcttgaattccccatctcagtgggagaagggggcattgtttag

SEQ ID NO: 10 - DNA, 327 bp, Homo sapiens
atgggtcctgcgttggcgcggggggacccctcgtttcgctctggcctaagcagcccctggggtcccgcgcgccgcaagagcgccagacttcctaacctgggccataccctgtccgctccacttcccagtccaagaaggaacagccgaagcccaggacgccaaggacctggctcgaagtctccagaattcccgccagagctgggctgggctcgcggaccgctcatgcgccgtctcccaggagcctccccccagcccagtttaatagccggccctgaccagtccaggggagagagaccgtgcagcctcccagcagaccgtgacctctga

SEQ ID NO: 11 - DNA, 240 bp, Homo sapiens
atgaggtcaaacctaataacagatatattacactctaacagatacattacattgtatagtagaattctcagacagcaaccttataaggtgatagatgcagaaacaggcccaaagagtaaaaataatttgttcagagttttccagccaagtggcgaaggtgagattcttacctgggtttgtctgaaaccaattttcactatctttttcagttatgatacactgcctattacgaatgaatga

SEQ ID NO: 12 - DNA, 207 bp, Homo sapiens
atggcccccgcggcggctccgtcctccttggccgtcagggcctcaagccccgccgcgacacccacctcgtacggcgtcttctgcaaggggctctcccgcaccctgctcgccttcttcgagctggcctggcagctgcgcatgaacttcccgtacttctacgtcgcgggctcggtgatcctcaacatccgattgcaggtacatatttag

SEQ ID NO: 13 - DNA, 167 bp, Homo sapiens
atgtcttccccttttactcatagtcaggacccagaaacttcaacggcctacaagactctacaccagtggcccccaacctttttggcaccagggaccagttttgtggaagacaattttcccactgactggggaggtggggtgggggctgtgtgggggtgtgagatggt

SEQ ID NO: 14 - DNA, 162 bp, Homo sapiens
atggaattttacaggtctgacatcttgacagaggtatattgtaaaataagatactctctccgggaaaagagaaggcagtttagaggacaggtagagaggaagtatacagataaggtgtgcaggtctgctcagggttccgaagcagtttcctggaaaacttga

SEQ ID NO: 15 - DNA, 225 bp, Homo sapiens
atgctgtttttcccttctcattccatcttgacattgtctattttgagatctcagctcagttgcagagaagctgccttccccatctggcaacccattatatgtgtcatagtaccatgttgtaccatagagctagacacaggtgccatgttgtgtcttgaaatgtattcaccagcttccaaaggtttacctcaatcccctttactcaagaagcctcagttctactga

SEQ ID NO: 16 - DNA, 162 bp, Homo sapiens
atgctagatgttagggataaatggttaacaggacacagttcttgcttatcttgctatggtctgtcaagccttatacaatctagccttatctctctcaccgtcttatctttatcacccgtagattcccttgttggccactggtttctttcagtccttaattag

SEQ ID NO: 17 - DNA, 306 bp, Homo sapiens
atgtttgttgtctgtggtatgttttgttactgtggcccaagctgttggacacagggacacccctgggctcccaggcaccacccttccctcacgctgctgctctcctggaacacggtcgtgcctcactgcccatccaaagccacatcccggctccacctgctcactccttgtttcttccactcgatggtcaaatttttctcttaccaagaagctggccaggtgcagtggctcacacctgtaatcccagcactttgggaggctgaaacaggcagatcatttgaggtcaggagttcgagaccagcctga

SEQ ID NO: 18 - DNA, 198 bp, Homo sapiens
atgacagtttcaagagcaccccacgtacttgacccccgctgggcagaagctaccaaaggagaccccgcgagggtcaagggagggtgtcgtcaggcccgcgctgagccctgcaggatgctcagggccagggccccaccctctactggcccttctccctcagagttgtccattgactggacaaaccatccccaatgctga

SEQ ID NO: 19 - DNA, 207 bp, Homo sapiens
atggagcctttcagaagggcccagccctgggggatctacgacccttggttcccttcaagaaagctcagacagcgaaggaagaaaagggcagggcagcctatgagagagagaaggcagagaaaaaggaaaggagatgggaggaaagggaacgccttctttagaactcaggaaaaacaacatcagaagtgggcatttctccctgtataa

SEQ ID NO: 20 - DNA, 201 bp, Homo sapiens
atgcaattatcaaacaaacccagtcaaccaggattgaaagtgtcaaacagtgcaacaatcaaggccattctttgtcatgatcaagagcacagagcctggagtcagactgacaaaggtttgagattgatgaaagattgtcaggtgactttttggtctctcctacatagtctggaaatcagaatttggagctttaaactttaa

SEQ ID NO: 21 - DNA, 195 bp, Homo sapiens
atgcctgggcagcgtggtccagcccctgcctcggggtggaaaggcagagagcaaagaggaggcccctcttattccggacccccaggccagagcccaggagtgggaaagcagagggcaaagaggaggcccctcctctcatccctgacccccaggccagagcctaaggttcaagtgcctcaggcccagtccccttga

SEQ ID NO: 22 - DNA, 192 bp, Homo sapiens
atgagcacactgagctctgcagggcctcagctggaaggcgccatccaatcgtccttcccctccccagcagaactccagaggtggggcggctgccgcagggcccaaggctgcctcggcagggggctttgtgctttttgttgtggaggccaccagggattcaggaagatcctgaatgggttgtcaattgactga

SEQ ID NO: 23 - DNA, 399 bp, Homo sapiens
atgacaataccccgtctctggtatctttgcacttgcctcttcaaagccaccttttcctttctctctctctggactgtcacagatcctcctctgtaccccaagaacaccccattggatttccgggctggctgcttggcctacacacctgggtcaggcctctcgcagggatgcgcctgccactgtaataaagaggagaaaacgtcacacgggaaaggcctgactccttcgtacaatcagaatactcaactgcacagagaagggacccctttaagccactttgggagccacatccaccactctgtgactcccacacaggctggttcccaggtatcaggtgtcctagtgttaacacgggccaaaaggacaaaacaggaccctaggtccttcccaaaacattag

SEQ ID NO: 24 - DNA, 225 bp, Homo sapiens
atgtgcatttctctgctgagattccacatgtattcattcattatgagaattttttttcttcaccccatgactatagttttaatagctgctctcaaatacttgactgctgataacaacatcttggacattttggggatagcttttaatgcctatgttttatcttgtgtatggataacatttttgtgtttcttctcatgtctcttaaattttaaaattgtattttaa

SEQ ID NO: 25 - DNA, 183 bp, Homo sapiens
atgaataccaggagtggaaaaccactagagactggctaccatggtcacttacacaccaacatgaaactttcagcctttccacctcctaccacttatactatatttaatgtttttcctgttatggactttaaaaaagaggtcatcataagcaataatatccatgaaatcatggatatcatgtga

SEQ ID NO: 26 - DNA, 165 bp, Homo sapiens
atgggattccctatccttgtttcccagtttcggtcatggtattgcttatccattattagttatccactgctgggtaacaaattactcccaaacttagcagcttcaaacaacaataaacatgtattatgtcacatagtttgtttcagaactttgggagtggcttag

SEQ ID NO: 27 - DNA, 258 bp, Homo sapiens
atggtttgtgttatacctgatctgttttcctgtatcaaaaataaaattaggatgaatctaaaagtttattgtcatagaaaaagtacagatcagaatccaggagacttcaaatcaagtgggaagagtggtaagaagcttgcactcaccagtaacagcacagcttataaggataaaggaggaagcattttgagcttttccattattggtccttatacattaagattttgttggggggtggcaaacagctgtttaagttga

SEQ ID NO: 28 - DNA, 240 bp, Homo sapiens
atgtttttagtagagatggggtttcatcatgttggccaggctggtctccaactcctgacctcaagccatacactggcctcggcctccctaagtgctgggattacaggtacgagccaccgtgcctggccaggaatctgcatttttgctagtaaccctggtcatctcagtgcacaccaaagtttgaaaccactgttggggtgtggagttcttgcttccagacttccagggcctatggattga

SEQ ID NO: 29 - DNA, 220 bp, Homo sapiens
atgaactcactgctgaccttggttttattcgtttgtttattttcaccttttcttgtgaaatgtgcaaacatactcaaaagtcaagagaatcatatggtgatttctcatagtcccctttggccagcttcagcaaagatgacattttgccactcttattccatccatttctgtcacgccacccctgacatacacacatgttctttctggagtattttcaagc

SEQ ID NO: 30 - DNA, 351 bp, Homo sapiens
atgaggaagatgacatttttgtctgcagatggtggaaataaaaatcacagagattgtgatttccttatggttttgtgggtaatggtggagtttaaacttacaacgaagtttctggtgacatgtttcctagttttcacagaaaacattctttttttttttgagacagaaagtctcactgtgtcgcccaggctggagtgcagtggtgtgatgtgtcggctcactgcaacctctgcctcctggattcaagtgattctcatgcgtcagcctccaagtagctgggattacaggcgcccaccaccacgcccagctaatttttgtatttttagtatagatggagtttcaccatgttga

SEQ ID NO: 31 - DNA, 162 bp, Homo sapiens
atggagtttcaccatgttgaccaggctggtcttgaactcctggcctcaagtgacccacctgtctcagcctcccaaagtgctgggattacaggcataagccaccacacccagccaaaaacattctttataatgatacaagtaatataccagacaagaaattac

SEQ ID NO: 32 - DNA, 183 bp, Homo sapiens
atgaaagctacaatgtctcttttgaacaagtcgtttatcaagagaaacatctatcagggactgccttgtcacattgatcctctaaaagttaggtccgaggtggaagctgctttgatgtttttgatcctaaatttaatacaacagaaaaaaagcaaggatattttgaccagtatagatttttaa

SEQ ID NO: 33 - DNA, 279 bp, Homo sapiens
atggctaataagcacatgaaaaaatgctcaccattcttagtcatcaaagaatgcaaatcaaaatatgagatagtattatacttcacacgcactgggatggctgtaatcgaaaagacagatgataacaagtattggagaggaggtgaagaatttagaaacagtctggtggttttccaaaagataaacatagagtcatcatataacctaacaattccaatcctagatatatatccaagagcaatgagaacatacgttcacacaaaagtttgtacattttaa

SEQ ID NO: 34 - DNA, 270 bp, Homo sapiens
atgtatgaccacgggttaaaaaagcttttttttttttttttttttttttttgagacagagccttgctttgtcgccatgctggggtgcagtggcgcaatcttggctcactgcaacctccacctcccgggttcaagagattctcctgcctcagcctcccgagtagctgggactacaggcacacgccaccatgcccagctaatttttgtatttttagtagagatggggtttcaccatgttggccaggatggtcttgatctgttgacctcgtga

SEQ ID NO: 35 - DNA, 291 bp, Homo sapiens
atgggctgcccggtgagcatagctgagatgcaccaaggccacataagtcacctactttgccttggctgccccatctgcatctatcaaaggaagccttggacacccaccagaggagccagcatgagggagtgtatgcctttatacaaattcactccaacttcagaaaaacgtccgcagctcatgctccccctgccagagcagcagtgtgagcagctgtgtaggtttggaagcaccccagtcacttgggcattgatatggtttggctgtatccccacccaaatctcatcttga

SEQ ID NO: 36 - DNA, 165 bp, Homo sapiens
atgctggattttacacacagatctggtttcaggaagaaacaggatgccagtgcagtagccctgtacactatcaccttaaaggacctccgaagttgtttctgctttatctctccataccaactccctccttgctgcacacttcagaattttggaaataagacataa

SEQ ID NO: 37 - DNA, 207 bp, Homo sapiens
atgatgcttgagacacaccaagatacctgcacactagaggctgcagtcacctcagaagaaagatgccccctgctctggctggtcaaacggaaccaagtccgtcttcctgagaggtttggtccccttcaaccagctacagcagggctggcaatgcccagtccttggagaaacagaagagattcaactgcaactgaaattacctactaa

SEQ ID NO: 38 - DNA, 186 bp, Homo sapiens
atggaactaaagaatgaaggagaaatgggtggacagggcacaaattggggaatgacagcctattcagtgaggataggaaatgcacataaaggacagatgaagatgatttctctgtgtgtgattagaaggatagcaattacaaagataagagtgaaaggagagggaggaaaggggaaaagggaataa

SEQ ID NO: 39 - DNA, 339 bp, Homo sapiens
atgcattttgggggacacattcagcccataacaagtaccacaatctaccttggttctttcgactttaccatggagaatttattttcccttccccaggacaggcaggattggaagacctttcctggtgcagcctgggagagccctgctcacagcccacagacaggctgcctgtcctcacaccctactcaggccagggagctgaccccagaatcacagggctgggcacagcagcctccctgctccccactggcccctgctgtgagtcacctcacaggtcctagaggacaaggttctcacttttcaaattatctttcttacatatacactttacccaaatga

SEQ ID NO: 40 - DNA, 187 bp, Homo sapiens
atgtatacatatatatatgtgtgtataagtgactataagtgtacatacatgtacataagatttcacatttattgttatctttttatttgcaatgaaatgtctcagaaaagaaataaaaagaaagaaagaaagaaaagaaagaaagaaaagaaaaaaaagaaaaaaagaaaagaaaagaaaagaaaaa

SEQ ID NO: 41 - DNA, 234 bp, Homo sapiens
atggaatcttggattgcccctccttttgcctatgaacatggatatatattttctaccatttttcatgagttaaaaatcaggcttgtaattttacacattttttttctaatttacttttggatctttttgttatttcaaggacctagggtatggtctgatatgatagaattgacagtgggaatgaatcagaggcaaggagcatataaagtcagtagagccaccatacattgctaa

SEQ ID NO: 42 - DNA, 318 bp, Homo sapiens
atgcagcagaggcaaaccatggtttggccaaaaatggaacagaatttacaagggaaaagggaagttcctggagtaatatgtgtaggtcagatgaggatggaactggtgaatgagcagaagagaaggttggaagagtgcagtggtgatgacagccctccgctgctccttccccagaggaaggagctccaagagattctcaactggctgaaggatacacaagatgtcttatttgtgtgtcacttgtttgtgttactaatgactcctgctgacaccacccttgcttcttcaggcaacaaatgctgtttgcagcacttgtga

SEQ ID NO: 43 - DNA, 216 bp, Homo sapiens
atggaaggtgactcctttgagctttgttgcaggtcagccatagcctatgcagtgtgcaaggtgacaccttccttaggggctagcgtccctttctgctcctttcccagcctggacgtcccctgctgttcgcttcggtctttttcggcctcctccctgatgtggctttgttctgtcttctgcttaatgccacaggctttggttcctgccaccaaatga

SEQ ID NO: 44 - DNA, 354 bp, Homo sapiens
atgaatatgctgtacattgctttgctgttgttaccccccttaaccatgctagaagaatcacccacagaagggtgtctccaccgtacacatactacttggtcagggaacagcataactaaaactctattgtaccatacttattatgggtgtatggggaatcgcttaggaacttgtacttatagtcaaaccacctactcagtttgtgacccaggaaataaccaactttatgtatgttatgaccccaagttttcacctggtgaatggtttgaaattcgtgcagtcaaaagaaggtctcctcttaaaccaaaccagggtccctcccttttactgagggactatttctctgtattttga

SEQ ID NO: 45 - DNA, 315 bp, Homo sapiens
atgaggaacgctatctatcagaacagattagctttagactatctcctagccctagaaggagtagtatgtggaaagttcaatttaacaaattgctgcctggaaatcgatgacaatggaaaggcaattatggaaataactgcaagaatgagaaaattagcccatgttccagttcaaacttggaaagggtggtctccagattctctctttggaggcttggtttttatttttctgagggttcaagactttaataggagtggttctggccatattaggaagttgcctaatactcccttgtctcttacctctccttgttag

SEQ ID NO: 46 - DNA, 153 bp, Homo sapiens
atgtctgtaatccaaacactttgggaggccgatgcgggcagatcacttgaggtcaggagttcaagaccagctcgaccaacatggcaaaaccccgtctctactaaaaatatgaaaaagaaaaaattaggctggtgtgctggcacatgcctgtag

SEQ ID NO: 47 - DNA, 162 bp, Homo sapiens
atggggaggtcacgctcctgtccctctcgcccgcgtcggctagtggtcagtctggcgcgggcagtgtcaccggttgctgggaagactgggaggccagcgagcagcgtaccaaccactcagcctctccggccccagctggagccgggaagcaggcggggctaa

SEQ ID NO: 48 - DNA, 441 bp, Homo sapiens
atggttccacatcacctttcttcgagggacagactcctcttcttgacctccgtctccatgttccctcatgacacttcactgtccttctccagcatggacactctttccatcactaccgccctggagaagctaggtcctcttctgggaagtcactccttcccaaaacctcacacgaattttctgaggagcagccccagtgcccccttctcagccaacacacccacctccattctctctgagcatccagttttggtcagcacagttctgctctcccccttccaagctgttcccaaggttatcatccagggatatgtaagtttccatctgaaatgcatcattcctcctctcctaggcagtcaaatctcatttccaggcccacatcatctgattaatttcttaaacatcagaataatcattaacaatgatacatcagttttctag

SEQ ID NO: 49 - DNA, 162 bp, Homo sapiens
atgtatatatgtatatatttatgtatatacaatgatgaaaaaaccgacatttttacactatatacatatgtatgtgtatatctatggacaaaaccaatattttctatgaattgtttattcctattaatcagctatagaatatgtatgttacataatatataa

SEQ ID NO: 50 - DNA, 294 bp, Homo sapiens
atgaatagcaggtttgaatcagtaacaaaatgtctcccaaaggagaaaagccctagactaggcttttatgctgatttctacccaatttataaagaaaaacaaacaccaattcttctcaaactattcccaaaaattgaagaggaaggaattcttcctaactcattgtataaggccagcattaccctgatatccaatcaagacaaggacacaacaaaaggagaaaactacaggccaatattcctaatgaacacagatggtaaaattctcagcataatactaccaagccaaatctaa

SEQ ID NO: 51 - DNA, 153 bp, Homo sapiens
atgaatatagaaatggaaattggtcccttttcaccgatcacaaactttgtacctcaattcttaaagtcagggatgtttaactcagccagccacctagctagggtctgtaacaaacagttggttatttttcaccctttgtctactcaaaattaa

SEQ ID NO: 52 - DNA, 261 bp, Homo sapiens
atggggctgctcccgcaggctggtctcgccggtctgggcggctggggctgggctgccaggccgtctcttaaagcgccgcggggcgccgtcgggcgagcacaggggcggtccgtggtgctgctggtgcccagctgcggcgcggggagccgcgcagtgcacggcaggggcagggacccagcgccccgaggccgcagtcccgccccagcccgtccctggcccaggcccaggcccggcggtggaggggcgcagagccgaccgtga

SEQ ID NO: 53 - DNA, 231 bp, Homo sapiens
atgcggcgctcgcgcacccgttcgctctcagcggacaaaccggtcgagagcgcaattctggagcgggttccagaagctcctgggttccaattcaggctcctcactcgccagccctgcgatttgggacaacttaacgttcgtaagctgggttcagtcttctcaattgggttaatgtccgtgaggggctctcgtaagggcggctggcctgccggtaacttaagaagcatgtaa

SEQ ID NO: 54 - DNA, 207 bp, Homo sapiens
atgctggtcatcataaggctctcctgcaaaacagccttgtatagtgggttgagagattactgtctcagttacccactcctgcttggaaccaggattctgtcagtgtccccagaggttgtggatcaaatcaatgtgcctgttagacacaagccaagagaaggaaaggtgtgcttgaattcattcacgatcatgcatggtaacgtttaa

SEQ ID NO: 55 - DNA, 201 bp, Homo sapiens
atgagcatgtgcgtgtgttacaacttttcatttaaaaaatcaactcctgggtcattgtccacttttttaaacttttcatttgctcataatttcagatttacagaagagttgcaaaatagttcctatatgcctttcacccagattccactagtgttagtattttacatgaccatggtatatttatcaaaactaagaaaataa

SEQ ID NO: 56 - DNA, 288 bp, Homo sapiens
atggccctcctttgtcacatttttctttttactgttctcttaccccctttcactctcacttcacttcctccatgctgctgtactaccagtagctcctcttaccaagaggttctatggagaatgtggcttcccagaaatattgatgtcccatcgtataggggtttttctaaaggagaccccactttcaccacccacaaccatatacccctgcacttcaggccatacatttcaatccctgtatctttaacctccttgttaagtatgtctcttccagaatcgaagttgtaa

SEQ ID NO: 57 - DNA, 234 bp, Homo sapiens
atgagaattaaaaggaataatcgccataaaaggcctgatactggcaataatatatttgacaaatatgagtttctctttttccctttattctactttacggcaactcctacctcctattctctttcttttgctactaagcagacatctgggtttgtttgtttgtttcatcagggagaggttctaaacgttgtatcctttaaccctcaaagcatcatctattctcttagagggtaa

SEQ ID NO: 58 - DNA, 189 bp, Homo sapiens
atgttgagtaaaaatattagaacttgtgaaaccctgtttgtatacacccaaacacacttacatatctatctatatctatctatagaaatatattgtgtgtgtgtgtgtgtgtgtgtgtgtatgtatttgtgtgtatgtgtttctctgtctgtcttagttcatttgagctgctataacacaatgccatag

SEQ ID NO: 59 - DNA, 267 bp, Homo sapiens
atgagtctaagagtccttcttctcagttcccgtcatatcctgtatgtatcccctttggagcacctccccaaacctttgttccctcggttgaccataagattttggagtgtagggcctgtctgcattctctgtatcccaaggccttacagtgcttgggaccaaccagaattggagatccagctcctagagcatttcaaaacacatgtggaaggacgccctgattcaggtacatacagtaaatctcctccccaacccatgagtctctaa

SEQ ID NO: 60 - DNA, 201 bp, Homo sapiens
atgtgcctagttctacagttctccacatcctttttatctgctgctgtttttcttttttcctttctttcctttctgcataagttgttgagactgtgggcccaactggagaagattttaaagccaagtctagcattattacattcaaatatttttaaaaaatgttttacaggatggttttttgttgttgcttttttgttttaa

SEQ ID NO: 61 - DNA, 312 bp, Homo sapiens
atggccgcgcccgccccgcgcccccgccgcgtcccgtcgccgcccgcccgcgtccccggtgagcgctggagtggggtcgggcccgtgtgcctgcctcccgcgccctctccgaatcccggactcctagtgaaaggaagggggagggcgcgggcgcggaagggaggtggatcggagcggcagcgcggcccagccgaacgggtaccgaccgtcaccccgcgatcttcctcgcagggggcgccggcgcccaagctgctcgcccggagggcccagagccgcttcccagaccagagccgggggaggcgcagccagtag

SEQ ID NO: 62 - DNA, 255 bp, Homo sapiens
atgcattcagatctcatgtgcattttctcgttattcaagagaattggacacttgatgaagggaaggacacggagagatgacaaacgcagactggcatgcagttgttttcagggtgggaagaacaaagtggaatgtcttcagttcttacaaaatatggtggcatttgtaatctctgagtttttgtatttggcctacaaagggggtcttttcagggaaacacgaatggaacccttcagagacatgtctacattgtaa

SEQ ID NO: 63 - DNA, 393 bp, Homo sapiens
atggcctttctccaattcttcctctccaagcccatggcaagggccaaatgggaaaaaagcgaaaccctaaaaggcgagcctatcatttatcccaaagcccaaagaacaaaggctatgtgggtacagtggatttgctccagaagccccggcctctgtcctcgtgtttctgaaaaccatcctttctggcaaggaaagaaagtctcagcgctttggatgaagctctctgttaggccaccactgcttctcacgccttcgactgctggtgcaggaagattccaggccactgggcttcaccggccctcaatgctcacttgtccagcccacagcttgacaaactggtcacttccaccccacctgctggagctgactagggcaagtaaggctgatgggtga

SEQ ID NO: 64 - DNA, 312 bp, Homo sapiens
atgggtgaccctgatcggccctcactggagtacagggtttccttctccaatctcctcaaaacaggtgaacaaagttctcaggccaagttgcctccagcacctccccagtctcagacaagtccatggaccatctctcctgctccatgcaggaggattctccaaccacagtgccagttctacaggtgttatcacccagctaggaaggattttgagaagagcagctctgtcctatgggctgtgggttcacatctccactctccacccagacagcccagaaggaaggctgtctccctttgtcgggctgaggtctga

SEQ ID NO: 65 - DNA, 240 bp, Homo sapiens
atgctgttttccatagtgggtgaaccattttacattccacagcaggtgcaccattttatattgcatagtgggtgcaccattttagttcctgccaacagtgcacagtggcttcaatatctcctcctcctcaccaacacttgctaccttctgtttcttgataatggccatcttagtaggtgtgaggcttatcttacaccatacacaaaaataaactcaaaatggattaaaggcgtagcataa

SEQ ID NO: 66 - DNA, 174 bp, Homo sapiens
atgtttgcaacatggcatagtattgctaattacatacatggggctgtacagcacgcctccagggctcttccatctcacgtggctgaaactctccacgttcctcccccaacccctggcaaccactattctagtctctgtgtctataagtttgactgttttagagacctcatatag

SEQ ID NO: 67 - DNA, 207 bp, Homo sapiens
atgaaggaaatccatagaaaaggtcctcttagtgaacaaaatttagttattaactttatagctatgaaatttccccgggcatttgtttttgttcaaacagactttaacctctgcatcatacttaaccctgcgacatgcgtacagtatgcatattttgttttgaaaaaaaatgtttcgttccagtctgttaagaatattcaaaaataa

SEQ ID NO: 68 - DNA, 243 bp, Homo sapiens
atgggcagtattagaaataatttaaaatgtatataccctttgacttactggtttccttctaccactttattctgtagatacacccacatctacattcattacagcattgtttatggcagcaaaaaattggaaacaatggcaggtaccaccaaaaaaagagactggttgaataaacaaaagcatagctatgcagtggaatgctatgcagctggtagaaagaaggggcagattgatatgtactaa

SEQ ID NO: 69 - DNA, 207 bp, Homo sapiens
atggagaatggctgggtttggtggcccatgcctgtaatcccagcactttgggaggccgaggtgggcagatcacctgaggccaggagttcaagaccagcctggccaacatggcaaaactccatctctactaaaaatacaaagcctagcatggtggaacatgcctgtagtcccagctacttgggagggtgcggcacgagaatcacttga

SEQ ID NO: 70 - DNA, 222 bp, Homo sapiens
atggcacttgctttcttccacatgtctcctaccaattttacagagctcaccccaaccccccacactcccaccccacccccagggatggaggctcgcagagattcatccatcgttcagaaaaagtcaggagagaacaagaagggggagggggatattgctgctgtttggtgttttattttgtgtgtgtgttttttgttgcctaggaaaaagaataggctctaa

SEQ ID NO: 71 - DNA, 300 bp, Homo sapiens
atgtgccaccatgtcttgctagtttttaaatttttttgtaggaacaggatctcactacattgcccaggctggtctccaagccttgagcaatcctcctgcctcagcctcccagagtggtgggattacaggtgtgagccaacacacctggcctttttttgcttacctttcaattctgtttgctgttttttgacatataggggttttaaattttgttgcagttggattacattgccttttgttacttttttcattgcttctataaagctaaatcttcttttcccattccaaattcagaaataa

SEQ ID NO: 72 - DNA, 153 bp, Homo sapiens
atgaaatcgagcattttttttcatattaattggccttttatatttttgtgtgaattgtctgttattacattttaccttttacattcaaatactatacagaatattatcctgttacagctattttattttttgcttacctttttatttatttag

SEQ ID NO: 73 - DNA, 303 bp, Homo sapiens
atggctaaaaggggccaaggtacagttctggccattgcttccgagggtgcaagctccaagccttggcagcttccacatggtgttggttctgtgagtatgcagaagacaagaactgaggttcaggaaactttggctggatgtatgaaaatgcctggatgttcagacagaagtttactgcagggatggagcccttgtgggagaacctctgctagggcagagcagaagggaaatgtggggttgaagtccccgcacagagtccccactgttgcactgaccagtggagcaaagaaaagagggctatag

SEQ ID NO: 74 - DNA, 183 bp, Homo sapiens
atggggcgctactataaggataccaaaaacatggaagcaactttggaactgggtaataggcagaggttgaaacagtttgaagggctcagaagaagacaggaaaatggggaaagtttggaacttcctagaaacttggaagattcagaagacagggagatgtgggaaagtttggaacttccctag

SEQ ID NO: 75 - DNA, 219 bp, Homo sapiens
atgatgccgggcgagacccattcggcggcgcccgggacggcggcggacctgtcgcgatgtcagggctgcgcctctctgcagcagaatttgaatgaatatgttgaagcattaattaccttgaaacaaaaaattatcaatacagataatttgttaacagaatatcagaagaaatgtgatgatatccttttactggctttttctgttgagttttggaactaa

SEQ ID NO: 76 - DNA, 186 bp, Homo sapiens
atggctgggaggccccaggaaacttataatcatggcagaaggggaagcaaacacatccttcacatggtggcaggagagagaagtgcagagcgaagcggggaaaagccccttataaaaccatcagatctcgtgggaactcactcattatcaggagaacagcactgggggactgcccccgtgatctga

SEQ ID NO: 77 - DNA, 156 bp, Homo sapiens
atgtcagtaaagggtgctaaaatgttaaacaaatattactttcagattttcttttcttctctttttattcctattctattttcccccttggttaattttatttttctcaaaattctccctttcttttctaaaccttatatttccccaaaagaatag

SEQ ID NO: 78 - DNA, 337 bp, Homo sapiens
atgccctgttttaagatttttttttcccacacagttttgcatttaagtaccagaatacccaaacatttttcattagtcttcatcaacactcccgtttgtgttctcagtcaaattaaaaatagcaatttttggccgggtgcggtggctcacacctgtaatcccagcgctttgggaggccgaggcatttccactaaaactacaaaaaaattagccgggtgtggtggcgggctcctgtggtcccagctgccctggaggctgagggaggagaatggtgtgaacccgggaggcggagcttgcagtgagctgaggtcgcgccactgcactccaacctgggcaa

SEQ ID NO: 79 - DNA, 153 bp, Homo sapiens
atgcagggttgtggcagaggcagatgggacctgcactgtgacttggccaacgtggactgtgccgattgtctccttcttagagcctcagtttcctcctcaggaatacagagcagctgcctccgtgactgctggattagaggagatggtagttga

SEQ ID NO: 80 - DNA, 321 bp, Homo sapiens
atggtgagtttctatccactagagagtcatagctgtctaaagggacaccatgcaccatttttccagccttacctggtcctccagatgccccgcagataccttgtggtgtggccttcgctcttgcagggtctcagccccctgggacatcctcctacatccacatcttcatggctgaatcccacccagacctcaaaaatgaaacgaaaggccaccttcaggaagccttcctcaatccagtgcttcccttttgtctcctcatcttcttccccattaaagggcttaagaatgatgtcctccaaatttgaccaccttggcttttaa

SEQ ID NO: 81 - DNA, 267 bp, Homo sapiens
atggaaaggctcccccatcctgtcctctacaccaccttgcagctgggcctcagcaactgggcttttaatttcagtctaattcaagtcagcagcatagggcagctcctgggaaattggtttacacatgcggacaagcccagtagcccagagctaacccactcaccatccctgaccacagaggagcagataaggaagcaagatttgtcgggagctatgacaagttccactgtggatggcaaggaaatgaggggggcagcaacctgttaa

SEQ ID NO: 82 - DNA, 219 bp, Homo sapiens
atgtatccatgtgttctcatcattcagcacccacttataggtgagaacatgcagtgtttggttttctgtgttagtttgctgaggataatggcttccagctccatccatgtccctgcaaaggacatgatctcattcctttttgtggctacacagtattccatggtgtatatgtaccacgttttctttattcagtttgtcattgatggccatttgggctaa

SEQ ID NO: 83 - DNA, 159 bp, Homo sapiens
atgtcttccacaatggttgaactaatttacatttccaccaacggtgtaaaagtgctcctttttcttcacagccttgccagcatcttttgtttcttgactttttattttatttatttatttatttatttattttgcactccagcctgggcaacacagtga

SEQ ID NO: 84 - DNA, 162 bp, Homo sapiens
atgggagagaacaaaggggagcggagcgagaccgcggcctgcgcccgccccccgcttgcgcacagtccccgccccgccccgcgggcgccgccgccgccgccctccctcccgcgcctccttctgaccccgagcgagagacctggtcgggagtccggcggctag

SEQ ID NO: 85 - DNA, 333 bp, Homo sapiens
atggcggccgcggccgtgcggcccgtgacctggaccactgggctgggacccacgaccccaccaccacgcctggaggacgcgcagcggcgcctcggccttggaaggggagtttcccagcgcgagccccgcccttccccgcccgggaggcgcctcggggcggggtcactggccgggaggccccgccttccgcccgaaagcgccggtgcaccccgcaggccctgcccgtgggtccacaggccggatccccgggttcctaggctgggactgcttctcagaaccactctgtcgtttttaagcagggtcacacactctagctcactgggtccattttaa

SEQ ID NO: 86 - DNA, 249 bp, Homo sapiens
atgttagagtcatcgaaagtttttaatcaggcaagtggtatgatcagttttttgttttgttttgttttttttgagacggagtctggctatatcgccaggctggaatgcaatggcgcgatctctcctcactgcaacctccgcctcccgggttcaagcgattctcctgcctcagcacctcgagtagctgggactacaggcgcgcgccaccacgcccagatgagaaaactgaggcacagagaggtgaaataa

SEQ ID NO: 87 - DNA, 228 bp, Homo sapiens
atgaaacccaatcccagcaaagaagtgaagagcagatttataacagtcccatccaaatttctctttggcttctctctttggtctttcatctctctgcctttctctctgtgtctcctctctactctttcttctctctctctcatacacatacacacacacacacacacacacacgtatgtgcttagtggtatgagttactgtcaaggagaagggctacctagatgctaa

SEQ ID NO: 88 - DNA, 207 bp, Homo sapiens
atgctaacacacacacacacacacacacacacacacacacacacacacgctttgggtggtgggaagttggagaaaacatttggggcaagagaaaacaatgaaggcggccaggtatggtggtttgcgcctgtaatcccggcacattgggaggctgaggtgggcagattgcttgagctcaggagttcaagaccagcctgggcaacatag

SEQ ID NO: 89 - DNA, 171 bp, Homo sapiens
atgtcccagcagagcaactgggaacctaattctgaccaacaaggaggaattgtttggctaattggaagtgacagaaaccttgggagaaagcaaccgcataccaaggataattataagaaggtaacatgggccctaaaggatagtgtcaagaaacctgaaacccccaaatga

SEQ ID NO: 90 - DNA, 171 bp, Homo sapiens
atgatcaagaaagggaaatgcccactagcagcttctactgtaaggttagagctgacagagaaagaactccttaactactatttgcttcattctctacaaaggaaactagagaagtgggttgacgtaatagaaagaacatgtgtttgtggggccaggcaaacctgggtttaa

SEQ ID NO: 91 - DNA, 252 bp, Homo sapiens
atgatgactatgttaccggtttttgtgcttactatactatactgtttactgtttagagtgtactgttcttacttatcaaaaaaattaactgcaaaacagcttcaggcaggtccttcaggagatgttccagaagaaggcattgctatcatagatgatgacagctccatgcatgttattgccccagaagaactttcagcggtacaagatgtggaggtagaagacagtgatattgatgatcctgaccttgtatag

SEQ ID NO: 92 - DNA, 168 bp, Homo sapiens
atgggaagtgctctatataaaagtataacttttttttatcttgtataccacatgttcactgtgccttttccatgtttagcaatgattagatacacaaatagttatcatcgtggtacaactgcctacagtattcagtatagtcacatgctgcggaggtttgtagcctag

SEQ ID NO: 93 - DNA, 225 bp, Homo sapiens
atgtccgatgctttttccagacagattttggctgcagagctggaaatcacaaagatcataatgacagctctaacagccgtgcctaggctgatgaaaaatgcctttgaacagcccgggctcacaggcattatctttgctaatcctataattggcctgacaagtggtgaaagcataaatgatttcggatatgtgcttttgtgtttaaaagactgggatctggggtga

SEQ ID NO: 94 - DNA, 174 bp, Homo sapiens
atgggtccccagtcattcccactggtattcagactaaaagcaacattggtggcaggaaagtccacaggaaaggctctatttcaggaagaagccagaaaccctggagagaatcctagttctgcccctaacctgcaggatggccttagatggatctctttttgtaccttactttga

SEQ ID NO: 95 - DNA, 213 bp, Homo sapiens
atgtggggcgaagttagagtaatatttttaggttttatggctggcttaggggaaaagggatttttgttttttatgacccacctcggggaagagggattcaatttctatggcttgcttcaggagagaatgagggatgagagacaggagggtaggagaaggtcagagagagaaactttacttctgaggtcttcactttggggtataattttctga

SEQ ID NO: 96 - DNA, 237 bp, Homo sapiens
atgccttccaaaaccacgcccatttccagtgatgatggtttgtgccacaggccaagtactgaacatcatgacactcatagacactcttcactgaagacgtggatagaccctggggccaccattctctcctctctggagtgttggaggggccctgaccagggcaggacccccatgtctggaagcagggttagccctggctcatgggggaccctgctgggcttagccattgccacctag

SEQ ID NO: 97 - DNA, 222 bp, Homo sapiens
atgcacaagaggcaacaaaggcaagtaaagaggaggctggtgggcttgcagaaacgccctgcagtacctatgctgcggttctcccacttcactgataacaaatcctcagtatccactgatacaaatcctgggactgctgggccagtcccaggaatgacactgggacagactccctggaggtggatcagaaagctgcagttttaccaggggtcctgctgttga

SEQ ID NO: 98 - DNA, 249 bp, Homo sapiens
atgccggaagagattgagattcctagagaatggaggagccgggaacaaaggagaaggatgggatccaggaacaggccaagagaaccagctcgatgcttcttcctctcccttctagaaaattctgttcagaagccattaggcttcaggctcgagacagagaggaaccgcctctgtgtttttcactgtcgtgtctcagtgctgggagtgtactccatgtggtcagtgtgtgatcaatttaactgtgcctga

SEQ ID NO: 99 - DNA, 201 bp, Homo sapiens
atggaggagccgggaacaaaggagaaggatgggatccaggaacaggccaagagaaccagctcgatgcttcttcctctcccttctagaaaattctgttcagaagccattaggcttcaggctcgagacagagaggaaccgcctctgtgtttttcactgtcgtgtctcagtgctgggagtgtactccatgtggtcagtgtgtga

SEQ ID NO: 100 - DNA, 258 bp, Homo sapiens
atgttgaccaggctggtctcaaactcccaacctcgtgatctgccttcctcggcctcccaaagtgctgggattgcaggcgtgagccactgcgcccggcctgtaccctccttctttttgaaaaagagagtaccttcttcctctgctttcattctgaagcctccatttaaaacactcctcctccttagcaatagtgagggtcccttcagactagataggatgtcaaaaggccaaagcattgcccccaacacactcctttaa

SEQ ID NO: 101 - DNA, 186 bp, Homo sapiens
atggttgtttgctcagaaaaaagccatctcatcccccctccctggcatctttctgatctgtcctgttctgtaggtggaatgttcccacctttgtttgcctgttcagacgtgtttgtccttccattatttttccggctggattgccagcagaatgcagagcagcctggagaaatgaaacagagctga

SEQ ID NO: 102 - DNA, 306 bp, Homo sapiens
atgaacacctcagccatccatgacagctgtttttcctcctggcctttaaaatcctccctcctcagccttcactcctactcaccacctcaacttaatttctcattccccacccaggaagaacctgtatgccccctgcatcccactgcacttccggatgccttttcccccaagactggaatggcctcaggcccccatctccaggtgagtggccctcacctacagactcaacatatggcctttggctcttcccacttccaagagtcttggaagggatgggtcgagcaagcagaggaaaggaagatgtga

SEQ ID NO: 103 - DNA, 297 bp, Homo sapiens
atgccccctgcatcccactgcacttccggatgccttttcccccaagactggaatggcctcaggcccccatctccaggtgagtggccctcacctacagactcaacatatggcctttggctcttcccacttccaagagtcttggaagggatgggtcgagcaagcagaggaaaggaagatgtgagttcccaaaatgctcctcacctttttcttctgagtgggctccttctcactggcattggagggcttgcggcgcagcatggtcctccaccctgggagactccgtccctgctctcctag

SEQ ID NO: 104 - DNA, 186 bp, Homo sapiens
atgaggttgtggggggaatgtaaaaacttgaaggacaaggtagctcagtccaaccagatgagcatccaagggagatttttgtctcatcctttgcagaaatctccatctagagcccgtgtaaaccaatggaaattcttacttgctttaacagcacactattcctattcattcattaatgtgggctag

SEQ ID NO: 105 - DNA, 252 bp, Homo sapiens
atgtttcgtgctgcagcaaaattggggggaaacattggcaaaatcttccaagaacacataggtcttgggatggccacatgcatttggctatctacattcctgcacatctgtggagagaaaaaaagtttcttttttaaaatacggagtcatacgtttattcttaggctactccaaagatgcaacaaggtgttttgtttttattttttcaaaatgggtttgacagtctttctgcctactgattgtcacagctaa

SEQ ID NO: 106 - DNA, 195 bp, Homo sapiens
atggtggaggccaagcagaagagtgacctcatggggaccgcgcctgggtttgtgtgcccactggagagctcgttcctattgcagggccataatgtaaagcagagggtgcttcttttcagaaagctcacctatagctccttcaggcctgtactcttaggagggaaaaaggaagggagtacaggttctggaatttaa

SEQ ID NO: 107 - DNA, 462 bp, Homo sapiens
atgtcccttaaaagtgcgacgaagtggggaagaaggtgtaattactattatcagcatctagaaagcatcatgaatttgctggagtacttcctagcactgacctccttcattctgcgttgttcttactggatctttccatcagccaacaatatggaagtaccaatacaaggtcaaatcattcctggattcatctggagttgcttaaaagttaaatcattggaatttttgatgataccttttctatatggattacaatttgatcgctgggaattctccaccttaaagaagactcttttgctatctggaaacccatgtccacctctgacttccactcaaaattgctttcctcacagtctgactgcgagagttgtgaaaaattgggatgtgcttctgaggtgggctgtggaatgccattaccctcaggtgactacagatgtgttaacacccagcatgttccggtag

SEQ ID NO: 108 - DNA, 201 bp, Homo sapiens
atgcttaggaaccactacaaatgctactgccttgagtctcattttgtttccctctggaaaccacatgtgtaccttgtttgcaacagtatgggctcacaggcagaaggaattttccttgtcttggatgagacttttgacttggacttttgggttaagttctggagaccagaaggccaaaatcaaaagtatgggcaggcttga

SEQ ID NO: 109 - DNA, 201 bp, Homo sapiens
atgctccagttctcttgtactggggatgtggtggcatcgcaaacattcacagttacggcgggatttaagaagcagatgagacctttccaaccaaaggccatctggctagtgtttgagcctcaggtgcagtttttcattgatttttaccatttaattgtgagatctgcaatatcaggtcttcctttcacagaaacaatgtga

SEQ ID NO: 110 - DNA, 411 bp, Homo sapiens
atgggggcctcagctgcctgcagttgcccagggcatggaagcacaaagctcatttctgttaccacatcactaactgtaggattggacctcaacatgcatactggttctggcacaaaaggagctgatttcttgttcttgcaactgtcccaggcaggggttcctgctcagcagatggtgcctctcctagaagtaattcagggaccagccaccttccatcctaggcctccatcacagcccttgggcctcgctgtggtgtgcacccccagcagtcagaaaagagggagactgggagcacatctctgtggctgcaggcgtcactttctgctgttctcttgtctacactttctgtgttttgttggacaatccagtcgtcaggccgtgcccacctgtcatgttggatgggaaatgtag

SEQ ID NO: 111 - DNA, 204 bp, Homo sapiens
atggctgcgcctcccttagcaaatcccctgcatttggtcgtgacaccaatgaggagcaggtccctgttttgtctctgcctgggctggcctgtctggaacagtcttctagggtcacagtgcacgtctgcctctgggcagctgcctgacccaggcctccacgagagcagggaagaggcacagagacgttccatgatggctgggtga

SEQ ID NO: 112 - DNA, 156 bp, Homo sapiens
atgagcagggaaatgttaaaaaataattcttggaggaagagtcttaccagcaggaccgatgtggctgggaagaggaaagccctccagtcaagaaggttgtgtgtgtggctcaccattgagtcatctttatcaccagtgagatcacccttcttctga

SEQ ID NO: 113 - DNA, 153 bp, Homo sapiens
atgaaacatttccatcattccgaaaagttccctagcatatgtttgcagtccatcctgcctcctgctcttagcatcatagaactactgatttgctttctgtcacttcagggaagttcacattttccagaattttatataaatagaattgtttag

SEQ ID NO: 114 - DNA, 192 bp, Homo sapiens
atgcccagtcagggaagccgaaggcctggaggggcttccgggagcaggggctggagttcctctgccaggcaggaggctggcaccagacacccggcagagggaggcggcgagggcccagcaaggattctccccagcccctgtgcctgcgtctcctgcggcttctgtgcgcggaccgtgtcctgggctgtgtag

SEQ ID NO: 115 - DNA, 153 bp, Homo sapiens
atgagcacgacgcagttaggaggctattggcccggcgcgggggaaaagggggaaggtcgggctcggggtggcaggaccccagagggcaggggtgactgccggggtgctcttggggcaaggtgggcgtcaaggcccccagcaaggttgggataa

SEQ ID NO: 116 - DNA, 168 bp, Homo sapiens
atgcaagacaaagttctttttactctttcctgttctctcctcaagcagaaagagggagtcacttttgtttctgcaagatgcactacatggggttggggtatgggtggtgcaagtacttccttagttaccttatctggtgtctcatttggtgtctccaagtccacttga

SEQ ID NO: 117 - DNA, 162 bp, Homo sapiens
atgaaggtgattttttttgtgtgtgtgtatagatggttatcaaatttagcattcctgtgggagataatggacaatcagtggaggcctctattcagccaccttactctgcccctagattgttctttacaaatacacacatgcttatatcattctgatgtttaa

SEQ ID NO: 118 - DNA, 162 bp, Homo sapiens
atggccatgagctcttgctgccatgccccgtttaggaacactgctctttgggctgagctcattaaacctgaaagactcagacagacagaaatacttttagaacacactgaagaacagtgtgaaatgatgtgtcatagctgtgaatggctggaaaaatcctga

SEQ ID NO: 119 - DNA, 231 bp, Homo sapiens
atggtgattgtattagaagggacttggtggaggcaagcctgctgcagcgatctgttccagccgtggcggtgtcagaatgtggcagttctgacgtggtggccctttgtgtctcaggagcaaagtgacaaaatggcaaggatgggggttgctcatctcaacctcacaaatgagacggcacctggagaaacacatttcatcccagacacctcattagcagagagacacagataa

SEQ ID NO: 120 - DNA, 198 bp, Homo sapiens
atgtctcctacttgtttaaaaaaaggaaagaaaaagatgagtgcttccaagccattcgaaccactgcaagaaggtttcttctattttaaaccccagattgtgtatcacaaggcacggctgaaggaaggcattctgataatgctttcaggaatggtaccaatgagtaattttgttgctactttccattttggggtatga

SEQ ID NO: 121 - DNA, 234 bp, Homo sapiens
atggttcagggtagctttttaacttcatacgaccctcacttctgttacctgaaaacaataaaatgtaatgaatacagtgttcaaaaatcaagtggcttgctttctgagatttcttgggtgtgtcctctctcacatgtatttagatcttctctttattattctctcttactaccagcactttttcttcaaagtggagtgctctcttggaaaaaggccctggtagaccaattttga

SEQ ID NO: 122 - DNA, 156 bp, Homo sapiens
atgaatacagtgttcaaaaatcaagtggcttgctttctgagatttcttgggtgtgtcctctctcacatgtatttagatcttctctttattattctctcttactaccagcactttttcttcaaagtggagtgctctcttggaaaaaggccctggtag

SEQ ID NO: 123 - DNA, 300 bp, Homo sapiens
atgggaaggggtgaggagcgctgtgggaaaggctcctccttggcaaggaaagggatgatttctccggagttcaccggactccccctttccccagcgccaagcatccgggtgagtgttttgcaatggggtctgtcaccatcaggaaccgaggagagtttagactctgcatttactacaccagacccacaaagcaccacctgtgctctcagtctcaggagaaacagcctctttcacttttcaaatcaatcttctgtgcatgtatttactataatgttacagcaccattggttccagctttaa

SEQ ID NO: 124 - DNA, 279 bp, Homo sapiens
atgaagacatcggttcagttgcctgtttccgcagctattccttgtcacactcaacgctgcttcagctgttcatgggacagcataaaaatacaccttccacactctgtctcaggatgtttaactgcagtcagggaaaggctgcattttcagaaagactctggcaggtctgaacacccattggccacactggtgcatggggtgcctgaaaacaggtgcaaacccgactcttcacctcccagagtcagattagaattggaggccagtctgctgttctattag

SEQ ID NO: 125 - DNA, 1092 bp, Homo sapiens
atgcaagttagctcttggccttctgtctcttgcaggattcccaggtcagaacacaggaaatacaaggtgctgtacaactcacagctgctgttccgcagccggctctatggggacctggaggccatcctgtaccacgtgcacctcttccagcccacggagctgctgctgcagcaggcggtgttcttcctgcgagacactgagcggaggcgggtcttccaggctctagccaggatccacgacatctgctataacagcaccaccctctgggacgtgacggtgagggacctgctgccctcctctgctatgataaaagacttgagccaagagtttgggatgcccctttcgcaagaagaactcacagatgagaaactgtttgccctaccacctcagcctgcccccaatcttgaggactaccacagtcggaactccaccctcaccttagagatccacgcccaccaggagaagtacctgcagtggcggagtgccatgcttatgaagaacaaagacaaaaagcacagtttcatccagaaaaatatcacagaagcctaccaggtcagcaagaagcctccgaagtccgtggcgaaggtgattaaaatttcagcccctgccaacaaggccgtctacaactatagtacccagaccatgaattctacagagcttgccaagaaggagctgtatcaagagatagccaaggagccaagaaagagattcacgtactcacaggattacctctcagccatggtggagcccctggacttgaaggaagaggagaagaaagcccagaagaaatcccgccaggcctggctcacagccaggggattccaagtgacaggtcttcagagcgacaccgaaagcagctttcaggatctcaagctgccacccatcaaagagctgaatgaggagtggaaggaaaactccctgtttgctaatgtactggagcctgtgttggatcgagacaggtggagctgggacaggcaccacgtggactttgatctgtacaagaaaccaccacctttcctcgagctgctcccttcgcccgcaccaaagcctgtaacagtcaggaagaagaaagggaacagccccatctcctga

SEQ ID NO: 126 - DNA, 261 bp, Homo sapiens
atggcagcagccaggccctgtgtgaggctgggctgggctcacctcgtggtcgtggttgcggagcccaatgcggatggagcggctggcccgcgacagcacggccgtcatgccatacaggttgatgaggatgttggccacccgcttcagtaccagctgctcctccatgatggtctgggatcacagaggctccaagtggggactcactacctagaccagtcccccacatggtccctccctgggctgcatctttgcctgtcttag

SEQ ID NO: 127 - DNA, 249 bp, Homo sapiens
atgttgagctactgcacacctgttatcaactccatgtttagtggcgtcatattgagaggtgacagcgggccagctggagttccgggtgggcatgggcttggcgggcctgcactcggagcagccggccggccctgccagccccgggcagtgagggacttggcacccgggccagcggctacagagggtgtactgggtcccctagcagtgccggcccaccggcgctgtgctcgatttctcgccgggccttag

SEQ ID NO: 128 - DNA, 234 bp, Homo sapiens
atgcctgagcctcccatccactccatgggctcctgtgctgcccgagcatccccgacgagcaccaccccctgctccacggcgcccagtcccatcgaccacccaagggctgaggagtacgagcgcacggcgccggactggcaggcagctccacctgcagccccggtgcgggatccactaggtgaagccagttgggctcctgagtctggtggggacatagagagtctttatgtctag

SEQ ID NO: 129 - DNA, 285 bp, Homo sapiens
atgtatatatgtacacacaaatatgcatatgtgtgcacacatatgtgcgcatatgcgtatgtgtgcacacatatgtgcgcatatgcgtatgtgcgcacacgtgcgcatatgcgtatgtgcacacatgtgcacatgtgtatgcgtgcatatgtgtgcatatgtaagtatgcatgtacacatatgtacatatatgtacgtatgcatgtacatatgtttgtgtgtatacacacaaaaaacccatacatacattcaaaaatgaaaagacccatgtctgattcttcttag

SEQ ID NO: 130 - DNA, 267 bp, Homo sapiens
atgcatatatgtatatatgtacacacaaatatgcatatgtgtgcacacatatgtgcgcatatgcgtatgtgtgcacacatatgtgcgcatatgcgtatgtgcgcacacgtgcgcatatgcgtatgtgcacacatgtgcacatgtgtatgcgtgcatatgtgtgcatatgtaagtatgcatgtacacatatgtacatatatgtacgtatgcatgtacatatgtttgtgtgtatacacacaaaaaacccatacatacattcaaaaatga

SEQ ID NO: 131 - DNA, 222 bp, Homo sapiens
atgcgcctgggtggaggctcccgagagaagctggcctgcgggcgggccggacgagctgcgcggacggggcggggcgaaggaggccggcaggcggaggaggaagcggcggggcggaggcggcggcggccgggaagaactagaggtattccccgggcggctggaggactgagtcgagccgggacccgagtcctccggtatcccagcagccaccggaggcagtga

SEQ ID NO: 132 - DNA, 204 bp, Homo sapiens
atgcaccggggacgggcgcccccatccgactcttcgaggggcggcagggccccatctgtgtctttcgctctcgagcccccagctagagttggcttcagcggaatacctactgtgcgggattattcaacaagccgattgatcacattcttcagctctagcagtgcaaaggcttcactgaaaaagaacattacaaattttttttga

SEQ ID NO: 133 - DNA, 195 bp, Homo sapiens
atggagaatggatataaactggtaggctttactcgagaaggttctggctgggcctttttgttggagagccatcaacttgcagtatctttaggtatttttggagggttggtaactttttccagagaggactccactaatcttttgtcaaaagaatctaagcctgcttgcaggttctgccagctgactggggactag

SEQ ID NO: 134 - DNA, 189 bp, Homo sapiens
atgattgttatttggaagttagaccctccttggtcaatcatctcattttattattattttttcttattttcttgttctagtctctggcagattttctcaactttatttttcaacccttcttttgaattcttcacttcctatattgtattttcaatttccaagagccttttttgtttgtttgttctctga

SEQ ID NO: 135 - DNA, 222 bp, Homo sapiens
atgatgccattgtttttgataaaaagaattaaacaacataaatgtatgctagaaaaagaactagaaggaaataagctgatatattggtggtctggttccagaggagagattttagtgattcttattttcccctttatacttttggtattttctaaatatttctctaataaacctgtgctaatcttatttatttcaaaatgtttctacttagagaaattttaa

SEQ ID NO: 136 - DNA, 204 bp, Homo sapiens
atggtaggttataaggatttcatgtgcttatttatttaccttttgtttgttcatctgtatttgctaatttttctatttttttatttttattttttgagacaggatctcactttgtcactcaggctggagtacagtggcacaatcatggctcaccacagccttgacctcctgggctcaggtgatcctcccacctcagcctcctga

SEQ ID NO: 137 - DNA, 162 bp, Homo sapiens
atggacacgtatgatatggagcaatttttgaattgtctttcacataggggaaattgcatcttatatacccataaattgggttttagtgaaagagccaggatggaacataatttcctaacttccatcctagtttcctttctacttgttcagaatggccagtga

SEQ ID NO: 138 - DNA, 201 bp, Homo sapiens
atgataaaggggtcaattcagcaagaggttataacaattgtaaatatatatgcacccaacactagagcacccagatattataataagcagctaataacaagcaaattaataataaagattggagcagaaataaatgaaatggaaatgaaaaaacaatacaaaagatcaatgaaacaaaaagtggggtttttgaaaagataa

SEQ ID NO: 139 - DNA, 174 bp, Homo sapiens
atggcaggaaggcagaaaagaaggaagagaagaccaaaaagcaatcagaaaactaataacaaaatagcaggagtaagcccttacttatcaataataacattgaatgtaagtgaactaaactctccaatcaaaagacatagaatggctgaatggataaaacacaagacccaatga

SEQ ID NO: 140 - DNA, 327 bp, Homo sapiens
atgaaggctatggtagggacaatccaggatcctaccagagcttattcacatgggtggtcagaaaagaacggctgcccatggcattggggcagagaaaaaattgtacaacaagccaaagaatgcctatcctttcatgaagcattttggtggggccattctgtgacctgtctatgcttaatgattctaggaatggattcagttacaggctggactcagggcttctttgcagtaagtgacaggctaccacatacataccacatatgcacactgtggtctgaacaagaacactcgcttaaaaggaaagtcaggaggaaaaggaaagggtga

SEQ ID NO: 141 - DNA, 186 bp, Homo sapiens
atggtttgtaagtggcagaactggggcgagaagccaagtggttgcctcctgtcccttccttccattgtaccacagcgctggcctctcctctcctcacctgacactccatgggttcaagtcattgctaccggatgccaaactctgcaggtgctgtgctggacaccgggaagccaagtgactggatga

SEQ ID NO: 142 - DNA, 279 bp, Homo sapiens
atgacaaaatgtataatttctatatcattatgtttatatgactgtcttatacaggcttttcttccaatgggttggatagaaaaatattcttttttttttttttttgagttggagtcttcctctgtcgctcaggctggagtgcagtggtgcaatctctcggctcactgcaagttccgcctcccaggttcacgccattctcctgcctcagcctcccgagtagctggtactacaggcacccgccaccacgcccggctaactttgtttttgtattttaagtag

SEQ ID NO: 143 - DNA, 237 bp, Homo sapiens
atgtattaccttcatgacagttttcctttccctagggaaaattgccaggaatcattaactgagtggttcttaatggaaaacctggattggaggatacttatattcccccccaccccccattcagtgtccttatattgggaggagtcagtggaaacctttgtaggggatctttgttgctttggagtttatctgttgctgttactatctggagtcagtgattgtggaaagaacatgtga

SEQ ID NO: 144 - DNA, 276 bp, Homo sapiens
atggctaccaagaggtggagtggtccagtcaaagcaaaagcagaccagtcaaaagcaagggtcatggcaacagtgttttgggatgcccaaggcactttgtttgctgacttgctggaaggccaaaaaacgatcacatctgcttattatgagagtgttctgagaaagttagccagagctttagtagaaaaatgcctgggaaagctccaccaaagagtccttctctaccatgacaatgctcctgctcattcatctcatcaaacaagaggaaatttgtga

SEQ ID NO: 145 - DNA, 159 bp, Homo sapiens
atgccaatagggtttgaaagtttagagaaaatagacaaattcctagcaaaatataaattatcaaaacctactcaagaaaaaatagaaatcttatatagtcacaaggtattaaatattcaattcttcctagagacccaaatatatgctttctacccgtaa

SEQ ID NO: 146 - DNA, 210 bp, Homo sapiens
atgtctccatatgaatgtcagactggcatctcaaacttaatgtgtccccccaaaatcttgattttcacccccaaacctgcacctctctcagtcttgtgcattttagtaaatgtcactggtactcattctgttgttcaagctaaaattctacgagctgtttttgatttatctcatccctttacctttaaatccttcagcaaagcctgttga

SEQ ID NO: 147 - DNA, 234 bp, Homo sapiens
atgcatccatggccggatgtgttggctcacacctgtattcctagtgctttgggaagccagaggcaggcggatcacttgagcccaggatttcgagacccagcttgggcaacatggcaaaaccccatctctacaaaaaaaattagctgcatgtggtggtgcgcacttttagttctagctacctgggaagctgaggtgggtggatcacttgagcctgggagtttgaacttaaaatga

SEQ ID NO: 148 - DNA, 174 bp, Homo sapiens
atgaagaccgcagaggtgaaaaaaaaaaagtgtgcgcacacggggcggaggacagggtgtgaatacttggaagaattctgcagtagttttgaattatttgattggttttgcccttttaaaggtcaccttatccttgttttgtttgtttctaggctgtctgactcttcttactag

SEQ ID NO: 149 - DNA, 378 bp, Homo sapiens
atgcggctgcggtggatccagggagctggggcccagatccaccaagatcagggaaggttcctgagggcccgctgtccctcagggcctgaagaaaatccattctccgcgtgcttgcgttggagaacacagtggcccccaccgcccgccctgagacctccacagggtcctgatccagaacttgacgaccggacgccatctcatctctccccacagaacccctgtcagcctgccgggggacgcggtgcccgccctccggcatccgggaaacacagcgtcccagccggcgcctgcgcaggtgccacacttcctggtcctctctggctgcgtctgagggccttagctctggccaggggcgctgcaacgggctcagccttctag

SEQ ID NO: 150 - DNA, 192 bp, Homo sapiens
atgagctttacaacacttccatcagagaacgctggagtgcagtggtgcgatctcggctcactgcaacctcggactccctggttcaagtgtttctcctcctcagcctcccgagtagctgggattacaggcacgtgccaccacaccgagctaatttattgtatttgtagtagggacagagtttcaccatgttag

SEQ ID NO: 151 - DNA, 288 bp, Homo sapiens
atgaaatcccataagtcaattttttctcgggattgtgaagttcactgcacacaaccagccttattttacatctctttcttttatttcaaccatctggggactgggcagaagtcatggtcagaggggcacatatgtattgctttgatgatgggggcagagaacaaacaggaagaactgggaaatgaaaattgctttacttcttatattctgactagaaaggcaacccaaatgggaatcataacagggatagttcagtggcaaaaagacacccttaaaaatagaagctag

SEQ ID NO: 152 - DNA, 273 bp, Homo sapiens
atgccttgcaaagaaccatgtgaccaccaacacatcagaggacaggagagtttcatcacaggatccttgatgctactctttatagccaagctgacttcctccttccccctccctgaggctggcaaccacagtatggttcctattttctataattttgtcgctttaagaatgctctataaatggaatcatacactatgcaacctttcgagactggctttttccgctaaacgcaattcccttgagacccatccgggttggcaagcatgtccatag

SEQ ID NO: 153 - DNA, 183 bp, Homo sapiens
atgagcacctccttagcggttggggccggggctgtggtcacggctgagagcctggaacccagccgcccttcagtggaggaaggccaggggaagggtaccgcggcagagggggtgtgtgtgtgtgtgtgtgtgtgtgtgtgtgtgtgcgcgtgcgtgcgcgcgtgtgtgtgttttgttttttga

SEQ ID NO: 154 - DNA, 177 bp, Homo sapiens
atgctctgggatctgacggtcctggacacctgtatggctgtgtgctgtggtctttgcctagcctgcggttcacttttgctctggccaccacctcccctcatgtacaaaccgcgtctctgctctgccagtcttggcccccgtcaggcagcggttcactccctcaccagggaagtctag

SEQ ID NO: 155 - DNA, 153 bp, Homo sapiens
atggtaagtgtggtagagaggggctggaattgcctgttggtgtatctgtgggaagcagctgctatccttgaggaagaaaagatggagaaggtaagtggctatcaacctgtcagagacaacacattctgtgaagttgtactctttcatttctga

SEQ ID NO: 156 - DNA, 213 bp, Homo sapiens
atggagtatcgccatgttgcccaggttggtctcaaactcaggctggtgagctcaagtgatccgcctccttggcctcccaaaatactgggattacaggcatgtgccaccatgctgggccacaagttcatatctggagtagaagttttactttgtaaatattataaagtagaagaaaccataaaccattttgctaaaatgaaaggttggggttaa

SEQ ID NO: 157 - DNA, 165 bp, Homo sapiens
atgagatttcagcaattcctttttgcattttttatttttattatgagtcttctccttatcagcggacagagaccagttaatttgaccatgagaagaaaactgcgcaaacacaattgccttcagaggagatgtatgcctctccattcacgagtaccctttccctga

SEQ ID NO: 158 - DNA, 255 bp, Homo sapiens
atgagtggccgtgtccccctggcagagaaggccttgtctgaaggctacgcccgcctccggtacagggacacctccttgctcatctggcagcagcaacagcagaagttggagtcggtgccacctgggacgtacctgagcaggagccgaagcatgtggtactcacagtatggaaatgaggccatcttggtccgagacaaaaacaagctcgaggtctctagggacacagggcagtccaagttttgcacaattatgtaa

SEQ ID NO: 159 - DNA, 201 bp, Homo sapiens
atgcgtgcggggcgtgccaccggagctccttccagcttgcgtctggggccaccatgtcccttcctcagggcactggatccatctcttcaagctcagtggtccaggccatggtggcagaatcaccccccagggtgtgacgcctcgtccctgtcaattccacacagctgcacccggaagagctgtggccgggcgccgctctga

SEQ ID NO: 160 - DNA, 321 bp, Homo sapiens
atggctttatcactcgcttcaagccctacaccctgcttcttcattcttagaaaaatggagttatcaagttttaactcagtttctcctttccaagtgaaattgggaaatcacaaatttacccctataacttactcatttctctccactattaggattctgttgaccctctctacccttttatgtctcttcctactcttgctgtacaaatatctcctctcctttattgtcagtcttttccccttcagtgactctccttatcctacaaaatgctcaagactcccacaaactactgcccatctcatgcaacccacaacagtctga

SEQ ID NO: 161 - DNA, 282 bp, Homo sapiens
atgagtctaggcagtggttttatttctaatgagcaatttgtgagacatactattaatcatgcaatacctttaacagttgaactttttaattgtgggtttgtgtgtgcgagggagatatttatgatttctactaaagccctcaaagctgctcagacagcccttttgtccatttttggtcagctcccatggctttatcactcgcttcaagccctacaccctgcttcttcattcttagaaaaatggagttatcaagttttaactcagtttctcctttccaagtga

SEQ ID NO: 162 - DNA, 192 bp, Homo sapiens
atggaaccacagagtgtgacatttattcctgttcctgcaaatttctttatcaaagaaatatactttgagataaatgttataataataagtcctagctttcttaatatttctaaaaacaaagatgaccaacttacaccaaagtaccaaagtagaattaaggttataactaccacggtaagttactgccattaa

SEQ ID NO: 163 - DNA, 189 bp, Homo sapiens
atgcacagtgatggtaccttgctctatttccatatatcacatttatccaattcacccagtcatctgtcttactcttttcttatcaccaacataacaattttagccttcataatggctggaaattggcaaacagcaatgagggactaccacatatcaaaacctatcacaaccacctttggtgtattctga

SEQ ID NO: 164 - DNA, 267 bp, Homo sapiens
atgggtaaagatttgaatagatgtttttcccaagaagatatacaaatggccagtaagtacaagaaaagatgttcaacatcattcatcattagggaaacacaaatcaaaaccacaatgagatatgtcacacccaccaggataaccacaattaaaaagacgataataacaaatgttagtgagaatgtgaaggaattagaactctcattcattgctggtatgaatgaaaactggcataactgccttggaaaacagttttccagttcttaa

SEQ ID NO: 165 - DNA, 201 bp, Homo sapiens
atgaggtctttctgtgattccagcagcctcaggaggacaacgtcagaaaccaaaagctcctctgcccagggatggggacccgccgacttcaagggcagaacaggtgcctggctagtgggggagttccccaatgtggatttaaactgtagtgtcttcagagaacataaccaacatttgtctttaggagatgtgtttgtttga

SEQ ID NO: 166 - DNA, 174 bp, Homo sapiens
atgtgtcagaatatgtgcaagctttcgacaggatccccgtttctccccatggagcttttggcaaatgaagggtgtggttcagcagctggtctgatgtcccttaagctcccccacaccagcacaccaggactgtctctgtctccacattccagcatatctgggtctcagctttga

SEQ ID NO: 167 - DNA, 396 bp, Homo sapiens
atgcctcagcctccctcagccttgccgtcactggctaaagataccaaagaggagacggaggttttcctccccctccccccaatgaactgggaagaagacaagcgatatactacagttatgggaccctgccttaggcaagcagcattggaaggggaactcttggcctgcccagtgatgcaagatcaacaaggcaattgggtgcatgagcccattacattgaacacttctaaggaaataagaaaaagcattagagaaaatggggccactagcccattcacaagaggattaattgaggcaatagcagataactaccatatgactccatgggactggtcagtgctagctaaaacaactttaggaggcgagtcaataccccctctggagggcagaatatga

SEQ ID NO: 168 - DNA, 378 bp, Homo sapiens
atgaataaggaccctacaacactactagcacaagtcttattcacccttaatttcttaaatttagataataaatttcaatcagccatagaaaagcactttgctaaaacctcccaagatacaaagccttcagtgttatggaaagatgtaaatagtaatctatggtgcagtccacatgatttgctaacatggggaagaggatatgcttgtgttcacatcccctcaggtcctcttgggattccagtgcaatgcatcaaaccataccatggcatggctgggacccaatgcagtactggaaatgaagaatgtgagcctgtaggacccgcagccccggacaatgcagcttcctcggacaacacaggtcccggatggggaatgtga

SEQ ID NO: 169 - DNA, 210 bp, Homo sapiens
atgtccaccatttgtggatttcacagctacttgtcaatggtgaatattgatcatcatcattatctactgagctgctaccatatcccagctactccttgcatgttgttcattattttctcaacactcagcatatttgcaatatgttatgtaatatcacagacaaggaaactgaacgcagaaatgttttatttcttgccaaacatcacatga

SEQ ID NO: 170 - DNA, 192 bp, Homo sapiens
atgttggttggccaggctggtctcgaactcctgacctcaagtgatccgcccacctcagcctcccaaagtgctgggattacaggcatgagccaccacgcccgaccaaaaaaaaatttttttttttcaattagccagacattgtggcatgtgcctgtagtcgcagctaccatgggggctgaggtgggaggatag

SEQ ID NO: 171 - DNA, 159 bp, Homo sapiens
atgcactgccaaggccaccaaaaggatgactccccaataaccaaagggaatcaagtggctgataaagctgcaaagcaggccactcaagaaacatatttacttgggaccttaatactccatttaaacttgtcagaatttaaactccactacactgaatga

SEQ ID NO: 172 - DNA, 333 bp, Homo sapiens
atgcctcccaggttcaagccattctcctgcctcagcctcctgagtggctgggattacaggcgcgcgctacctcgcccagctaatttttgtatttttagtagagacagggtttcaccatgttggtcaggctggtctcgaactcctgacctcaggcaatccgcccgcctcggcctcccaaaatgctgggattacaggtgtgagtcatcacgcccagcctctattttcaggaactttaaaggcagaaaaaacacttctctgaccttaccattctttagtgtacttgcaacctggtgtccttgcatccagatattcttactaatcctgatgcaatga

SEQ ID NO: 173 - DNA, 207 bp, Homo sapiens
atgcggcacctcaaagctacaacattaatgcaagttactgatagagaatgctatcagagtgaaaaggaaaaacttgaagattattttggcttttttaaaaatagcaaagattacattattttattatattttgcttttgagagtaaagttggaataattactatatacaattgtttctacctaattatcaaggaatttaatcattaa

SEQ ID NO: 174 - DNA, 306 bp, Homo sapiens
atgtttcaagaggatgtaggtaagtatggtgttcacctagctttccagaagtctcatattttgtgtaaaattggtatgcttattcatggttactttgggttagtagaagctatacttcttttccaaggaaagacagactgtcttaagattctaactgtgcatcttggtgataagaagttcttagggttgttaaatagagccaggaagcgtgaagctttctctctagatcatgacaactcatctagtaccttgcattacaaaggagctctaagtccatttactttgcaaggaatgaagttccagtga

SEQ ID NO: 175 - DNA, 153 bp, Homo sapiens
atggagacagggaccggctgctggggtttgggaggggcagagattgaggagccagttgctgtgccctgttctggcaaaagaacaagatgcgttgcaagccagaatgatgaattccacttttcacgtcgcctccgtggcattctgcctaaatga

SEQ ID NO: 176 - DNA, 213 bp, Homo sapiens
atgacttgtatggatttaagaaatgtagggaggagtttcattctaatcaaattcttcctgacacaatggttcaccctgatttaccagtttttgactacattgctgagcagagagaaaattgcagagttgacttgcagtattttgactgattcatgtcctagtgcttcggggaaattgcttaaggaagttggcgttgctcaaaagtatagttga

SEQ ID NO: 177 - DNA, 177 bp, Homo sapiens
atgaccctgcttggcttgtgtcaaatcattcttggccaaggtggctggtttacccaatatacactgcccatgagtgaaaggcaattatcacaggaggggagatggatcttgggtgtgggaaacaggtgtagaattcctactttttacagtttggtagaggtaatgtttagaacctaa

SEQ ID NO: 178 - DNA, 243 bp, Homo sapiens
atggccaataagctcatgaaaacatgtttaacatcactaatcattagggaaatgcaaatcaaagctaaaatgagataccacgtcacactcattaggtgttattttttaaaaataacacaacaacaaaaaataagaaatgtgggcgaggatgtgaagaaattgaaacccttgtggactgttgttggggatgtaaaatggcgcagctgctggggaaaacagtatgggagttcctcacaaaactaa

SEQ ID NO: 179 - DNA, 165 bp, Homo sapiens
atgttcacagctgagccaaaaggtgggagtaacccaaatgtccatcagtggatgaatactggcaataaaagctgtgtctggaggccactccgtttcacacagctctccacacgggtgagggcacagccttctcgtctctttcctatgctgcatcttcagagctag

SEQ ID NO: 180 - DNA, 222 bp, Homo sapiens
atgctcggtctctctcttcagcctcaggcccacctgtcttctcgagagacattgcattttgcgtgtcccacaaatatctcaaacatgtccccaaacgaactcatccattctgaagctgcctggcttaacgaatggtgccattgtctccccagaagcctagaaagaaatgtgggagcctcagtccatgaccatgcccaatgctccccaccgaccctcgtctga

SEQ ID NO: 181 - DNA, 213 bp, Homo sapiens
atgactatgaccatgcccaaggctccctgctgtccctggcccaagctctggatgtctctgtctgagactgcttactcccagctggtctccctgtgttcaccatgtccacctttaacccattgtccacaccgaagatccaaccgtaaacctgatccaatcctgtttaaactcttgcgtgtttcccattgtccttgctggaatcccgagtcctaa

SEQ ID NO: 182 - DNA, 288 bp, Homo sapiens
atggcatgtaaatctagattcacttctagctggaaggaggcaggccagaaattcagagttgtaggtccttggcctgggtggatgtgctacaacatgaaccataaacctccatcacttatcttggcggatagaaaattggaactttggaggaaagtttcgcatcaagcactcagacccaataaatactcccactccttgcagagctaccgaacattctgcactccatttatcaggctacccaaggatgcttggaatgtgctggccggagccaccatcagcacttgctga

SEQ ID NO: 183 - DNA, 210 bp, Homo sapiens
atgatcactgctcactgcagcctcaccctccaggactcaagcaatcctccccactcagcctctcaggagctaaggactggctcaattgactataaagaatcgagaatgtcagctgaccaggcaaccaggagacgctttcctgacttccactatgcacgtgggctgcataattgtgtctgtgaagtaatgaagaacgtgcttgctctgtaa

SEQ ID NO: 184 - DNA, 219 bp, Homo sapiens
atgctatccctcccccaggcccccacccgcaacaggccccagtgtgtgatgttccccgccctgtgtccaggtgttctctttgttcaattcccacctatgagtgagaacatgaagtatttggttttctgtccttgtgatagtttgctgagaatgatggtttccagctttatccatgtccctgcaaaggacatgaactcatccttttttatggctgcatag

SEQ ID NO: 185 - DNA, 168 bp, Homo sapiens
atgagctttgatttagggaagaacattgttgggcaccctgagtccacctgccaccagccgcggagggcatgttataaacacaccagacacgccaaggatgggaacattggacctaatttagcaacaacaaatcctttccctcatgcatacttctctaattacaaatga

SEQ ID NO: 186 - DNA, 168 bp, Homo sapiens
atgctgtgttcgtgtataatatatcacagtggttgctggagaagggaacttcccagagggatcgctgactttaagctgcaacatagctcaacggtcagaggtgatgtttccacccaaaatctaactgtgattactgcaggctacaaacttatttgcctcatggaatag

SEQ ID NO: 187 - DNA, 216 bp, Homo sapiens
atgtttggaccaggtgcagtggctcacgcctgtaatcccagcactttgggaggcagaggcagccacctctcaacaacccatgaagtgtctggagctccaccctctgctcaagaggaacggcacatgacccaggcctatgccaatcagcatttcacattcctcatggttggttcaaagctttgtatctgggtacacctgtttcctcctgagacatga

SEQ ID NO: 188 - protein, 89 aa, Homo sapiens [sequence omitted]

SEQ ID NO: 189 - protein, 87 aa, Homo sapiens
MMHWMKKIWYIYTMEYYAAVKGNKIMFFARTWVELEAIILSKLTQEQKTKHHMFSLISGSPTMRTHRHREGNNTHLGLSWGWREGEH

SEQ ID NO: 190 - protein, 82 aa, Homo sapiens
MRFPISLHPHQNLLLSVFFILDVLEGVEWYLIVVLICISLRTNAFEHFFMCLLAICISSLEKCLFKSFVHFLIGLSFYCCMV

SEQ ID NO: 191 - protein, 61 aa, Homo sapiens
MFIQILCPFFNWIVFLLLHGISSLYILDTSLLLVIPFANIFSHSVRCLLTFLMCPLKHKSF

SEQ ID NO: 192 - protein, 66 aa, Homo sapiens [sequence omitted]

SEQ ID NO: 193 - protein, 54 aa, Homo sapiens
MTYLEIFIQRIYYSIFQCATATTACFWSECSATNIAILLGKRSAGCWIVEVAHI

SEQ ID NO: 194 - protein, 84 aa, Homo sapiens [sequence omitted]

SEQ ID NO: 195 - protein, 73 aa, Homo sapiens [sequence omitted]

SEQ ID NO: 196 - protein, 160 aa, Homo sapiens [sequence omitted]

SEQ ID NO: 197 - protein, 108 aa, Homo sapiens
MGPALARGDPSFRSGLSSPWGPARRKSARLPNLGHTLSAPLPSPRRNSRSPGRQGPGSKSPEFPPELGWARGPLMRRLPGASPQPSLIAGPDQSRGERPCSLPADRDL

SEQ ID NO: 198 - protein, 79 aa, Homo sapiens [sequence omitted]

SEQ ID NO: 199 - protein, 68 aa, Homo sapiens
MAPAAAPSSLAVRASSPAATPTSYGVFCKGLSRTLLAFFELAWQLRMNFPYFYVAGSVILNIRLQVHI

SEQ ID NO: 200 - protein, 55 aa, Homo sapiens
MSSPFTHSQDPETSTAYKTLHQWPPTFLAPGTSFVEDNFPTDWGGGVGAVWGCEM

SEQ ID NO: 201 - protein, 53 aa, Homo sapiens [sequence omitted]

SEQ ID NO: 202 - protein, 74 aa, Homo sapiens
MLFFPSHSILTLSILRSQLSCREAAFPIWQPIICVIVPCCTIELDTGAMLCLEMYSPASKGLPQSPLLKKPQFY

SEQ ID NO: 203 - protein, 53 aa, Homo sapiens
MLDVRDKWLTGHSSCLSCYGLSSLIQSSLISLTVLSLSPVDSLVGHWFLSVLN

SEQ ID NO: 204 - protein, 101 aa, Homo sapiens
MFVVCGMFCYCGPSCWTQGHPWAPRHHPSLTLLLSWNTVVPHCPSKATSRLHLLTPCFFHSMVKFFSYQEAGQVQWLTPVIPALWEAETGRSFEVRSSRPA

SEQ ID NO: 205 - protein, 65 aa, Homo sapiens
MTVSRAPHVLDPRWAEATKGDPARVKGGCRQARAEPCRMLRARAPPSTGPSPSELSIDWTNHPQC

SEQ ID NO: 206 - protein, 68 aa, Homo sapiens
MEPFRRAQPWGIYDPWFPSRKLRQRRKKRAGQPMRERRQRKRKGDGRKGNAFFRTQEKQHQKWAFLPV

SEQ ID NO: 207 - protein, 66 aa, Homo sapiens
MQLSNKPSQPGLKVSNSATIKAILCHDQEHRAWSQTDKGLRLMKDCQVTFWSLLHSLEIRIWSFKL

SEQ ID NO: 208 - protein, 64 aa, Homo sapiens
MPGQRGPAPASGWKGREQRGGPSYSGPPGQSPGVGKQRAKRRPLLSSLTPRPEPKVQVPQAQSP

SEQ ID NO: 209 - protein, 63 aa, Homo sapiens
MSTLSSAGPQLEGAIQSSFPSPAELQRWGGCRRAQGCLGRGLCAFCCGGHQGFRKILNGLSID

SEQ ID NO: 210 - protein, 132 aa, Homo sapiens [sequence omitted]

SEQ ID NO: 211 - protein, 74 aa, Homo sapiens
MCISLLRFHMYSFIMRIFFLHPMTIVLIAALKYLTADNNILDILGIAFNAYVLSCVWITFLCFFSCLLNFKIVF

SEQ ID NO: 212 - protein, 60 aa, Homo sapiens [sequence omitted]

SEQ ID NO: 213 - protein, 54 aa, Homo sapiens
MGFPILVSQFRSWYCLSIISYPLLGNKLLPNLAASNNNKHVLCHIVCFRTLGVA

SEQ ID NO: 214 - protein, 85 aa, Homo sapiens [sequence omitted]

SEQ ID NO: 215 - protein, 79 aa, Homo sapiens
MFLVEMGFHHVGQAGLQLLTSSHTLASASLSAGITGTSHRAWPGICIFASNPGHLSAHQSLKPLLGCGVLASRLPGPMD

SEQ ID NO: 216 - protein, 73 aa, Homo sapiens
MNSLLTLVLFVCLFSPFLVKCANILKSQENHMVISHSPLWPASAKMTFCHSYSIHFCHATPDIHTCSFWSIFK

SEQ ID NO: 217 - protein, 116 aa, Homo sapiens [sequence omitted]

SEQ ID NO: 218 - protein, 54 aa, Homo sapiens
MEFHHVDQAGLELLASSDPPVSASQSAGITGISHHTQPKTFFIMIQVIYQTRNY

SEQ ID NO: 219 - protein, 60 aa, Homo sapiens
MKATMSLLNKSFIKRNIYQGLPCHIDPLKVRSEVEAALMFLILNLIQQKKSKDILTSIDF

SEQ ID NO: 220 - protein, 92 aa, Homo sapiens [sequence omitted]

SEQ ID NO: 221 - protein, 89 aa, Homo sapiens
MYDHGLKKLFFFFFFFFETEPCFVAMLGCSGAILAHCNLHLPGSRDSPASASRVAGTTGTRHHAQLIFVFLVEMGFHHVGQDGLDLLTS

SEQ ID NO: 222 - protein, 96 aa, Homo sapiens
MGCPVSIAEMHQGHISHLLCLGCPICIYQRKPWTPTRGASMRECMPLYKFTPTSEKRPQLMLPLPEQQCEQLCRFGSTPVTWALIWFGCIPTQISS

SEQ ID NO: 223 - protein, 54 aa, Homo sapiens
MLDFTHRSGFRKKQDASAVALYTITLKDLRSCFCFISPYQLPPCCTLQNFGNKT

SEQ ID NO: 224 - protein, 68 aa, Homo sapiens [sequence omitted]

SEQ ID NO: 225 - protein, 61 aa, Homo sapiens [sequence omitted]

SEQ ID NO: 226 - protein, 112 aa, Homo sapiens [sequence omitted]

SEQ ID NO: 227 - protein, 62 aa, Homo sapiens [sequence omitted]

SEQ ID NO: 228 - protein, 77 aa, Homo sapiens [sequence omitted]

SEQ ID NO: 229 - protein, 105 aa, Homo sapiens [sequence omitted]

SEQ ID NO: 230 - protein, 71 aa, Homo sapiens
MEGDSFELCCRSAIAYAVCKVTPSLGASVPFCSFPSLDVPCCSLRSFSASSLMWLCSVFCLMPQALVPATK

SEQ ID NO: 231 - protein, 117 aa, Homo sapiens
MNMLYIALLLLPPLTMLEESPTEGCLHRTHTTWSGNSITKTLLYHTYYGCMGNRLGTCTYSQTTYSVCDPGNNQLYVCYDPKFSPGEWFEIRAVKRRSPLKPNQGPSLLLRDYFSVF

SEQ ID NO: 232 - protein, 104 aa, Homo sapiens [sequence omitted]

SEQ ID NO: 233 - protein, 50 aa, Homo sapiens
MSVIQTLWEADAGRSLEVRSSRPARPTWQNPVSTKNMKKKKLGWCAGTCL

SEQ ID NO: 234 - protein, 53 aa, Homo sapiens
MGRSRSCPSRPRRLVVSLARAVSPVAGKTGRPASSVPTTQPLRPQLEPGSRRG

SEQ ID NO: 235 - protein, 146 aa, Homo sapiens [sequence omitted]

SEQ ID NO: 236 - protein, 53 aa, Homo sapiens
MYICIYLCIYNDEKTDIFTLYTYVCVYLWTKPIFSMNCLFLLISYRICMLHNI

SEQ ID NO: 237 - protein, 97 aa, Homo sapiens [sequence omitted]

SEQ ID NO: 238 - protein, 50 aa, Homo sapiens
MNIEMEIGPFSPITNFVPQFLKSGMFNSASHLARVCNKQLVIFHPLSTQN

SEQ ID NO: 239 - protein, 86 aa, Homo sapiens
MGLLPQAGLAGLGGWGWAARPSLKAPRGAVGRAQGRSVVLLVPSCGAGSRAVHGRGRDPAPRGRSPAPARPWPRPRPGGGGAQSRP

SEQ ID NO: 240 - protein, 76 aa, Homo sapiens
MRRSRTRSLSADKPVESAILERVPEAPGFQFRLLTRQPCDLGQLNVRKLGSVFSIGLMSVRGSRKGGWPAGNLRSM

SEQ ID NO: 241 - protein, 68 aa, Homo sapiens [sequence omitted]

SEQ ID NO: 242 - protein, 66 aa, Homo sapiens [sequence omitted]

SEQ ID NO: 243 - protein, 95 aa, Homo sapiens
MALLCHIFLFTVLLPPFTLTSLPPCCCTTSSSSYQEVLWRMWLPRNIDVPSYRGFSKGDPTFTTHNHIPLHFRPYISIPVSLTSLLSMSLPESKL

SEQ ID NO: 244 - protein, 77 aa, Homo sapiens [sequence omitted]

SEQ ID NO: 245 - protein, 62 aa, Homo sapiens
MLSKNIRTCETLFVYTQTHLHIYLYLSIEIYCVCVCVCVCMYLCVCVSLSVLVHLSCYNTMP

SEQ ID NO: 246 - protein, 88 aa, Homo sapiens [sequence omitted]

SEQ ID NO: 247 - protein, 66 aa, Homo sapiens
MCLVLQFSTSFLSAAVFLFSFLSFLHKLLRLWAQLEKILKPSLALLHSNIFKKCFTGWFFVVAFLF

SEQ ID NO: 248 - protein, 103 aa, Homo sapiens
MAAPAPRPRRVPSPPARVPGERWSGVGPVCLPPAPSPNPGLLVKGRGRARARKGGGSERQRGPAERVPTVTPRSSSQGAPAPKLLARRAQSRFPDQSRGRRSQ

SEQ ID NO: 249 - protein, 84 aa, Homo sapiens [sequence omitted]

SEQ ID NO: 250 - protein, 130 aa, Homo sapiens
MAFLQFFLSKPMARAKWEKSETLKGEPIIYPKAQRTKAMWVQWICSRSPGLCPRVSENHPFWQGKKVSALWMKLSVRPPLLLTPSTAGAGRFQATGLHRPSMLTCPAHSLTNWSLPPHLLELTRASKADG

SEQ ID NO: 251 - protein, 103 aa, Homo sapiens
MGDPDRPSLEYRVSFSNLLKTGEQSSQAKLPPAPPQSQTSPWTISPAPCRRILQPQCQFYRCYHPARKDFEKSSSVLWAVGSHLHSPPRQPRRKAVSLCRAEV

SEQ ID NO: 252 - protein, 79 aa, Homo sapiens
MLFSIVGEPFYIPQQVHHFILHSGCTILVPANSAQWLQYLLLLTNTCYLLFLDNGHLSRCEAYLTPYTKINSKWIKGVA

SEQ ID NO: 253 - protein, 57 aa, Homo sapiens
MFATWHSIANYIHGAVQHASRALPSHVAETLHVPPPTPGNHYSSLCVYKFDCFRDLI

SEQ ID NO: 254 - protein, 68 aa, Homo sapiens [sequence omitted]

SEQ ID NO: 255 - protein, 80 aa, Homo sapiens [sequence omitted]

SEQ ID NO: 256 - protein, 68 aa, Homo sapiens
MENGWVWWPMPVIPALWEAEVGRSPEARSSRPAWPTWQNSISTKNTKPSMVEHACSPSYLGGCGTRIT

SEQ ID NO: 257 - protein, 73 aa, Homo sapiens [sequence omitted]

SEQ ID NO: 258 - protein, 99 aa, Homo sapiens
MCHHVLLVFKFFCRNRISLHCPGWSPSLEQSSCLSLPEWWDYRCEPTHLAFFCLPFNSVCCFLTYRGFKFCCSWITLPFVTFFIASIKLNLLFPFQIQK

SEQ ID NO: 259 - protein, 50 aa, Homo sapiens
MKSSIFFHINWPFIFLCELSVITFYLLHSNTIQNIILLQLFYFLLTFLFI

SEQ ID NO: 260 - protein, 100 aa, Homo sapiens [sequence omitted]

SEQ ID NO: 261 - protein, 60 aa, Homo sapiens [sequence omitted]

SEQ ID NO: 262 - protein, 72 aa, Homo sapiens
MMPGETHSAAPGTAADLSRCQGCASLQQNLNEYVEALITLKQKIINTDNLLTEYQKKCDDILLLAFSVEFWN

SEQ ID NO: 263 - protein, 61 aa, Homo sapiens
MAGRPQETYNHGRRGSKHILHMVAGERSAERSGEKPLIKPSDLVGTHSLSGEQHWGTAPVI

SEQ ID NO: 264 - protein, 51 aa, Homo sapiens [sequence omitted]

SEQ ID NO: 265 - protein, 112 aa, Homo sapiens [sequence omitted]

SEQ ID NO: 266 - protein, 50 aa, Homo sapiens
MQGCGRGRWDLHCDLANVDCADCLLLRASVSSSGIQSSCLRDCWIRGDGS

SEQ ID NO: 267 - protein, 106 aa, Homo sapiens
MVSFYPLESHSCLKGHHAPFFQPYLVLQMPRRYLVVWPSLLQGLSPLGHPPTSTSSWLNPTQTSKMKRKATFRKPSSIQCFPFVSSSSSPLKGLRMMSSKFDHLGF

SEQ ID NO: 268 - protein, 88 aa, Homo sapiens
MERLPHPVLYTTLQLGLSNWAFNFSLIQVSSIGQLLGNWFTHADKPSSPELTHSPSLTTEEQIRKQDLSGAMTSSTVDGKEMRGAATC

SEQ ID NO: 269 - protein, 72 aa, Homo sapiens
MYPCVLIIQHPLIGENMQCLVFCVSLLRIMASSSIHVPAKDMISFLFVATQYSMVYMYHVFFIQFVIDGHLG

SEQ ID NO: 270 - protein, 52 aa, Homo sapiens [sequence omitted]

SEQ ID NO: 271 - protein, 53 aa, Homo sapiens
MGENKGERSETAACARPPLAHSPRPAPRAPPPPPSLPRLLLTPSERPGRESGG

SEQ ID NO: 272 - protein, 110 aa, Homo sapiens
MAAAAVRPVTWTTGLGPTTPPPRLEDAQRRLGLGRGVSQREPRPSPPGRRLGAGSLAGRPRLPPESAGAPRRPCPWVHRPDPRVPRLGLLLRTTLSFLSRVTHSSSLGPF

SEQ ID NO: 273 - protein, 82 aa, Homo sapiens
MLESSKVFNQASGMISFLFCFVFFETESGYIARLECNGAISPHCNLRLPGSSDSPASAPRVAGTTGARHHAQMRKLRHREVK

SEQ ID NO: 274 - protein, 75 aa, Homo sapiens
MKPNPSKEVKSRFITVPSKFLFGFSLWSFISLPFSLCLLSTLSSLSLIHIHTHTHTHTYVLSGMSYCQGEGLPRC

SEQ ID NO: 275 - protein, 68 aa, Homo sapiens [sequence omitted]

SEQ ID NO: 276 - protein, 56 aa, Homo sapiens
MSQQSNWEPNSDQQGGIVWLIGSDRNLGRKQPHTKDNYKKVTWALKDSVKKPETPK

SEQ ID NO: 277 - protein, 56 aa, Homo sapiens
MIKKGKCPLAASTVRLELTEKELLNYYLLHSLQRKLEKWVDVIERTCVCGARQTWV

SEQ ID NO: 278 - protein, 83 aa, Homo sapiens [sequence omitted]

SEQ ID NO: 279 - protein, 55 aa, Homo sapiens
MGSALYKSITFFYLVYHMFTVPFPCLAMIRYTNSYHRGTTAYSIQYSHMLRRFVA

SEQ ID NO: 280 - protein, 74 aa, Homo sapiens [sequence omitted]

SEQ ID NO: 281 - protein, 57 aa, Homo sapiens
MGPQSFPLVFRLKATLVAGKSTGKALFQEEARNPGENPSSAPNLQDGLRWISFCTLL

SEQ ID NO: 282 - protein, 70 aa, Homo sapiens [sequence omitted]

SEQ ID NO: 283 - protein, 78 aa, Homo sapiens
MPSKTTPISSDDGLCHRPSTEHHDTHRHSSLKTWIDPGATILSSLECWRGPDQGRTPMSGSRVSPGSWGTLLGLAIAT

SEQ ID NO: 284 - protein, 73 aa, Homo sapiens
MHKRQQRQVKRRLVGLQKRPAVPMLRFSHFTDNKSSVSTDTNPGTAGPVPGMTLGQTPWRWIRKLQFYQGSCC

SEQ ID NO: 285 - protein, 82 aa, Homo sapiens
MPEEIEIPREWRSREQRRRMGSRNRPREPARCFFLSLLENSVQKPLGFRLETERNRLCVFHCRVSVLGVYSMWSVCDQFNCA

SEQ ID NO: 286 - protein, 66 aa, Homo sapiens
MEEPGTKEKDGIQEQAKRTSSMLLPLPSRKFCSEAIRLQARDREEPPLCFSLSCLSAGSVLHVVSV

SEQ ID NO: 287 - protein, 85 aa, Homo sapiens
MLTRLVSNSQPRDLPSSASQSAGIAGVSHCARPVPSFFLKKRVPSSSAFILKPPFKTLLLLSNSEGPFRLDRMSKGQSIAPNTLL

SEQ ID NO: 288 - protein, 61 aa, Homo sapiens
MVVCSEKSHLIPPPWHLSDLSCSVGGMFPPLFACSDVFVLPLFFRLDCQQNAEQPGEMKQS

SEQ ID NO: 289 - protein, 101 aa, Homo sapiens
MNTSAIHDSCFSSWPLKSSLLSLHSYSPPQLNFSFPTQEEPVCPLHPTALPDAFSPKTGMASGPHLQVSGPHLQTQHMAFGSSHFQESWKGWVEQAEERKM

SEQ ID NO: 290 - protein, 98 aa, Homo sapiens
MPPASHCTSGCLFPQDWNGLRPPSPGEWPSPTDSTYGLWLFPLPRVLEGMGRASRGKEDVSSQNAPHLFLLSGLLLTGIGGLAAQHGPPPWETPSLLS

SEQ ID NO: 291 - protein, 61 aa, Homo sapiens [sequence omitted]

SEQ ID NO: 292 - protein, 83 aa, Homo sapiens
MFRAAAKLGGNIGKIFQEHIGLGMATCIWLSTFLHICGEKKSFFFKIRSHTFILRLLQRCNKVFCFYFFKMGLTVFLPTDCHS

SEQ ID NO: 293 - protein, 64 aa, Homo sapiens [sequence omitted]

SEQ ID NO: 294 - protein, 153 aa, Homo sapiens
MSLKSATKWGRRCNYYYQHLESIMNLLEYFLALTSFILRCSYWIFPSANNMEVPIQGQIIPGFIWSCLKVKSLEFLMIPFLYGLQFDRWEFSTLKKTLLLSGNPCPPLTSTQNCFPHSLTARVVKNWDVLLRWAVECHYPQVTTDVLTPSMFR

SEQ ID NO: 295 - protein, 66 aa, Homo sapiens
MLRNHYKCYCLESHFVSLWKPHVYLVCNSMGSQAEGIFLVLDETFDLDFWVKFWRPEGQNQKYGQA

SEQ ID NO: 296 - protein, 66 aa, Homo sapiens [sequence omitted]

SEQ ID NO: 297 - protein, 136 aa, Homo sapiens [sequence omitted]

SEQ ID NO: 298 - protein, 67 aa, Homo sapiens
MAAPPLANPLHLVVTPMRSRSLFCLCLGWPVWNSLLGSQCTSASGQLPDPGLHESREEAQRRSMMAG

SEQ ID NO: 299 - protein, 51 aa, Homo sapiens
MSREMLKNNSWRKSLTSRTDVAGKRKALQSRRLCVWLTIESSLSPVRSPFF

SEQ ID NO: 300 - protein, 50 aa, Homo sapiens
MKHFHHSEKFPSICLQSILPPALSIIELLICFLSLQGSSHFPEFYINRIV

SEQ ID NO: 301 - protein, 63 aa, Homo sapiens
MPSQGSRRPGGASGSRGWSSSARQEAGTRHPAEGGGEGPARILPSPCACVSCGFCARTVSWAV

SEQ ID NO: 302 - protein, 50 aa, Homo sapiens
MSTTQLGGYWPGAGEKGEGRARGGRTPEGRGDCRGALGARWASRPPARLG

SEQ ID NO: 303 - protein, 55 aa, Homo sapiens
MQDKVLFTLSCSLLKQKEGVTFVSARCTTWGWGMGGASTSLVTLSGVSFGVSKST

SEQ ID NO: 304 - protein, 53 aa, Homo sapiens
MKVIFFVCVYRWLSNLAFLWEIMDNQWRPLFSHLTLPLDCSLQIHTCLYHSDV

SEQ ID NO: 305 - protein, 53 aa, Homo sapiens
MAMSSCCHAPFRNTALWAELIKPERLRQTEILLEHTEEQCEMMCHSCEWLEKS

SEQ ID NO: 306 - protein, 76 aa, Homo sapiens
MVIVLEGTWWRQACCSDLFQPWRCQNVAVLTWWPFVSQEQSDKMARMGVAHLNLTNETAPGETHFIPDTSLAERHR

SEQ ID NO: 307 - protein, 65 aa, Homo sapiens [sequence omitted]

SEQ ID NO: 308 - protein, 77 aa, Homo sapiens
MVQGSFLTSYDPHFCYLKTIKCNEYSVQKSSGLLSEISWVCPLSHVFRSSLYYSLLLPALFLQSGVLSWKKALVDQF

SEQ ID NO: 309 - protein, 51 aa, Homo sapiens
MNTVFKNQVACFLRFLGCVLSHMYLDLLFIILSYYQHFFFKVECSLGKRPW

SEQ ID NO: 310 - protein, 99 aa, Homo sapiens
MGRGEERCGKGSSLARKGMISPEFTGLPLSPAPSIRVSVLQWGLSPSGTEESLDSAFTTPDPQSTTCALSLRRNSLFHFSNQSSVHVFTIMLQHHWFQL

SEQ ID NO: 311 - protein, 92 aa, Homo sapiens [sequence omitted]

SEQ ID NO: 312 - protein, 363 aa, Homo sapiens
MQVSSWPSVSCRIPRSEHRKYKVLYNSQLLFRSRLYGDLEAILYHVHLFQPTELLLQQAVFFLRDTERRRVFQALARIHDICYNSTTLWDVTVRDLLPSSAMIKDLSQEFGMPLSQEELTDEKLFALPPQPAPNLEDYHSRNSTLTLEIHAHQEKYLQWRSAMLMKNKDKKHSFIQKNITEAYQVSKKPPKSVAKVIKISAPANKAVYNYSTQTMNSTELAKKELYQEIAKEPRKRFTYSQDYLSAMVEPLDLKEEEKKAQKKSRQAWLTARGFQVTGLQSDTESSFQDLKLPPIKELNEEWKENSLFANVLEPVLDRDRWSWDRHHVDFDLYKKPPPFLELLPSPAPKPVTVRKKKGNSPIS

SEQ ID NO: 313 - protein, 86 aa, Homo sapiens [sequence omitted]

SEQ ID NO: 314 - protein, 82 aa, Homo sapiens [sequence omitted]

SEQ ID NO: 315 - protein, 77 aa, Homo sapiens
MPEPPIHSMGSCAARASPTSTTPCSTAPSPIDHPRAEEYERTAPDWQAAPPAAPVRDPLGEASWAPESGGDIESLYV

SEQ ID NO: 316 - protein, 94 aa, Homo sapiens
MYICTHKYAYVCTHMCAYAYVCTHMCAYAYVRTRAHMRMCTHVHMCMRAYVCICKYACTHMYIYVRMHVHMFVCIHTKNPYIHSKMKRPMSDSS

SEQ ID NO: 317 - protein, 88 aa, Homo sapiens
MHICIYVHTNMHMCAHICAHMRMCAHICAHMRMCAHVRICVCAHMCTCVCVHMCAYVSMHVHICTYMYVCMYICLCVYTQKTHTYIQK

SEQ ID NO: 318 - protein, 73 aa, Homo sapiens
MRLGGGSREKLACGRAGRAARTGRGEGGRQAEEEAAGRRRRRPGRTRGIPRAAGGLSRAGTRVLRYPSSHRRQ

SEQ ID NO: 319 - protein, 67 aa, Homo sapiens
MHRGRAPPSDSSRGGRAPSVSFALEPPARVGFSGIPTVRDYSTSRLITFFSSSSAKASLKKNITNFF

SEQ ID NO: 320 - protein, 64 aa, Homo sapiens [sequence omitted]

SEQ ID NO: 321 - protein, 62 aa, Homo sapiens
MIVIWKLDPPWSIISFYYYFFLFSCSSLWQIFSTLFFNPSFEFFTSYIVFSISKSLFCLFVL

SEQ ID NO: 322 - protein, 73 aa, Homo sapiens [sequence omitted]

SEQ ID NO: 323 - protein, 67 aa, Homo sapiens [sequence omitted]

SEQ ID NO: 324 - protein, 53 aa, Homo sapiens
MDTYDMEQFLNCLSHRGNCILYTHKLGFSERARMEHNFLTSILVSFLLVQNGQ

SEQ ID NO: 325 - protein, 66 aa, Homo sapiens [sequence omitted]

SEQ ID NO: 326 - protein, 57 aa, Homo sapiens [sequence omitted]

SEQ ID NO: 327 - protein, 108 aa, Homo sapiens [sequence omitted]

SEQ ID NO: 328 - protein, 61 aa, Homo sapiens
MVCKWQNWGEKPSGCLLSLPSIVPQRWPLLSSPDTPWVQVIATGCQTLQVLCWTPGSQVTG

SEQ ID NO: 329 - protein, 92 aa, Homo sapiens
MTKCIISISLCLYDCLIQAFLPMGWIEKYSFFFFFELESSSVAQAGVQWCNLSAHCKFRLPGSRHSPASASRVAGTTGTRHHARLTLFLYFK

SEQ ID NO: 330 - protein, 78 aa, Homo sapiens [sequence omitted]

SEQ ID NO: 331 - protein, 91 aa, Homo sapiens [sequence omitted]

SEQ ID NO: 332 - protein, 52 aa, Homo sapiens [sequence omitted]

SEQ ID NO: 333 - protein, 69 aa, Homo sapiens
MSPYECQTGISNLMCPPKILIFTPKPAPLSVLCILVNVTGTHSVVQAKILRAVFDLSHPFTFKSFSKAC

SEQ ID NO: 334 - protein, 77 aa, Homo sapiens
MHPWPDVLAHTCIPSALGSQRQADHLSPGFRDPAWATWQNPISTKKISCMWWCALLVLATWEAEVGGSLEPGSLNLK

SEQ ID NO: 335 - protein, 57 aa, Homo sapiens [sequence omitted]

SEQ ID NO: 336 - protein, 125 aa, Homo sapiens
MRLRWIQGAGAQIHQDQGRFLRARCPSGPEENPFSACLRWRTQWPPPPALRPPQGPDPELDDRTPSHLSPQNPCQPAGGRGARPPASGKHSVPAGACAGATLPGPLWLRLRALALARGAATGSAF

SEQ ID NO: 337 - protein, 63 aa, Homo sapiens
MSFTTLPSENAGVQWCDLGSLQPRTPWFKCFSSSASRVAGITGTCHHTELIYCICSRDRVSPC

SEQ ID NO: 338 - protein, 95 aa, Homo sapiens
MKSHKSIFSRDCEVHCTQPALFYISFFYFNHLGTGQKSWSEGHICIALMMGAENKQEELGNENCFTSYILTRKATQMGIITGIVQWQKDTLKNRS

SEQ ID NO: 339 - protein, 90 aa, Homo sapiens [sequence omitted]

SEQ ID NO: 340 - protein, 60 aa, Homo sapiens
MSTSLAVGAGAVVTAESLEPSRPSVEEGQGKGTAAEGVCVCVCVCVCVCACVRACVCFVF

SEQ ID NO: 341 - protein, 58 aa, Homo sapiens
MLWDLTVLDTCMAVCCGLCLACGSLLLWPPPPLMYKPRLCSASLGPRQAAVHSLTREV

SEQ ID NO: 342 - protein, 50 aa, Homo sapiens [sequence omitted]

SEQ ID NO: 343 - protein, 70 aa, Homo sapiens [sequence omitted]

SEQ ID NO: 344 - protein, 54 aa, Homo sapiens
MRFQQFLFAFFIFIMSLLLISGQRPVNLTMRRKLRKHNCLQRRCMPLHSRVPFP

SEQ ID NO: 345 - protein, 84 aa, Homo sapiens
MSGRVPLAEKALSEGYARLRYRDTSLLIWQQQQQKLESVPPGTYLSRSRSMWYSQYGNEAILVRDKNKLEVSRDTGQSKFCTIM

SEQ ID NO: 346 - protein, 66 aa, Homo sapiens
MRAGRATGAPSSLRLGPPCPFLRALDPSLQAQWSRPWWQNHPPGCDASSLSIPHSCTRKSCGRAPL

SEQ ID NO: 347 - protein, 106 aa, Homo sapiens
MALSLASSPTPCFFILRKMELSSFNSVSPFQVKLGNHKFTPITYSFLSTIRILLTLSTLLCLFLLLLYKYLLSFIVSLFPFSDSPYPTKCSRLPQTTAHLMQPTTV

SEQ ID NO: 348 - protein, 93 aa, Homo sapiens [sequence omitted]

SEQ ID NO: 349 - protein, 63 aa, Homo sapiens [sequence omitted]

SEQ ID NO: 350 - protein, 62 aa, Homo sapiens
MHSDGTLLYFHISHLSNSPSHLSYSFLITNITILAFIMAGNWQTAMRDYHISKPITTTFGVF

SEQ ID NO: 351 - protein, 88 aa, Homo sapiens [sequence omitted]

SEQ ID NO: 352 - protein, 66 aa, Homo sapiens
MRSFCDSSSLRRTTSETKSSSAQGWGPADFKGRTGAWLVGEFPNVDLNCSVFREHNQHLSLGDVFV

SEQ ID NO: 353 - protein, 57 aa, Homo sapiens
MCQNMCKLSTGSPFLPMELLANEGCGSAAGLMSLKLPHTSTPGLSLSPHSSISGSQL

SEQ ID NO: 354 - protein, 131 aa, Homo sapiens
MPQPPSALPSLAKDTKEETEVFLPLPPMNWEEDKRYTTVMGPCLRQAALEGELLACPVMQDQQGNWVHEPITLNTSKEIRKSIRENGATSPFTRGLIEAIADNYHMTPWDWSVLAKTTLGGESIPPLEGRI

SEQ ID NO: 355 - protein, 125 aa, Homo sapiens [sequence omitted]

SEQ ID NO: 356 - protein, 69 aa, Homo sapiens
MSTICGFHSYLSMVNIDHHHYLLSCYHIPATPCMLFIIFSTLSIFAICYVISQTRKLNAEMFYFLPNIT

SEQ ID NO: 357 - protein, 63 aa, Homo sapiens [sequence omitted]

SEQ ID NO: 358 - protein, 52 aa, Homo sapiens
MHCQGHQKDDSPITKGNQVADKAAKQATQETYLLGTLILHLNLSEFKLHYTE

SEQ ID NO: 359 - protein, 110 aa, Homo sapiens
MPPRFKPFSCLSLLSGWDYRRALPRPANFCIFSRDRVSPCWSGWSRTPDLRQSARLGLPKCWDYRCESSRPASIFRNFKGRKNTSLTLPFFSVLATWCPCIQIFLLILMQ

SEQ ID NO: 360 - protein, 68 aa, Homo sapiens [sequence omitted]

SEQ ID NO: 361 - protein, 101 aa, Homo sapiens [sequence omitted]

SEQ ID NO: 362 - protein, 50 aa, Homo sapiens
METGTGCWGLGGAEIEEPVAVPCSGKRTRCVASQNDEFHFSRRLRGILPK

SEQ ID NO: 363 - protein, 70 aa, Homo sapiens [sequence omitted]

SEQ ID NO: 364 - protein, 58 aa, Homo sapiens [sequence omitted]

SEQ ID NO: 365 - protein, 80 aa, Homo sapiens
MANKLMKTCLTSLIIREMQIKAKMRYHVTLIRCYFLKITQQQKIRNVGEDVKKLKPLWTVVGDVKWRSCWGKQYGSSSQN

SEQ ID NO: 366 - protein, 54 aa, Homo sapiens
MFTAEPKGGSNPNVHQWMNTGNKSCVWRPLRFTQLSTRVRAQPSRLFPMLHLQS

SEQ ID NO: 367 - protein, 73 aa, Homo sapiens
MLGLSLQPQAHLSSRETLHFACPTNISNMSPNELIHSEAAWLNEWCHCLPRSLERNVGASVHDHAQCSPPTLV

SEQ ID NO: 368 - protein, 70 aa, Homo sapiens
MTMTMPKAPCCPWPKLWMSLSETAYSQLVSLCSPCPPLTHCPHRRSNRKPDPILFKLLRVSHCPCWNPES

SEQ ID NO: 369 - protein, 95 aa, Homo sapiens
MACKSRFTSSWKEAGQKFRVVGPWPGWMCYNMNHKPPSLILADRKLELWRKVSHQALRPNKYSHSLQSYRTFCTPFIRLPKDAWNVLAGATISTC

SEQ ID NO: 370 - protein, 69 aa, Homo sapiens
MITAHCSLTLQDSSNPPHSASQELRTGSIDYKESRMSADQATRRRFPDFHYARGLHNCVCEVMKNVLAL

SEQ ID NO: 371 - protein, 72 aa, Homo sapiens
MLSLPQAPTRNRPQCVMFPALCPGVLFVQFPPMSENMKYLVFCPCDSLLRMMVSSFIHVPAKDMNSSFFMAA

SEQ ID NO: 372 - protein, 55 aa, Homo sapiens
MSFDLGKNIVGHPESTCHQPRRACYKHTRHAKDGNIGPNLATTNPFPHAYFSNYK

SEQ ID NO: 373 - protein, 55 aa, Homo sapiens [sequence omitted]

SEQ ID NO: 374 - protein, 71 aa, Homo sapiens
MFGPGAVAHACNPSTLGGRGSHLSTTHEVSGAPPSAQEERHMTQAYANQHFTFLMVGSKLCIWVHLFPPET

SEQ ID NO: 375 - DNA, 2966 bp, Homo sapiens
aaaaacagcaaaaaagtggataatctatgacatatccatttgaagctaagtataatagaatgttgcttttagcctccatacttgggctgtcatatgggttttgtacttttcccttaaggttctgcatccattgctgaaaattttaatattttgcattttgaggtgggttgggtttcccaggatgtaggaatgttagtgctaaaatctggacaatgccatacaatcctcaccctaagactggtagatagctttagatataattggcactcaacatacctaagatgtaattttctttcccgtatctctgcttaaaatatattttctgtcttgccatagtgcactaagttgatcaaaccagaaacatgggaatcattatgttaatgtctctttctctaaaccccttttccagaaattatgaagtgctatgaaatttttaaaattcttctttatttttcattttattttctgaacatggactgccttcccatggttccaaaaattacaaatataaataggtttacagaaaaaagcctattttctttccaccgtgtctccagtctttccagtttttgcccaagctaaagcagtaattactattgtttgttttatatgcatctttatggacatacatgcaaatacagacttttaaaatgttgtcccactttgcaataatcactgctatttataaaagataagaatgggcactatatagcagtaagagatctttagcaattctaaattttatctgggcaaatatcgccagcctattgtcctaaagttaggaaaattaccttgaattaggtcctggttctgctctttggatatggcttcccagtttttgtgcaccatggatcttggctttattctcttgaaggatatttcatttccattcttcttcttcttcttcttcttttttattttattttgttttttttggagacagacttctgctctgttgcccaggctggcatgcagtagcacaatctccacttactgcaacttccacctgccaggagaatcacttgaacccaggaggtggaggttgcaatgagctgagattgcaccattgcactccagcctgggtgacagagtaagaccccatctcaaaaaaaaaaaaaaaaaaaaaaaagaaagtcaagaaacaacctgctggtaaggctgtggataaataggaacacatttacactgttggtgggaatgtaaattagttcaacgattgtggaagacactgtggtgattcctcaaagatctagaaccagaaataccatttaacccagcaattccattatagggtatataatcaaaagaatattaaatcattctattataaagatacatgcatgtgcatgttcattgcagcactattcacaacagcaaagacatagactcaacccaaatgcccatcaatgatgcattggatgaagaaaatatggtatatttacaccatggaatactatgcagccgtaaaagggaacaagataatgttctttgcaaggacgtgggtggagctggaagccattatcctcagcaaactaacacaggaacagaaaaccaaacaccacatgttctcacttataagtgggagcccaacaatgagaacacatagacacagggaggggaacaacacacacttgggtctgtcatgggggtggagggagggagagcattaggataaatggctaatacatacagggcataatacctaggtgatgggttggtaggtgcagcaaaccactatggcacatgtttacctatgtaaggagcctgtatgtcctgcacatgtatgctagaacttaaaaataaattacattttttttttttttaaaaagaggccactagaaggtggcccctagaagctagaaagtcaagaaaaccaattctcatcctggatgttctagaagggaactcagctcttctggtacctttatttttagccttgaccccttggttttaatcaagtgaaatccatttcaaacctctggaccccagaactgtaagggaataaattcatgttgttttaagccactacatttgtggtgatttgttacagtagcaccaggagactaatacaatgagcaaagtccagttattatttggaaaagatgccagcattgttttgtgttttcggtagtttcataaaatgttacgagaatctagaagattatatgaaactcaatttttgataccagagccactagaaaccagggtgagcattttttcattattgagatacctcttgatatagtgcaaaaagcatgattgttaacaaggtttgactaaatcaggtctacaatgcaataagccaacacatctattaccaacattttcttatttgctttcctacatggttgtaacctctgaattggtaaatgtaattttattcatttttattaaaaaatagtcaagttgagtgtagcatgcatatttgttcttaaggtatacccataaactacttcattggctattttcaaaaatattggtgcctttgatggaaataataataataaacaacctttggctaatttagaaaacttgcctgtttcagccaggacttataacatttaatctccctatatgctttcacatcccctgttctaccatgcctaatcccaattttctctctgcctattgtgtaggatgctttgttgtggttacttgcctctctaaaccagttacatgtatctttccatatgtcattaaaataatacggttgcttgggtgatgctagcatctgttttgtaatcattctcattttcatcatttgaactcttagaaaacatagccacaatattcagaatgttaaactagcattacctcaagacaaagcacatagtaaatccacaggcaatgtctaaaacgattgttaatgcttatgattagaatgcacttagttgaatacataaatcacatcactcacaatgatgcaagaatggattttccatttctgtgaatggcagattatggtt

SEQ ID NO: 376 - DNA, 2706 bp, Homo sapiens
tcagaagcaaatcgatcagatgagctgcaactgtggctctgccacagaccatgagctaatgcaggctgtacccagtgagccagtgccgcgggcactggagccggcagtgggagagggtgagccgacatagctaatgcctaacgccaggagggagagacaaaggggcctcttctttaacactgtgaatagccccagagtgaatacagccttcgacagctgtggtttcattctcatcaccgctgggcgagttgggggtgctaattgtgaagctggagagctgttaaaatcagaccagatttgttaaaggagaggagtaagatgatacttcagaccagtatctgcagtggcacaggcacaaaagccctatctcaggagaaggaatgaaaggaaggcaaaaaggccgtgagaaaatgaaagaagctggttgattgacccctcctctgtgtcgcctcagcaacttgtccatatcattgttacagcacttaccacacagttgtaagacggtactgcaagcttactttccaacgtggctgcatcgtttcacattcccaccagcagtatgaggtttccaatttctctgcatcctcatcaaaacttgttattatctgtcttttttattttagacgttctagaaggtgtggagtggtatctcattgtggtattgatttgcatttccttaaggactaatgcttttgaacattttttcatgtgcttattggccatttgtatatcttctctggagaaatgtttattcaaatcctttgtccattttttaattggattgtctttttattgttgcatggtataagttctctatatattctggacacaagtctcttattagttataccatttgcaaatattttctcccattctgtgcgttgtcttttaacctttctaatgtgtccattgaaacacaaaagtttttaattttcacaaagttcaatttatctacttttttcttttgtcacttatttgtatcatatctgagaaaccattacctaatccaaggtcaggaaggattactcctatgttctcttttgagagttttattgtttttactcttatatttaagttgaatatccattttgagttagttaatttttgtatatagtatgacataggatcgagaggatcttgttaatcactttttcctgatatacatcagcttcaaatgtgaagctataaatctccctgtaggtttcctgaaagccctcattatataaagcaacttctgtgacaaatttgacttgattaattaattaattagagacacagtcttgctctgtcccccagactggagtgcagtggtgccatcttggatcacagcaaccttcaacctgggggcggaggttgcagtgagctgagattgcgccacttcacttcagcctgagtgacagagtgaaattccatcttaaaagaaaaaaaaaaaaaaaaggaaagaaagcagcaaagagaattttttggcaagattatgagccaattttaagagcttttattacatttcaaatgtgattttaaattctcaaagttggacagatagcagggaaagaatggtcacagacttttccctaatattttaagttgcttcaaaccagttattaaatcttatatgtattttttaaagacttattccacaggggtttgattaatttacacagacttcttccaaaactgtatctaaaccacattgttgatatctaagaaactctatgagcttcttcaaaagccctcttaaaaatacaacaaaatcatgttagcaactccagattccaagttgaaatcctcccgctttagtataagaaccatcagttttgaatgagtctggatacgaaaacctattttcccattttgaggagaaaatccctttttagagactctttcttgaatggatatttgttataacatatttgggcttagggctaaaacctttggtttgtaagctgaggttttcatttgcacttaccatcaacagcgtataagttgaaaagaatctcaggtctggttgctgtcctgagctactgcaaagaagaaaccactgagaataagagcatttcaattattgtacatcacatttgctatttgctgccgttattcttctattaaaggaggataatcaactgatatgatattgtagatctcagtgcctagtgcattgtaggtcctcaatatatgttgattcttcttttctttacatatttttgaggtacagaaagaagttcttaaaatggtttttaactgtaatcattttggaaaattagctatgaagatggaaatttgtgaacaaaggagaacttgcaaatgccattttactgagcatgttactgaatggttatataaggagtaaatattctctcagccttagtaagtaaatatgaaccaataagaacacaggtgaggactcttttcctaaccagaatagatggctaaggtttacagggcaataaattagactatttgtatccaaagcaatattgttgtcaccaccaattgtaataagcatttttataatttatgcctttaatgtgtttattatgaaaaagcttctaagtatattgcacaagtggtaatctgctccagaactggaaatttgggagacttggtttttggactcagcctattcactattatttggttgataggtttattattattattattattattttttaaaggcctgagacagtagagactttctaataaaat

SEQ ID NO: 377 - DNA, 1764 bp, Homo sapiens
catatgggaggccacgtaatcacttattacagtggttacataatacactggctcactgcagactctcttgttttttgatacagtttcgtgctggcttcatttgccaattgtgttgtttagttcggaagtaagagggtcttgagattgaggggtagggagggctacactgactgatccgtggcttaagacaggagattatctctgtactccagtggcatctccttagccaagatgtgaaattaaaatcatagttcgcctcatttaaaaattctaataaagcactcaaactttgaaaagcttttacttttccctcctactaaaagaaatgtatgtacctcatagccctgtgtcatttagtgttcagcacttttgggaacatcagttggtgaactttaaattttgctgtctactcactgggcacggtggctcacacctgtaatcccagcactttgggaggctgaggcaggtggatcacctgaggtcaggagtttgagaccagcctgaccaacatggtgaaaccccgtctctactaaaaatgcagaaattaggtgggcgcctgtaatcccagctacttgggaggctgaggcgagataatcgcttgaacctgggaggcagaggttgcagtgagccgagattgcaccactgtcgtccagcctgggtgataagagtgaaactccatctcaaaaaaaaaaaaaaaaaaatttttttttttttttgctatatactcttagtttacatcctctcccccaatcttcacacacagagccaaagagcagttatattcttgggataccatgatacctctgggaaaggaattatattcccaaggagaggtcccattggataagaacaataccatgcgacttgagtctgaatgccaacttactattcacacatccttcttaaaaacgaacacactgtttcttttcctgacctagagcattctaaagttctgttcaaataaataaagggcaaaataaagtagatttaaccaagtgccaggtggaattcaaaaaacacattcttccagataaattctacctttatggtatggatttgaaagtactttgcaggaaaacagtcagtactctttaaaaagggactgcagggctgggattacaggcgtgatccaccacacctggcccttgcaatcttctactttaaggtttgcagagataaaccaataaatccacaccgtacatctgcaatatgaattcaagaaaggaaatagtaccttcaatacttaaaaatagtcttccacaaaaaatactttatttctgatctatacaaattttcagaaggttattttctttatcattgctaaactgatgacttaccatgggatggggtccagtcccatgaccttggggtacaattgtaaacctagagttttatcaactttggtgaacagttttggcataatagtcaatttctacttctggaagtcatctcattccactgttggtattatataattcaaggagaatatgataaaacactgccctcttgtggtgcattgaaagaagagatgagaaatgatgaaaaggttgcctgaaaaatgggagacagcctcttacttgccaagaaaatgaaaggattggaccgagctggaaaacctcctttaccagatgctgactggcactggtggtttttgctctcgacagtatccacaatagctgacggctgggtgtttcagtttgaaaatattttgttgccttcatcttcactgcaattttgtgtaaatttctcaaagatctgaattaa

SEQ ID NO: 378 - DNA, 1673 bp, Homo sapiens
gtcgccggctgggccgcgccggtggccaggagggagagacaaggaggaggaggaggaggaggtgacactgatgataaaaatccatccaagaatttatatgtgacatctgcagcaggttcaaatctctcagctatgagacaaactggatacttcctaaggattgccatctattatgcacagagagccaaaatccgttactgaaattgtctgggtgagccgcctgctgggtccagtgatgcactggtagacaaaggagttgaaaaagaagtgtggcatattctttggatgactggcagagtgtgacaccagtatggcaccaagccctactatgattccaaggacttcaggactaaaaatatatatatatataaatgaaaaaaaattttggtggtattggttggtgtggttgaagtaaatttgagctttacttggggaatgtgtattatattatcattccataatgtaattcctttggtaatcaagctttaaagatcttatatctatgaaactcatatagttatcaccttttagtattgggttatctcactgtaagggaaatacagtaaagaaaaacatctgttgactaaaagggttaatttcaggactatgattttcatgtggaagtgtttgcctggaggattcttgaaattttgccttcatctagagtatttaaaatgtttctatttgcaaaaagctattatactcagaattttctaggaaaacatccttttcagtggactcttcaatagtaaagagtcacactgtatggtgttagtgtccatagaacagttgaattcataaattaatatttttagacctatcaactaaattaaattcacttaaaatgatttggcaagcaggtatgaactgaaaataaacctatgtatttttgttttttagaagtattgaatcagaagaaattggaacatcaaagtattctctgaaccaaattgatgttttatttggttgagagtccttacttttggtatactatttataccagtaatagagtatttgaaagcagtgctgggaaatgaggtgcatggattctctcaatgttgaatcccatttttttccctcagtgaatcttcaacatttttaaatcttagcattcaacaaaaactctacagcctagatataaaccatagattactttttatatagcatatacagttcatcaacaaacagtaaagtttccttcatgacttaccttgaaatctttattcaaaggatatactattcaatttttcagtgtgccactgccaccactgcatgtttttggtcagagtgcagtgcaactaacatcgccatcctgctaggaaaaagatctgcaggttgctggatagtggaagttgcacacatctaatttacaagtcagattagagatcagaatcttacaatagctttatgtggaattgatttcatttcatggtttttaagctgttttttcattgaagcaatgcctgtaactgaaagacaggtgtgttgtagcatttgaataccttaaactcttttgaatctggaatgaaagggatgttctggattctaaaactcagagattttgaattaaatgccatcattcatttattgtgccataattgaactaccttgagaactgaatttggaattactgtgattaatatttggaaagctttgccattttcaaatacaag

SEQ ID NO: 379 - DNA, 1987 bp, Homo sapiens
acttttttcaccatcactcacacacacagagacatactgacacacactgaatattttctccagcatcattgttaggcttttaaattaagcagtgcatataaggtacctgctcatcgttattattgaggagaagatagctgatcatcattatggaaggctctgtagggcatcgtcaaacctatcctgatcttctctttgcttcttcctatagagaaaactttaattttctatatctatctgagagcaacctggtagcattttgtaatattgggttgagactctcttagtcttagatttgtagaactttataatcttggttctactttgttctatatgcaagtacattttgagtgtaacaaagcccacaaatcttttcctgattttgtttacatgaccttgaatttattgagtgatgagagctctgtcagaatcagatttcagagctccttggcccaagccaggaagtgggcatgcatgactgaccaagcccagagtggcaggaggccaacctgctgtgtcttatcttgctttcccatagctcaagaggacaagagcagtgttctaggagaagccaaattattttcctaacaagaaccataaagcattagaagtaatttcagaaaaatgtaagatatgtattcctataattctgacacttttaagagatatgactgtaaataaaacacattttacaaagggtgcatattaccagcatgctataaaatgcttctagaacttattttaataacatctcccccttcagacatatagcacttttactcttgagaccactttcacatccatccgtttatgctatctcacaacatagagagggaggacagcctggtcacgctcttgcacttgactgaggctctgaagagtatgatacgcacagcctctgtgccaggactgcagtggggggaattcagatttgtctttattccaggtcatgttgatgattaagaagacctgcccaacactgtccatgcctcagggacatgggctgatttttattcttctgatttttatgatgctacttctagcacctgtcgtagcacaaaatccatgatcagtaaaattccatgaaatgaatcaacttggcttaactatcacttagaacaaacttttttttttttttttttgagatggagtctcactctgttgcccaggctggagtgcagtggtgtgatcttggctcagcgcaagctctgcctcctgggttcacaccattctcccacctcagcctcctgagtagccgggaccacaggcgcccgccactacacccagctaattttgtttttgtatttttagtggagacgggttttcactgtgttagccaggatggtctcaatctcctggcctcgtgatctgcctgcctcggcttcccaaagtgctgggattataggcatgagccactgcgccaggccaagagcaaacattttaaaccactctatacaatattcatcctatataagcctcaaataattttacagtcctgtgtgagggtatccttgcaaaatgtgggtatgtgaaactaaaatcatggtaagctatcctgaagggaactgatgggcatatattataatttgatgtagttcttggattaaaccttcactttcaagtttgatttctgagcaaatgtcttcccccaacaaatgcatagttgtttcaacctcctcccctttatctctagggtatgtttcgaacatctcttttaaaacgttaatattctgagattgtagttgtcttattacagttttcagtataaatgtcattgcccttgtcactcttattcaattgagagctcctaaaagtcagagacagagtcctaatggatgaaggctcaataaatgttttttgggtaaaatggaacagaagggattacaatggaagaaatgaaatgaattagaataatctaagtgcaaaatgaatatccagataagctacaaagaagagagattcaaaggaggtgacaatgatgtaaact

SEQ ID NO: 380 - DNA, 2397 bp, Homo sapiens
cgtggacgccggggctcgcagcgtggcggccgcaggagctgagggagtcggccgcgcttgcgcggggagtgtcttttcgtctccgggcggcggcagcagcgcgtgtgtccggaacttttctgggactaagtcctgcacccacgagtagaaaacggctcctgggagcttggggctccaagggcaagtcaggggacgctggggatggaggggcgccctcgacgccctcggccgcggtccctcgccggcccccctgctctgtggctctgccctcggcggtctgcactctggcgcgcgcagctctgagtgcctcttccctgcgtaccgggcggcgctccccagatacggcgctgctcggcgtcgggagtccccgtcaccactggggggacgggagcgcagggtcccgccccgaaggacagggccatgccggcccctggaggcgagggcactaggccgggtggcggcgcagccagcatagcggccccccatcccggcacaccgcgtcctcccgtctgagcgggaggggtagatatgggtcctgcgttggcgcggggggacccctcgtttcgctctggcctaagcagcccctggggtcccgcgcgccgcaagagcgccagacttcctaacctgggccataccctgtccgctccacttcccagtccaagaaggaacagccgaagcccaggacgccaaggacctggctcgaagtctccagaattcccgccagagctgggctgggctcgcggaccgctcatgcgccgtctcccaggagcctccccccagcccagtttaatagccggccctgaccagtccaggggagagagaccgtgcagcctcccagcagaccgtgacctctgagctccaccttccccttccttccctaagatgaggatccctgagttggggcagagaaagcaggcagggagaaaaatggggacctgcggcgctgggggcccagctggtggagatctccagctaggagatccccagcacagcacttgccctgccctctgtcttgttcagggatgtaacctgcctcctcctgcttctccgcggcacctcagggcaccgcctggggagggtcttgtagtgcacactcagcatctctgttccctggagcgcatggggtgtgaaacgcctgatgccagccagctccccagtcttgagaggctggttgaatggcacatctccctaggaggctccctccccagggtcccctctgcccctgctgtccatgcagtcggtcctcacccgtcaggaaaacgtagcctgcttgcctgggtggttttccccatcaggaactgccaggtactgggcctggatgggcttgaattccccatctcagtgggagaagggggcattgtttagtgcagggcactttgaagccgtctgtaagtccctgtgtccacaccggtcagtgggcactgcagacatcaaatgcagacagagtttaagcacaactaggcctgaacctaggcctgtgctgccaacagacatgaaggggccccttttccacccatcccccaggaggcaagagggggatacagaaggatagtccttgcctccccacccagggttggacctgagctggagtggacagttggtcagggtcagaaagaactggactttttatggcccagctgtaggactttttatggctcagctgtaggactttttatggcccagctgtaggactgtatgtcctagtcctggtctggacattggttctgagcaccagatgaccctgcccgatgctggctagggagggtgaccccggagagggtgcaagaaacagtgctcctctcgttaatgctgtggggtcccaggtgtcccggcaaggccaggcaggaggtggtgactgccccatctgtgctagtgcctgtttacaggactttgaatgacactgaaagaggagggaagccctccccgcagtctccaccctgcccaaagtgctgggtgtgaggaaatgtggaacataccctcgcctgcagttgggatgacccaccatccctggaccagaacaaggaaagcctggcagcggagagccaacagcaggactgacacaggcaggtgctgtctgcgatgctcgtgcagcccaggcagggtgggatcttagataaattttcaacaattaaaatagctgtgatcaggcgcagtaactcacacctgtaatcccagcactttgggaggccgaggcgggctgatcacggggtcaggagatcgagaccagcttggccaacatggtgaaaccctgtctactaaaaatacaaaaattagctgggcattatggtgcatgcctgtaatcccagctactcgggaggctgaggcaggagaactgcttgaaccagggagtcagaggttacagtgagccaagattgtgccactgcacttcagcctggt

SEQ ID NO: 381 - DNA, 1921 bp, Homo sapiens
aagcgcggcccctgtaaagcgcggcctgattggccctttgaaccgggcgtgggattggccgtcgcctgcccaccccgcggctgcagcgttccctggggcctagccaaccgcgggcagggctgggcaaggcgggaggagcgcggacccaagatcggctacgctgcgtctgctcaggctgcgagttcccggctctggggactcaccttgcggagcttacccaggcggactctccgcagcccctgatgggtgtgtgtgtcagaccgtttactaaacaccaggactgttgtaggcgactgtaagaaataagaatattgcatggttacaattcttgaatgcttaccttgtgtcttgttccttactctacagaaattttctaatcccgtggcgaacccccgtacttcaacgtatgacattctgtaaatctacaaaacagatgggtccctgcctttgagaagtttttacaatttagtgggtactggacacggacctaatttaattctcacatgccttccctttctaataagctgcatgttatgtatccctattctacagatgtggggaatccgaaatttttagcttttactgggacacacagctagcaagcaactaaagcgagaattccaacctgagctccaatccgaactattttcataaaaggcaggatcccaggctgagggagttcgaggtttgggagacagtttagcagtcgctgagtccaaacatgttgttttcctagaaaagaaaactgagatccaggaaagggaagtgattttccaaagacacacctgtgaaaggtgaggagaacagcccagtgaggctgaggccacacaattaaccccagtctttgcactacaactctaccagggatcctggtttggctcattttctttcttctgcaccatgattgttcaccttttcattaccagcaacatacccaaagacctagctatagactcgttggaatctgttacttagcagctgtgtgaatcaagtgttcaataggaataataagatgtattcatcttaccttttcagaacattatgaagaacaaatgtggtaagacatatgaaaatgctttgtaaagtgttagatgcttaatacacagaagacattgcttccagatctgtttcaacgtaccccaagaatggaataagtggaatatgctggtttcatgcactgtggcaagggggtctcatcttggctagacttgaataagtggatttgttgcctggcctctgtggatggctctttctctgggcttttgtgtatttgattttttatttgctccagcagccgtgttgaaccatgaaaggacctcaggaatgaaacccacacacagcaaagcaacaaagtgaaagaagcgtggattcctaatgcaggaggcactgaaccaatcaatacccactgccctccagacctccacatgaaagaaaaaaactgctgtcctgtgtaagacacttttattttgtgtttctattgtatgaggtcaaacctaataacagatatattacactctaacagatacattacattgtatagtagaattctcagacagcaaccttataaggtgatagatgcagaaacaggcccaaagagtaaaaataatttgttcagagttttccagccaagtggcgaaggtgagattcttacctgggtttgtctgaaaccaattttcactatctttttcagttatgatacactgcctattacgaatgaatgaatccttttttttttttccatttacttccatttgattcatttattccagggagtgacactttttctgtaaaggaccgatagtaaatattttagactttgtgagcaatacagtctctgttgcaactactcaactttgtaatgtaaaagcagccatagacaatagtaaatgagtaggtatgactatgtttcaataaaacttta

SEQ ID NO: 382 - DNA, 3234 bp, Homo sapiens
gcggcgcccggggctacgcgccgcactgcaccgagcggcggcagcggcaagcttgggtgtgagcccgggagccgctttgcttaccgtcctgccggtcccagccgtcgctaggaggtccgcgggccctgcggcaaccctcgctacagacgctgggcgggcggcgacacctggctcatggcccccgcggcggctccgtcctccttggccgtcagggcctcaagccccgccgcgacacccacctcgtacggcgtcttctgcaaggggctctcccgcaccctgctcgccttcttcgagctggcctggcagctgcgcatgaacttcccgtacttctacgtcgcgggctcggtgatcctcaacatccgattgcaggtacatatttagagccatgactaagctaacggcctccggggccagcatgatggccgactcccagggtccgttgcggcgcggcggagcagccaatggcgagccccacagtctcgcgagagtgctcaggcgctcttcgtggctgccctcttagctgctagcggagctcctcagggggcggccgggagcctacaatccctagaaagagaatacgctgttccggaaacagaactgcagttaagaccctcgaaaacatctaagaaagtgtgcatcctaaaacacctgacgaatttcagaatgtgacaaagcgcagaggatgcattatttcaaaacaaaacagaaggctaaaatttgcaggaaaaagaaaatcagtaaaccgggaatcctcggactggattgtaagcaagatttcaatgaataagaagctgaaggtattaaggctgtgatatagaaggtacatatttcatcccacaagagaaaacaataataatcagaaattttcggtgaaaaaaacgcaaaactgtacaggaaaatcatcctccaagtaccagacataaaatgctgcaagcttttgaactaatggcgagagtgtaagaaaatgggctctacttcagtgatcctgtggcaggacgtggatcaagacttggaaccgcagaaaacgaaatcccatagtagcacaaagcttggctgttcagtgaataacatttaaataatcgtaaaatacaaatgttgtttatggtttttattgtttaagggcatacttaattatggttacaaagtggagtgcaaatgttatttaccatgttttaaaaatacagccggaaaatacaagtgggaatgttgaaggagggcgggggaagtaaatggaatgggggttatgtccttataaagtggaaacttgaaaggtactgtctgttgttgagtggggaaagacatttttattacttacagggtagccataaagtttctaaaacggtaatatattaaagagggagagtggtaggggagaacagtatgaagtcaacaggaaatggctaaagatggagagctcaggtagaatagtttaaaaaatgaaaattgatagagtacctggtgtatttgaatacagcctagagagctttcattttccaaagagtgtggggataaattagtgaatgcatatttttcaaaattaagaaaacagaacatataattttgaacgctgagaaaaataaaaatttaaaaactacactgtatacacggtatacctcaactgtgaataatagtagtcatgtaaagtcagtcataattatgtaaatactgaatactgattttacaacaaatgattctataacgctgggaggatgggagaaaagagatgtgtgtgtgtgtaatggtggtgagccaatcaagatctgatatagaaaaatgcagaaaaaggagtatacacatgttttcttcggattaaaaaaactaataataaatcaccaagagtggtaaagttttaaatcaagatctgatatagaaaagtgcagaaaaagtagtatacgcattttttttttcaggaaaaaattaactaataaaccactaagaggggtaaagtgttaaaatagttgcattgttctatattccactcattgtgtccagcaaacagtgttctatggctttaagtagtaagcatcttgccccgtccaacaccatgtctggcctagggtaattgttctttctagtctttattctttgtctccagccaaactccatttgggcacattctcctatagccttttctacctgaagatactctgtgttttaaagctaagcttaggtggcgctttttccattaaatttttcctggattccactgaccatataggagctcacttcttttaatccataaggccattttcataggttgccttatttttccctaatcgtgcatcaactgtctgttttatatacccaagacaggtttcctagactgcgataagccaaaacattttagtctaaaatatcagaagtgtagtttaatcaatgaaatagtaataccaagggatttagaatcgtggacatcactgtttcccagagcactgatgtcccaatttgtaacacaaaagactgtctagtcttaatcctgaaatggtgacagagtaggatgctccatttgggtgactatgtgaacatattcctataacttttttcctcacatcagtcatttgttaaaccgaagatgaagaaacagactttgtatttattacatcctccaacataattggcagctctgtgagggaagacactatatcctattcaattttacacccggtagtaacagtatgtgtgtgtactgtatatatacatatatatatacatacacatatataaatttaagtagtaactatatatgtatatatgtatagttactacttactatatatgtatacagtatataagtatatatgtatagttactacttacaatatatgcataaagttacagcttacatttacctcgtaatatatgtatatatatatattaggagttaaatgtattaggaggtaaatgtaaactctgtggtttatcatttattcacaattgctcttaattcttggggtatagtttgcctcctacatgatgcaattcagtgaaattcccaggagacaaaacaagattttgacctgaataaaccacttattgttgtaattcacaagtgagtttattcttcctggcacacttccaggtaatatccattaggctaaggattttgtttgttcactgttctatccccaagcctagagcactgcccagtgcggaatagctaataaatattgttgaatggatagatgaatggcaattttgttggtatgctctacatagtcagcagtcaataaagga

SEQ ID NO: 383 - DNA, 2407 bp, Homo sapiens
aattttattatgtttgtatttaactgggagaaaagtaacctctggatgacctcaatgtccctaaatctgaaattctaaactttgtacagatgtaataaataatctcatggggagtgggggaaggggagggtctttatttttaggcatggcaaagtttccctttccttacttaagattttattttagcaaggaatttttgaacaacttcttaaactaaccagggacatgcatctgagcaaagctttatgttaaaaaggcaaggaatagccgaattcatggaagtacttctctataatattttgcttcattttcagttagggtagaatgaagaagaaggaattgatggcagtctacttatttttcattccactagaattatttatcagttgttaaatttcacagctgtgtgctgttcacatggaattttacaggtctgacatcttgacagaggtatattgtaaaataagatactctctccgggaaaagagaaggcagtttagaggacaggtagagaggaagtatacagataaggtgtgcaggtctgctcagggttccgaagcagtttcctggaaaacttgaaactgtggccaaaggggtaagtaaggcagtatatgaggaggcaagatgctggctatggaagtcaagcacagcttcacaatgaaaatcagattttctttcatgaaagtaattgaaattaaataaggtattaatcagcaatgttaccaaataattggatcttttgaggaaataaaactcttgctgagaacctacaaatctggcacttttattaaagaatttgtgtatgtaaatttgacagttaatttgacagctgaaaatgtggcaggactgttaaaataaatattccaaatgtagtctcatccaattttgactgatttactaaattacaaaccaccaagatatcatgtaccttttatgacacattgttttggttatagagttaaattcaatggcatcaatactttgttgaaaatctttaaagtaaaaccctcattccaattttcaggagataaagttttataaacaatatctggatgtttataaattctgctcctaggagaaagtaaaagtaaaaataaaaaagtgaaaacaaaatcagaaaaatacttataactcagaatttgtgataatttctggtggcaaggactaaagttaaagttgacttatttcctcaatgaactactgatatattatgcaatacttttctcttgattagtgaaagtattatttcttggtagcactttgctctttaaatattcataagtaacaacactgcatttaaaaaatattttcctattatgagtactttttaattttcaaactaatctttttccccaaatctgtagtgagtgagagagtaggtgatgtttaattctggtagggtcaatccaatatgctatgaatctcagttctgctatgtacttgccagacaagttacttgaagtctgagcctcagtcctctcatatgtaaatgtggctgagagctaccctcactgggcagctggaagaataacatgagtaatgtaagcaatctcttacatgttatgtcatcagcatttaacaattgatccttatctttgagacattcatcacttgcattcctggataacacactcctcagcttatctatctactctattggtcactcactttcaaactcattttcaaatcattgattcttcctcatctccctgatttcttaatattttactgccccaaggcttagtgtttgaacttctgttctctatttacattcactcccttggtgagttctttcagtctcatggctttaaagaacatttgttaatgaatcccaagcttgtatttcctggtaggagctctccattgaactccagtcttatcatctacgtggatatttaatagacatctcaaactgaatctaatatcatctgagcttccctcccaaactgattgcttccccatcttagtcactggcaacttcaagttgctcagtccctaaattttacggttgtcctttactttctctctcaccccaaatattggaattgtttggaaatcctgttggttctaccttcaaaatatatctagaatctgtcattgcttatgagccccatcctcctccaaaccaccattatttcttaactagtttccctcattgtgccttagcacctacagactattcacacagagcacccagagaaatcctttaatcatgctagctccctgctcaaaacctcctaatgtcttccccttttactcatagtcaggacccagaaacttcaacggcctacaagactctacaccagtggcccccaacctttttggcaccagggaccagttttgtggaagacaattttcccactgactggggaggtggggtgggggctgtgtgggggtgtgagatggt

SEQ ID NO: 384 - DNA, 2366 bp, Homo sapiens
ctgctgcgccctgcggagctccgaacacgtgcgcagaggctggctgtggcagatgcaactgcaggatgacttgaaagtagggcatccttcacccatctgaagggaggaaatagtggcaggtgacagtctgcatgtgcagttttcagatgccttcacctgaatgacatctacctccatcaggaccccagatgtctgacagccctgtgtgacaccaagataagtaacgatcccgttgaagataccacattacacttaggctcctgttggccatgacagcttcttagacttgtttttaatgaccttgacagttttgtttttttgtttgtttgtttttgagacggagtctctgtcatccaggctggagtgcaatgtaatggctcaatctcagctcactgcaacctctgcctcccgggttcaagtgattctcctgcctcagcctccccagtagctgggattacaggcatgagccaccgtgcccggctgcctcctaaattctcaaaacattccatttttattctgtggtcttactttattctgtcatgtagagaatagtggttgagcctgttgtcttttgagagtggatgctgtagtccttacatggctgactttcagcccgtaagcttatagtagaggatggggattttgttatcagctgctaaaagggagaagccaagcccagaactaagggttctgatgagttgaaggagagggaagatgcccctcaaggcaaagagcctctgagttagaatctgtgcttcaccagagtgacaatggtagaacttctccttctcagtattaattctcctggagagaactctagggttgtctaaacctgttcaactaggctgactaccccagcaagtgatgggttggacataaggcaaagaacaggaggggtgaaagggggcagaacagacaagttctgtcccagcctctgctacctctaaccccatggcattctatccttttctacactgggcttccatttcttaccccaacaatgatctgttcttccaggtgccgtcatttaatttcccagacacttgacctccttctgatttgtgtactccctccaaggctgagttgcagtgagtgacaataatctgtgctaattacttatcttgccagaagactcaaagggtttatggcttttactaactgaactctatgctagatgttagggataaatggttaacaggacacagttcttgcttatcttgctatggtctgtcaagccttatacaatctagccttatctctctcaccgtcttatctttatcacccgtagattcccttgttggccactggtttctttcagtccttaattagccttttgtcactacctgtctacacatgctgtttttcccttctcattccatcttgacattgtctattttgagatctcagctcagttgcagagaagctgccttccccatctggcaacccattatatgtgtcatagtaccatgttgtaccatagagctagacacaggtgccatgttgtgtcttgaaatgtattcaccagcttccaaaggtttacctcaatcccctttactcaagaagcctcagttctactgacagttggtaataataacctctctgtcataatgtacccaaaatagagtaagaatatcatgcttttcagtaatactccagtgaatgaggctaagagtaccatttttgttcttataaaagaatttttttggacatgaatacaaagatgtcaggttaccaaatcatttgctagtagatcctaacaatatcacctataggaaactgaacgtagcctttaaacattaagtgatgataatggatttggccgggcgcggttgcctataatcccaacactgagaggctgaggtgggtggatcacttgaggccaggacaggaccagcatggccaacatggtgaaaccccgtctctacaaaagaaaaaaatacaaaaattagctgggtgttgtggtgtatgcctgtaatcccaactatttgggtggctgaggcacgagaatcgcttgaacttggaaggcagaggttgcagtgagctgagatcgtgccactgcactccagcctgggtgacagactgagacagtctcaaaaaaaaaaaaagaaaataatggatttgcagagacttgctatttagatttcagacatctgttaactaaaacacatgtgtaggcttttgttacttatttcagtaatctgtaaatatctttatatttgagaaaatttgtgagacatctttgtgtaaattataacttgaagaacctctcttacaagcaggcatattggtaagtagctgcgaggatataacttataaccagattgaagtgtataattataatatgttattattctggggttctataa

SEQ ID NO: 385 - DNA, 2268 bp, Homo sapiens
tgggcagcgtgggacaggccccaccatgctccgctgacacctggcacaccccagggagggccccttccttacctggggccagctgtgggcacttgtggccatggcctcctcggggccggactctccaagtggcctccaggagggcggtgcagggcagcggcggcctccgccgttccctcccgcttttcttctgcacgggggcctggggctggtggcggcttcagactaatctaactccagaaaagagagaagaaaacagtgggatggagtctgcgcgccagtgagtggaaaaacagcctcccggcgccaccaggcagaggagggcgggccccgagggcggcgcgagaggagaggagccgagaggaagttgagatgcctgttctgcagctgctgcgcgtcccaccgacccgctgctcgccgtccctgggccgtgctggccctccgccctcggcccagacccggaccgggccatgctccacaccaatgggaccagtcgggagggccacgtggggatgtggaggcattggggagatctgacccagacctgccccccatgacagtttcaagagcaccccacgtacttgacccccgctgggcagaagctaccaaaggagaccccgcgagggtcaagggagggtgtcgtcaggcccgcgctgagccctgcaggatgctcagggccagggccccaccctctactggcccttctccctcagagttgtccattgactggacaaaccatccccaatgctgactctttgacctatagtcagctaactccccgggacctgcttcggatccctggagggctgaggattgaattcaggagcctagggattctccgggtcctgcaggcagagctccaactggtgggtgaaagttacaaggaggcaggtttctgaaggaagccctgtgtaataatcagagctgcccagcctgtatctaaaagtggtcaagatgcgggctgaagcgtcacgggtcaggaatgctgcacaggagagtctacggacaggctagatgacccttagcatggagggagaggacccccgacatctgtcagctctgcagtgctcgtggttccgtcatggtggagctgccgagtgacccgctggcctccctgagctgggcttgaccatatgtacgaaggaaagaaaaggcatcccatggatgtctcctagttccctcactgtggcccagtgttgtatatcaacgtcatcatggatacacctcctgtgggaggtgattaggccatgagggtggagccctcgtgaatggaattggtgcccttatagaagggaccctggaccctggacctccactctgcaaggatacagtgagaaggcagaacccagcctcaggtacactcatctcagacttgcagcctcctgaactgtgagaaataaatgtttgttgtctgtggtatgttttgttactgtggcccaagctgttggacacagggacacccctgggctcccaggcaccacccttccctcacgctgctgctctcctggaacacggtcgtgcctcactgcccatccaaagccacatcccggctccacctgctcactccttgtttcttccactcgatggtcaaatttttctcttaccaagaagctggccaggtgcagtggctcacacctgtaatcccagcactttgggaggctgaaacaggcagatcatttgaggtcaggagttcgagaccagcctgaccaacatggtgaaaccccgtctctactaaagatacaaaaaaaaaaaaaaaattagccaggtgtggtggtgtgtacctgtaattccagctactcgggaggctgaggcaggagaatcgcttgaacccaggaagcggaggttgcagtgagctgagatcatgccactgcctgggcgacagagcaagactccctctcaaaaaaaaaaaaaaaaaaaaaaaaagccatgtcacagagctaagagcaacccaaccagtgatctgggttcaagtctcagtctggccacacactagccagggaccttgggcaagtcatctaacctctgggtgctgcagcgttctcatccgtgaaatggggagaatgatagcgcctgcctcccaggtggttgtgaagattcaatcagctgtgcaggtcaagagtttcgcagtgcgccaggcacacaaggagtggtcgtgaaccatctttacaacattgtaataataatagtgatcatctctgcacctgagcttcagtagtgcattttcaaaatctgtattagttttctattaactgctgtaacaaatgtcca

SEQ ID NO: 386 - DNA, 1921 bp, Homo sapiens
aaacaaagaaacaaaagaacccacccgagtggggctctggaatcaaactggcaacgctcgagatcgtttctggggcgggaggctggaaaggggagcaggcagggagcggaagcctgagcccgaccaaagacagctgtgcacgccggctccctcccactccaacctgggctcgccgtccgggctacagacgggaccctgcggggcgcccttcgacgaccgagttcgagtcccgccgccaccgcgtcctccccgccgagcgcacacgggagcggcctcgcttccccaggtggggagcttttctcgcaaccgactggggctgcagctgccaactgcagtctctgtaggaaacaaactttctcctaaatgacacgtttctccgcgacgcgcctcctgcatctggagtcgccttcaagcctattcattcacccaagtggaaacctggagggaggcagttgtgcctggttgtggcggagcccagggttttgaagctgccaggaatgggatctgggggctgtgtagttgcctaagtgggaagttgtgtcactgggcccggttgggttcagtctgtcacctgactgctctccagagaccatggagcctttcagaagggcccagccctgggggatctacgacccttggttcccttcaagaaagctcagacagcgaaggaagaaaagggcagggcagcctatgagagagagaaggcagagaaaaaggaaaggagatgggaggaaagggaacgccttctttagaactcaggaaaaacaacatcagaagtgggcatttctccctgtataatctcacagtatgaccaggagaaaagctgttacaatagagagcaattgtgagcatccaatgaaaatgtactcaaaataactggaagaagatgcatggtatgatcagggttgagcctgtaatgtattattaatcatttctattagcaatatactcatgaactttttctattgctcagccctggacatggcctagctctttctaccaaagcaaatatgacgtactggtgactgactgaagtagcatattaatagttgaaaagcaaaacacagctccttagcaatttagaagttttgaaaaacccattgttctccctaaggacacatacttcagtggcctctaaattttaaaatgaaattcttcagggagatacaaatacttgtgacttttttcttatgtctctaagacagttaaattttaaaaggggaatgaattcctaagtaaaacctgccttgagtacaacttacactgctctgaaaactggaggggtattattgcaaaaaggattgatttgtgtgtgtgtgtgtgcatgtgtgtgtgtgtttagcaaatgttttgtaatgattaagaacattgatgggtattcgtgtcaaatagatgggcaccaagttttgtttgtgccacataatgagctctgtgactctgagcaatttatgtaattctctgtgcctcacagttttcttatcctgaaatggagagaataataatgccaagttaattgagctataaatggtaataataacaaacctgtattaatggcttactatgtgccagacagtattcaaatactctgctagtaaattacttagcattttgtctggagcataataagacctgcatgttagctttttagtattagtattattactaccaaagggctggcagatgcatcaaagaagtttgcagccaactgggagttgtaccaaatgcacagagaacaagttagtgaaagaccaggtgatgcatccaaagatgtaacatgttggagatttctgaaaggctgctaagtaggaaatcaaaatctaatgaggttaattaggaaagactttttttttttttttacagaggtgaaatttcagagttcagaacttaattggaatgggcttgaagtggtagttctaaagaa

SEQ ID NO: 387 - DNA, 3344 bp, Homo sapiens
ctctggcccttcatttcctgtcctacactggtggatgtttggaagccttcctacaccagagcggcttgctgaacatctactggatccagttttagtatgggttcttaaaagttttactacatagttaggtaatagtcaaaattaacacaaatgcaattatcaaacaaacccagtcaaccaggattgaaagtgtcaaacagtgcaacaatcaaggccattctttgtcatgatcaagagcacagagcctggagtcagactgacaaaggtttgagattgatgaaagattgtcaggtgactttttggtctctcctacatagtctggaaatcagaatttggagctttaaactttaacatgggtaggatgccaactatgctttaaagttattttctttgatgcagaaagacataatttttagtatatcataacattaattcacaccattaatctttttttctctgcatattgaatcataggacttcttttcacaacaatttttgttgttataatgttattttaatgttactaattagccctgcagaacccctatgacatcctttggcatcgtctcccatcgtctctctctctctttctctctctctctcattttaataataactgttgtataaaaattgcatgaggaataagttcccaactcccgtgatctgcttataatagctgtactttcttcatatagtcttaagaggcatgtctagtttaagtaccttccagagaaggtgtgtatttatgcaaaacttttcaggggcttgttaaaagtgaagcttttgcatgatttgtataagaaagtaattaggatatctatctaaatgataataagaattcaatgagtacagattggcaattccactttgaagattatggaaagatagagattttatattttaaaacaatttgttaggaatatcaaagcagaaaatattctagatttggatgaacaaccatatattttgtttaaaaagcaagaaattgccccagattacttcttgcattagcaatgtatatatcaaagactccggaaaaggaatttttttttttttaaccactaaccactccttgcaaaaataaggagcattaggactttcctccctaccctttacttttttaattctactaattgttcacacaattataaaatatgtagaagattgttttattatgtttttatctgtttgttcagtgatagtgttatatagtaaaagaacactggtttgggattaagcttaacggtgttttagtctgagcctcaagagactttattcatactctttaacctctataaacctctgcttcctgcactccaaaatgggactaactatactaccttgcctacttctaagtttgctgtgagtatcaaatgacaaaatgtgtatgaaaatgacaagccaatgtgtcatccttgcaattaccatagttggttagaaagatattaagcaacggttaaacttttaaatcctgttttacttgtttttattatgatagagttctttcttctattatttatagagttatatatatgtatcatccgtatccaaactaatattaatgttatggcaacttgtgctttttgagtctttccaaagatttcaacatgattttcataaagcatatcaatttttttcactcacattgtgtgacatcaggttaaaaagttactaacttaaatttcttagaaatattgactagaagaatatataacaatataaaatgtagagacgagtatgaaatatatttaaaactcattacgatactttagttcatttgctatttgtacaaaggaagctcaagtaatttcagtacctatggcaagatttatacattctgtaattagatatcttttaaaacctacggtgataattttcttaaaaattggtcttaaagtaacattgttaacacattggtaataagagtattcattgcctttgttttcttttcccctcattttatatacaattttgttaatgactgtagatttagtggtatatgtggtagctcatgtaatgctctagtatgctcagtttaattatagatttttttaaaaggataattgccaagcttatttgtcctgcacaacatcattgggaatactttaaaaagttttcagggaatcagagaaaaccaaactaaagataatggcctcagaacccaaattatttttactctgtcatgaattttgctaacaaaatcaaaataatgagtactaactgacaaggaccatttaataatctacccagctaaggttagcagtcaatattttacatgatatttcactgataacttatttatcatattttacatgataacttaagattctgtgggtcttaagttatgcatattctcttttattactttaataaatattaaaccacaacatgatgtaattttagcttattactaagtattataagagcacttagaatataactttttacaattaccaaaaattttttaatgtcttatcaacatgaaatacttagtcatttatttagaaagtataagtttattagtggatttactgtgtcctgatttaggtcagttaccttaaaaatacgttcatttgtctgtttttattaatgctacatgccaaactaatgacatttaaaatcataggaaaaagagatttggcatttgatttatatttcatggcaaaatgcacaggtttgggtaatctcaggttcttaaattctttccacattaaatagtgagctaattctaacttttttactgtacttcttcgtgttactgcattaggtttttgaatctgcttggatgactcccgaagagcagtttagtctttgagaaaaccagatctccattgcttccatgaagatcttattaatatttagataatggagagtaacgaaaagaatttggacattgaatatgtctttgtcttctagctatcattctccccaacttaatctgtgaccctgagaaagttacttcatttctcttgagtcctcaacaaatttttcttacacagtatatgccatcttaattatcttaagtatgtacttgatcatctctagaggttcctttctagtcttaggttgtaatagtctttgtcatactggaaaaacatttttaaaaaccatcaaaatgtattctttttttctacactggaattgacatatggcactataaagatcttgtgatggtagaagtcatacatttgaacatgagaatattaatgacagcttgtaaccacagtatttcttatcctattctaagaaataattttagttattaagctggttttttttggtatgtcttgagtttatttgaacgattgaagtatttggaattgcgtcatgtaatagttcactgc

SEQ ID NO: 388 - DNA, 2197 bp, Homo sapiens
cgctgggagactccgggccctactgtctgctcttcacaaagccaggctgctgcaggaacagttcccgcggtcctaaagccggggcggggaggctagtgggtggaacttgagggttgggtctggaggaagagatgcctgggcagcgtggtccagcccctgcctcggggtggaaaggcagagagcaaagaggaggcccctcttattccggacccccaggccagagcccaggagtgggaaagcagagggcaaagaggaggcccctcctctcatccctgacccccaggccagagcctaaggttcaagtgcctcaggcccagtccccttgacgcttcctgtttgggagttggattttataccacggattttatagcatttttatataattgaggattgtcagaattggaaagggccctggagatcactagtactaccctcctcaccttcacagataggtaaattggagcccaggaaccgagatgatcacacagccaggtagggacagtacaagaactagaatgaaaatcctagctcctgactcagtgttctttgcacgatgccacactgcttcccaattcgctggtcagttattaaaatttgtcaggcctgccgaaggattgacatgctgggcaatgcaggccaccacggtggatctgtgactcctgaaggtggcaaagccctgcacactgccttgggatgagcacactgagctctgcagggcctcagctggaaggcgccatccaatcgtccttcccctccccagcagaactccagaggtggggcggctgccgcagggcccaaggctgcctcggcagggggctttgtgctttttgttgtggaggccaccagggattcaggaagatcctgaatgggttgtcaattgactgaaaagtaaattcccagtgagtttgaacctccacggtttttgagacagtattaaaagcttgagaacggagattatggatggcaccaggctgggcactgccattcttttactttccttcatgcattttttcggatccagagtattaacaccactttttttactctgcccattcaaaaacagtaggcaaattgggggaaccaatttataatcactcaagcccactgccaaagttcggataagccagggcagaggggttagattcccccccctccgtaggcagagagaggcctcaggagagtggacatctttactgagacagccccaaacacaccagtggctctcaaagtgtgtccctgagaccagcagcatcaccttaggaatctgctagaaataaaaattcttgggccataccccagggttactgaatcagaaacttggttggggcctagcagtgttttcatgagccctccattgatcttgatgcagctgaagtgccgggcaccagtgaaagggaagggctgcggtagtgattccattgattcctgctttgatggaagttatggcctcacaggttagacgttgcaagttgagttggccctcttgcctaggtgtgcctccttgccttgggattctgacatgaacaggtgaagtctcttcatgattttttttgttgttgttgttaatggtatgcttgctattatggccaatatgtaaatgttactctgaatacatatttatataccatgttacttagtgacattattcaaacatttgtatgcgtatttgtgaaattgtttgcatcagtcatttaaaaaatatttcagcctaagttttctaagaggtatgttgaatacatttttaaaagccagcttaagggtcacagcagccttagtttaggagaagagctaatcacaggtgaccactcaagttctttcaatgaagacctgatgatgtatttagttcccattttccacggtgattgactcaatttaagtgtattgtggctatatgtaatttacaggctgctttgcgttgcaggtgggaaaatctcccaggctggtgtgaggctgaactggggtaaaagaccccctgacccctcctcattcttctaaggtcaccacctcttagagggtctcttattttctttcagagatcaaatgcacagttatgctgtacaactaaattctgcagtcttgggaatccttacgagtaccaactattagtcactttttaaagtccccaaagtggagtgaggatagggggagtggaagcagtgtttggttcttactttacccaaactgtgtataaagaata

SEQ ID NO: 389 - DNA, 3015 bp, Homo sapiens
gccttgccaccaacgggctacgagcggttaagagactacagctcccaggatgtacggtgatggcacccgtttggactccgcccctctgcgcgtctagagctgttctcccgtgcggaggggatcctggctgttcccaaatctcgcctgcctgcgaggagccagcgtggcccgggcagctgatctcaacctgtgattgtgacatcacccctacctgaggaagcctcttccagcctgctcaaggttgccgtagttaggtttaccctcccttccttccctcaccccgtcctttccctcatccctgccccttcctctgtcccacgcggctttcagacatgcatagtgcagcccctcagtaaaggtgcagctaatggcccgtgggcctagcagttgtcaggccttttgggagcccatgatgggagctccctggtaagtgaaaccgtcaattaactgcctaaaatgacaataccccgtctctggtatctttgcacttgcctcttcaaagccaccttttcctttctctctctctggactgtcacagatcctcctctgtaccccaagaacaccccattggatttccgggctggctgcttggcctacacacctgggtcaggcctctcgcagggatgcgcctgccactgtaataaagaggagaaaacgtcacacgggaaaggcctgactccttcgtacaatcagaatactcaactgcacagagaagggacccctttaagccactttgggagccacatccaccactctgtgactcccacacaggctggttcccaggtatcaggtgtcctagtgttaacacgggccaaaaggacaaaacaggaccctaggtccttcccaaaacattaggaaagtttgtgtactgaggtaatgtacaatgtctttttgccctgaatagggctcccttaaaaactcttttaataatcatttttaaatatatctctggaaccacttttagtaatttgctgaaggaacataaaagtaattaccttaattcatgtttttcttttagtttttcagattaactaagtaattcatattgtacatttctgaaattgaagtacgcacagcttcaattctgtgcagggaggatgcagcaggagagaatatcctgggcacatctttctggagaatcacttttactacaagatttgtaagaaacaaatttatttccaaggtaagaaattaaaactctaaaacaaggctagaaagtcatctgcctttaataacccttagtcatctgtgcctgatatgtgagactttctccaagataaatctctcaaggataacctattttccatcattataattagtaaaaattagaaattttagaaagtcaattggaagccctgtcctgccagtttcttaaatcacaatgtggcctttgtacgattttacttggattttctttttggaatctgttgactgcttggatataaaatgtggggttttgacaaatttttgcaagttttcagctattgttacttttgcaccccatttaatttttctgtcccttcactctttctaggacacaatctgcccacagattgtaagactctgttcattttcttcaaactttttttactctttttttcagattgaataatttcaattgttctgtctttttctaatctatgaatcaacctaaaagtactatttaaaatttcattatctttctatttggttctttttaataatgtgcatttctctgctgagattccacatgtattcattcattatgagaattttttttcttcaccccatgactatagttttaatagctgctctcaaatacttgactgctgataacaacatcttggacattttggggatagcttttaatgcctatgttttatcttgtgtatggataacatttttgtgtttcttctcatgtctcttaaattttaaaattgtattttaaaaactgtaaataatacttatagagactctggatcctgttgtattcctttgaagagtgttgttagtttttgaagagggagttaatttggctggattcaaattcaaatgcctgtctccctttccgtgggcacagctaaaattatcattcgcttcttatccccacatatatcatatgtatgatatatagatgtgtgtttctatataatatatgacattgtatccagattttaccattatttgtaagagtagtgttcaacaagctactccactattactgaaagccaaaacctcaattttatattctttttggattttacataaatgacattatatagtatgtatacttttacatatactttcttttgtacatcatatttgtaatattcatcaatcctgctgtagatatgtaagtactgagattacaggcatgagccaacgtgctcagcctaaaatattttaggaggttaaggtgggaggatcacttgtggctgggaattttagaccagcctgggcaatagagtgagaccctctctctacaaaaaaattaaaaattagccaggcacggtggcatgtgtctgctattccagctactcagaagactgaggcagaaagatcagttgagcccataagttcaaggctacaggaagcaatgattgcactcctgcactcccatctgggtaacagagcaagactctgtctcttaaaaaataatataaaaatttaaaataattttatacattatgttaaaatacacataaaattaactattttaaccattttaaaggtttccattgagatgaattaagtacactcattattttgctaccatcatttccatatataaaaagcatattccattttttgaaactgaaactgtacccattaaacaactccttattctccctgtagcccctgggaaacactctcctacttggtgttttctatgactttaactactcttaagtaccacatatgagaagaatcatatagtaaagaaatcatgaggaagaatgataaaaaatgtatcacatgcttattaattttcaataaaaacaccaacagagatcagtagtacctgttgattataaa

SEQ ID NO: 390 - DNA, 3206 bp, Homo sapiens
gtattccaggtagaagaaagcaaatgtgtggccttgagtggtcacttctctttggcttatcgtatatatctatgagctaatgagaatgaaaattccttttttttcaaattgttaggttagcttaaagagatattgactcattaaatacaaaacacaagaagacattaatataaaattgtatacattttttaaagcctatattttctcccttgaattaaaaataaagtgcagcatgttgaaatgttgaaccctggccatatgacaaaattggaagtctctctttgcttacaccttggggcagaggcaggcatcagacaaagtcctttttattttgtatccttaagagtatttcaaggtcaagttaaatgaaaaactagtcattggctctgttagattttcaaatatatcctcaataaaatgtacactattaatcattttcatattggtgattttaatcaacttttaaaccattactttttagaaaagtttttcacaatttctggtttctagggtaattgaaatttcagtatataattatagtagtataaatatgatacttaaaagtattattaaaggtcattaaaatattttaaaaaaactggtagggcaagaagactttactacaagttaatgaaatgggggtaatttagttgaaaagaaacaaaatgattacaaattaaagattactattctgtggcactctttctttagttgacagtgaataaggtagagttactatatttttattcatattatgatttggcccctttctagaaaagtgaaaattttatgtaactctgatacatggaatatattttgaaattaagaagtatatgtacatatattagtcattattattactgcaactattcctagtgaaaaatatacctcattcaaagagaaagataaatttcaacaactgttatatatgcacactttttgcccataaaactaagcaagcctaataaattaaaatttatctaagtcacaaccatccaattagggacactttcttgcaatcagattacaaaggatatttttatctctttcaatttagcagtcaggttagggctccctaatagaaggctgcgtagctcattagtcctaccactttggatggcctttactcttaacttcattctccgtaactcaaggggagtcagtgtgtaactgtgggtgcagagcagcaatgaggatgcttgagtcttcattctgaagggatcacctgtggaaagaatgatcagctttcagaaaatctcatctggttaagtcatatttctctgctttaaaatgttacaggatttctcattgcccttagcacagtttctcaaactttattgaacatcagaattacttgaagggttttcgaactcatgaattattcggccttgccccagagtttctaattcagtaggtcttgggtggggaacatactttgagaattctgacctataggataaacactagagttcttattatgtcttacaaggcccatcatgatctgaaggcagcggtggttgctggtggaggatgtgagctttggaactggagctctttgattcaaagctctgccgcttactcactggataagaccatgatcaagttatttcatctgaaaaattgagttgactgcaggtaactgaaactgcggaaagtgaaactgcagataagagggaccactgtaaataagtgacatgaatgatactgcaaaggaccatagcctaaccagccaagcaaactggaagtacctttagactcacgctttgccaagctcagttccagaatcacatgatgaaaagaatgatggaaagaaaatgtttgtgatattaaaattaaggaaattactccaactcagagtttcatcctaaactgagagaccaacatcaaaagaatcttctcaagtttctgagtgacggcaagaaactagactactgttaccaaggaactttttaaagcaaactgcttttgctgatatgaggagaaagttaattaataagaaaataccttaaatgtgttttgaagctgttaaaggtgtgtaacaattactctgaatttattatagcaccagttagagacccaggaaaactataaaattgcacatcagggcagagctgagagacgacatctgctttgggattgaaaaaaaaaaaaaaaaaaaaaaaaacagaaagggaacaagagaaatagcaagtcatttaacatttggaattcaggactccagactcatctataggaccaaactgctcttgaaatatactagatttcctttagctccagaggttttgccttcatcaaatgcctgtaacactcttattgtcaaaaggaataagtgatggtgaacactgtgccagataggaggtaccacacacaggttcagcaaaaagcagaagcaagaggtatattttttgttagatcttagttcatccctatgtaacagacatttcagggaagggaggagaaaataacaaagctacctatgagcaaaggtaaatgggattccctatccttgtttcccagtttcggtcatggtattgcttatccattattagttatccactgctgggtaacaaattactcccaaacttagcagcttcaaacaacaataaacatgtattatgtcacatagtttgtttcagaactttgggagtggcttagctgaatggttgtagctccaggtgctttttgtggttgcagtcaagaagtcagcccattcacacagctgcagacaaaaggactcggttccttgcaggctgaggcctgattaggcagggggcctgagtgtctcgccacatggacctctccatagggctgcttttcgtggcatggcagctgactcccctcagaacagatgatcaaaagcagcaaaagagaagcctcacatgacttttgccctagccttgggaatcacactcttccatttctacaaaatcctattggttacatagcagccctatccagtgtgggagggaactgtatacaggcatgaataccaggagtggaaaaccactagagactggctaccatggtcacttacacaccaacatgaaactttcagcctttccacctcctaccacttatactatatttaatgtttttcctgttatggactttaaaaaagaggtcatcataagcaataatatccatgaaatcatggatatcatgtgatctttatatttatttttatttttatatattaatttccaaat

SEQ ID NO: 391 - DNA, 3225 bp, Homo sapiens
tggacctggtcgcgctgtgccgggtgtgacactacggtaggtgtaggttaggatgtgtcttctggtcgtggtctactagttggggccgagggaagaagatgacgattccaatttcagtagccactctctaggcttatgaactgaaagaccaaggagcacaatttccgagtttcaaagatactttcaaactgaaaggaactgttcgccagctgctcgcgaagtggcccatgtgacccctagtgaacggtctaaacttccgctagatgctttgggaattcgtaaagcgactgcgtgcgcggcagccaagccacagctttacagggttcgccttctgtgtgggaacgcagcttttcccgagaaattgggttagggattaaagagaagaactgcatcctttttactgccctttttccacgtctgtcctgggcttagggccctcctcagaggccagaggggtaggcgagtccggggcggtagggggctgcgtccggaagtccaggctgcgttctg[450 bp omitted]gggtccttctaagccctctggcagaatttcgttgtggttgttggactgaggtccctttttgtctcgccagctgttggccagaggccagctgtgtgtcagctagaccttttacaggccttctaacacttcccatcttcgtggctttcgggagcccccagccccttttaaaggctcacctgattaagtaagactcagtcagatctcccttttgatgaacctagtcccagaagtgatatctgctcatatttgtaggttctacccaaactccaggggaggggattatgtagatgcgctctctaggggccaggaatgtggggaactagcttagaattctgcctgtacagcgttgtatatatcatgtttcgattagttggaagagtaaggcatgggtgtgataggtaacaggttgacttgacccagtaaaatttttttccttaaatcctttaatctctgtgtgtgtataaatcacaatctttgatatgaagagcaatgtcagggaaagtgaggagtaaagaagcaagggagttggatatccttcaccccttccactgggagcttgggtagagctagaattttggcgtcacgctggtagaccatggtgcccatgtctttccacataaaaactccctctctttctaaggaattttatgttaaaactcagcactttaggaaaggttttcttgagtttcatgatgatgtgctttttaagttacctgaagctacacataattgttagtataacctggaaattctagaaattaaaaggctggcacaattacgatttttctatgtacagaggaagtactaaatatctgtactctatgaggagacataagacatgaggtttagaaggaatggtctgtgtcttttgaggagtttgtagtctactttggtttaaatatttatactttttttttaaaggaaaattggaagctcatgagtttggccttatctggtttctgcctgcctgggaagccccctatcagattactttcctcatctgtctgcttctttcagtaacttggccctccaatgttcttttccaccttagtgctttccataatgtatattccttctctacctagaaatttcattttatctgctcagattcctaattatattcacactctttcttaacttaaatgttactttgcttaaagacattttccttggcccttcagactgtataaggacctctctgaagtattttcatagcatctcatagtgctatttcagagcatttattttattgtaaaaaattgtgtaatggtttgtgttatacctgatctgttttcctgtatcaaaaataaaattaggatgaatctaaaagtttattgtcatagaaaaagtacagatcagaatccaggagacttcaaatcaagtgggaagagtggtaagaagcttgcactcaccagtaacagcacagcttataaggataaaggaggaagcattttgagcttttccattattggtccttatacattaagattttgttggggggtggcaaacagctgtttaagttgatttgtctacagttgattggcttaatttcacggaatcatactaacaaaaatgtaaagcttatgttttatatttatgattagagcatttcagggaaatcaggatgacttaagttttggtttatgtggttatgggcagttggtcttgatgtatatctaaactgtgacttccatttttattttatttttttttaacacctgtaagatccatgatggcatttagtgggttctcaacaaatatttgttgaatgaatgaatatttgcttaggaaagctttatgtaagaatccaaaacattttctaattgaagagtaggttggatggatgaatgaggtcacttttagagaactgtgaatacctatctgtgttgattgaattggaggtaccagggttttctaggttcttaggattgacataattaaaggggcattttttttttcaaatgttaattcagtaatggttgtagaatggattgtgtacaggacagtctaggttagtggcctcaaacttgatcatgtagaagaattacctgtgtgttttgtaaattcctggcttttattccaccttccccagaactggttcaggccacacttaagcacctgaagtatgctgatccaaattgtaacatgctgcaagtgtaaaataccagatttcaaagacatctaaactaatggatataaagtattatgttaatatgttttctaattgattacatcttgaaataatattttacattcattgaatta

SEQ ID NO: 392 - DNA, 1820 bp, Homo sapiens
ggacctcgagcggcatgcacaggtccagcaggccgcataggaactccacgcgctgaggcgacggcagctccgagaaccagcggatatgatccaaggtgctgaaaacttcctaacaaaactcttcagccgaatctggcacgcctatggttccagctactgggtagactgaggcaggaggatcgcttgaggccaagagttcaaggccaacctgggcaacatgagtctttgcagataattaaggtaagagtcaagataaaatcacactgaattatggtgagccctacatgcaatgagacagtccttacaagatacagaaaagaacacatagagacttcgggggaaaggagactatgtgcaaggatggcagagattggagccgaggggtgcctgggcctaccagaggctgcaagagacagggaggaccctcccctagagcctccagggggaacagccctgcccacaccttgactttggactcctggcctccagaactgtgagaggacctaggaccctacgagaggagggatctggaggccaggacaggagcaggacaacacaaacagaggcctcagaagaagactcagagtgggacaggggttccttaagtgacgggggctggaagtgcacagagaagcctggtgcagcagggaattttctctccctggggtgctgccaaggtgtctgttgcttgccagcttttccagccatgactgcagacaaagtccacctagagtcataaatgggcaatcagccccgaacaacgtccagccccggtttctgcacggcgaggcgtttttgtccgtggcagacctgcttacccgggtgtctttggtgtggggctagatgcagcatttccccagtgggaatggcctggggtcttatttaaagtggagggtcctaggcctggccccacgtatggagtcaggatccctaggggtaggaccttggaatccgcattttttttgagacagggtctcactctgtcacccaggcagtggcacgatctcagctctctgaaacctccacctcctggttcaagcgattctcctgcctcggcctcctgagtagctggtattacaggcacgcaccaccatgcctggctaattttctatgtttttagtagagatggggtttcatcatgttggccaggctggtctccaactcctgacctcaagccatacactggcctcggcctccctaagtgctgggattacaggtacgagccaccgtgcctggccaggaatctgcatttttgctagtaaccctggtcatctcagtgcacaccaaagtttgaaaccactgttggggtgtggagttcttgcttccagacttccagggcctatggattgaacaacagcagccacagtagtaattagaattttcagtctgaaccacaagcctgccatgcaacaagggtttcctcccgttttacagatgagggacctgaggcttcataaggctgttattgacctgaggtcgcacagctgatggctcctgggttcttagcgttaagttttgcagccttatttctgttgtatatttgtttctcatctttctgtgtttattgcactgcaattgtgatatcctctaaaagccaaggatgaactcactgctgaccttggttttattcgtttgtttattttcaccttttcttgtgaaatgtgcaaacatactcaaaagtcaagagaatcatatggtgatttctcatagtcccctttggccagcttcagcaaagatgacattttgccactcttattccatccatttctgtcacgccacccctgacatacacacatgttctttctggagtattttcaagc

SEQ ID NO: 393 - DNA, 1948 bp, Homo sapiens
atactgacccctttttcttccaataaagttattgcagtaatcacatcattgcagagaatatttcttaaaagcttttttcaatatttacttctttaatgaaaaacttaatccagattaagccacatacatagacgtcttgactttattattttgaatacctgagatttccaaaatctctttaactgtcttcacacaatttctagtctgtcattttaattaattacaactgttcatttcttctgcttttgtttcctcttcatgtgctgcctagcgttgctagagtttcatcagtttcctgccttgagaggtttctagtacaaattaacattttcccttcctagtttatgcaggttccttcctggtaatttttatctttttgatctgtttcattctgcatgttagttatttggaaccattctactgtgctcaaatgaacagtcctatccctagttctcttattcctttttcatttcatattttataccctttttcctcagtctgtcttaaagaataaagtgttttcatttcaaaagcttattatctctgtttttaatcttagcccttctttcctctactatttttcttcctcttaatgctccatgctgggttctctgccttatcagacttaatcttgaaaagtacgttacttccttcccattatctccaaggttgttatagtccattctaggtgtctccaatttctcaactcttactcaattactttctgaagtttgactattatttctactagtgttcagctttcttaagtgtaagtaaacacatttcttacttaactaggtagataaacattagctggacggatctggccgtaaacatgctattatttccaagataaaactgagttccaaatggctaaatctttgtcttttcacttttcatccattcctgtcttttttagcatttgtttttttgctgcttaacattcacttctcctttgtcttctgtaataaaccttaatggtttttctcctctctctggctaaatgtttgttttctttgttactgctttcttcttttatataccacttatatataaattgggttgaagctttgctcttagctctttttcattacacatcattttgaggagatggtctcgtgttcattatgtagattatcacaaatttgatgactcagaaataccacatttttcttagtgacctgttttaccactttctcagtcgccaggtccttagcaccccaaaattgttatttcccagtggaaagtttgtcttgtacaatgcttgctatatggttggaactaagtaaatgctttttgaatcatgaactaactccttatctgtctttttttttcttaagataatggttttaactgtccacctattctcatgtcacaagccctgtaatatctctgaccacttcttgtctacacttgttaatattagttgtcatattttggtcctaagtttgactaatgaggaagatgacatttttgtctgcagatggtggaaataaaaatcacagagattgtgatttccttatggttttgtgggtaatggtggagtttaaacttacaacgaagtttctggtgacatgtttcctagttttcacagaaaacattctttttttttttgagacagaaagtctcactgtgtcgcccaggctggagtgcagtggtgtgatgtgtcggctcactgcaacctctgcctcctggattcaagtgattctcatgcgtcagcctccaagtagctgggattacaggcgcccaccaccacgcccagctaatttttgtatttttagtatagatggagtttcaccatgttgaccaggctggtcttgaactcctggcctcaagtgacccacctgtctcagcctcccaaagtgctgggattacaggcataagccaccacacccagccaaaaacattctttataatgatacaagtaatataccagacaagaaattac

SEQ ID NO: 394 - DNA, 2659 bp, Homo sapiens
cgtcttcccttcaaaaacttggaatgatttcaaatcataggcaccttcacttaaccctagcttccattcatcagcaaacacatcggatcgatgctacgctaacctatcgggttctctctccgcgcgttcaggttaaatgaatacctgacgaaagggcccacgtttcaaggcagtgacatttgatagctgagaggaaaagtggctttaatgaaaagcaacctttggaattcctgcttgtgagaaatccaattcagctttttgtgctgccagcaagaaatgatcagtagcaccagtgtttatgattttgtttgtctcaaatgtgcggcctaaaggaaccagagctacaaacctaaaagactagaaagtgggcaagtgctcacactctggcagctctgctgttgcaaattaaaagtgtgttgtggtcaggaaatgaaagctacaatgtctcttttgaacaagtcgtttatcaagagaaacatctatcagggactgccttgtcacattgatcctctaaaagttaggtccgaggtggaagctgctttgatgtttttgatcctaaatttaatacaacagaaaaaaagcaaggatattttgaccagtatagatttttaaaaatgtttcctggaaccaacaagatctatttattaggatttcactatgtgtgaagttttctggacgtaacagttgaaaggttgaaggggaaatcccagactttataaattgatgattgttagaatatacatttataatatatttagtgatggctggcttcttggtgcacatggaatggacgttttgaacacagattgttcaaaaggaaaattattttgaaagattattttggaagaatatgtagagagtcaagagtttttcattcaaattttagttatcctcttctgttcttcctctcttttactccttcatgcttgtttcttatcttcattatatttgtgacaaattagcttttatttggaaaagaaacattatttctacaaaaaccatgtcccaaaacatgcttgtacacatcgtctctattgtttattaattgtgcatgtttatggttatcaatataaactgcatggacacataagtcctttggaatttcagcttaaaaatagaggtatcagattaaacatgctatgtgttcttgaataatgaatatgttgtatgcattatttaaaattgaaaaggtattgttaggaaaatgaccaagttgaaacaaaattgtgaaagcagttagaagcagcctgcttcattttgggaggcaaagataggaaagaagtgtttcttctttaaaatagagtttttggatacttgtcttacactgctggaggcaacttgttatttcatgtgggtctatcatcaaataagcagggaagagtagggtgggagttttgagaagtgtaatacttgctaattaatcatgtctgaaatgaaagaatcatagcagggtaataaagtctcattgttgttttcctggaagagggtaagacttctgtttggaagctgtatgtgtgagttgatttgcttttctcttctcctaaaaacacagtttatagggtctctttggaattatttcaagaaacatcttttttttttttctgaaaatagagtatttttctttctcctggctgtatttcatcagtaagattcttaaaacagtatctgatatcttctgagtgaaaatagggggttttattctttagttatatttaagactcctgtagctgagtttccagggctgatatttttttccttcccataaatagaaaatattttttgtttttgtttttattcatgttaggcaggaactttgaaaacctttttgtaggtagttacttctgatagatatacaagagtgcagaaatttcattaattacttttagatgataatataagagtgtaaccagagagaagaaaataggtagtatttccaagaatctatagagaccatctgtattattactttgcatttctaagatagtttaagatataaaccacctgaaaagataaatgggtaatttcattataggttaattattttttcctgagcttctggttgtcaccttttagaatttagtatcttactcaggacattcatagggacagatccacaaagtttgagagtctgcactgtttttcagaatgttcatgtcaaggtgaagacagccatgctaatacttccctgtttttgtctgtgtgagtgtttggatgccttgaatgattgcctaattaagtaatcacttttagtggcccaaaatcaacagaattactagatgtatttgaaacaattgcaggcttttaaggtttgaactttctagttcttagaaccttggtttaatagaaatagaaaggtgctattttacctttgatcaatatattcttgagtgcagatttcacatactactctttataaagtactgtgttcattgttgaagagcaaataaatttaattaccaggaatttaaattgatttataaatttttgattggaagaaactattctgcagatgtttgtgtgaaatattgacatatgctactgatcgatagaacttgattaatcagcatatctgctgtttttcttgagtcattgataaactagaataaacaaatggttcagaaaaagctataaattttaactctcaataaaaaataaatgt

SEQ ID NO: 395 - DNA, 3214 bp, Homo sapiens
tcccctgattcagaaccactgcctagaggatatggcataatggtaactttatgtgatggttagggaatttgaaaaaaaaaaaaaaaaaaagaaacaaggaatggataagattcaacatctgtgaagtgacactcagtaaatgaaaggcggatgaattagcattactaccagctggtgtcctacttatgttatagaaatgtgacagcaaatgtgatatgaactccttagaagcatttttatacacattgctggctttaattgtgaatgctacagtaactctcctgactgtttttcttcttctgcctttagacaattacccaaggttaagatgcaataaacatgtgcctattttctatccaaaatctcaatcagattaaaatgcttctctaaagacctgaaaggcttatttgtttatttgtattcatgtaggatttggggggatgaaagaattggtgattaatcaacatattattttcccaataaacagactattctgacaaatgtttagtggcaaataatagcaacagcaataatggcaacaataaacttagaacagaaatggatttatttttatttatttattttttaccagctctttctctttcactcgcttctgcatgttgagatttctcacacatgcaacctagatcttcttgtcttctttatattctttctccaaaggaatctcattcactgttgtgcctttaaatagcaagcttcaggtgacccccacatttctatctccaaattgacatatccaaatgcctactactcatctcccctctgatgtcttatagacattcacatttaatagcatattgtattgagtctaaggagccattaattgtaagtcacatttatgtccactaagattaaaaaatactgccaactgaactatgacaaaatgctgatatccttgattctaagatacatcttgatttcagagactttaaaaataggcttaaaaaataatgtgatttactagaatagcaccaaaaagaataaaatacttaggaattaacttaccaagagaagtgcaaaacttgtattctaaactctaaataacattgttgaaagaaattaaagacctaaataaatggaaaggtatcttatgttcatggatcagaacactttgtaaagatggcagtaattcccaagttgatccacagattcggtgcaacccctatcaaaatcctgtctggtatctttacataagcttattctaaaatttatatggaaattcaaggagcccagaatagccaaaacaaccttgaaaaaagaataaaattggaagattcacatttcccaatttaaaaacttattacaaagctatagtaattaaaacagtgtggtactggaataagattagacatataaatcaatgaggtaggattgagagtctaaaaataaaccttcatatgtatagtcaattgattttcaacacaggcaccaaacaaattcaatggggcaaggagtcttcagcaaatattactgggatagctagatatccacatgtaaaagaataaagttgaatcctatcctcataccatatacaaaaattaactcaaaatggatagaagacctaaatgtaagtgctaaaacataaaagtcctaggagaaaacataggagtaaatatgtatgaccacgggttaaaaaagcttttttttttttttttttttttttttgagacagagccttgctttgtcgccatgctggggtgcagtggcgcaatcttggctcactgcaacctccacctcccgggttcaagagattctcctgcctcagcctcccgagtagctgggactacaggcacacgccaccatgcccagctaatttttgtatttttagtagagatggggtttcaccatgttggccaggatggtcttgatctgttgacctcgtgattcacctcaggtgatccacctgcatcagcctcccaaagtgctgggattataggcatgagccactgtgccaggccaaaaaaggctttttaaggtatgacatcaaaagcattagtgacaaaagaaaaaaaacagataaattgaacttcatcaaaattaagatactttgtgcttcaaaagatgccattaaaaaaaaaaaagaccacctacagaatgggagaatatgtttgcaaattatatatctgataaatgacttctatctagaatatataaagaatgcttacaacttcatactaagaaagcaaatacccaattaaaaaataggtgacggatctgattagctacttctccaaacaagatgcccaaatggctaataagcacatgaaaaaatgctcaccattcttagtcatcaaagaatgcaaatcaaaatatgagatagtattatacttcacacgcactgggatggctgtaatcgaaaagacagatgataacaagtattggagaggaggtgaagaatttagaaacagtctggtggttttccaaaagataaacatagagtcatcatataacctaacaattccaatcctagatatatatccaagagcaatgagaacatacgttcacacaaaagtttgtacattttaagcagcattattcataatagccaaaaagtagaaacaacccaaatattcagcaggtgatgaactgagaaactgtggtatggccataaaatgaaatattatttggccataaaaaggaatgaggttctgatacatgcctcaacttggatgaaccttgaaaacgttatgctaagtgaaaggaggtccttccttgagcccccatctgaatgaggagtccttgtacttttcatcctttgaatttatcatgcaatattatgtattgtttcatgaaatgttttacatatatatgtatatataatgtatatgtgtatatataatgtatatgtgtatatatataaatttgtagaatttctcctttccatttgattcctaaaacaataacctgtaacacctgtgctgtacttactatctaccaggcactgttatatatgctttatatattttgattcatttaaccttctcaacaactcttatgtagctgaggtacaaaatggctaagttatctaagattacacagctagtaagtagaaaagctaagatttggctcctggcagtctttttcctgaggctgagccgtggtcctgtgtatactataagggtggttctgtatctgttctttttatccctgtgtagcctgtatttagcatagcatctggcatgatgtaggtacc

SEQ ID NO: 396 - DNA, 1595 bp, Homo sapiens
cgaaagggggatgtgctgcaaggcgattaagttgggtaacgccagggttttcccagtcacgacgttgtaaaacgacggccagtgaattgcgcgcaattaaccctcactaaagggaacaaagatgtgtaactataacggtcctaaggtagcgagtcgaggtcgagctctatttaggtgacactatagaaccagaggggcggggcctccgggggaggctggaggcgatccagaccctcgcagtgcagccctcgagcggcccggaccgtccgctgcctcccgcagccgcgcgcccgccctgggcccagcctccagcccgcgagcgaaaacctgcctgttgctttgtcttcttccttcacagttttcccaccctgagaaaagtgagaggtgctttccctggtgtttcctctcaggaagggccgtgtgactggagccgcgagttgggcgccgtctcgccggggctggaggtacgcgggcttcgggacacgcacctaggcgtccctcggggtcccggcctctctgaccgggctcccccggcaccgatccgcggccggagcgtttagtcccggggacccgtcgagccctgaaagggccatttccccctgaggcgagtccccccaggcgccccgcagcctggcctcgggactgcccccaccgacagtgcgtgcgcggggagaggtcacttaaaactgcattttcgaacattagtgccccggacccgcaccttttttcctccgtcctttgatctgtgccgcagaggcaagggctggaacggcgccctcccctggtagagggacggagagaccgcaaatctttccccctgggaggtccgttaagtagatccaagcacctggaggcggaacctgtgtggccggctacagggtggtcctgtgtatgtgttaccgccttattgttccaaagaacagtctttttcaagtgacaggtagaagacgccccaataacgagaagtgactgacttggccaagcttagagatgggagtggagtcaggttccctggtggccccacgatctcgcccaccatgtcagcatgggctgcccggtgagcatagctgagatgcaccaaggccacataagtcacctactttgccttggctgccccatctgcatctatcaaaggaagccttggacacccaccagaggagccagcatgagggagtgtatgcctttatacaaattcactccaacttcagaaaaacgtccgcagctcatgctccccctgccagagcagcagtgtgagcagctgtgtaggtttggaagcaccccagtcacttgggcattgatatggtttggctgtatccccacccaaatctcatcttgaaattgtagtcccgcagttcccaggtgtgttggaggaaccttgtgggaggtgattgaattatgggggcgggtctttcctgggctgttctcgtcatagtgaatgagtctcacgagatctgaagattttaaaaatgggagcgcttgttgcacaagctctctcttggcctactgccatccgcttaagatgtgacatgctcctccttgctttacgccatgattgtgaggcctcccaaccatgtggaactgcaagtccaataaac

SEQ ID NO: 397 - DNA, 2524 bp, Homo sapiens
ggactaaagatctctcaggggaagtaaaacacattaataaagacagtctttaaaaggaagtgttccccaacctttcagcagtaaagctgtctgaacagggctaagaaaatccttcacatgaaaacagctgaggagcagaggctccaggagaagttttccagtgattgatggaacgtaaccttgaaagtctacctcgtcagttcttgagattggggttcttcaggcttcatcccggcttatttccctgctggaaatcagagatagaaacaaatgaagcatcaggttgaaaacagaagtgcaaaacagtcacaggaaacacaacccatacatcacaccagcagccggaggaaagcccctgcccaccagagatgaccaggcaacaagccctggccaggcaactctcacacctttcccagcttcgtggataattaagaaacagtggtgacctagtacctaggaactaatcagaacgtagagtaataactgtttttccaaccacggatcttggtaggagcttctcagaggtgatccttgtcacccgtcagaaatggttgcctctcattatcaagaaacagaaggagaaacaaaacgaccagtgaactcaccatccactcaggcatgtgaaaagctgaggtgccctcattaccgtcccacttctccatagtgcaggcttgggtaagatatgtcctgaagcaaaagggaaagctggtttggaaatactggggttatctgagtgccccaaagcagacccaaccctagtgagattcagaagttcacagtggctagcagaagctgcatcccttttcacagcagcctccgattcaaatcaagagcattagtcaactgcctgacaaccccttactggccggcttcaaagccttctgtgtctgatttcaaaatcagagctgatgcaaagtgctgcacccaggcactgtgcttctcacagggcactcactgttttaatgctacccaaacactctacccgcacctccccagtggggactttctgcagcaatagttggacttccttctttccctgtgtgtgaggcacttgtgaattctctcccacccttcaggacacgtcactggaggtgcggataccaagatcaataaagactgaggtcaggaaggcactcccatttctcaagagttgcttgatttggatcagtaaacaggcgtttccttgaaatcaagagctcctagggctttcaagtttaaacagctatgcagaagctccccatctgctcacaccaggggtctggcaccaaactctcgcatatctcaagttagagtcttattgcatgggaggttcggatggagcctaggaagatctttttcaaaatcagaatttcttagcagctctaagaaagttctgggacgacagctccctctgctggctccctgtgctacatacacgaaggcctcagggcccgcagctacagacacacaggtgctgattgcaaagtgacttcactgaggctagtcagaagccaccacagtgtaggtttagaggacagccagggtttccaaccaagactagtgctgcctcaaagtccctaaccaatgctggattttacacacagatctggtttcaggaagaaacaggatgccagtgcagtagccctgtacactatcaccttaaaggacctccgaagttgtttctgctttatctctccataccaactccctccttgctgcacacttcagaattttggaaataagacataactagaaaaaaggtaagctagcctatctcacctgcttagttcattcctagagtatattatccaataagagtaccacaggtttcaggcacaaaggaagcctaacaagatatgcctgctttggtccctttaaggaccaggactctttacagtgttaattcccattgaagaggatagtttctctacagctaacagtgacaatattataattatgattataggctctgtgaaaggaaaacatcttgggcccccaaaatcactaagccaaagggaaaagtcaagctggaactgcttagggcaaacctgcctccgattctattcaaaatcacccctctgctcactgagataaatgcacatctgattgcctactttgaagaggccaatcaaaaactcaaaagaatgcaaccatttgtctcttatctacccatgacctggaagccccctccctgcttccagatgtctcgcctttccaaactgaaccagtgttcatcatacatatgttgattgatgtctcatgtccccttaaaatgtataaaaccaaactgtgttctgaccaccttgggcacatgtcatcaggacctcctgaggctgtgtcatgagtgcacatcctcaatcttggcaaaataaactttctataaacttttttttttttttttgagacagagttttgctaacaagagagaaactctgcaccaaaaaaaaaaaaaaaaaaaaaaagaatgaggaggtaaaggcctaaacatgaaaggccttataagctaaggaatttgaattttattctatgtataatgggcattatt

SEQ ID NO: 398 - DNA, 2230 bp, Homo sapiens
gaggaggcagggagtgaaactgcctgaggaacagagaggagaggggaggagaggagaggagaggagaagagagcagaggagagatcagatctgtagctaggtcacatctggacaggacccaagtccaaggtaaatcgagaacaagtgatggaactaaagaatgaaggagaaatgggtggacagggcacaaattggggaatgacagcctattcagtgaggataggaaatgcacataaaggacagatgaagatgatttctctgtgtgtgattagaaggatagcaattacaaagataagagtgaaaggagagggaggaaaggggaaaagggaataagtaagaagacaaaataagcaatgagtgtctaagtaccgatctgaatgcttagaaaatcaccaggctgttaggctgagccaaaagaagagccacagccgtacccaagaggggaccatcggaaagacagaagcaggcaatttaagtagctagctactggggtaaaatgaaaaaaaagaaaaaatagaagatggaaaaggtgagaagcaaaagagggaaaacccaaaggagacactggcccagaaacctttttaactttctgtaagaatcttcaaggccccaaactaggccaagattctgactgacatttctgagctcaccagctgccgtttccttgagagaattctgacaggaaggcatagaaatgcgaaccaacactgccagctctggaaaataaataaataagtaaataactatccacatcctaaacacctcagtgggggccgaagcctgctggggaagaaagcccagcgaggaggagggacccgcctgcaacttttccccaggcttagacaaatgatgcttgagacacaccaagatacctgcacactagaggctgcagtcacctcagaagaaagatgccccctgctctggctggtcaaacggaaccaagtccgtcttcctgagaggtttggtccccttcaaccagctacagcagggctggcaatgcccagtccttggagaaacagaagagattcaactgcaactgaaattacctactaaaactttccttgagaaaacaattctggttttttcaccatattcttatgctcccaggaaggaagagaagaggagtcctggagtcaaaagtgagcaagattttaatgctcaacacacactgttctgtgcctttgcactcacagctgcagccacttacatgcttttgacagacacacagtcaagaaagagtttgaggatggagtttgtgccaaccctaagccagccaggggaagagcatgagacatagaagacagggctcctgctcactaggggctaacagtctaaggaaagaaattagacatgtatatatggtgtgatccaattgaattgcaagttatagcatggctagttccaaggcataattcttacctatcccaccaaatatgcctagcacattatgtatcatcaagtctctattgatctattcagcccaagatatctattcactaagcaatcatttctcagttccctgatctgtagaatagggataatagtacctgtcacaccaggcacagtggcttatgcctaaaatctcagcatttggggtggctaagacaggggattgcttgaggctggtcttgaactcctgacctcaagtgatccgcccgccttggcctcccaaagtgctgggattacaaatgtgagccaccgtgcccagcctatagggtatttttatattccaactattaattctactagcatgtagcatacatgcattcaccctagtacatatgactagcatataatatgctttacttttaaaaaatgctttatacatgagactgatcttaccatcttcaatatcctgtctggaaagccttcatttgtgagtgctgttgagatcacatgtcctcccaagtaggacatggagtgtgaaggccattgagtgggtgttccaaagggcagcacagaatggagtttagttgaaggtgctggggacacactacctgggctcaagtcccagctccccactctccagctgtgccacttgagcaagttgcttgacctctccaggcctgggtttcctcatctgtaaagtaggaatattgataataatatctacttcatagagtttcatgagaatacatgtaaaggcctgaaaccagtattgggtacataataatacattcttaagagtctttatttctgacaatgtacctgaaatgttcctgaaattaag

SEQ ID NO: 399 - DNA, 2253 bp, Homo sapiens
tgcttgtcttctctcctcagagaagtttgacatctccccagcacagatatttggacggagcaggctcctgaggtcttcaggatacccatcccagcctcctggccccaccctcctggtcctttcctgctgctgattctgctgcggggatgcacaggtgagcactgccttgggctagaaccctttctctctgcttgcagccctgaaatccctgcccctgggctttcagggattccagacaaacacaagtgctcccaaggtaggtcaaggactgggcattgtctgagagcagaagatcagggagctgagcactttctctgtggatctgcagctggctaccaagggatggaatcaggtgatccttcaaccagcaccataattagttacattttaaaaataatatttcatttgacaaacacagaagaatcattatttaaatccttaccagaagcagacctggttcctgtcaaatattgagcagtaggttttgggaacccttctgctgccccaaactgaggagcaggagccttgaggctggagccttgaggctccaggaggcaaaaccatctgcccaaggccacagagtgagctgagagacagcctgacagggtgtgtggctccaggctcagccctctgttcagcaccctgaggatgtgaagtctgaaacagtgcctgagttgggcaggggagcaatgtttcccggggatgttcagccactgtccttcttcaggatgctgcatactttgccctctgttccccctgcttcccactcctctgtaatctcagtgttaccacccatttttttgtgccctcttcctctttaagaaggaatttgctgctttctgggccctgtaatcctttcctagggcttgcataacaaagtactacagactgggcagctaaaattacagacatttacttccttgcacttttggaggctgaatgcaagatcaaggtgttggcagggttagtctgtactgaggtctttcttactggctcgtagatggccgtctctgctctgtgtcttattacgtgttcttccctcagtgtgtgtctgtgtcctcatctcctcttcttctgagatcgccagtcatattagattaagacccccccaatgacctcatgtaactaaatagcttcttaaagaccctgtctccaaatacagtcaaattctgaggttctggggactggggcttcaacacatgcattttgggggacacattcagcccataacaagtaccacaatctaccttggttctttcgactttaccatggagaatttattttcccttccccaggacaggcaggattggaagacctttcctggtgcagcctgggagagccctgctcacagcccacagacaggctgcctgtcctcacaccctactcaggccagggagctgaccccagaatcacagggctgggcacagcagcctccctgctccccactggcccctgctgtgagtcacctcacaggtcctagaggacaaggttctcacttttcaaattatctttcttacatatacactttacccaaatgacagtggtaagagttcagcaattgagctcttcctacaaggtagacacagttcttagtaatttgtgtattttaactctacttaactctactaatcctcacaacatccctatttgaaagctacacctattaactcctatttgaaaacaaggagtcaggcagagggaggagcttgtccattgtcccctacctcagtcagtggagcagctgggattcaagtccaggtggaccaactccagagttcattcttttagccatgttatgctacttgtagaaatattagcacatgcagggaagtaaacataggaataatcatcacccatcacaccagtcagagaagtgacactcagagttaaaacgctggtgtatatactcccaacatttgttacaaatgtgttggatgacttttatgtgccaaatattttactgagattttaacaggtttcataattttatttaatgcacaaaacatctttatatagtataaggacagaacaaacttttatatatatttgtgaatgcatgtgtatatatgtatacatatatatatgtgtgtataagtgactataagtgtacatacatgtacataagatttcacatttattgttatctttttatttgcaatgaaatgtctcagaaaagaaataaaaagaaagaaagaaagaaaagaaagaaagaaaagaaaaaaaagaaaaaaagaaaagaaaagaaaagaaaaa

SEQ ID NO: 400 - DNA, 2591 bp, Homo sapiens
gctgccatgtcctgaagatactaaggcaaccgtgtgcagaggaactaaaataccgagttcaacaacaggtctcttgctatccttcaagcaaagctatctccaaatctgaagaagagaccaagaaatagaaaaagtagcacctcttgcctgcttccctgtctaatatttgaaagagtggtgagtattgatgttattattgccttttcctttttttatattgctaccaaagccatgagtattctctttttttctttctctttttaaaaaacaaaaaatactaaactcagtttttaaaggggtttataacccttgggtcttggtcccatccaaaagcatcttcatttttattcactcggaaaatctgactttatgtggtgagcccaagaagatttgggttgaggtggtctgaggatttgggttgaggccaaaagccagtaccagggctgaatagtggcaaaaattggggccaaaacctatagcaagtgcagaatgatgggaactcccaggtgtcctgtggttctttttgttcccaggaggctctgtagcaccatttgaacttcttgttctttagaatcatccccagcttttgcagtattctcagacacagcaggaaaaacagccacgcagcttgaacttagggtgcacactgtgatgctcacatagagctgggaacactcttggcatggtgcacacaccttttaatggatgcagccgtcatccaccaagtgccaatgacgacaggcaaccagctgtcatagcttacattgccagggccttgtagaaaaggtatgacacagaaaacatcagtatccaggaagtgattacgtccattttctgtgactctgggccagattatgcagtctctgtgttcttcatgccctttgttgattctccagattctggactgggggcctcgtggtcctgttggtaggtgatactgggggaagaaacaatggtcacattgtataagctatcagttactggcctggtcaaaactctcggagaataaggctgaaatcatgggataaaatgaaatttgtggatatacaaatataatttgtcctttatgacagtactcagaaatccacaaggaaaatgttgtatgggagttgggcattgtcagtgtttaggtaggagagcctttattagggtgactggattttcttttctaccttttttttggtgatataatatacgtagcataaaatttaccatcttaactatttcaagtgcaccgttcttgggcgttcagtacatttacattgtagtgcaaccatcaccagcatccacctccagaattcttttcatctcacaaagttgaaactccatacccattaaacagctgtccattatctcctctcccgctccctggaaaccaccattctactttctgtctctatgaatttgactactctaaggacctcatgtaagtggaattgtacagtatttgcctttcggtgactggcttatttcacttagcaaaatgccttcaaggttcatccaagttatagcatgtgccaggattttcttcctttttaaggctgaataatatttcattggatgtctctatcacattttatttatccattcactcattgatggaatcttggattgcccctccttttgcctatgaacatggatatatattttctaccatttttcatgagttaaaaatcaggcttgtaattttacacattttttttctaatttacttttggatctttttgttatttcaaggacctagggtatggtctgatatgatagaattgacagtgggaatgaatcagaggcaaggagcatataaagtcagtagagccaccatacattgctaaatttgggttcaagtcctggactactatagataagaaaattacttatcaatgatctttcaataattcctaaaaaaaaaccctcccttgaatgaaatccaaatttggtttatactgagttctcattagaaaataacatttaaaagaaaaagtgacattaatattgaaagagcactgttatagaaacatttagaattttcttatatgtaacaactttcttttcttcttaaaaaattttttttttgagatggagtgtcactctttttgccaggctggagtgcagtggtgcgatctcagctcactgcaacctccacctcccgagttcaagtgattctcctgcctcatcctcccaggtagctgggattacaggtatgggccaccatgcctggctaattattgtatttttagtagagatgtggttttgccattttggctaggctggtcttgaactcctgacctaaagcaatccacccacctggcttcccaaagtgttgggattacaggcgtgagccactgcatctggccaataactttactttctatcagtctggaaatattaataaaatattgtcaacttgcaatttgtcacaaaaatcactgacatatttggaagtggtcattcaagcaggtgtgtgtagcgatggccaagaggaatcgcagagctgtgtgctcaatatggtggccactagccatatatgactaatgagacttgacaagtgggtattctgaaatgagatgtgctgtatgtgtaaaatacacctt

SEQ ID NO: 401 - DNA, 1562 bp, Homo sapiens
aacggcgcagctgggactctgtgctgtggcagcctaagcgaacctcttcacgggctaatgacaattgcaggcattgttgggctgtgtcctaaggtgaatcaacataaagaaaagccctggctgctcgtgcactgataacttcatcttttcggacatctgggaactaccgccacaatgtctggaaaaaaaatgacttcaaactaatgcagcagaggcaaaccatggtttggccaaaaatggaacagaatttacaagggaaaagggaagttcctggagtaatatgtgtaggtcagatgaggatggaactggtgaatgagcagaagagaaggttggaagagtgcagtggtgatgacagccctccgctgctccttccccagaggaaggagctccaagagattctcaactggctgaaggatacacaagatgtcttatttgtgtgtcacttgtttgtgttactaatgactcctgctgacaccacccttgcttcttcaggcaacaaatgctgtttgcagcacttgtgaaacccatttattttaacatgaaataagcttgttcctgacagattgcagcaagaaaggaggacgtttgctgaaacgtgatctaaactcacttcaagaagaaaagtacatgccgggaattgaaaccaagagctttctttccatcctggaggcccttggacctcatggaaggtgactcctttgagctttgttgcaggtcagccatagcctatgcagtgtgcaaggtgacaccttccttaggggctagcgtccctttctgctcctttcccagcctggacgtcccctgctgttcgcttcggtctttttcggcctcctccctgatgtggctttgttctgtcttctgcttaatgccacaggctttggttcctgccaccaaatgagccctcctattgttaaactctgagtctttttttaggctatgcctgcctcctctgcaccctttactcttattttcattttattgattgcctatgtggtttttctcccaaaaatctgaaagtggtatgggagtcagtctgcctagttctctcttcttccttgttttccccaagataaccgcaaagtcccttttggttttaagggtttactgttaataactacaaaaaagcccaaacctagcagtgacaataaatacagtgaagtcacaaacttaaaaaatccttcaaagtttctgtagcattggtctctaggctataattatggctccccttcaagatggtaactgaaaactatgtttgatttctgttccctgtttttatttttaaaactgaagcccactaaacccttctgtccctaacacacatacatcgtccaccctttagttagcgcatgaaccgtgtccatccctgaggaccttccacactaagtggtcttcatctgtcttgaactccacctcatggcaatgggtcctttggatataaatatttcagcaaacatttaatctctacctaagttcccatctttaggatcccaatttaatttggtccttcagcttatttacaccaagggactaataactactttctatgttacctatctagaggc

SEQ ID NO: 402 - DNA, 2350 bp, Homo sapiens
tttggatgtgagtccactgagctggcgtgtacctacaatgaacaatccttctgtactctcatatcagtctctctggtcctcagtttcctgcaacatttttggtgaccacaaaaggaccagagacagcaggtttactgtttcctttggctctgggcgctggagccctgggcctcaggagacctgtgaccccaggtaccactggaagaacttcagcccggagaggaaatcagctctcccctgacccggcgcccctacccagcagtgcaatggaacctgaaaggagctacaggacaatttcagaaacagcacccttcaggaactgcgttggctgaaaccggcagctcaggacaagtgaaccagccagcaggacccagaccatttgactcggctgatcttgtgacaagactgagttgctgctgagtacaacagccctgctctggtcactccagaggctgaccagtctacacacgggtgaagcttgaggaaacaacaagctctgctctagtcacacaccagaagctgactagtctatgcacagctgaagcttgaggacttgtcaagcaagtaaatgtagttagaaatcttaggactagtagttttcctgtaatactgttttactattgttccgtcgctgtgctcaatcttcttccccaggtaaggacctcttttgtccttgctggatatgaatatgctgtacattgctttgctgttgttaccccccttaaccatgctagaagaatcacccacagaagggtgtctccaccgtacacatactacttggtcagggaacagcataactaaaactctattgtaccatacttattatgggtgtatggggaatcgcttaggaacttgtacttatagtcaaaccacctactcagtttgtgacccaggaaataaccaactttatgtatgttatgaccccaagttttcacctggtgaatggtttgaaattcgtgcagtcaaaagaaggtctcctcttaaaccaaaccagggtccctcccttttactgagggactatttctctgtattttgatgctcgttaggtagcataccaagactcacccatctgtggtaacctacctttggaaagatactatagaaataactacaaatatgtatgcacgccaagagcaactccacattgttcccctgggaccccagagaaagattgttggtattgcatatcatggcaccctagccacaacaaaggtcaattatgcttaccaaaatgctggtggactgcaggttcctccttacatgtctacatacacacatacccagcaaccttcatatatgttatcaaaaagggaacccagacccgtctggcccagcaaggcacaaaaatgaggaacgctatctatcagaacagattagctttagactatctcctagccctagaaggagtagtatgtggaaagttcaatttaacaaattgctgcctggaaatcgatgacaatggaaaggcaattatggaaataactgcaagaatgagaaaattagcccatgttccagttcaaacttggaaagggtggtctccagattctctctttggaggcttggtttttatttttctgagggttcaagactttaataggagtggttctggccatattaggaagttgcctaatactcccttgtctcttacctctccttgttagaagcattcaatcgagtatagaggcaatagtagctaggcaaactaccactcagctaatggctctgcataaatatcaacctttgtctaaataagaaaactcgtcccttcaggcagaactaagtaatagtgatgccttctattaaacttcttttataaaaggcatcaaaggggggaaactgaggcagaaatttaaaaataaatatgcattcattcactccaagaaaagtaacagaaaaggcaagggttaaaaagaaaagaacaagttttcctctgcctagcagaggaaagtgcagcaagctcacttcaaggacagtaataagataatactgtttgagaaaccaaggccaaaggaatggactccagataccccccaacaccagagcaaggttgaaggaaaaaaaagaaagacaaattcttttactgttactcctttcccagtcttcttaagcatgattatgttttacaaatgtctgtatttagccggttgttgtttttctttcaatgcagctacaaggccaccacctatgcaaggccacaagttatgctatgctatagattatatgacctatcatttggttaactgcttttgttttatttattgtaagtccacttataaaaacccgattctgtctttgtttaatgctcagctttgtggatgtgagtccgctgagccagtgtgtacctaaaata

SEQ ID NO: 403 - DNA, 1667 bp, Homo sapiens
aaaatttatcccccagggttgaaagtaagtaccacaactagtagcttttaaaaagctttttgttacatgtttcctttgaattctctctatatattcaaataacattattattattattattatttttctcataaaggcaactataattgctgtttcattaaacctgccagctatgttttcttgtccggtgaagttgtgtcagtttaaatcacatgcattctttccacaggaaaaaaataaataaaagtttaaaagttttctaaagtgattacttaaattgttaaataaaagtaataagaaaaaaatccaatcaatattaataaacgtatgtattagtcaattatagcactgctataaagaaatacttgtggccaggcatggtggctcatgtctgtaatccaaacactttgggaggccgatgcgggcagatcacttgaggtcaggagttcaagaccagctcgaccaacatggcaaaaccccgtctctactaaaaatatgaaaaagaaaaaattaggctggtgtgctggcacatgcctgtagtctcagctaggctaaggcatgagaattgcttgaacctgggaggtagaggttgtagtgagcagagatcatgccattgtactcccttctgggcgacagagtgagattgtgtccaaagaagaaggaggagaaggagaagcccaaggagagagaaggggaaggggaaggggaaggggaaggggacacttgaaactgggtaatttataaagaaaagagatttagttggcttatggttgtgcaatctatacaggcctcaggaaacttacaatcattgtggaaggggaagcaggcacatcttacatggccacagcaggaggaagagagagaaggggaggtgctacacacttttaaacaaccagatcttatgagaactcactaactaacaggcgaacagcaaaggggaaatctgccccaatgatccaattacctccaacattagggattacaatttgacatgagatttggatggggacacaaatccaaaccatattaatgtataaatttttaaaagacaaaacttaaaatattgaaggtacttaataattttacaaactcataatattgagaaaaagcttgggctcatttacacattttaaaatatctggctggaaaaaaataaaataagttaacatttaaaatgttaattacctctatatttggcatcttgaaaaatagtatatttaaacaactgacttgcttattcatttttcattcaattggtttgttctgttgtagacactaaatttttgtaaaagaaaatattttaaaatatattaatgcaatgaggtagccctcatatactactgatgggaatgtcgaatcctttgatttatcatatcaacttttaagaatcacttgtggagcttttaattccagaccaaatgaaaaaagtgcattccaccgcattcctcttgctgactgtggctaaaaatcttgacaaaatacataaggcaaatctgaggagactcaaggaggtgaagagaaggatgccaactggcctcaggacttgagcaattagcaagtagtgagtttcctgggattttttttatgcctccagtatatctcagtctgggtgctagaaaagcttgccgcccagaaaccctaaaggatgtgcagccagtgcaacaaga

SEQ ID NO: 404 - DNA, 2143 bp, Homo sapiens
ccagtcgcccgaggcagatgggagaagactagccacagctgaggactgctgccccgagccacccttgccctcccctgcccttcagtcccctcaccggtcgccgactgcctcctcagacaccgcgcggttcccactcagccggctgccgacccccgcctcccggactgcgggactggggcgggggagcgaggaaggggctaacggcgcgcgcggcctccgagtggggggagcggggaggcgcccgagcgtccggcccctcgggatggggaggtcacgctcctgtccctctcgcccgcgtcggctagtggtcagtctggcgcgggcagtgtcaccggttgctgggaagactgggaggccagcgagcagcgtaccaaccactcagcctctccggccccagctggagccgggaagcaggcggggctaaaagagaaagcctgcggggggactccactcccgcctcccgcgcgggccaaggccccgccctctcttctggcttaaccaattcctgcctcccgctttgtctgttggcccacccctgggcctcgtcccgcccaatcagaggaaagggcgccgatccctgcgcgcgcctcagcccccagccgcggagtcccgggagctaggcggtccaccgtctagtcttccagcagctcttcccgcggccgctggcgggacacagacaggtgtacccttcggtgcttccggcgcggtagccggtgcccggcgcggacggggccgcggcagccatccccgtagttaagttagcgccttcttattaagtgaggccagactggatagctattatctctcaaccctcaccacaaccctgccaggggaaatatgagcatttatcctgtattttaggtaaggaccccacaagttaatgatacgcccaaagtcgcaatgccagggataggggttactcctggtcggcttgccagctgcctctccatgagcttcctggttgaactgcctgtccaaatgttgaccctaatgtcttctactaggaaatgctactagtattcctttccggaaatgcattttgggaacctggccatcctttagtgtgcctatatcatcagaacagacgcccagggatggccctcaaggtcctttactgctgtaatcttcctttggaggtcacttagtaccactcttctgagctctgctcagggggtcccctaattagtgtacttcgacctgccatgcttggcctggaaaactcttaccctcttctctgttttaatttgtaaaaccttagtcaaatgccatctccataaacctatgctagaatgcccctctctgtcctcagtcctggggacatatctatgctgcagcgcaacctcccgcatgttgatactgttgttatagtcagatgaatactgttcccccatagcttaagggacaggccttgtcctactcatgaacaattgatcccttgaagcatgagcagagggccagcacgccacagatgatcaatgagtgtttgctgacccgcatatgacaacacggattaaggaagacaaatctggtagcagcaggcaagaaagctgaaaagcaagacacaggatgcaggtctggagtgatcagggcctgaaccaggaagggataacattgagagagacatggaaaatgcgcttgaagtatatatatttttattgtaaagaatcatacacagataaggtagtattatatagagaatgattagttgaggatatttccagagtctttaagccagagaaatgtactaatttagacatttctggactcattgctcagtactgatgtcatgtttttaaggacactgacacacttggctaatcccagagacatgaagaggatggtgactagggtggtcacataattaaaggggctggggatatttaacctagaaagagaaacattttttgtgcacataatgactgtacacaaattctaacaaactttcatgtgaaagaggactggattagatgactttcttctgtgtagctttggagggaaaaactcaaacaatggggggaaagaacaggaagctggatttaggtttcctatgttatggaagtcatcacagtgggggatgataattgtaacaggaagatggagggtagtgaaagaaagttcaaggatgaagttatgtgtatatttg

SEQ ID NO: 405 - DNA, 2565 bp, Homo sapiens
ccacctgcacccaggtgaaataaacagttttattgctcacacaaagcctgtttggtggtctcttcacacagacgcgagtgaaatttggtgccatgacttggatcaggggacctcccttgggagatcaatcccctgtcctcctgttctttgctctgtgagaaagatccacctacgacctcaggtcctcagaccaaccagcccaagaaacatctcaccaatttcaaatccgttttcaacagctcataaggagaaggagttggaatctgcatctcatcccttcccaaaggcctcaggcctgagaattgtacttctctccagaaaccaagatggttccacatcacctttcttcgagggacagactcctcttcttgacctccgtctccatgttccctcatgacacttcactgtccttctccagcatggacactctttccatcactaccgccctggagaagctaggtcctcttctgggaagtcactccttcccaaaacctcacacgaattttctgaggagcagccccagtgcccccttctcagccaacacacccacctccattctctctgagcatccagttttggtcagcacagttctgctctcccccttccaagctgttcccaaggttatcatccagggatatgtaagtttccatctgaaatgcatcattcctcctctcctaggcagtcaaatctcatttccaggcccacatcatctgattaatttcttaaacatcagaataatcattaacaatgatacatcagttttctagattccatcttaaccaaaacaacacacaaaaaaattacaaacaacttctgggaagaaattcttttgccaataggaccaaaatgcttaaactattctctccaggataatttttattcattcttaacactcagctcaggtgtcacctcctctgtggagcctgcagcaaattccttgagacagagttagtcatttcattttctgggcttcacaagatcattgtagcatttaacacgttgtattataattacctgctaggaagtctgcctttcccactgggccatgaactccacaagagcgagaactgtgtcttattcatctttcaatcctccatttcaattcaattcagcaactattcaacaccaagcactgtgtcaatttcaaagttagtacttaataaatatttcaggagagaatgaaagagtgaagggagataagtttacattatcttaaaaattctttcaaaaaagttctggcagaaacttccttttttatgtctacttccaaggaaaaagtgttttcccaaaatccatatttagatagggaccctcaagatttgtttttccagtttgttttatgtttataaacttaaatttaagtgataaatatcttgaattatatggcatagtacaagtgaccttggaggcaatctcatccaaggagacagctaatatatggcactcatggcctgctctccacctcctgcatccatagcagacagcacgaatggatcatggtgtacttccttattaagcccatactcagcctagaattctcaactcagcacttaacaagctgctttctggagctggtagtcaagatgaatcctgcttgccatccttgatctagtttattctctcttattgcatataagcagagtgaggtccagaaaggttaaatgaatatgcttagattccaaagctaattattagagcctgaactagacccaggtcacctaagcctctttccaccagaatacactttaattttatagctataggattgaatacttcatagaaaatgttagttgtggtcatcacgttatatgtatatatgtatatatttatgtatatacaatgatgaaaaaaccgacatttttacactatatacatatgtatgtgtatatctatggacaaaaccaatattttctatgaattgtttattcctattaatcagctatagaatatgtatgttacataatatataatatagccacataacatttgtgtgtgttatatattacattacatattataatatctatgtttttcagtcaaaagtgaggtagctctaaaagtgtgccacaaaaaaacagtaaaacccaaaaaaatacaatgaaaaacaagcattggtcaatttaactctttgatatttttcttatagcttcttagcatatgcaagacctatttggaaaaaaaatagctgtggaaattagaatttaaggctggttatgtatattattttagtcaagggttatggagttagtaacatatactaattttcactgtgtaaattcgctcatcctcctccatgtgatgacactgccaccaatacaagggggaatcagagaggatcccggaactgtcttaatgttactgtttaggcaactccaatgctagaagtatctctgtttatcacacacaaatgtgtaaaagaatgtatgttagagaaaacaagaaaatattaagaagtgagtacaaaccattgttttctttttcagagcatgagtttaaaatcctccaaagggatagggttgtttcttccaagtgggtcagcaggggtatgagaaagaaacagaaaaa

SEQ ID NO: 406 - DNA, 2234 bp, Homo sapiens
gagctcatgaccagaggatctgtcttatgcaaaggtcagagaaggcttccctgataaaggaaggtcatccaagatgtgcaaagagtagcaaaaaatttcggtcgccaggcataaacatttcccacagagactgaactggggcagtctgtcttcttgtttcagcactcataaggtgttacaccacagtgaacttttagtgctggcttccgtctcagcatctgcttcccagagaaatcaatgagaatgatctcttatcctatcaaggatcattttacctccttctcaccaaatgttacacatcagtgagcagcactgcctaggagataacaggaagccgggtttcacagcagccatcctaaccaactaattctgtgatgattgagagactgtgagtgacccgattggccagacccttactgaacccaactgacaactgtgtcctgattaaaggggaatgaaatttgttttcacaatcccatatagacctataaacttgtcctgacctgtcaagagtgccagttttcaaactgccattagcaaaccactgtgaaaccacccacaaaagccttacattttaggcttttcattttatgtttactatgaaacattaaagaatgcatgaatatagaaatggaaattggtcccttttcaccgatcacaaactttgtacctcaattcttaaagtcagggatgtttaactcagccagccacctagctagggtctgtaacaaacagttggttatttttcaccctttgtctactcaaaattaagaaagtaaagacgataaaaacaatattctacagaaaaaaaagacaaatgttaaaaaagtaataaaagagaactttctttcatattcatgtgagaaggatagatgtttagaaataaagaataaaatttgtgtgagggatgcctcttgcattttaagaattagaagaggtgattggtaaaactgtagacatggacgagctggttttccaaggcacgggatcagctacaagtacatagaaaaggtccctcatataaaaacaattatgtcaaggtgaatttgtgtgtattatatatgtactaaatgatgtgtacatgcaactggttgttacaggaaaaaggaaaaacagaaaaaaactgaaaaaattacatgaaaaaaatgtatcaagaacccacagtagttgtaaatagcatgatactgttattgagtacctgaaatcaattgcagattgacatgaggaggagctcaatgtaacttctcccagagatgatcacagaacttaaagaggctgaattcatagaacagcttctgtgtagaagtcactctatagttcctcccagaggaggggactgacttttcaatttgtccattgggccatgctcggaccacagtacttgaaacagaaccttaggaaaatgcatcatattttaattctgaaccaagagttggctcatttttaaagataccatgcttagttttcatttattcagatttaggtaaagggttcaatattcaggtcaggatttgaaatgggcatcaggaaggtgcccttaggtgaggaagatttgtgatgtgggcaacttcttgctaataaaagccttcagttcaacctgtactggttcaagaacttctgaagttttacagaggcttgcctggaatctgggatgagggcctggggttgaggtcaggcagcttcgtgaacggagaagtgttgtgctaatggggttgcttgagggaggacaatgatatccaccttttttcaatattttgcctaaggccaccctggccctgccattcaaaatcaccttttctgtcattttggcgtttgtggtacattgcagaaccctgacttaggccaggaggccatttaggaaattattcctgacattttgttttaaattaataaaatacataaaatagggttccttgtaatagatctcacacatcaagcataagtagatcgcattatttatttttaaatgacatatgctgtagctcagttttgtagaattcaagaatatacttcatcttttgtgtaggtaatttcattcaatttcccagtcccctctttcattttggagaaataaaatttgtgttttatgtctatatacactctccaatggggcaattcaaaaataaaagtgcatccttaacactgcatttctttgcttttggcctgcactagaacttttccctcatttcaatgtagcttttgtatgggtgaatgcaataaaaaaggtaataaac

SEQ ID NO: 407 - DNA, 2497 bp, Homo sapiens
gggcggaggcaagtgctggaagggtcgcagaggggccggggctgggctggggaggcgaggctcgcttacccgccatggggctgctcccgcaggctggtctcgccggtctgggcggctggggctgggctgccaggccgtctcttaaagcgccgcggggcgccgtcgggcgagcacaggggcggtccgtggtgctgctggtgcccagctgcggcgcggggagccgcgcagtgcacggcaggggcagggacccagcgccccgaggccgcagtcccgccccagcccgtccctggcccaggcccaggcccggcggtggaggggcgcagagccgaccgtgagctcccggaaaccgcagggctccagccagggcgggtcccgggcgccaggggcgccaggggctccctgcggcttggggcagggtggggtgagagtaccacccccatcccggagagcagggctacagccttcaccgccgcgtctgacacagggctccgcacccgacccctcacacctggtggccaggactgagcccccgccctcccagatcctcctcaagccgaggcccctccagccgcagctccgaccccggctcgggggagccttcccggatcccggggtggagtctccgcgcgggtccccggccccagcgctgcctgcctgatggccttgggcagggatgcggcgctcgcgcacccgttcgctctcagcggacaaaccggtcgagagcgcaattctggagcgggttccagaagctcctgggttccaattcaggctcctcactcgccagccctgcgatttgggacaacttaacgttcgtaagctgggttcagtcttctcaattgggttaatgtccgtgaggggctctcgtaagggcggctggcctgccggtaacttaagaagcatgtaaagagcacttagagcggaggagcggcgcggcccagcccctgttacggtggttatcggatggaggccagcgtcgcctgggtcccctctgtacacccagcgcctattattatggtgattattagaacgcagggcccgtgcccactagtccaggctggcgaacgcgccttgcgggaaaggcgggagtaaggtcgctgcgtgtcctctctggtcacacctcggcgcgccctggtcgcgcccctatggttcccccgccgaggcctcgaggcgtctgagtggggagcgaaaggaatgtgtgtatctgtggtgcaggggagtgtgtgtgtgtggggggggggagggggtggttgtgggggagtgcgtgttttggggggatggaccttctcgttttacagccttgaaggttatgtccagagtcatagcctgggtcccacacccccgcgtcacctgtgcctgtcctagggggcagtcagtgtgcgtgaggctccaccctgcctgggctttggctcacctaaggcgaggagggtggtgaactggggtgccccactgggccgggtctgggagccctcagcgccagggtgcagaggccgcagccactccccttctagggtctgggcggtgggggcatgagaaggcggagacccaggccacttcacccagtaactgtcctcagctgtgcgcttaactggcgagatctccgctctacttggagacagtctggggtgtctggagaatcctcccccacttctgcttaccccaggtcgctgtgggagcaggtgtctcaggaaatggggcctgggtcccacccaaggcctcgggtctgagttagatgtctgtatgtttgttgttgcccctgcaacaaagtaccacagattgagtagtttaagccacatgcatgttatctcacagttcggggggtcgggaatctaaactgggttggcagggcctgtgccttctggaggcaagggagctgtttcccagcgtttccagctcctagaggctgctagctagtggccctttcctttgtcttcaaagccggcagcacagccttttctaacctctcccactccaggtcatttcacatcaactctttctgcctccctcttgtaagtgtgataccctaccttgttttaacctgaattgactgtcccttagctgagagagccagacacactccattttggctccttcacttgcagccccttacccaccccccttcctcaaggacttaacttgtgcaagctgactcccagcacatcaaagaatgcaattaatggataagatactgtggcaagctatatccgcagttcccaggaatttgcccagttgatagtaccctaagcccccacatttgtgtccggttgatggtacccaaagcccccgcatctatcgccttgtgatggatttaaaacccctgcacctggaactgtttgttttcctgtaaccatttgtcttttaactttttgcctgttttgcttctgtaagagtgcttcagctaggctcctcctcccctttctaaaccaaagtataaaagaaaatctagccctttcttcagggccgagagaattttgagcactagccgtctctcggttgccgg

SEQ ID NO: 408 - DNA, 2851 bp, Homo sapiens
ggcgcagggactggacgcatacggacccccgggagccccggaactcgcgttcccacgccagacctcatccccttctgacagagacgccctcgtcccaccgtcgccgcaggcgccaggggtccctccccaccgccgaggtctagccgccgcccgcccgccttcagggcagcgcggggaccccctggccctcgcccagtgctccggggctcacccgccgctcctcgccgcctccggaggcgagggattgtttcccacaagggactggagtccagggaaggactcgcctggagcgggaggcggggatgcacatgcgcagagggagaggccgagcgcccgagttaggctggtggatgcgatgcggggcttggaagcggcgctagacggtgcctgtcagctctggccgcaaccctcagttgtaaaatgaggctgatagtgcctacctagcaggggttttatgagaattggaggcgcttacgttgtaaaagcatttaaaccggtgtctgacacataaattgttcagtatatttctcacaattatatatcatgctggtcatcataaggctctcctgcaaaacagccttgtatagtgggttgagagattactgtctcagttacccactcctgcttggaaccaggattctgtcagtgtccccagaggttgtggatcaaatcaatgtgcctgttagacacaagccaagagaaggaaaggtgtgcttgaattcattcacgatcatgcatggtaacgtttaactgaaagactggaagagaaaactaaaatctgtgaccagtatggttattctctggaataaggaaaaacagaagctagcaattcaagacccaaacataaaattgtttatatgtttaaaaaaaaaaaagccgtttgagtaacaaatttgaagaagaaagcacgcactcatttcccaaaaccaaccatactttacccagaactacactacacgtacattcttttacgtgcacatctggcattttcagcacactatgagcatgtgcgtgtgttacaacttttcatttaaaaaatcaactcctgggtcattgtccacttttttaaacttttcatttgctcataatttcagatttacagaagagttgcaaaatagttcctatatgcctttcacccagattccactagtgttagtattttacatgaccatggtatatttatcaaaactaagaaaataacataggtacaagtactattaactgaactgcagagtttattcagacttaagcagttcttccactaatgccctttttctgttccagcaaccaatcaaggatattgtcaccgggggtccttgctcccagagctcccaagatggtggtgggctgcttccgagatgggggcaagcctcatgttctctgacctggggttcttggcctcatggattacaaggaatggaattttgggctatgtggtgagtgttatagctcaattagaagccgtgggtcacggaagagaactgtggaacccagtgactagtgttcagctcgattaggaccaacctgggacttagctgtgcaggaacaatggcaagcctttagcccaacttgggagcagcaatgggcacctcgctggatcaggagcacagcggacaccctgccagatccggagggatggaagtcagcggcgggtctgcgaaggccgcaaacagcagcggtggacagcgagcgaaagctcagctcaagccgtaacaaacacggaccagaagagagtgcagttgcaagatttaatagaatgaaaacacagctcccatgcaaagggaggggacccaaagtgggtagctgttgccggctccaatgcctgggtttctatcccgatcattgtccctcccgctgtgctcttaggcaatagatgattggctatttctttacctactgtttttgcctaattagcattttagttagttctctttactacctgattggtcggatgtgagctaagtgcaagccccttgtttaaatgtggatgcagtcaacttctcagctaagcttagggattcttagtcggcctaggaaatccagctagtcctgtctctcagtatcacagtacatttacttgtcatgtcttagccttggccagtttgtaatagtttcttgctcctaccttgtctttcatgaccttgctatgaagatgaaagcacgctctcttgacttcagtcctcccaccttggcttcccaaagtgcttgggattacaggcatgagcaaccacacccagcctatagtttaactttaaaacaagaacgataatgatcccttcctgaaactaaccccctccttgtttggggacaaaaaccgtttttgtaaaactagtggaagtccacaagtttagaattttggtaggaacctgaattctgtaagatgcaggcataaactctaaccagtttttgcttttataatctgcctttttgtaactgcttactactccagagtcacatagccagtggtcacaagatttataacatctccaattgtcactatagataatgtcactattgtaaaacctaaaactagtgtttgagttatttttcagactttacattctgatggaccaactggtgccactgggaccagtaacccatagcaaaaaatggactcagttgatcctgtgacccccttcaagaaccgattcaacacaagaagacagtttcaacagccctatgatttcatccctgacccagccaatcaccattccccattctctagcctcctgatgggcaggaggctagagaggttcaaagtttttcatccttgaaagactgtagcctccaaattctcaggaaggcagatttgagaattatctcttatacttgcttggccaccctgcaatta

SEQ ID NO: 409 - DNA, 2585 bp, Homo sapiens
accatcttgggaactctaagaacaaagacccgccggtaacatttggtggcccgtacggcgcttctccaaagcgatacatcctgggaaggaccctacccagtcattttatctaccccaaccatggttaaagtggctggagtggagtcttggatacatcacgctcaagtcaaaccctggatactgccaaaggagcccgaaaatccaggagacaatgctgggtattcctgtgaacctctagaggatctgcacctgctcttcaagcgacaaccatgaggaaagtaactagaatcgtggatccccatggccctcctttgtcacatttttctttttactgttctcttaccccctttcactctcacttcacttcctccatgctgctgtactaccagtagctcctcttaccaagaggttctatggagaatgtggcttcccagaaatattgatgtcccatcgtataggggtttttctaaaggagaccccactttcaccacccacaaccatatacccctgcacttcaggccatacatttcaatccctgtatctttaacctccttgttaagtatgtctcttccagaatcgaagttgtaaaactacaaatggttcttcaaatggagccccagatgcagtccatgactaagatctactgcagacccctggaccagcctgctagcccatgctctgatgttaatgacatcaaaggcacccctcctgaggaaatctcaactgcacgacccctactacgccccaattcagcaggaagcagttaagagcggtcgttggccaacctccccaacatcacttgagttttcctgttgagagtggggactgagaggcagtactagctggatttcctaggctgactaagaattcctaagcctagctggggaaggggaccgcacctacctttaaacacggggcttgtaactcacctcacacccaaccaattaggtagtaaagaggactcactaaaataccaattaggctaaaagcaggaggtaaagaaatagtcaaatcatatattgcctgagagcgtgggggagggacaatattcgggatataaaccctaggcattcgagacgggagtgggcaaccccctttgggtcccctcccattgtatgggagctctgttttcactctactaaatcttgcagctgcacactcttctggtccgtgtttgttacggctcaagctgagctttcgcttgccgtccaccactgccatttgcagacccgccactgacttccacccctccggatccagcagggtgtccgctgtgctcctgatccagcgaggcgccctttgccgctctggatccagctagaggcttgtcattgttcccacatggctaagtgctcgggttcgtcctaattgagctgaacactagtcactgggttccacagttctcttccatgacccacggcttctaatagagctatagcactcaccacagggcccaaggttccattccttggaatccgtgaggccaagaaccccaggtcagagaacaaaagtcttgccgccatcttgggagcagcccaccccatcttgggagtggcctgccaccatcttgggagctctaagaacaaagacctgctggtaacagctggataagcccaactgctttgtagctgcctgtgtgtggcaaacaactaacactccttaggcctcaattttttaatttataaaatgaaagcagtaaatatcacatcatataagttgttgagtatttatgagaattaaaaggaataatcgccataaaaggcctgatactggcaataatatatttgacaaatatgagtttctctttttccctttattctactttacggcaactcctacctcctattctctttcttttgctactaagcagacatctgggtttgtttgtttgtttcatcagggagaggttctaaacgttgtatcctttaaccctcaaagcatcatctattctcttagagggtaaagggcagagatgttatccctgttggtttctaaccttggagaaggtcagaaatgtcacgagctctctatagatcctcttatcagcttctcttacacaatcctttggttctgtgtgtggtgaaaactggattgggggaaaatcagatcagtagaaaccaaatgttgaagcaactggtgaatagagaaatacagattgcgtatccctacaatggaatgttattcagcaatagaaagaaatgaagtatgcacccatgcatggaatgatgcacggatgtttactacagcatagaaggaccttgaaaacacagggctaagtgagaggagccggacaggaaggatcatgtattgtatggtctgctcatgtatgatctgctcacatattgtgtgatctgcttacatgaaatgcccaaggtaggcaaatccacagtcaggaaatagattggtggtggccaggggcagggacttttgggggaaaatagggagtgactgttaacagggacctgctttcttctggggtaatgaaaatggtcaaacattgattgtagtgagggttgtgcggctctgtgaatatgcaaaaaacccattgaattggacactttaaatgggtgcaaattatattccaa

SEQ ID NO: 410 - DNA, 1668 bp, Homo sapiens
gaaaggtaaactggtgggggcgatggggttcagatattctctgagaccgggaacgtcactgctctccccgggaagctgctttaggcacagtgctcctgcctgactcgggaagactgctctcctggcctcctggaagtcatatgggaaacagcagatacccagtttttcagctccattaactgaaagactcttttcctgaatgttgtccttgcatcattgcacaaaatcagcttaccataaagaggatttactgaaatcctgaaacttaggagtcagcaagtttttcatttgtgtttgtcaaatgtcaagaaaagctgaggagttacaagaaactcagtgatccctgacagctctctgcaattgcagcaaaatctgagcaagactgacagaaaccactcggggacaaacatctgacttccacagcagttaaaatcattgaatacagaaccaaaatgtcttcatgtgaaaaactccatcaagacccaagaaaacttctgactcattagtagtttagggtacatcatgtggatgcttttgaaaccccattctttgaagtcttccccacttaacatatatctaaaaaccatacatacattcattagatataatcttagttaccagttttaaaggaaataaaagcatatagaacacaatagttacatctccaaaataaagaagcttgtaagatgtgtaaaagttaagaattcattttcactaacaattattctcaaacttaaatgaggatactgaggacatgaaaattacagagctagtatcatgatgctataaaatactgatatatttaatatcccaacagggactgattagacttatatatttaaaacatatttaagttgcttagaaattttaagtcaataaaattcacatacaataatatacattataaaatatgaatttaaagctttggaatttactgatcagacatttccaatagctctttgaaatgttcccatacatatattttgttgtttgcttgatggcaaagtttaaaaatgttgagtaaaaatattagaacttgtgaaaccctgtttgtatacacccaaacacacttacatatctatctatatctatctatagaaatatattgtgtgtgtgtgtgtgtgtgtgtgtgtatgtatttgtgtgtatgtgtttctctgtctgtcttagttcatttgagctgctataacacaatgccatagactgggtgggttattaacaatagaaatctatttctcagttctggaggctgggaagtccatgatcaaggcctcagattatgtgtctggtgaggtcttgtttcctgatcctaggtgatgattacttgctgggctttcacatggtggatgtacaaaaaggctcccttgggcctctcttagaaaggcattaatctcattcatcaggctctagcttctggacctaatcaccttggagaggccccatgtcctaacaccatcaccttggattaggattgcaacgtatgaatttcagtgggacacaaacatttaagaccataccagcctccttcttcccttttctacccccaacaccatgcccatccttccaaatttgttagtagttttcactcacagtttattttatttttttcttcatccattttccttagactgaatacatctttatgggtctctcctttatgctaataaa

SEQ ID NO: 411 - DNA, 2582 bp, Homo sapiens
aattgatctgattactcaggacttttcttagccaggactgggggcagagaggggctgcagttgcttttcattctatttaatttctgttttccaagaatgaatatcaagggagcagctttgattaaacaagcctttaaaaagggggagaaaatgccaggttttacagagagccttgctgagtcgctggcaaatggccacatgacacaatatttttagtctttggatattatctcctctctaagtacaaaactgctattcccattaacacagctcttaggttcagagaaaaaagaaattagcctggtgttaaaaagaaaaaaaaaatcaagattccaggaaatcatcctcttcatagaaattaaaaagaaaattctcatgtgcctagttctacagttctccacatcctttttatctgctgctgtttttcttttttcctttctttcctttctgcataagttgttgagactgtgggcccaactggagaagattttaaagccaagtctagcattattacattcaaatatttttaaaaaatgttttacaggatggttttttgttgttgcttttttgttttaagattctctaaggtgggaatgttggtcttactaagatgggaaatgaacaagaagaaaaccccaatatttaacatcgaacaaatatttattgagcatctattatgtgctagtcactaaggatatagcagtgaataaaatgaagccctttccctcatggatcccttctaatgggacagcagtagttaacatttattgagggcttgccatgtaccaggcattgttctagcactttatatgataacccgtggggcatgccctagtattattactctttgcaggcactcacatggctacaaagtgatgaaccagggttcacatccaggtattctggctccagagtcaccaaacttaatttattaccctaatttgtgactcttgtattccagatgagtctaagagtccttcttctcagttcccgtcatatcctgtatgtatcccctttggagcacctccccaaacctttgttccctcggttgaccataagattttggagtgtagggcctgtctgcattctctgtatcccaaggccttacagtgcttgggaccaaccagaattggagatccagctcctagagcatttcaaaacacatgtggaaggacgccctgattcaggtacatacagtaaatctcctccccaacccatgagtctctaaaaccctgtcattctatcatatgtagcttaaggtgctgatttatggatatgcgaggattctctgcagggaaggctctgctcaaagacagaggactgctctgtgtgtgggcaacttcaggcctaggcaattgctgtcaggccgtgagctggagtgttaatggggggcttgggatttaaaccccacacctgggagtactgccaaggtgcttgcacaatctgtggggctgggagaagggtgctctgtggcatcacccaaatatttccccttaaaaatgagcagaaaacacctaaatgatcagcaagaaagatttagtaaaataaacccctgtggtaggatgctgtgccatcgttaactaaccaggctataaaataagacttaagaataggggtaaatgctcaagatataaacacaatgaacagatattataattttgatgataacatcattgctatttttggagggtatacgtttgcatagaaaagaacagaaggctaatcaccaaaatgagatttaataggtggcatatttcatttcttttttatacttttctgtcttcctcagttttgtataaaatatggtcaatacaactagaaaaacattattaaataataaacaaaaggaaactttaaaaatggcttaaaaacgttatattggcctcaaaggagacctttaatctattttctgtgcacactgtcttcttgtgctgggagctcagcagcgatcataacacagcaaatcgctgggttggaaaccttggcaggtgtttcctttgattctagacttcaggggctgtggctgtggttgcaaaacctccactgatgatctgatccagagatctaaggccttgaggatagaacatccctcatcctagcaggaaataatgaaaccttctaaccttaaagccctttggaagaaatgaatataaacatgtgagtctctagcgttgctgcgatccttcatattttacggcacgcattctcatttaatcctcaataacatgaaggagcaattggtcacatcagccctaagttataggcaagaaaggcacttgtcccagatcttgtggtgaggaccagagcttaagctagacgcagtggctcatgcctgtaatcccagcactcagggaggcagaggcaggtggatcacttgagctcaggagttcaagcccagcctgggcaacatggtgaaaccctgtctctacaaaaaaaaatataaaaaaaattagccaggtgtggtggcacacacctgtagtcccagccactcgtgaggatgagatgcaagtatcacttgagcccgggaggcggaggctgcagtgagccatgatagcaccactgcactgcagcctgggtgacag

SEQ ID NO: 412 - DNA, 1554 bp, Homo sapiens
ccgccgccgctgctgctccatggccgcgcccgccccgcgcccccgccgcgtcccgtcgccgcccgcccgcgtccccggtgagcgctggagtggggtcgggcccgtgtgcctgcctcccgcgccctctccgaatcccggactcctagtgaaaggaagggggagggcgcgggcgcggaagggaggtggatcggagcggcagcgcggcccagccgaacgggtaccgaccgtcaccccgcgatcttcctcgcagggggcgccggcgcccaagctgctcgcccggagggcccagagccgcttcccagaccagagccgggggaggcgcagccagtagcgagccctgagcgacctctctccccactcgcccccctgccccagtgaatttctgtcagttgaagggagcggggctcttgcccggggaaaaggaaaacagttcgtggcctgggatctgggaccccttgccgcccccagcgcccctcccgctccccggcgcccttgcggcccagtggctcctgcgcgccggagtccgccgcgaagactgagcggccgcggggatcgccgcgggctccgcgcgtattcctgggctcaacactgccactatctgacacaacaggagcagtgtcgggggggtactgcgcgcccagacgtgagctgtgataggctctcgaaactgggcgcattttttttttaacctggtaattatatttcactttcagggtttttttaaattattattattattttattgttgttgttaagcgacttgtatgcattcagatctcatgtgcattttctcgttattcaagagaattggacacttgatgaagggaaggacacggagagatgacaaacgcagactggcatgcagttgttttcagggtgggaagaacaaagtggaatgtcttcagttcttacaaaatatggtggcatttgtaatctctgagtttttgtatttggcctacaaagggggtcttttcagggaaacacgaatggaacccttcagagacatgtctacattgtaaaacaatataggatagatacggattcgggaaggtctgcaaatggttccgctcaaagaacttggggtaaagttgaccgaggactcggattctgagaggactcctatcacgttcttcatgagagttttaggctatttggactgaggaatgatttggaggtacgtcgggggcattccctcactgaggccatcagtggctgcctccctgcattggctgctggtttgagttccttcacagaacttcttcgtcaagtttttcataggaacgctgcagtgaagtggagtgaaagagactggggattgaagggtgaaccccttccagccccaatcccatcccgattcctgtctggatgcagattcagtctgactcagaacggatagttcacccttttactgatccaagtgctctgtcctctttttttcagaggaatgttctgactggctacaaacttttcttctgtcactgcaggacctcccgtggagcaattttcgataggtcttctactatgcttttttaccgcgtcattgttattgctgag

SEQ ID NO: 413 - DNA, 2703 bp, Homo sapiens
caataggccagtgacagaaacactggtacaaagcccctcgctcagtttggctgggagaaaagcagccgaaagagtcagcatgagtgtgaggaagaaagaagaaaagaaggtgccttagaaagaagacaaagggcaactccagtctaagagtctcaaccaccgcatagaaaaatactttttggggcaacaaaatgggttgggagaaagaagtatttgacaggagtggaaggcgaggggcagctcttctgtgtcctaggcacatctccctaccctggagaacctgtcctgcttgtggcctgggaagagacttcaaatggcgccaccaacaacctgaatcaataattacctaaggcggcgtcctacacaggacaaagaaaggcagcgtttccgagagtccctgggataaggggttgcttctcccagctgcgcatctacatttttaatgttgtgatttactccgagggaagctttccacaagtaactaaaatcctaggatcccagagctgccttcttctctggtcaccagctaaagtaggggtcaaataaatatttccagatttttcttaaaaaaaaaaaggccatatttcaggtgggaaagaaaggttggtggctccgtgtgacacaaaactacaacctttggaatgcctgtgcctttggggaaggaaccaaaccaaaccaactcgcagaatacctgctgactgaagacagaaattccccagtgctctcttcttcatcagggaactatcttccaatggcctttctccaattcttcctctccaagcccatggcaagggccaaatgggaaaaaagcgaaaccctaaaaggcgagcctatcatttatcccaaagcccaaagaacaaaggctatgtgggtacagtggatttgctccagaagccccggcctctgtcctcgtgtttctgaaaaccatcctttctggcaaggaaagaaagtctcagcgctttggatgaagctctctgttaggccaccactgcttctcacgccttcgactgctggtgcaggaagattccaggccactgggcttcaccggccctcaatgctcacttgtccagcccacagcttgacaaactggtcacttccaccccacctgctggagctgactagggcaagtaaggctgatgggtgaccctgatcggccctcactggagtacagggtttccttctccaatctcctcaaaacaggtgaacaaagttctcaggccaagttgcctccagcacctccccagtctcagacaagtccatggaccatctctcctgctccatgcaggaggattctccaaccacagtgccagttctacaggtgttatcacccagctaggaaggattttgagaagagcagctctgtcctatgggctgtgggttcacatctccactctccacccagacagcccagaaggaaggctgtctccctttgtcgggctgaggtctgaagagtgctgaggccccgacacacaccgcgttcccaatggtcagggctgcttgggtgatcaggccctggtgatgcgttctgggctcctgccttcctctgcggggtgactcccggctggtgctcacagcacacgcctagagatgccctgagaacactgaggagcacacaaggggcagcacatgcctctggaacctttagttcagccttgtgtggagggagacaccttaatgaatgagaccctttgggaagagtcactgccacggtgtagccaggctgacagaagccgtcctgtagaattgggtgaagggtccatttacacagcaaactggaatgttcctaaagactgaagaggaaaacgctgtatttatttagctggtgtcaggccaactctacttctatttccaaattcgtctttcctctctcatttcttcatcttctgtacttaggacttggggcttggggatgggggacgcagtggctggaggctagactttcctctttgaaaggtctgacttcagctcccgtgactattgctaaaccgcggcagccacactcgggccgctgccttgctggttgtcaccccgatacgcctggcctctgggtaggcagacgcttcactctgcagcatggatgggctcaaacccccagaagccaaacagccctcccgaagtcctttcttatttcgtaattctatctacgttttctttatcatttataaagcagatgttttggatgatatgtttccttttgttcattttgtactgggttgaatatcatcccccataatttcatgtccacccaaaacctcagaacgtgaccttatttggaaatagggtcatgtagacatgattagctcaggtgaggtcatattagatagagtgagccctcattccaatgactggcgtcctcaccagaaggccacgcaaagactcagagacaggaagggaggccacgtggccacacaggcagagactggtgtgatgcagccaccagccaagaacggcggcagcagcaggagctggaagaggcaaggaagggtctgtccccggagcctgcagaggaagtgtggccctgctgacactttgactttggacttctgacctccagggatgtgagagggtaaattcctgttgtttcaagacacccagcttgtggtaccatgttacagcagccccaggaggctaatataatcctttcatttttgttttgtgttatgtttagacttatgaatataaaacaaatacaataaaaaaata

SEQ ID NO: 414 - DNA, 1825 bp, Homo sapiens
accctgacaggggacttttccacctggcagtgccaatatccggctcctacgcattctccatggaggatcaccagtccagtgccctttacacggtgaaaaacgtgaatacaagccatcagtagaaaaagaagaccctctgaaacaggaatttccaagacagtgcattgaggaagtccaaggaggaaaggatggggaatagaagacagcagcgaacaggactggggactgtcacaggagccagagaaagggctgattctaagaggaaggagacaccaaaaatgtaaaagggtgcaaaaatactggagagaggaggcctttatgatggccactgggtgggggcattggcagccaccaaggcctctgagagagcagaaaatgtggttccaccaaacactcttctggaacctgctcttttttttgtaaggtgagctgagcacttagaaggatcttcctaaaatatccccttatgaaagatttctaataaccctttctctctgtcatcttcattaaccactaattttaaatttttaaatttttaatttttattgtggtaagagaaacacataacatgaagttgccctcatactagatgtttgcaacatggcatagtattgctaattacatacatggggctgtacagcacgcctccagggctcttccatctcacgtggctgaaactctccacgttcctcccccaacccctggcaaccactattctagtctctgtgtctataagtttgactgttttagagacctcatataggtagactcatacagtatttgtcattttgtgactggcttatttcacttagtataatgtcctcaaggttaatctgtattgtagcatgtgacagggtgccccttttaaaaggtggaatgatattccattatttgtgtatacactttctttatccattcacttcccagtggccatttagattgcctccacctcttggccattgcatataatgctgcaatgaacacagtgtagatacttcttggagatcctgttttcatttttttttctgatgaatacttagaagtgggattgctggagcatatggcagttctgtttttaaggctttgaggaacctccatgctgttttccatagtgggtgaaccattttacattccacagcaggtgcaccattttatattgcatagtgggtgcaccattttagttcctgccaacagtgcacagtggcttcaatatctcctcctcctcaccaacacttgctaccttctgtttcttgataatggccatcttagtaggtgtgaggcttatcttacaccatacacaaaaataaactcaaaatggattaaaggcgtagcataagacctgaaagtataaaacttctagaagaaaacaggagaaaaactctgttgcattggtcttggtagtgacttcttggataagtcctaaaagcacaggcagtgaaagcaaaaagagacaagcaggattgcatcaaacccaaaagcctctgtgcagcaaaggaggcaacgagcagagtgaaaaggtgacctatggacttggagaaagtgtctgcaaaccatgtgatatggtttggctgtgtccccacccaaatctcatcttgaattcccacctgttgtgggagggatctgttgggaggtaattgaatcatgggggtgggtctttctgtgctattcttgtgatagtgaatgagtctcatgagatctgatggttttataaaaatgggagtttccctgcacaagctctcttctcttgtctgctgccatgtgagatgtgcctttcaccttccaccatgattgtgaggcctccccagccatgtggaactgtaagtcc

SEQ ID NO: 415 - DNA, 2315 bp, Homo sapiens
gcaatactgtttggaggttcagacgaggtgtccatttatattgcccgacaatgatgaagtcatctacggaggcctctccagtttcatctgtacagggctttatgaaacctttctaaccaatgatgaaccagaatgctgtgacgtcaggagagaagaaaaatcaaataacccatccaaagggaccgtagagaaaagtggctcctgtcacaggacatcgctcacagtgtcatcagcaacaagactgtgcaacagcagactcaagctgtgtgttcttgtactgattctcttacacacagtgctcacagcctcggcagcacagaacacagccggactgagctttggaggcatcaacacgctggaagaaaactcaaccaatgaggagtaacggaaggacgagtgtcaccacagcagcagctggcccgccgtgaaaaatggcaactgctgtctcatgtaacagaaactgggtgcttttaccctcgaattacttattgcaaggcctttagggtaaaatttaaacagatgggcctgaatccaaacaaggacacaaccacagctttttattgactaaaaggctggaaagtgactttaaatttctcacaccattttatacactgtgttttaatgtttggaggttttatttgctttcgttttggtttgggtttatttgtttgtttattttttgcacttgttaatacaggatttattttgggggatggtttctcagaggtaaactaagtcttttcactgtctctatctctctatatatttctagtcattgtgtgtgttcatcagatagttctgtctttatgtcctgtcagcttctattagaggaatgattgctatgacctcatggtatagcaaaaaacaacaacaaaaaaagaataaaaaataaaaaagacaaaaaaaagaaaacaacaaaaaaataaaaataaaaaaaatccctaagtctcccttctacccacagaaccaacaacacccttcccggcctttcctttccctcgccctcttctcgtcccctaagcaaacaacatccgcttgcttctgtctgtgtaaccacagtgaatgggtgtgcacgcttggtgggcctctgagcccctgttgcacaaaccagaaacagagcggagccaagggggcctgacaagagttcctttttagctgaacaaacaagtgctctccataataggtggaatcagacagttaacacatttttatgttgaaaacaaaataaaaggaaaaaattaaaaaaaactatcatgaactgtattgctccagttcccatccccaagtggcccagccctttcttgctggtccagttggacaggagcagctatctagaatcaggatgcggggagtgaggaagtttttcttttgacaatgaaggtgggctttcattgtgatttttgttctgttgcagtaatataggagcacattttggccattgtaattacagggaacaaacaaagggattgcggacacatatctggacttcttttcctcccttattgttgtggaagagacactagaaatgctcaaacacctgcaatatacagaatatacacaattttattccagtatttccctaacatatggtttaaaattattccaggtatacagtgtatgcaattctgcattatcacagaggaacaacttcttttttaaaaaataaataggtcagccatttttattaacgtgcaaaaactttatcactctaacatgctctaggtagttgaggaaaagaggtctgatcactgtttgtattttattttctttgtgggaacatttcacctgctgagtgtacatgaatttgctttctataaaaggcttttatgagtttacagtagaatcagtggaaggaagagttaataagggctgtttttaaaaaaacaaacaaacaaacaaaacaaataattaaaaaaaaaattttacattccttcctattctctaactacacttgggaagtgcacttcagataagtttgcagtgtgactgagagatgaaggaaatccatagaaaaggtcctcttagtgaacaaaatttagttattaactttatagctatgaaatttccccgggcatttgtttttgttcaaacagactttaacctctgcatcatacttaaccctgcgacatgcgtacagtatgcatattttgttttgaaaaaaaatgtttcgttccagtctgttaagaatattcaaaaataataaaggtattgcttaataaaattgctagaattgtttagcagtacatgcacaatattttactagattctttgttttaatagtgttttgttgagactgaaaatcttaaaatggtctg

SEQ ID NO: 416 - DNA, 3012 bp, Homo sapiens
ccactagaacaaaaatgcggactcccctaaatagaaaaacaggcaaagaaaacaaatcacagagtgaatttaacataatagaaacagtaaaaataatataaaataatatgacaggcttaagactaaatatatcagtaatataaatatataaatgagctgaaaatcacctaataaaaggaaaatattttcagattgactttcaagcaaaaccaattcattgctctgtagaagagacatacctaaaacaaaaggattcagaaaggataaaactagtaggatgacttaaaggcatacaaagatgctcttagcagtgttaattataagacccaaataactaccaaaatgctcttcagaagtagaatgaataaacaaagtgtggtgtattcatacagtggaatactacgtatataatgagagtatgaatatgaattttacaaaacaacatggatgaatctcatagtaaggagtgaaagaggcatgaaacaagagtgcatcctatatgattccatttatatgaaattttaaaagaggcaggaccaatctatggtgatagtaatccgtatcatggtcacccttatggaagggtaatgactggaagggaagccaaaggggctttcagagcactagcgatgctctattttttatctgggtgctggttgcatgattttgttaactttgtgaaaattctttaagctggatacttaagagttgtgttcctttctgtatctacgttacattttaataaaaggtttacatttaaaaatataaactatttacaagcagaagatgaggcttggggtaaagtcccatggaatagctggacttagagtcatcagagagaatgatttttaattccaatttctatgaacactgcctctaggctctaaaggccattttcttttctttatgacacttcaaggttgctccctagatttgtttttgccatctccaaattaaaaatagagtctagaaaaacagggtagagaatgcagaagtacagccgaatacacaaaggcatttggcatagagtaaaccatggtatttcaaattggtgggatgggagagaggaatggatttttcaataactggttttgagacaactgggtagccatttgggaaaaaattaaattggatccctgtcttcctccttatatggaagtaaattttatattgattaaaatatacatgtacaaaatgaaaccataaaaatattaaaagaaaatatagaggtatatatttttaaaccatggaatataatgtaagcctgactgaaaattcaggagctatgtaagaaaagattgacaaattttaaaagttctacatggttaaaaataatatcataaaaccaaaagatgaactgggaaaaatatttgcaacacatgagaaggggctaatttcctttataaagtgttcacacagtcaacaaaaagtcaaaaaagatcagtaatccaaagaaaaaatgagtaaagaacatgaactgttgtttaaaacagactaactaaataacttactaacacccgcaaagacttattgcaacatggaaacctatgaacataggaaatgaatgatgccaggtcttattcataatagaggaatacaaattaaattgcaagtacagtatgtagtcatttttcacatattagataaaaaacatttgctcatacattgcattctgagtacagagtagcaagaagtctcctatattgttgaatgggcagtattagaaataatttaaaatgtatataccctttgacttactggtttccttctaccactttattctgtagatacacccacatctacattcattacagcattgtttatggcagcaaaaaattggaaacaatggcaggtaccaccaaaaaaagagactggttgaataaacaaaagcatagctatgcagtggaatgctatgcagctggtagaaagaaggggcagattgatatgtactaatatgaaaaatctccaagacacactgttaaatgaaaaaaatgtggcacacgtgccacgttttaaaaagggaaggggcatatacacagatgcttgtttacacagttagtatttcttgaaggatatataagaaaatggtttcctcagaggtatagacctttggggaagggtagggatgagagggaaacttacttgtcattgtttactgttttgtattgagaatgtttcccccaggtgaatgactgttatacctatttaggaaaacagacgaaaccaataaaaccaaactatttctatacagtgattattagcagagatgccattggcattttagatgcattttgggactgttccacacatttaggacattacattcctttccctttgcactaaatgccagtagcacttccctagacactgacaaacaaaaagtgcccctgtagatttctgaaaaggccagggtgtggattggggtggtggtgttgtttgagtctaaaagaatgtaacttctcttcagggtttcccagtttccaggttatgttggcctggcagggagggtcttgaccgacacggatggttatggacctttacttagtgaaaaactcaaatgaacttcctttctagaactcatggttttcattcagtgacactggactttgccctggagcagtgactggtcagggagctgtatctaagtgggagttgcacctgtgtgggcaaaagtgtggcactcagattttcgtaacaatgggcaagatttcaggaagcaggattttggttcccttactttaaaatgcatggagaatggctgggtttggtggcccatgcctgtaatcccagcactttgggaggccgaggtgggcagatcacctgaggccaggagttcaagaccagcctggccaacatggcaaaactccatctctactaaaaatacaaagcctagcatggtggaacatgcctgtagtcccagctacttgggagggtgcggcacgagaatcacttgagcccaggaggcaggggttgcagcaagccgagatcacaccactgcactccagcctgggc

SEQ ID NO: 417 - DNA, 1444 bp, Homo sapiens
ataccttcacgatagttgcttccagagaaaggcatgcaagtctaattttaatggtaagaatcacttactctgttacagtggtgctgctgaaggaattatcctgaaggctgaaacagaaacaagaaaacatcagccaagtctggccggggagccgtctccctgattgctctccgggagtgagcacatacacaaacatggcacttgctttcttccacatgtctcctaccaattttacagagctcaccccaaccccccacactcccaccccacccccagggatggaggctcgcagagattcatccatcgttcagaaaaagtcaggagagaacaagaagggggagggggatattgctgctgtttggtgttttattttgtgtgtgtgttttttgttgcctaggaaaaagaataggctctaaagcagggctggctgcatcacacctgatctgtctagagcgctcttatttcagtcccagagaggaagcgcccagccccggactacagagctgtgaatgaacccacgcagggcgagtggagaatttattccaggggttagctggtgattaggacaccgtgcaaggggtggggcttgctatgcctagagcagcccgcttgagctcagctccctagctagcacctttacaaaggatggagagacggaatccgcaaacgctatccctttgaataaaggcagccttgttagggaaaggaaagactgtcagtccccccactggcttctttcccttggggatatttgttctgccctctttgtattgttcatttctcctgccatgttgacctggatatgggatgtagcatctattaaagattcttatttgaatttgaattattttatttgaattttgaaataagattcccatccccctttttaatctttctactcccagaagcagctaggatagtggtttttagtccaaacctatgtaagttaatgagttaacataacaacaaaaaactcataatttttctaattgggcaattaaaaactttaccaaaacttggaaataatggggaagagcatgacctttcaatgtcaaactgagacatcggttaacaagtccacccaggggccagttaattctccagttgtgtttggctttgctcttcactattgattagattccagccacagtgaagttctgtggtcacttgcacattttattcagcctgttcagagaaaaagataatcgcagcgtttatgattttattgcaatataggaagataaccaattttgcatctaaccaaataatcattttgcatctaaccaaactttctttctaaatacctgtaaatccacagatcctcaaaatgcttttggaaccagcttcatcatcttacagttttccccattaataagtgaatagatctctgctgcctgcctatgtttgtatgagagttgtattttaactttttaaatttatacct

SEQ ID NO: 418 - DNA, 2439 bp, Homo sapiens
aatttcctcaaatggttgtctcacctcacttcatgagctttcctttttttgttgtccatctttgtgatcggtgaaatgaatttcggtgtttgctcatgaaatcgagcattttttttcatattaattggccttttatatttttgtgtgaattgtctgttattacattttaccttttacattcaaatactatacagaatattatcctgttacagctattttattttttgcttacctttttatttatttagagacaaggcctccctctgtcacccgggctggaatgcaatgatgcaaccatagctcactgcagcctggaactcctgggctcaagtgatcctccaaccctggcctctgctaggaccacacatatgtgccaccatgtcttgctagtttttaaatttttttgtaggaacaggatctcactacattgcccaggctggtctccaagccttgagcaatcctcctgcctcagcctcccagagtggtgggattacaggtgtgagccaacacacctggcctttttttgcttacctttcaattctgtttgctgttttttgacatataggggttttaaattttgttgcagttggattacattgccttttgttacttttttcattgcttctataaagctaaatcttcttttcccattccaaattcagaaataattttctactaatttagttttcttccttttttttttccttttatatttagctctttaatctccacagaattaattctggtgaatcttatgaagtaggagttaattttactttttctagaatattatctggttcttgtgtttattcagaatcccagtatagctgtgacatgaaccaaaacaagtgatttttcttattttagtgctttgattttcccattaggaaaaatgaggaagctgtgagtaccagtggaaggaacgagccaggaagagggactggaaccatctcagaagccatgcccctcaggctggaacttgccctgtctcctcgcagatgaggtaggaggctcagcacgggctggtgggagcagcccacagcagcagtgagggtcagtgggcctgtgagttaagaagaaggtggcacagagcgagggtctctggatcctgactgtttgacttttccatgattaggataagtagccagggcttggctagttggagagttactcgaacctcaggtgacagttgtaaggcagcacatagtgaaaaagagtcctagcctgggaaagtccaaaaccttaggtctggtttcagttcactcacctatctctgtgacatagcaaagtctgttaatttctctaattttctgattagtagtatagttgcaggaatgaaataaaaatggtcctgattactcagagattcatttactgattactctttttgtgacctgaataaagagtacaacatctctcaaaaggtaacaatatgatgtttcaggaaattagagaagttaagagactttgctgtatttactatacagatagttgactgacagctgagcccggacccaagattcatttataaaatgaaggagttgagaagtacatactctgaggtttatcctaaataaactgaagtctaggcaagtggctgagctaggtttgccatgaatcaatctgctgagtatatttcttggtaactagttcatctttccttaattcagtcaacaaatatgatatcaagtttagtaagatgatgacagttgtcatgtaatactccatctggaagattgtatgcttctcagcttccctaacttttcttaatctaccttaagcttcctttctgaaaggtttctgtctttcctaacagtctcttctttccgccagctacctcttgtgtttaagaattaaaggaagatgagctaggaatcaagaaatacgcagaactgattcctgtcactggcgccaccctgccacttgcccagagctgtggtataaatttttttaataggctgaaataaaaatatgaatagcattttggtattaagcattaaattgataaaggctatgagatacacctgctctcaaccatgttaattttttattattggtattaataaattattacttactaacatattaacaagaattgcattgagaaacaaagcatccacaggccaaatcttctgaatttcaaatgtttatattaatgcgttgtattctagaaaagtagaattgtcttaagtagctttgtaatataaagtcatctatcagcccattacacctattagaatgttttaacctttatttcccacttttttgtttctagactgagtgtacttggtctggtatttgtggcataacttacggaacacataagaatgatacagatactatttaatgatgacctaatacaagcttagatagctaaggtgaaagcttctatggccttaacattttcctcttgaagaatgtattttctgtaataaaata

SEQ ID NO: 419 - DNA, 1944 bp, Homo sapiens
ctgcctggacctcaccataagtctcaggtatttcttcatagcagcatgaaaatggactaatacagcaaattggtaccaggaatggggcgctactataaggataccaaaaacatggaagcaactttggaactgggtaataggcagaggttgaaacagtttgaagggctcagaagaagacaggaaaatggggaaagtttggaacttcctagaaacttggaagattcagaagacagggagatgtgggaaagtttggaacttccctagacacttgttgaattgctttaaccaaaatgctgatagtaatatgaacaatgaagtccagcctgaggtgatctcagatgaagatgaggaacttgttgggaactagagtaaaggtcactcttgctatgctttagcaagagactgtcagctgtttgcccctaccctagaaatctatggaactttgaacttgagagatgatttagggtatctggcagaagaaatttctaagaagcaaagcattcaagtggtgacagaaaataaaagtttagaaaatttgcagcctgatgatgcagtaggaaagaaaagcccattttctgggaagaaattcaagttggctgcagaaatttgcgtcagtaatgaggagccaaatgttaatcaccaagacaatggggaaaatgtctccagggcatgtcagagaccttcacagtagcccctcccatcacaggcctggaagactaggagggaaaaatagtttcctgggccagttctaggggccccctgctgtgtgcagcctctggagttggtgccctgcaccccagccactccagccatggctaaaaggggccaaggtacagttctggccattgcttccgagggtgcaagctccaagccttggcagcttccacatggtgttggttctgtgagtatgcagaagacaagaactgaggttcaggaaactttggctggatgtatgaaaatgcctggatgttcagacagaagtttactgcagggatggagcccttgtgggagaacctctgctagggcagagcagaagggaaatgtggggttgaagtccccgcacagagtccccactgttgcactgaccagtggagcaaagaaaagagggctatagtcctccataccccagaatggtagatccaccaacagcttgcactatgcatgcacctggaaaagccatagacactcaacgccagcccatgaaagcagctaggaggggagctgtaccctgcaaagccacacaggggtggagatgcccaaagccatgggagcccacctgttgcattagcgtgacctggatgtgagacatggagtccaagaagatcattttggaactttaaggtttaatgactgccctattggatttcagacttgcatggggcctgtagcccctttgtttcggtcagtttctgccatttgaaacaggtgtgttttcccaatgtctgtacccctgttgtatctaggaagtaactaactagctgttgattttataggctcataggcagaggggacttaccttgtctcagatgagattttggacttggttttttgggttaatgctggaatgagctaagactttgagggactgttggaaaggcatgattgtgtttggaaatgagaggatatgagatttgggaggggccaggagcagaaagatacagtttggctgtgtccccaccaaaatctcctcttgaattgtagctcccataatccccacatatcttgggagggacccagtgggtggtaattgaatcatggaagcagttacccccatgctgttctcgtgatagtgactgtgtgtgtatgcctgaatgactttcatattcttttgtgctcctagaacctaatttttggtttttggaaaattcaaagaatggagaatcaaatcaagagatagaaaacgtcatgctattctgatatatagaactcaaacatttttgttga

SEQ ID NO: 420 - DNA, 2801 bp, Homo sapiens
ggggccgcgccgggtagcgtttctttttagtgcctgaggcagctctggctcggagagccttttgctagccccacggggacctctgtgcacggatggacccgcccggacctggcgggaagcggcctggcaggcggcggccccggcggcatcagcagagacaggacggggccgacgccgcgggcccctgaggcgtgcgtgcccaccgggcccggcggcggcaccatgatgccgggcgagacccattcggcggcgcccgggacggcggcggacctgtcgcgatgtcagggctgcgcctctctgcagcagaatttgaatgaatatgttgaagcattaattaccttgaaacaaaaaattatcaatacagataatttgttaacagaatatcagaagaaatgtgatgatatccttttactggctttttctgttgagttttggaactaacttatgtttaattcctgaaagagatgtgagaattgttcctcataggccttctggaagatagtacttaacaggcttcagtagggagcatgtctcacaagcacagtaaacagacagtccttttattcttaatccatgtttaatgatgagttttaaaaatgagattacttttcttgttgattgtgattttcttaacgtctcataagctttttagtgggtgggtttctcttaatttgtactgaggatgtggcatgttttttaggtttacgaataatgtgaaactgaatgtgatacttgctgaatgttatttttgaatctgcaagaagtacagtatgggaaattcaaaatgttgggcaattgactttcattaattatttttgtgatttacagggaaatgtgtgtgtatatatatatctccaaaatcatagaaatagcgttgctgtatattcatgaacgctgtgggcagtcagaagacaattcttggccttacagtcaccacttgttcggtagccatttaatgagctcggttgtggagttagtgtctggtcagctatttttttagcagtgggaaaaagatgtgtataattgatccttccatgtcttgctgtggtaataccttgatttaaatctctcctgtaaaaacttaaagtttctctcataaaagtaaacatacgtatactcctttagggagtccctgaaatttagcttcatgttcgatgtcaggacagattcagctgttcagctttggcaataacttgaggcctgcttcaatatgatttatgtcatacctgaacttacatgtcatcatcagttatttatttgtgttggtaccatctgtactagttgattctcacgctgctataaaggactacccgagactgggtaatgtataaaggaaagaggtttaattgactcagagttctgcatggctgggaggccccaggaaacttataatcatggcagaaggggaagcaaacacatccttcacatggtggcaggagagagaagtgcagagcgaagcggggaaaagccccttataaaaccatcagatctcgtgggaactcactcattatcaggagaacagcactgggggactgcccccgtgatctgatcacctcccacaaggttccaccccagcacctgggaatcacaacttggtttacaattcaagatgagattttgggtggggacacagccagaccatatcatcatttctaatttctgtgaaaatcgtaaaattaagagatatcctttagatttatttttactatctgaatttttacattcaaaaataaaggtcattggaaatcttcttatgtgcctcatctgttaaaaccatcagtccatttaactatacgtgcatattatggcagaaatgatatcttcaattgttagtgaaaggtgtatggaaacaattatcacaacttttagaaggctaaatagggaaatagatgggcagttccagtattaggagagtatgtgttcattgtatcattcttggaaaaaatatgcacactctttgagttaacttatacatgtaacctagcagagaaataattgttttacttttctaaaggttggccagaaaaattttaaaacaaaaattatcagaatcctttattaacggataacacttttgattattcctggatttcagaagttcattctgtaatatgatatttggcttcattgtcagctacagatagatacatacctttaaaaaaatatgtttcacatttgctccaagtatgcatttgttgtacctcaaatatgcagttgtttgctctgcaacttgaagtgccgatgtcacagatttaacatggcttttaaaaatataatttgattccttttgctactaaagtagcagtgcattttcccatttttttcaagatgggaaagttatttcttccataactgcactatgataattcagtatttcagctcttttttgtgtaattctgaccactttattatcacctctttctaaatggcaagaaatgtattttatttcatcaggtattttttgaactttcctattttgcaaagattaatttaggatgaataaaatatagttcttgttcttcagaaaccttcagaaacttacattctaatggaggatgcacacgtttttatgaacgtacatgtcctatattgtgataaaaatacaggaattcctaggagatgaaacataaaggaattgttttttgctatcttagaatattttactagcagtatgagatggtaaaataaggttatcaaattacataaattgtaaaaaatgagagttttgtagattttatgatctgttttctacaaaacatgcttatatatggtttttgtaagtacataacttttgtttgctg

SEQ ID NO: 421 - DNA, 1911 bp, Homo sapiens
taagtgcgcactctgggcctttgtgtatacggtatgcatctgagatataaagaagaaaaagccacagctccttccttcgtgatgctcatagtctggtagaggaagaattcaggtaagcaaaaaagccagtcatatgatcatctctttctgcattttgctcaggatcaacttctacacgactgatctatagcttatggcccctgtcctctttcctttcttgctaagtcatagacataccagttgaatttatacaattcatctgtggggaaagtttcacaaggctgatttttccatatatcttctctgtataatttattgttaataaattcatttaagtattttaaaatcctaaaatatatgtcagtaaagggtgctaaaatgttaaacaaatattactttcagattttcttttcttctctttttattcctattctattttcccccttggttaattttatttttctcaaaattctccctttcttttctaaaccttatatttccccaaaagaatagtaccagatagtctcatatcaatttgatcttttttggcctggttccttatattgactttcctgtccaagtctttctacttgttctaagacttcatctcattttttattattggcctttcaagatttgttagacaaacagttttcagtgaaagagcttaatttagttaagcactaagtttttatctgggctagaaattttggatcttacctttactcttttctcactataaccccaaacaacattcattcatcaggaaaatctgtcaatatttcctctttgaagcattttacatctgtttccaaagctttattcccatatttagtgtcacagtccatgcccttatcttttctcagctgataatgggataacctcctaaatatttcccatttcttctgacattaatcttagtcttttgtttttcaaattatgattcatgttcctgtaggttataaactcaatttacttgttcatgatcataactttttagtgatgtaaaatagaacatatacacaatattggagtacatggcacataataagagatttcatgaaattgttttaattatttacatatgtatgctaagtgttaacttaccacttgtaatgggagattataattgtgttggttgtcctttggaggggcatctatctagcatctgaacctttccccatgattagaatattctgtattatatgagacttggtaggatgtagggcctgcttccattatagaagctaaaaaatagcccaggtcttgtctttcccagcatcccttgcagctaggttgcttacctgtaacctaagcttgcttaatcagatgcctggtttgttgaactggcacttacagaagcagggaaaatgaataccaaggctgctggtggtggcggacaccagcatttggcttttgaggcagcagcggctgacagccagtgatagcaacagggtcctcatcagaatggttctggtcatggtttggctatgattctggctacctcacttccttttgctcctgcctattttctgagctggcactttaaacttcctggagattttatgagctactctagtttctttcaaccaatttcttttctacttgctatggactgaattttgtcatctctctactcctccattcatatgttgaagtcctaacccccagtgtgactatatctggaggtaggatctttggaagtaattaaggttaaatgaggtcataagagtgggaccctaatcagataagaccaagactttataggaagaggaagagagaaagagaaacctctctctttcatgtgaggatgcagcaagaaggcagctgtctgcaagccagggacctcaccagaaaccaaaccctgtcaagccttgattgtggactttccagccttcagaatgtgaaaaa

SEQ ID NO: 422 - DNA, 1900 bp, Homo sapiens
  modified_base (697): a, c, g, or t
ttgggaaaagatgggggaaaatgtttttgcttagatgaaggagcaaaagagggtgatgagagcaggagggggaaaaagggtggccagggataaggggaaaagatggtgggaagaaacagtggggaaggggtttgggtaaatggatggggaaaaggtgttgagcaggagagtagagaaggctttgtgaaatgatggtggggaaaaaaatggtgaagaagtttgggggcagatggaagaaaaagggtggcgaggggaagggggccaaaggtgctcgggaaaagaaggtagggaaataatggtgggggacaatggttttgagtagattttttaaataagatcatttgtatttttgtttttgaatagtctgagatcttcatatattttgtgcatgaatcccttgcctgatgcatagtttgcaaatacttgcttccattctcttggttgtttcttcattttaaaagatttttaatttaatttaattttttttagatggagtctcattctgtcactccagcctgggcgacagagcaagacttcgtctcaaaaaaaaaaacattaaaaaaaaatgtattttcatttgtgactcatcttgtaactagaggatcgaaaggactttgaatgatcctcctgacaaaggagggaagcagtaccttaatcaaccctggggtccaattaagtacttttataaaccccttgncacatacttcatataatttgtggcctagatactgacttataaactaaattttttattttccttttataccccttacaaaatcatatgcagaagtccaatatctagttggtcaaaagatgtgctgatctggtttcagtgggggaaaggtcgggtgaggaagggatctgcaatcttgccttatactctgttatttgttgagatgttcatgaaaggccttttttagaaatccgtatggatgccgattactctttatattgagttggcaaactgtgtgaaaggccagatagtaaatgttctgggttttgtgaaccatatggtttctattgtaactattcatctctgcccttggagcaccagagcagccgtaattaatatgtaaatgaataaacagagttgtgttccagtaaagcttcatttatggatactgaaatttaaagttcatataatttttttttttttttaagaaagggagtctcgctctgttgcccaggctggagtgcagtggcgccatctcggtttactgcaacctccacctcccgggttcaaacagttctcctgcctcagcctcctgagtagctgggactacaggcgtgtgccagcacacccggctaatttttttttttttttttttttaagtagtgatggggtttcaccgtgttggctaggatggtctcgatctcctgacctcgtgatctgcccgccttggcctcccaagattacaggtgtgagccaccacgcctggctgaagttcatattatttttatgtgtcataacacataaaattatttttatgtgtcataacatctttttaaagatgtgctttttagcttatttattgggataaaatgttttgaatttgaatatagatgccctgttttaagatttttttttcccacacagttttgcatttaagtaccagaatacccaaacatttttcattagtcttcatcaacactcccgtttgtgttctcagtcaaattaaaaatagcaatttttggccgggtgcggtggctcacacctgtaatcccagcgctttgggaggccgaggcatttccactaaaactacaaaaaaattagccgggtgtggtggcgggctcctgtggtcccagctgccctggaggctgagggaggagaatggtgtgaacccgggaggcggagcttgcagtgagctgaggtcgcgccactgcactccaacctgggcaa

SEQ ID NO: 423 - DNA, 1716 bp, Homo sapiens
gtcgaggctgtcggaagggaagggagtcccccaaagactccagtactcattccttcaagcctcccacttagatttccttctccatctctttggatgccctcggacaaattctctgcttctccgagcccagttttttgcatttgtaaacagtttcactgaactgtgattgagttgggggcggggggcggggtgatacagactttggccgtgcacagcgtctagctctgagtgagggcctaatactgtgggggaagggaagaggtcggccatcctaggctgttaaggtggactcacctctgcccctgccgtgtctctctgggaaagccactgggcttctcagagcgccccccccccgccccgccccttttgcatatctgcacaaagaggacgatgacacctacttgagggggttttcctggggatcagctgagaaacaggagactggcttagcacagtgccaggcacctgaaactattgaggctggcagtggctgtccctgcttccatctgcccctgtcgttcactccagtgggcattcacagagcatctgctctgaaccaggtccatcgagaacatgcagggttgtggcagaggcagatgggacctgcactgtgacttggccaacgtggactgtgccgattgtctccttcttagagcctcagtttcctcctcaggaatacagagcagctgcctccgtgactgctggattagaggagatggtagttgaggtcacacaagacctgagtaggggaaactcactctgaacttccccgaggttctgtcactggccctttctccaaggttgtggtgggacagggcttcaaaaaacccaggggagaatggtgagggctcgggttccaaaagcagagtcaatggacagtttccccctgtgtaaatagaggggaacagcagtgctggttcctccgggaggattcagaagggacaacactagcacggggtctggggtagggtgacgtggtgaaccaaagccttcccctagagcatatgccgggacctgacctctgcgttcccttttcattcattcactcattcattcattcattcattcagtgtcagtggtctgacactgttgggggagaaaaggaaatctcagcctgaactggaaacctagggacagcagggagagttaagggatcttgagcagccactgcttccaaacagccattcgttggccctgtgtgggaggaaacatgcaatgatggatctttgacctcgaatcctccctcctcaaactgtggtccacagaccagccacatggccatcacaggacagactcttgtccccaggccagacctactgaatcaagtgtgcatttgggatgatatccaaggtcagcaaatgcctggcacagagtaaatgcaagacagggttgactatcattgctcttttcgagtggggatagagctcccaacttttggctgggcatgtgggtgctcacagtgaaagttgcatttccccagcttcctttgtagctagatgtggcattgagaagtaaagggaagtgtcttgtggggcatctgggatagttggtatgtctgctttaccctctgtgtccctttctccatctggctgccaggaatgtaggcgtcaccatcctggaccataaggtggaggccaccacagagcagagcaacaagacaaaaagagcctaagttcctgccgggcacagtggctcacacctgtaattgcagcactt

SEQ ID NO: 424 - DNA, 2226 bp, Homo sapiens
tgagagcccccgcactctcacctcacagacggagcagacagatcaaatggaaaggctcccccatcctgtcctctacaccaccttgcagctgggcctcagcaactgggcttttaatttcagtctaattcaagtcagcagcatagggcagctcctgggaaattggtttacacatgcggacaagcccagtagcccagagctaacccactcaccatccctgaccacagaggagcagataaggaagcaagatttgtcgggagctatgacaagttccactgtggatggcaaggaaatgaggggggcagcaacctgttaatgagactcaagagtgtcaagacccatttttgttatctggattttcaaggccgagttcaaacatgaggatcctggaaaggttggcagacgtctgcggaagctaccttctctgttgtaaaagtccacaaatgagaagtggctgcagtgtataaacctgtggacagctcttcttgtactgtcacccactctcatagctcaccatcctcaggcaccctcttatctctgctcacagagtacccacctgaaatgcctaacctgctaactccacccagtgaaattctgtgaatcccacaaggtgtgtcttatatgcctcctttcctctgctcattccttccacagacattattgtacatgccttgggtgctcaacggcagtctctggattccccaaacagtacagtagtttcttttagcccttcccattgcacttgtcaagggcatttgcttttggcattcaaatttgtgtctagtacataatggagacctggctacagaagtattgcagtactgaaaacaatctctgctatccacgggcttgaaatttaccctctgcaagtgtaactggaggaaaacacagtagtcaccaactacgggcagttactgaaattcaaattagttgaaataaaataaaaccaaaaatacaaatccttattctcacagaccacatttcaggtgctcagtagctacatgtggctgtgactgttatatcacccggtgcagagagaaagcatttctacccttacagagcattccattagacagtgttgctcgggattctatatccccctgaacctctgctattccctgtcactgggtccgtttcctttaaaaccctaaccaattatttgcacacttatttgttttatggtgattccccgcaagtagactgtaagctctgggagagtaaggactatctttatctcttttaccacttccacccttacacttagaagagacctggcaggtgacaaaaatatattgctaaagtgaacaagtgggatggtgagtttctatccactagagagtcatagctgtctaaagggacaccatgcaccatttttccagccttacctggtcctccagatgccccgcagataccttgtggtgtggccttcgctcttgcagggtctcagccccctgggacatcctcctacatccacatcttcatggctgaatcccacccagacctcaaaaatgaaacgaaaggccaccttcaggaagccttcctcaatccagtgcttcccttttgtctcctcatcttcttccccattaaagggcttaagaatgatgtcctccaaatttgaccaccttggcttttaaactgcaactctctggaagcttcacacctgttgccttattggtggctgctttatacacatttgttcatgtttcttatccactacttgagagcagggtctatgtctagcttacaatcatcttcccaggctctgagaaaagcatttaaaatacagcagaggcttaaaacataatatttgaatgaatatatgagtaaataaatgatcatgttttaaagctatgattttactatttttaaatgatcactaccactaaagaacatcatttctttattttttaatccatggctttttatttttattttttaaagattcataccaaacaatacagcataaagctctataacttataaaatttgtaaaaccaaattctaaaattgagaatttattatacacaaaagttaaagaacctagggctggaagaagcaaatttctatactgaaatgtctttaggaatgggcacgtattcatttttattttgcaacaaaggaatttcattaaaataagaaaaaatgtgaaccattagtgttaacttacttggtttttagatgaatattaatcttatatctagaagtatatgtcttgcatcagcatttcaa

SEQ ID NO: 425 - DNA, 2566 bp, Homo sapiens
tacaggttcaaggaaaagttagtggaagccgttatccatgtcaacattgaaccagaagaaatagaagcagaggaagcagtaactcattctatgaggccagcattacacaaatatgaaaactaaacaaagacattgaaagaagaaaactatagaacactatcagtcatgaacacagatccaaaattcctcaacaaaatatcagcaaatggactccaacactgtataaaaataattatataccatgaccaagtacaatttgttcaaggagcatccttcatgatgattagtctgttgataattttgtcaagcaaatgacacctgttatcacctccaaggaaggagtcccacttcagctgaagcagtagtgccaagttttaacccactccatctttcagggtggccaagagagataggactctatttagcctggcaaaccctctgcaggagtctccccaaaagtacagtgccccaaaccattctgagatggggccagctgggattcaaaagaaagaagcaccaaatattaagttgattagtttgaagcattcattaggagaaccttacttacagaatgctgcagcatatcctcaggcagaaagggaaagaaaggaatgttttacctgaatatgacttcagtgacgaggtcagggtaggaagcttatataagagtttaatgaagttggctcacggagaggactagtttcttttttcctttttgatttttttaaatcaacttttattttaagttccagggtacatgtgcaggatgtgcacatttgttacataggtaagtgtgtgccacggtgttctgctgcacggaacaacctatcacctacgtactaaggccagcacccattagctgttctccgtgatgctctccctccacaccacccctctgacaggccccagtataggtcgttccccagcatgtatccatgtgttctcatcattcagcacccacttataggtgagaacatgcagtgtttggttttctgtgttagtttgctgaggataatggcttccagctccatccatgtccctgcaaaggacatgatctcattcctttttgtggctacacagtattccatggtgtatatgtaccacgttttctttattcagtttgtcattgatggccatttgggctaaatgcatgtctttgctattgtgaatagtactgcaatgaacatacatgtgcatgtatctttataataggaagatttatattcctttgggtatgtacctattaatgggatcgctcggtccagtggtatttctgcttctagatctttgagaaatcaccacaatgtcttccacaatggttgaactaatttacatttccaccaacggtgtaaaagtgctcctttttcttcacagccttgccagcatcttttgtttcttgactttttattttatttatttatttatttatttattttgcactccagcctgggcaacacagtgagaccctgtctcaaaaaataataataataaattttaaaaaaacaacttttgctccatgaagaacctcattaagaagataaatagacaagctacagggtgaaagaaaatatttacaaaccctatatgcaacaaattactaagatgtagactatataaaggattcataaaacttaatagtaagcaaaaatccaattatggaatgaacaaaaaacacaaagaagtaacttctcaaaaagaatatatagatgacaaataagcttataaaaacatgtttaacatcgttagccattaggaaaacgcaaattaaaatcacaatgagatattactatatatttctcggaatggcctaaaaaaagtaaaatatcaaatgctggcaaagatatggacaaactagatcacacgtacattgataatgggaatgtaaaattatacaggcattctggaaaatactttgatagtttatttaaaaaagtaagtgtacaactactttatgccccattatttgtacctttagaaatttatctcacataaatgaaaatatgttaatacgaaaacatgtgcatgaatatttatagcaactttattcacaatatctccaaaaaagacacaactctgatgttcttcaatggttgaatgtttaaataaactatgacatatgtctagcatggaatactgctcagcaagaaaaaggaatgcattattgtcatgcacaacaatctggatggatcttaagagaattatgctgactgtaaaaagccaacccaaaaatgtcacataaggtataatttcacttttatcgaattattgaaatgtcacagttataaaaatgaaaaaaagattaatgattgccagaaattaaggaagggttgggggcatgagggaagcaggtgtgtttataaaaagacaacatgagagagactatttgttggaaatagtctgtatcttcacagaatcaatgtaaatatcttggttgttacattttaccgtaatgttggaaggtgttaccattgaaagaaactgggtaaaactggataaatgaaatctctctgtattatttcttacaaatacatgtgaatctacaatgatctcaaaataaaaattttaa

SEQ ID NO: 426 - DNA, 1152 bp, Homo sapiens
gggacagggagggtcgtggcgacactcgcgccagctcccgggacggctgggctcgggctggtcgccgacctccgaccctccactagatgcctggcagagcccggcgcgtccggagcccgcgcccctcagagggaaggggtcgcgcgtcgccgcggcggccctgaggctgacggtcgggaggtgaaaggccggagtgcggggtcaggacgcctcctggagggggcgcggcgcagggggccgcggggaagccgccgagggcgcggagcgggaaggaaggacagtggccaccccgcctccgcctgggcacggggcagggacccgcgcgaggcggtgccggggaggtcctcacgtactcaaaaacaagcagaaaaggcatccagggccccttcccgaactccccacgggctgccagggtgcagagaagaagcgcctgaggaggaagtgccacacaatgggagagaacaaaggggagcggagcgagaccgcggcctgcgcccgccccccgcttgcgcacagtccccgccccgccccgcgggcgccgccgccgccgccctccctcccgcgcctccttctgaccccgagcgagagacctggtcgggagtccggcggctagagttgattgcaaggggcctgttgctcgactgtgcgttaaagaggctagagggagcctaggtagacgtggggcgcaagctggagccggcagcttgcgagggggcctttacgtgatgaggctcaccatggggtctccagcgaggtgtctgcagagcttcgatggtgagaggaggaactcgatttctcgttttctggagttgggatgtagacatagagagatctgttacatctgagatgaaaaccctgcacaattcacgttagggttggccaattgtttgcttgaccttaaatggttagtgtggtttaagcttccccaaaatgcgctgtgagctgagcttccacttttggaatgattgcagaattcggtgcttaagagatgagaaacttcgttctaccagtaatggaagaaacgcttcaccttaatcaccctccttgtctttcccctccctttgactatgtaggactcgataacacaacatttgaaagattctactctatgaaataagcacagattgaaattcagtatttgccaaggaatat

SEQ ID NO: 427 - DNA, 2299 bp, Homo sapiens
cccggacgcgggcaagatggcggccgcggccgtgcggcccgtgacctggaccactgggctgggacccacgaccccaccaccacgcctggaggacgcgcagcggcgcctcggccttggaaggggagtttcccagcgcgagccccgcccttccccgcccgggaggcgcctcggggcggggtcactggccgggaggccccgccttccgcccgaaagcgccggtgcaccccgcaggccctgcccgtgggtccacaggccggatccccgggttcctaggctgggactgcttctcagaaccactctgtcgtttttaagcagggtcacacactctagctcactgggtccattttaatttctattaaacatttttttttttttgcaaatgatgtagtaggagatccaaggtgtttggttaatgatttattcactcattagtcattccacaaacttgtcttgagcacctgttatgtacccagcactgtgctggaatgctgaggagacaggagtgaagtaaaaagacatggttccggcaggaaacaggcaaggagagccttgacttggtagggctaacacagtgctacacaggagggacgcccgctcggtgtcgggggtcagcaaagcttccttaaggaccactaggttgagacaagaaggatgagtatgggattagccaggaaagggaaaagggcagtgggaactggaagagaagagccttctaggactgggaaagagcaagtgccgggtcaggagagaggacatagcagtgaggaggcagtaggtatggatgaatgagcagaggaagtggggagagattaaggtgctttcagaaacgggttggaaaccagtttagtgagtttggatggcatttacctaaaggcaatgttagagtcatcgaaagtttttaatcaggcaagtggtatgatcagttttttgttttgttttgttttttttgagacggagtctggctatatcgccaggctggaatgcaatggcgcgatctctcctcactgcaacctccgcctcccgggttcaagcgattctcctgcctcagcacctcgagtagctgggactacaggcgcgcgccaccacgcccagatgagaaaactgaggcacagagaggtgaaataaggaatgaaatattactggatgaaataattatactgatcctagccttattttaaaagggcccccagaatgctcttctccaagacttagaaaaatcaaatgctatccatccctcaagatacatccctccagcccctccttgaagtctcctgcagctagaagtagaagtagcatctatccccatctggactccttagcactttgctgtacctgccttagtccccatctctttacacacacacacacacacacacacacacacacagagtattataatactattacctattatattacattatattattgtagttctacaggaactacaaggaattctactgtgaattccttaagggtgggatacattttacaatcatcactgtttctctcattagttgtggaatcaaaaaaatgagtgcacgatgaaaaattattcaaacacatacatgctttcattccaattgaaaatgtaaaaatattactgttaaaacaatattaaaggacatgtaatacaattcaaatattcctaccatgcacgattgttttcacttctttgtgcctctttcaccatttatttgtgtgcatactcagcatatttgtttgtgtaacaggatgttaaaaatagaatttgttgcctgtagtttttgctaaagctctactagactggaacagaacaataggtggtaaatactccattcctcagtgagatgctacctacctatgcagagctggaaaagattttgcaacctgaaaacccaatcctttctgagatataaaagaacagaagagtctggaagtgatttcttcggagaaattcattttcttattccagagaagaaacttcaagctcagaatattggctactacctgtgataaacatttaaattattggaacgagagagtttttataactaaatttgaaagaagcaattttttttatcaaagaccaacccgaagattcatgaccactacctgtgatttcatccatgttgcaaggaaaatcctgccccacttccccaataggaagagtggtgggggaataataaagttgttttatgatgcatcctgtggacttctactcgacagaatggtaacattcaactgtaatcaggatgagcaagccactttattaaactaaaattttaaactttaataaatataatacaaactgaaaaat

SEQ ID NO: 428 - DNA, 1643 bp, Homo sapiens
ctatttaggtgacactatagaaccaagaaggaagcaagatggctgccctttaggatttgttagaaaggagacccgactgcaactgctggattgctgcaaggctgagggacgagaacgaggtcagagcgcttctcttatgccgcgaaactctccctttcttctccccttcgctttttctcgggcttccagggactggggagcaaaccctgtagtgtcacccacaaataccaagagggaagagggaagcttcacaaattactggagcctcttcaacatggctgacaaatatagttttaattccctctaccccttttaaacctgtagttctgtgttctcttctctcctcctaatgctcgtcccctcatctcccagaaaacttacctttgtgcctccgacgagccggtttcccggccttttttaatcctcagaaaagtgatttttaaatttgctttcctttctaaaatagttcagctttgggggcactacttttccctttaatcctcttcccctgtttctttcgtgtaagtgaaacgagtctcccgtttatcctgaacaacctcagagagaacactgatagggtgtttttcgacccttttatcagctgtagggtctgggtctgggtttgtgtctgcctcctcctaccttcttatccccctttagggggctgtacgaagtgaatgtcacagggagtggaattggagtacactgagtgggtttttttttttccttaagtccgcgcgttttgttagcggcgctgagtgaaagaggaaagaatagtttctctggttccccaaacaagaccagaactcacttttctcaaggtacataagtcagcgctgggctgagccttccagcctggggaatgtatgtaagagaatttatggacaaatctgtgtcccggctttgtgcttctcccgaatcagcttcgtttggttccttggtaagtgacaggcagacacaaaggcaggcgcaggcccggggagggggcgggagggggtggggagcgcagcgttgagagttgcaagactgcaaggtcaggggcgcctaaagaaatgaaacccaatcccagcaaagaagtgaagagcagatttataacagtcccatccaaatttctctttggcttctctctttggtctttcatctctctgcctttctctctgtgtctcctctctactctttcttctctctctctcatacacatacacacacacacacacacacacacgtatgtgcttagtggtatgagttactgtcaaggagaagggctacctagatgctaacacacacacacacacacacacacacacacacacacacacacgctttgggtggtgggaagttggagaaaacatttggggcaagagaaaacaatgaaggcggccaggtatggtggtttgcgcctgtaatcccggcacattgggaggctgaggtgggcagattgcttgagctcaggagttcaagaccagcctgggcaacatagtgaaaccccgtctctacaaagtatacaaaaaaattagccaggcatggtggtgcgtgcctgtaatcccagctacttgggaggctggggcacaggaatcctttgaacttgggaggcagaggttgcagtgagctgaaatcacactactgccccccagcctgggcaa

SEQ ID NO: 429 - DNA, 1869 bp, Homo sapiens
caccagtgcctccctgtgagggcctggccagcttggagcaacacctgcctacctgatagtatcggtcagttcctgcagctcctctctagaggaccgatggtatcagccaggggcagctcctctctagagctgtgtcttagttgattatatctgtttgcagatcctccctactgcaatctgttctatgaagtgttgccatattagtagtcttccttaaatatgcctttaatgactgattcttatttctctctctctttttttttttttttatttgagacgaagtttcgctccagcttgggcaacagagtgagaccctgtttccaaaaacaacaaaaactggtttcaaatatattgctacattggaattctccataaaggaattaattgttgataacaatgccctcagtatactatttgtcaatacctataacaatgcaacaaacaaaaaagaaaatttaaatagctggaccacattaaaagttttattgggctaaaatcaaaaacatctgacaagtttttgacttgtctttctcatgatttaatgtcccagcagagcaactgggaacctaattctgaccaacaaggaggaattgtttggctaattggaagtgacagaaaccttgggagaaagcaaccgcataccaaggataattataagaaggtaacatgggccctaaaggatagtgtcaagaaacctgaaacccccaaatgaggtgagacttgcaaaaaatgctaaggaaggacaaaaaacaggccttttaaaattctgttcagaaccagaagatgatcaagaaagggaaatgcccactagcagcttctactgtaaggttagagctgacagagaaagaactccttaactactatttgcttcattctctacaaaggaaactagagaagtgggttgacgtaatagaaagaacatgtgtttgtggggccaggcaaacctgggtttaattctgtttcaacactgcttaacaaaatttatgggaggctattgttttggattgggctcctgcaccaggcccctccggaccaaaccaaaatggagtcactcatactaaaactccaggtcactgaaccaaaactaagttgtttatctgaccttccaagaaatcaggagggagaaaacaaccaaatctccaaacaggccagttttaatcagcgtgataaggaagtcctctcttttttaaccctataaagaaagtaactttttgaaatgatcaatacactttgtattccttagttctgctttctttagcccctttctgcctataaagcccacttcctctgctcaacttactgaagcagtattccattttatagaatgagatgctgcccaattctggaatcactaataaaagccaattagatctttacatttgttgaaattttgtctttgacaacattactagttatattattctgggtatccttttccccatctgtaaaatggacatagcgatatccttcccatcagatttttctcattaatagaagtaatacattcaaaacactgagccaggctcacaccagtgagcaacttgttaatattactcagaagcatattatagatattgacagaaagcaatactgttcctaattaggggaagaaattttaagagagtacctaagagtttgaatttagattataacatttgctctgagtattttacattacagcctttggggggaaaagtacaaatgagatctgagaacagtggtactcatctttgaggaattatggaaaacgtaatagaacactaaacatgggaaaacatcggccttcaggttgaaaagtggaaatctcaatccctgaatttttttttttttttttactaagtaacttttttgcccattggtgtcatttaa

SEQ ID NO: 430 - DNA, 2433 bp, Homo sapiens
ctgggtattaggtgtccaagaagaaagctcagatctgccgaccgcaggggtccgaacgcagcccgggatcaggaagaaagcaggtcgtttgcaatctaccggagcctaagggcagaaggcaggggtccgaatgcagcccgggatcacaaagaaagcaggtcgtttgcaatctaccagagcctaagggcagaaggcaggtattaatatttgcttcataattctgagactcctccacttgaacagaaatctaagaaaacaatcatggcaatgaggatagcactggaacacacatcactatgaaacaccatgctcaatacactgtttaaactgtcactgtgaatgaaagcaattgtatttgtttgaggaatatggacctaagtcttatacctttatagtagcattttgcacttgaggataaagagaatatggaaatattaagaatattttagctttgttagatttttgcctccagatcaaacagaacaacgcacctctacacatgtattcaggtagctgaaggcattgacaatcaaatcaaattgttcattttgaacaaccgaaaagggtgaggcaatttcaataaagaaaggttgggagacttctagagtggttgtagaggaaatgccaaatccaacgtcgccattcacacaaaagccacttgcctcccattgggttatcgtatcaggaatgaggaacgaaagattggcagagcctgaggaactggaagaagaggttatcggtaaaacacattctttgtctgaaccaagaacttaaaattaagtgcagagcaagcagaaaagtattgactgaagctcaaaagtgagttatattgtttggatggggcattagataaaaataatgagagctggatttttataaaatttgagtatagaaaggtcatttttctttaaacgccacttttttttttaattggggcaggttaaatctgtctgcataaaatgaatagaagagaaactcttggtgaagggaaaacaatgctagagaaaaaaataaaataaaatgtatgataagtacatgcaaatcaatgatatatattttacaaatatggtactgttttatatacccaggaataaacctgaagaaagaaaaagaaaatagaaataaagacacataggagagaaaatggttaaaactcactcgacactgacgaggtcccacatccatgtctctgggaagtttgttcttactgtttgaattatagcctgttctacatagtcagcattctcccctctgattccactagctaaaaaaaaaaaggaagaagtaccttattcagaccttggactctgcaaattatagtctatatattatcagggtgtaaggtctccataaaacacctcactttcctgtatcacagtcacttactatgactgttttcctgtatcaccatacattgcctgttcccctacacagtcaggtaccacataatgacactgtgatcaatgacagaccacttagactacagtggtcccataaagttagaatggagctgaaaaattcctaccacttattgacatcaaagccatcataacgttgtagtgcaatgcattggtcatgggtttgtggtaatgctggtgtaaacaaacctactgcactgccagtcctataaaaatatagcagacacaattttgtacagtacataatacttgataatgatgactatgttaccggtttttgtgcttactatactatactgtttactgtttagagtgtactgttcttacttatcaaaaaaattaactgcaaaacagcttcaggcaggtccttcaggagatgttccagaagaaggcattgctatcatagatgatgacagctccatgcatgttattgccccagaagaactttcagcggtacaagatgtggaggtagaagacagtgatattgatgatcctgaccttgtataggcctagattaatgtgtgtgtttgcatcttcgttttaaacaaaaaagtataaaagttaaagaaaatttaaaaataggaaaaagcttatagaataagaatgtaaacaaagaaaatatatttgtaccctgactcactcagagtaacttttaaccttgcaagctccattcatgggaagtgctctatataaaagtataacttttttttatcttgtataccacatgttcactgtgccttttccatgtttagcaatgattagatacacaaatagttatcatcgtggtacaactgcctacagtattcagtatagtcacatgctgcggaggtttgtagcctaggaaaaatgggctgtaccacatagcataggtgtgttgcaggctacagcatctaggtttgcgtaagtacactctatgatgttcgcacaacaatatcacctaacaatgcatttttcagaacaaattctccatcactaagtgaagcatgactgtatttctgacattgtattttatcaa

SEQ ID NO: 431 - DNA, 1623 bp, Homo sapiens
tgctgggcagtctacgctaatcatcttattcattcctcaaaaacattcggagatgcaggtctcaatagacccattttacaagcaaggagataagagttcgagatggtgtacatctcatcaaaggaaaggcaaatgctgcagagcccggattctaatctgggtgtggctagtttgaaaacctgagttcttttccactttggaggaaaagtgattaacggacaactcccccttcacacctgtatccatgtccgatgctttttccagacagattttggctgcagagctggaaatcacaaagatcataatgacagctctaacagccgtgcctaggctgatgaaaaatgcctttgaacagcccgggctcacaggcattatctttgctaatcctataattggcctgacaagtggtgaaagcataaatgatttcggatatgtgcttttgtgtttaaaagactgggatctggggtgaattctccttttgtctttcaagtccatttgtagttactttttacaaaaaaatttgtttttgttttgtcatgtagacattttaaagcattttgtagtccaatttgttaaaactttcatttatgatttgttttataaattatagaagaaactacagaactaacttactcatttttcttgtagtctatttatttattttacatttggctgggcatggtagctcatgcctgtaatcccagcactttgggaggccgaggtgggaggatcacttgaacccaggagttcgagaatagcctaggcaacatagggagatcccgtctcaacaaataattttaaaaattatccagcgtagtggcatgcgtctgtggtcccagctactctggaggctcagataggaggatcacctgagcccaggagaataaggctgcagtgagccatgattgtgctgctgcactccagcttaagtgacagagtgagaccctgtctcaataaatttatttatttatttatttatttattattttaagtgtttggcccattagtgatacatacaaattcagttttcttttcttttttttttttttttttttttttcaggtagcctagctaatcatatctctattgattgaccaactgaaggacgagtttactactgatgggtccccagtcattcccactggtattcagactaaaagcaacattggtggcaggaaagtccacaggaaaggctctatttcaggaagaagccagaaaccctggagagaatcctagttctgcccctaacctgcaggatggccttagatggatctctttttgtaccttactttgagtttatagctctaccactagggagagaggaagaggaacaaagtaatctctggcactgataattatctttgcataacccttgaattcttgtgataaaaataaatgtatagaccgtgagctactgtgcccagccttacggacatccttttgaattatctttttcactcatagaatatgaatacatttatttagactttttctagaactttcctgttttcatgtctttgcttcatctggaattggcttaacacccttttataaagtttgtgtttgtaaaatttccattgtgacatcaatacgcaatatattttgtaatataggagtttctat

SEQ ID NO: 432 - DNA, 1738 bp, Homo sapiens
atctcaatgaacttcatggctcagttgtaaataataattatattctctaataatgactaaagaagttaatatttatagagcacttaatgcctggcatataacaagtattatatgtgttcattaaagtaatgccataaataaaaataaaaggataaatattggtttaaccagaaatttcaatatgactatattgggagaataagaggagaaattgtggatttggtgtgtgtctttgtgtttctaagcgtgggggtggtggttataagatcattaagtgcttatgtttgaatgaaggtaatgaacagatactaagtaaaattaagcaagaagtataagtataaacagcataacatacccttctatgaatccagtcagtggtctctaaagggaattattagtaaaccagcattaggtgaaacatatagaagtctctttaatgcattcctgtggaaatctctctagtccatttctgtagaagtctctctcacctgttccggcagaagtctatttgaaaaagaaaactagccaaaaaatttttatttgaccttgcttattcctttggttactgtatttctataacctcatcattattgatagtctttacttttacctcttttctctctgtcatcttctcaacttgaggcaattgatttttctctttctttccaatcatctacactcttgaagcctggaagatgcccttgtgtggcagaaagggggtgatccctttccttcccattgtaaaggtcatgaccaacttgtaacaagacacaagttaaaaaagaaaagcataaaaaattatctggttatagttttatgtgacatgggagcttttagaaggaagacccatatatacaggaaaagatatatatttttatgcttagggtcaatgaataatgaacagctgtgtagactgtgcagaactgattgggcaaaaaacatatgatataatgctaacaaactgagtgaggaaactcagcaaggcctgtccagattcttcttggcctctttgcagcatcaattcctccacgtattgggcagagccccttctggaacggggtcttaggatggacaatcagacaagggaggtcagagaattctttatgaccagtttttaaacagaaatgtggggcgaagttagagtaatatttttaggttttatggctggcttaggggaaaagggatttttgttttttatgacccacctcggggaagagggattcaatttctatggcttgcttcaggagagaatgagggatgagagacaggagggtaggagaaggtcagagagagaaactttacttctgaggtcttcactttggggtataattttctgagcccccacactctcttaatgtcactaaatggactttactcactcttacctatttttatttttgaatttagaaacagggtttcactatgttgtataggcttgtctcgagctactagcctcaggtgatcctcctgcctcaaccttccaagtggtattacaggtgtgagccgctgcactcagcacacacattttatttttttatcttgaaaagttttcaaactatagaaaagcgaaaagaataatacaatgaatacccacctgtcaccttaatttaccaaattgctaacattttgccatgtttgctgcccacatcccatgtgtgcacacgtgagtgttttcacagagcaatttgaaggaaagttgcatttatcatgacttttaatctaagtgcttcactctaaa

SEQ ID NO: 433 - DNA, 1985 bp, Homo sapiens
atcctgggccgcgcagctaccttacgtcagccgcctctcaaatcacacggctctggccagttcttttcagcagctttatgagattttctcaaatctcaacaatccggtatattttcaaaagtacatactgagcatcaaggtggtgcagcgaacctcccagatctgcagctccagcagtcttgcttgagcgtccactcaggatggaggtgaactcgcaaagtaactgagattggttgggcatgacatatggtggatctagggttgaatgacctccccagggaacgcaggcttgaagtggaggctgagaatgcaatccgagtacagccataggccggtcaaagggtcttgctgtgtcacccaggcttagtgcagtggcgtggcgtgttcatggcttactgcaacctcaaactcctggactccgaacagctgggaccaaaagcatgcacagcctcaccaggctaatttttaattttttttttttttttttgtaaagatggggtcttgctaagttacccaggctagtttggcctcaagcgaccctctcactttggcctcccaaagtgctaggattataggcggagcgccagtgcctggctgggacgtttataaaaaggagcaatgcacaagaggcaacaaaggcaagtaaagaggaggctggtgggcttgcagaaacgccctgcagtacctatgctgcggttctcccacttcactgataacaaatcctcagtatccactgatacaaatcctgggactgctgggccagtcccaggaatgacactgggacagactccctggaggtggatcagaaagctgcagttttaccaggggtcctgctgttgaggcagccggctcaaggaccaagcccaaataagacactgagaggaaggctgcaggccagggtgcaggagtaatgaccaggatcagtgctccaactggccccactgtgtcattaaaaggctctgcaatggaatccatcctggtccaggcaggggagtgtgcctccagcaaaggaccaggcgtttcactgcatacacagcaggtgttgcttagtgaagacaaaaacccagagccagaggtactgtaacattcttcattatgcataaagtgacagtctgttagctgatgtgatcctgcaaatgaaggctgtctacccaccaagggtttctccctctgctagtaaaactgagactttgtcagtggaggctcaggtaagtccccacagtgctcatgccttccaaaaccacgcccatttccagtgatgatggtttgtgccacaggccaagtactgaacatcatgacactcatagacactcttcactgaagacgtggatagaccctggggccaccattctctcctctctggagtgttggaggggccctgaccagggcaggacccccatgtctggaagcagggttagccctggctcatgggggaccctgctgggcttagccattgccacctagggatcaggcagccttgtttcttcctactttctccaggagaagccccagagggacacttttctaatggtgagatggaacacggagcacctgccaacaggccttcagttatggtcagaggcccaaccaccagtctgtgttagtacctaatcaaacatttagatttaggctttagttattgcctgtttttacttgaaattcttggcttagaattattcccaatggggccaggcacagtggctcatgggaacaaatttaaaataatcagattagcatgtcaaaagatctcacgttatagaaagtgaagatctaagaccaaataaatactgataaaaaatacaggccagcagaagcttggatgatttaataaattaaaattgggagttggggagtagggaagaaggagaggaacatctctttagcatttgttcattctaacattcacatgtttgcttctccatggagttgtttcactatgaacaggttcactaaggagccagttatgtccatatgaaatgagaata

SEQ ID NO: 434 - DNA, 2269 bp, Homo sapiens
ggcccttggagagctccaggccatcagagatggtgcaggaatggggtaggagacagaggaagccctgcaggaagccagcaaaaacctcacctaccttgagaactcagcaatgccggaagagattgagattcctagagaatggaggagccgggaacaaaggagaaggatgggatccaggaacaggccaagagaaccagctcgatgcttcttcctctcccttctagaaaattctgttcagaagccattaggcttcaggctcgagacagagaggaaccgcctctgtgtttttcactgtcgtgtctcagtgctgggagtgtactccatgtggtcagtgtgtgatcaatttaactgtgcctgagggttctgagaagacctcacaaatgaagatggaaagaatcccaagagaccagtgaagcaagaaattatcaaagctgcggtatttacgaagtcactttgtgccagtgccccagcaagctgccaccatggctgccaagccctgtcacccttgctgatcagtacagagaggggctcgctgcgctgggtagaggaagcaggaagtttgctgaactcagacggaagaaaaaggattcctctggagaatcccagcaggtttctgattggacatgggtccatgtccttgtgaagtctcctcctcctgaacctgccctgctctgtgacttgcttttgaccaccagcatgctgcagaagtgatgccgtgtgacttctgagtgggtcataggaagccttgcagtgtggcctgggtcttcaggaatgcttgtttgaggaaaaccactgtcatgtaagaagtatggctaccctgagactgtcatggtgtgaggactcctaaatgagtcacatgtggatggccacctattcaggcctcagttcttccagccattgcgactgtggcccacgcatgtgggggaagaggccatttttcacgcccatcccagttgagtctttagatgatcccttcctgggcctccagctgactgtaaccagactccaggcaggaaccacataggaactgaacccagtcaacccacaggctggggaggatgatagtccattgttgtataaagccactgcatttggaatggtctgttgtatactacctaatcagaacaagactcaatcctttcttcatagctggtagtcttaagcatttattaattcatattttagtcacattatgaattttctatcacaataagcatttattgagcactttttatagagggactatgctaggcttgaagaataaaagacacataggacacggccctgcttatgttcaggtagctgtggtctaaacgaggacgcacacataaggtgaagaccccatgagatggaatacacggggataacaagcctcacagtggactggtcgctttaccaggggtgaggcgattctgcctataaggtagggaaggactttctgaaagggtgacactgagttccaccttgaaaaatgcctgagaattcacccatagacacagtggaagaggggtgttccgaggaagggagatcaatgtgcagagagacagaaatcccggttcgcttattcccaacaaggcagagaacaaacctctcccagtgatgccctcccttgttcccgtccccaatcttttgataatagtgggaagggtttcaaagtgagggttaaaaatgggctggttttgccaccccttagtaagtcttgggcaggattctcaactcttgacactcattaaaatcacctgggaagcttttgcaaaactccagcatggtacaactactttagaaactggtgttatccattaaaactaaacatttgcttatcttagcaattccactcctaggtttatagagtaaacagaaacacatacgtgcctgtgtcaaaaaacatgtataggcatgttcaagaccacactgtttggccaatgctgggattgcaggcatgagccactgcgccaggcccatgaatgaatcttaaaaataaaatagtgaatcaaggaagtcagacacaaagagtacatactctacggctgcatttctataaatatcaaaaagaggcaaaacaaatgtgtggtgttagaaatcaggagagtgttactatcctggggaggaaggagaagacctgaaggaacttgaggggggctttggcctgctggtagagctttttttttttttgtctcgatctaggtggtagttacatacggatattcacgttgtaaaagtttgccaaggctttaatgtaattatctgatgcatattataattaaaatgg

SEQ ID NO: 435 - DNA, 1772 bp, Homo sapiens
ggggaggggctatggttgtttgctcagaaaaaagccatctcatcccccctccctggcatctttctgatctgtcctgttctgtaggtggaatgttcccacctttgtttgcctgttcagacgtgtttgtccttccattatttttccggctggattgccagcagaatgcagagcagcctggagaaatgaaacagagctgacaggtctcttaatgaaagcttgtttgcacactctgcctctcctgtgatcgctctgtgcctgagagcctctaggtgtggatttttacatgagcgtttgtctttcttttgattaaggcctgtagaagtggattggtttttgtaaaccaggcatttctcatccctcactcttgctcttcctccttccacaccttctctgcccaccttcttctcccctccccgttccttctcttgctcccacagcttttggcaatcttaagcaagctaaattagtttgcccatcgttgctcctcttctgtgacctccctcccaaacatgcaccaccatcacgatgcaaagtgctagaaaagggctctgctatcaattattcacatgttggagtaaattttttgatgtgttattcctaccgggagcctttgcaccccggttcacgatatggcatgaaaagttctgtctcctctttaaggaatgacaaagaccagctgcttactcactgaggtgatcttctctgtctctttaaaattacagcatgcctttgcccctgttggttttcactccaaaaaaatggggtgccctcttggctttgttgttttcccacagccttgtggatgacttggtaccctacttctttttttttttttttttttttttgagagtctcactctgtcacccagggtggagtgcagtggcatgatctcggctcactgcaacctccacttcccgagttcaagcgattctcctgcctcagcctcccgagtaactgggactacaggcatgcaccaccatgcctggctaatttttgtatttttagtagagacggggtttcactatgttgaccaggctggtctcaaactcccaacctcgtgatctgccttcctcggcctcccaaagtgctgggattgcaggcgtgagccactgcgcccggcctgtaccctccttctttttgaaaaagagagtaccttcttcctctgctttcattctgaagcctccatttaaaacactcctcctccttagcaatagtgagggtcccttcagactagataggatgtcaaaaggccaaagcattgcccccaacacactcctttaatgtagtcccttctttccaggtgtcttcctgaagagagaaaaatttgtagatcagagagatgcaattgggagctccgtagtgaatctagttagaaatgagtagcagaaaaaataggaaagacccgtctctcaatattatggttgcttcccaaaacaaaaccagttgatacagcatgaaggcaatttaaaatgatagatatggacatgctgccttacatttcatatcacctctttactaaatgagaattttttggatgcatagggaaagtttgcataatattgaaaagtttgactttctggaatctatgtgagctcaggaatatatttggggatcttagcagaggccagtggtagcctattaaatctaaaacaattggctttgtttttaaaaccagctgctattttgtagtttcatttaaagaaaaagtaccttctattcatttgcagaagttgctttattggattctatggtcattgtatatcataactata

SEQ ID NO: 436 - DNA, 2262 bp, Homo sapiens
agagctggggcaggatgcacaccccactgccccgagtctcagtctttcagctgccttggcctccatctctgagctactcccctgacaagggggatccaccatagggcacctgggctgagttccagggaaaattggcgggggatagcctgggcctcttggacctagttcaggcctgctttgggactgtcctagcatcccattggatgtgtaggatatacttgctgattgtctgggagaatccttcaattgaattaaagaattaggtctagaaatcctcaaacccaaccaaagaaatctggaaatcctcaaattcaaccacaaaatcctagatctggcacagccttcaaggattcttccagtccggtctctgccccagggggatgaacacctcagccatccatgacagctgtttttcctcctggcctttaaaatcctccctcctcagccttcactcctactcaccacctcaacttaatttctcattccccacccaggaagaacctgtatgccccctgcatcccactgcacttccggatgccttttcccccaagactggaatggcctcaggcccccatctccaggtgagtggccctcacctacagactcaacatatggcctttggctcttcccacttccaagagtcttggaagggatgggtcgagcaagcagaggaaaggaagatgtgagttcccaaaatgctcctcacctttttcttctgagtgggctccttctcactggcattggagggcttgcggcgcagcatggtcctccaccctgggagactccgtccctgctctcctaggtgtcaagatgcagaggcctcttgcttagcctcaccagaactgcccgggggcacggcatgaaccgagccttcagctgccactctgactgctgcagtggcagcttgggggtgcgggccatggacacccggcagcagcagcatacaggaagccctgccacgtgacctactcttacagcaatcgcagcccctgccggcccctagggaggaaggaagtccaagcttcagtctccagagattctgatgcagacggctcttgggttgaacagtcagagaaagtccctaccttgttgtctcatgtcgatcatcatcagtccttctgctccatcttcctagaggtggacaatcagctcgacattatatggcgtacccaagccctttatctcactgaccagaatccagaaaccagaagttggaccatctggctgcctgttggatcaatgctcccggtcaactcacgtcttcaaggtcgtcactgccatcactaccactaccatcacccctgtgaccatcaccactgctaccatcactcctatcaatagccatcaccatacagccatcacccctacagccatcaggcctgcagccatccccgctgccatcagcacacctatagccattgtcactatggtaatcacctctacagttgacatccttcagccatcacctctacagtcatcaccattgctgctatcacccttccagccatcatccttacagctgtcacccttctggacattacagccaaccgcactcatgaccctctcacccctataaccatcgtccctacagtcatctctcctacaaccatcatcacatccgtgcagccatcagccctgcagccatcactcctacagccatcagtacacctaaagccatcatcactagagcagtcacccctccagtcagtacccctcagccatcatgagtatatttatcaccaccacagctgaaaagcacttacagccaactccacacctctagcaagtatccaacaggcatttatcaaacacctatgcagtatattcaatttgtgggttgtctgtggcagattttaataactttaataacttcttggtattacaaaagcaacttgtatttgtcatacaatttcacaaacacataaacaaaaaaaatttcaaaatttcccatcattgtcctctacactgttttaagcatctatctactctctgtctataaacattttttttctttttacaaaaatcatactgctcagaaaacagataattggtttggggagaaggggaggggagagaagggaagggagacatcacacagggtaacaggaagcttttaggagtgatgaatatgttcactatcttgactgtggtgatgatttcatgggggaatacacatgtcaaaacgtatcaaattgtatacttcaaatatgtgcagtttattgtagctccattatacctcaataaagctgtttaaaatttttt

SEQ ID NO: 437 - DNA, 2081 bp, Homo sapiens
ccttgggttctacacggtttccctcccaagccaggcagcgcatctctcaagaatagggaaaggtctccatcaactttgccgtcacggagcagtgaccgcgggcgggcgcggggcacacacctgcctgcacgcgggcccgctccccacccggctccccggtccctcagcgctccccgggcgcgagattcctaaggacacggcgcctcattcccgggctctaaatctccactcgcaagctgtgcagcggggaaagggcagtggggacacttccaacgcggaaggccagcgggagcgagacacttatttcattggccttgtttacagtacatttttattcccaagcactcgcggccgttccaggtctctgccccgttaggaagtgtaaatcgacgccagacccttcctggtgcacacaaagagaccaatcgtgcgcatgtgtctaatcctcccgcagcctagagacaggcaggcgggcgaggcagccgggcgccgacgtcctctgctgaaggagtggagaagaccgaccgacccgagtggttgatttatgcatgcgttttcgttaactaggactgaaagtggctcctggctcttctgggaaaatggagaacgtggttgttatcaatggataccaatgacttcctgaaatgctcaacttgtacaaacaaaaaatgaaaaactttctccccttaatgtagaaacacatggcattttaaaagtaggtttttcattattgtaaagatgttttaatttttaggattatgtttttgtgaaagattcgaggaaaaagagcgatgttgagttttggccggaggcaacatatggatttttactataggctagaacatttagtacttacttctaaggatgtttctaattagtaaaatgtataaagattgacaatttttaaggtgactcctgcaagagttatatttagtaaaagttcaaaagatgatgtttgtttattacggtgagcagtatttttccaggttctcatttaaaatttcctaccccatttgaaacacaataatagaaagaaaggaaacttacttgaatgaggttgtggggggaatgtaaaaacttgaaggacaaggtagctcagtccaaccagatgagcatccaagggagatttttgtctcatcctttgcagaaatctccatctagagcccgtgtaaaccaatggaaattcttacttgctttaacagcacactattcctattcattcattaatgtgggctagcctttttttttttttaaataaaaaaaccttccaccgtgcaaagtaccagatgctggggcagatcagatactaaggataacatggtaaaatcctttccgctgtcactctggaatgggcccaaactctgaccttcccagaaaatttgcataaactccctggaatgggctcaagctctgaccttctcagaaaatttgcataaactctctgataactccttgtaattctttcctctaaatacctggcacagcttgtctattccacccacttcttactatttcatacgctgcatggccattttttttagaaatgtaaagactgctttaggtttgtaaatgctttaaataagtgatttcattcagccctctttgcaaccacatatgttaaatgtggttgccagttactagaggaaattgaggctcacagacattcagttgcagagccacacgtttcaagattgatttgaggtctacagtgatttctatgaccactgactccttctcctatttatataatcatctgtttcacaagtcaattatcagttctttgaagtcagggattagttttgtctcagcaacaatcttagtgcttacataagtaacaggtgtgatcaataatcgtagcttttaatgattatgtatgtgatcactgtgcctacattcatgtttatcaacgtctttgtaacaccctagcatgacttcaatattctcatttatcttttatttttaaatttgtgatacagcaatttttatgtatgtgtgtccctctttttatgatctttatttgcttcttgaaagcagtaggttatctgtcatgtaaattttcccagttctggatttggttgcat

SEQ ID NO: 438 - DNA, 2817 bp, Homo sapiens
tgaagatgagtacaagagagaaagacagggctctgaaaatactgccataggctcaagttccaaagtgctggagttacaaaagtataaaggacaacgcaaaggactttttagccaaagaagaaccagaaaggtaaaacagaaagcaggaaagagtgtgggctctggggctggctgtatctgtgtttgaatcttggctttgtcctttacttcctggtgaacttgggtggcttttgagcctgcatacaccttgacttgagtgtggctttcatttgtaaaatgttgatgatccgtgggcctacctcagaggtttgacgtgaatgaaatatatttggtggagtgcgtggactgtagcgagaattcatcacatatttgctcttatcacgattattaactagaataattaagaccatagtgtaagagcatttaaggaagggaagcatacatttaaaaagtaagctacaaaatagtataacagtgtgattacacacacacacacacccactttaaaaagaaatacaccaaaatggtaattttaaaacacttggctggtaggtaaaaataaactggaacacaatcagaggagaacaatcatatctcctgaggagcagctgaaaatgggtctattttccatggagagaagtggacacggaggaattttaaggggttattctggttttgtaggattgcagcggctacatctagaatcaacgggtggaagttatatggcataaatctcagatcagtgaggtcagtatcatttgatgcttatctgagctttcagaaggaggaatactttatcaaatgagttctttatgccctgggccctgcactgaatgagttacatgcattctcttatttaaacgttctctccaacaggaagagtcctagggaattggagacgtcgcgaaggaatgttgtaaggagaattcaagcctaaaacggtgataaatcacagtatctgcaatatctttttgaaactcatgggctttggcctccatgttctagccttggcaggacagaagggtggtaagagcagtatctggtagggggtcaagagtctggctcatggtggaggccaagcagaagagtgacctcatggggaccgcgcctgggtttgtgtgcccactggagagctcgttcctattgcagggccataatgtaaagcagagggtgcttcttttcagaaagctcacctatagctccttcaggcctgtactcttaggagggaaaaaggaagggagtacaggttctggaatttaaagaaacaaaaaggattattctgaaacataaagactttagaggacatgtgggttcatattttatgtctttttatttctttattccctttgctttccaggcttcaaccatatatctggccctcacgtccaaaagctcgagttgatatgttttgttgctgtttgtttcttattgtccacagaacttgaggcatgggccacagtactggggataaggaaggtgtagggagtaacagccagagtagttccttctctccttttcccaatggcaaggctgagaaaccatttatcaagcatttttattgactgggacagcttgaaagcactgtcattacctgggcccatttctcttccattttcctggtaatttctgtagtcactgaggaagcccacattctggttggtccacggatttccttattgcaatcatttatttgcctgcactataggcttaattctccttcctcttttgtaccaagagaggtgctaaaaatcctatcaatatcacaattgactcctaagctgaagaagaaagttattttcttatgtgacgtgtaaagtgatcctcaatataatccctgaagttgaacttcagaaatgtttcgtgctgcagcaaaattggggggaaacattggcaaaatcttccaagaacacataggtcttgggatggccacatgcatttggctatctacattcctgcacatctgtggagagaaaaaaagtttcttttttaaaatacggagtcatacgtttattcttaggctactccaaagatgcaacaaggtgttttgtttttattttttcaaaatgggtttgacagtctttctgcctactgattgtcacagctaattgctccttcattcgcatgagaagagaaaatagaagagagggaacgtgatggccctttacagcagcttctaggaatgaatgtgtataaaataatctactgaatggagcacagaatgaaagcaacacataagaattcaaagggtacattttaatgatgggtgcatatatattaccaacttgggggagaagacatggcacctgctactctgccttcactcaaagtgttaaatgaaaacctctcaaaaattaaaaaccagctgtcttatgactgacatttaattttgaaagatgtgattttcctccgtatttcctgtgctctttacaaacctgtttatcaaatgtatagaagacttttgtttatgacagtttatgattaatcattcaccaaggacacgagtcctttgtacctattattgtcaagcatttgtttggactgtgtttgaccctttgtaccgaagtggtctgtgttggaaccttttgaaaacaatctaccaggctgaatgtgctcatgccacttactgaaatatcatggctctgttgctgtgacatcctgttacatgggagagaccaaaattacaattttagtgacaatatcaaatctattgttacgccaggataaatagattcatacccagttagttgtaagtattctagagtgaaattttaaaaaacattttaagaaatgtatgactgtctggtgaccagagttaca

SEQ ID NO: 439 - DNA, 1876 bp, Homo sapiens
ggtgagcacgaggacgtggtgtagggaagaggacgagtgagcagcgcctggctgtagggtcagagggcgcctggtcatcctgagagccttgccttcgcagtttgagattaggggaagttcggggcctgagaaggtgggcgcgatgctggagctgggaattcttcctcggagcccctggaccgagttgcgctgcgcctgcagcaacaggccgggaaaaagtgtccgaacgcctcgtggactgcagcgggggaaatgtcccttaaaagtgcgacgaagtggggaagaaggtgtaattactattatcagcatctagaaagcatcatgaatttgctggagtacttcctagcactgacctccttcattctgcgttgttcttactggatctttccatcagccaacaatatggaagtaccaatacaaggtcaaatcattcctggattcatctggagttgcttaaaagttaaatcattggaatttttgatgataccttttctatatggattacaatttgatcgctgggaattctccaccttaaagaagactcttttgctatctggaaacccatgtccacctctgacttccactcaaaattgctttcctcacagtctgactgcgagagttgtgaaaaattgggatgtgcttctgaggtgggctgtggaatgccattaccctcaggtgactacagatgtgttaacacccagcatgttccggtaggagactttctggatggggaagatttccaggtgtgaatcatggcaatacagtgaaagacagtgatttactgcttttgagggcgtgcatgtatatgattaacggatggaagtgcaggactccaagatttacttccttccctttccagcagaattacctgagacgagatctgcatttactgctcaaccacatctaatttgatgtcctctgcagatttaaaatgtgtgccttcttttccgtcaccaagtcatccctgggttactactgaacatccttctcaattccccccgacccatggatggctgttctccattgtctgtttcaccagatgtcctcaaaacaaacagacagaagaaggaagtggctaatggagctgtggagtccaagtgtgactgccaagaggaatccagcaaagccaaaaagcccaagcatgtagccctgcccgaagcacgccacacgcatggaaaacccagaggaaatgagtgaggatcaatgggaagaagagagccagccaggaagttgaagatttgtccaggagcagatagctgaagagagagagagagaagagagaacggcttacagctcaggtcctctctccatgcttaggaaccactacaaatgctactgccttgagtctcattttgtttccctctggaaaccacatgtgtaccttgtttgcaacagtatgggctcacaggcagaaggaattttccttgtcttggatgagacttttgacttggacttttgggttaagttctggagaccagaaggccaaaatcaaaagtatgggcaggcttgatttctttagaagactccagcggagaactgtgtctccttgcttctgattctacatctccatccatgggccactgtttcagcaacctcagccagtgcaacacaacctcagccaagaagagtatgcagagaaaggagtcccctaccctgcacaaaaactgtgtctgaaaactgtctcatattgtctcaagttgtcattcattgtgaattagacctgtttaacatgtaatctgcaacatgcttcactgtctaattttccagagcccctcatataaggaactgtattattggtataatcatcatggtgaagaagttggtatgtgggggagagatgacagaaacagagagtaagtcagagctggctgcctga

SEQ ID NO: 440 - DNA, 1934 bp, Homo sapiens
aggaggattaaagatggccaccaacagctgcgggaaacggcaacaacccctcactttccgggatggtccctgcgggtcggcccggccttgatggagagaagaaacccgaggagcgccgaggctgaggcggcggcggcggggacccagcgaggacgaggacgcggcggagcagggacgggggcaggagaagggaaaggcggcggcgtcgctgcccctgctgcctagcaccgctgcctggcccggcggaccggttcccatacctcgcggccgcagaatcgagctcgggccccggcccccggcccgcggcgcggggctcccgggccccgccgcggacgtcgcgccggtcgccccttccccgtagcccgtgcgccctcggcgcggagccccggcccgccgcggtcccgtctcctgggcctgtcccgcccgcgccctccgccggccctcaggtgagtacccccgccatcctccccgccccccgcgctcccggagaggcggtgctgcctcggcccggcgcgcctggccgctttgttcgtgcggggcggaggcgctgcccactcgcggccctgcgcccgctccccctagggacaggccgcggcccagctgctgtcctccccagggtccgccttggccgggagccgccgccgccgccagggtgggcggcctggccctagtcctccgctgcctcctggggagggcgccgtcgcggacgggtcggggtcggggtcacgggcggcgagcggagcggagcggaaccgggatatttcgggggataggggaggagagggctggacgttacctgtgggaagcagtgggggcgtaggcggccctggctcgcaggggtaggggatcaggccctggcccccccccgctgcatcctgcggggtctcagctcccggagccacccggacctgagcgcagccgagcgcggcctccatccctcgggcgctgtctccagggcttcgggcctctccccctacgccctacgctggaagcccttcgattatggaaggagccagagatgctccagttctcttgtactggggatgtggtggcatcgcaaacattcacagttacggcgggatttaagaagcagatgagacctttccaaccaaaggccatctggctagtgtttgagcctcaggtgcagtttttcattgatttttaccatttaattgtgagatctgcaatatcaggtcttcctttcacagaaacaatgtgatacaaccagattgaaaggatatttactgcatatgtgaacccttgcatggacttccagtaccttaaaaatggttaagtgttttctgttggaaaatggtgctcatgcatacgaaaaactatttttgttcagtaatggttgccaactggtttactatgtagtttgaacaccaaacaattaaaaaggaaaaacaaaaccctattcttcgggctttttaaatagtggcatatttccaggttggtatttggtagcaggttgagaacaactggtttcgactatcaggcgacgtaacaaacataacataaaacagaactaaaaatgtgctaatttatagaaaaacatgttgtattagtgcataagtctagtaaaaaaatccgactttaaaaaaaataaaactttatcatgagactcttaagtataggaattaggttggtatgtttttatgtttaaaaatatatattgcattagaattgagaactcttcctatgatatttgagcaatagtaaagtatgttaatactttgaaccattctatctcaagctgctagaaagaggtttctactgctagtttctttccatgagttaaaatggattcatctgaaatatatgtatgcttttggtgggtcacagtgggtcatgcctgtcattccagcactttgggaggccaaggctgggggatcacttgaggccaggagttagagaccagcctgggcaacaaa

SEQ ID NO: 441 - DNA, 1888 bp, Homo sapiens
acagctctgattggattttcgagggattagcaagaaggagtggcagagcacgaagctggctcccaattcatggctgcgcctcccttagcaaatcccctgcatttggtcgtgacaccaatgaggagcaggtccctgttttgtctctgcctgggctggcctgtctggaacagtcttctagggtcacagtgcacgtctgcctctgggcagctgcctgacccaggcctccacgagagcagggaagaggcacagagacgttccatgatggctgggtgaagaagcaggcagtcggggtgagttaggttaaaacccaaagccccttctgcccggccctttcctggctgtgtgctgtggggcctggactccccctctctgaagctcagtttcctgtctctaaagtgggtgtcacaatgacagccgccaccattcctccactaccacaagaatcataggtggtgacctgcctcatattcccgaaacgtctctgcgcggcaacatcctcctttgagcctcgtctcatcccatcgttgcctcctcctgaaggtccttctgctgctgacacatcaagtgcatggggacatccgtagcacgtggccagccaggaggacactgaggcctgcctgaggccctatctgtgggtgtgatcataacgccaccaagaacagctcttgtgcttacagggtgctaagtcctgaccatctcaggacatccttcacactgccctacaaggtagggactctcgctgcagatgagggaggtatctctcagtggggtaaagggtttggccaaggcggtacaggtcgccctcaggctggccagcgcgtagcactccacagccacagcagacagcatcacaatgaactactcaggagcaggagggcaggctcaggtcccaggaagaggagtgtggccacaagcaagattacccgcctggcctggaagggacaaccgagcagaatttgaaataagcagcagggaagagattctggtttctgtggggccctcatcagaaaccccatagggcccttcaccagaaaccctctgcctgcagagaccacctgcagatgggggcctcagctgcctgcagttgcccagggcatggaagcacaaagctcatttctgttaccacatcactaactgtaggattggacctcaacatgcatactggttctggcacaaaaggagctgatttcttgttcttgcaactgtcccaggcaggggttcctgctcagcagatggtgcctctcctagaagtaattcagggaccagccaccttccatcctaggcctccatcacagcccttgggcctcgctgtggtgtgcacccccagcagtcagaaaagagggagactgggagcacatctctgtggctgcaggcgtcactttctgctgttctcttgtctacactttctgtgttttgttggacaatccagtcgtcaggccgtgcccacctgtcatgttggatgggaaatgtagtccagcagtatgtccagagagaagggagaaatgattttggtaaacaaccaaccatctccactgcaggtgatgcaggcaaacccaaaaacgggggctcagcctgagttggttcttggcttcattcagggaagaattcaagagcaagccaacagtgaaagaaagcaagtttatatagcgtagtgaacagcaagatggctgcttcattggcagagcagggctatcccacaggcagaggggcccagagtagcacttgtgggttgctggctggctctatttatacccactcttaattatatgctaaataaggggtgggttattcacaaactttctggaagaggggtagactttatccagtctatcactgatgggcacttgggttggttccaagtctttgctattgtaaatagtgctgcaat

SEQ ID NO: 442 - DNA, 3041 bp, Homo sapiens
agtatcctctctgctgggggtattttgtcacagtgacaaagtgtggtgttttgcaaagctgattgactactgtcacctcttagaaggaggtaaacccatcataaagattgattgatggcagggtcagactgactatggcttacagttggagctctgtttgagttaaggtcttgctccctaactcaaagagaagtgtagccctggacaggtagcattagcatcatctggaagtttgtgaaaaagcagactcaggcctcatgccaatcctactggatcaaaactccatttgacaagattcccaggcaatttacatgcccattaaagtttaagaggccctgggttagggtgcttcttttgaaaagaacataagcatctcttccagaggccagtcatttacattgtaataagatagcttaatcagaatacttctcatttatcctttagctaaaatatgccagaatgggaatttttgttgcaagggaaagaacatcaccaaatccccaactcctgaccactactgtaacttcagctcttaccatggggatctagcacatagtagcatcaacatatcttttgtcatttggctgaataactcattcacaatgagaaagaatatgtggttttctgttattcagtatttgacatatgctcaggtgtggagggcagaataacagactacaaatgtccatgtcctaatccctagaacctatgaatatgttaccttacacagaaaaggggagtgggtttgcagataaaattaaggtggctaagcagctgattttaagatagagagaatgtcctgggtgatccaagtgggccccatataatcacaagtgtcctgaaaagtgaaagagggaggcagaagagaagatcagagtcagagaaggagatgtgagaaggacaagtgctgcccttgctggctttgaagacagaggaggagggccatgggttgagaaatgtgggtgatctctagaagctggaacaggcaaggaaactctcctagagctctcagaaaagaacgcagtgctaccaacactttgatttttgcccaataagacatgtggtagacttcttacctacagaactgtagggtaataaatttatgttgttttaagctattaagtgtatagtaatttgttatggcaacaatagaaaacctgtaaaacaggaaacagttcctttgtttggaatatgaaaaacagacaaaatgctagaattatgtatatttttttctttttcatatcttttggaattcacttgcacatgtttgaggtaatcctgcttctcagttcgtggaatattatcatttattcatagaagaaccaacctttttgtgaaaacatttttttaaggtttattgtttttttcccttcatctatcattcctaatcccactgccctccttgcacaggcatccactataatacatttaatatagttcttccaatccatccaacagacgtttatttagatatatgagcgtccttacaaaaatacacaaggccgctttatgtgtgtgtttgatacataaatggaattgtgctataaatcccattctgtttttgcttttttactcttaactccatttttgagatcaatccaagtttctaaatgaaaatataattcattatcctattgtatacacaaactatattttacctgtacattccttggacatagacacctaggttgcctctagcagagtgatagtgaagcttgttaggaacatcttgtgttccctgggaagaatttccctgggtgtatacccaggagtgggcttggtggatttaaggtgcacaattatccttcagaacggctgcatctgcagtgggtgagggctctctttccccacctttttgccaatgcttggtaatatctgacttaacaagtgatatgcaatgggcaacaagtagctgtgaaaggaagtgctatatagatgacttagtggaaaccatgctgtaaatgcagaatgagcagggaaatgttaaaaaataattcttggaggaagagtcttaccagcaggaccgatgtggctgggaagaggaaagccctccagtcaagaaggttgtgtgtgtggctcaccattgagtcatctttatcaccagtgagatcacccttcttctgactacagctatctggaactcagtctagtctatttccagcctgagctttttgctaaccttccctggacatgtgagggactctcttgactcagcttccaactcattctctgctttaacttcctctttgccactttacaaatttgtgtgatttaatgacttttgacagacacatgcccctgggaaaccacgattattaacacatgaaacatttccatcattccgaaaagttccctagcatatgtttgcagtccatcctgcctcctgctcttagcatcatagaactactgatttgctttctgtcacttcagggaagttcacattttccagaattttatataaatagaattgtttagtatgtactcttttgtgtctggcttttttccactcagcataattattttgaaatttattcattttattgacatatcaataatttatttgtttttactgctgaatagtattctattgtatgaatatacggcattttgtttcttttcttgagggacatttgttttgggctattattattaaagcttctatggacattcatatataagcttttgtgtggacatatgttttaattttcttggataaatacttaggagtccaatggctgggtcatatgacaggtctatgtttaacattttaagaaactgctggccaggtgcggtggctcacgcctgtaatcccagcactttgggaggctgagacaggtgggtcacctgaggtcaggagttcaagaccaccctggccaatatggcaaaaccccatctctaatagaaatacaaaagttattacggtgtggtggcacatgcctgtaatcccagctacttgggaggctgaggaagaagaactgcttgaacctgggaggcagaggttgcagtgagccgagatcgcaccactgaactccagcctgggt

SEQ ID NO: 443 - DNA, 2103 bp, Homo sapiens
gacagaaccggaagagaccgggggcgaaggcgacaggggtctgtggaagagacctgtcggcggagagcggtccacgttttcctggagaaagacgaggccccagggcaggagcgcgggctgcgctgggcctttacttcgccgcccgcgggcggggagaccggccccgtacccgaggggacgaggggacgaggggcccatgcccagtcagggaagccgaaggcctggaggggcttccgggagcaggggctggagttcctctgccaggcaggaggctggcaccagacacccggcagagggaggcggcgagggcccagcaaggattctccccagcccctgtgcctgcgtctcctgcggcttctgtgcgcggaccgtgtcctgggctgtgtagggaacgctgcctctctgctcgggacgtggattcctttcccctcctcctcgcctggctacttctgacgcaggtcgtcaggactccgcttgggtgtcacccgtgcaggaagcctcccttagtcaagggccctcggcacccgccccatatgtgactagcagccctcctgtgtatttcatcgcgcccccgttgtttatatcagctattccactcaccacccttctgggcaaccctttatctcccgctctgaaatcccatcgctgagggctgggacccctcctcagtgcttatccctgtttccccgcgcgagtctggcgcctggcgtgtggaaggcgctcagtaaacgtttgtggagcgaagaaacgacgcaaaggtgatgagcacgacgcagttaggaggctattggcccggcgcgggggaaaagggggaaggtcgggctcggggtggcaggaccccagagggcaggggtgactgccggggtgctcttggggcaaggtgggcgtcaaggcccccagcaaggttgggataaagtttctctccgaggcacagactgccgcctgcgggctcagcatttacctctcccttcctccttcctaacgcaacttcagcggagggctgagggctctactgcgcattacctggaaaacttatttcactccacggccctaagaggtgggccttattattagccacggccagggttagggtgaggccactcctccagggcagaatttcagggggtgcccacccccccgccctaaatcattcagcaatcaacataaattatagttcaacgcaatatatcttatcttgtgggccgcaagccacccagctgcccaggcaagagcctgaaggcacaagctgttccagtacagcaaagaaaataattagaataagaaaagttttactagagataggaaacggataggattatatctgactattattaatcattagtttgtagcatcactctttgttctattaccataatgatctctgttctattatgattaccttgggggaaaccaggccacacagagttaggagctgaagggccacagtgagaggtgaccagaagacgagagtgtgagccctcattcacgcccagagaagggccgctggagggctccttggcctagcggtaatgccagtgcctgggaaggccctggttacttagcaggccttggtctagcggtggccccagtgcctgggaaggcacccgttacttagcagacccggaaagggaatctccctctctccaggggagacagagaacgctccgctccaccacctcttgtgggaggtctgacattagccaggccggcccgcagtcatccggaggctccaacgtctgtctccctgtgatgctgtgcttcagtggtcacgctccttgttcactttcatgttcagcctgtgcacctggctcctccttttaagttcttagaagacagcagtagcagaactagtaggagtaccacagtcttcgatctttctgataagtgcatagaagaaacgctgacgtttgctgtcctccctctccacctcggctaccacaaagggaaaggccccctgtccagtggacacgtgactcgcgtgacctatcgatcattggagatgactggcactccttaccctgcccccttgccttgactacaataaatagcagcgcctccaggcactcggggccactacctgtctgtctccgcgctttggtggca

SEQ ID NO: 444 - DNA, 2887 bp, Homo sapiens
aaagcccaagggctcttcagttagcagatgatgagtgcttccaggactgggttcttctcttcaaggcaacaggtttccttctggcccagggtatgtctagaaatgttgttttggatctagggcctataatggggtcctcatgagtctgtctggtcccctattatattgtggttgaactagtgtctaagatgcaagacaaagttctttttactctttcctgttctctcctcaagcagaaagagggagtcacttttgtttctgcaagatgcactacatggggttggggtatgggtggtgcaagtacttccttagttaccttatctggtgtctcatttggtgtctccaagtccacttgactccaagcccagcacagcactaggacttgcccagacattgcagtccttgtggcctagactgcctttcaattttatttaggactctagagtactttagcccatggtggcaaggcttgctgaaactcaagtttcaacccctaggatggacaattctcctctccctagggctagtctaaatgttccctctgtgggcaggcattggctgagtttagcctgattttgctttctgctgtgatagggaagcactgaattcaatgcactggctcacaggactctgtgttcttcctcctctaagcactcagattctctttccacaccatgtggctggtgccagggaatcagggagagtgggttcggtgattcaagactgtctttcctaccctcttcagtgcgtctttcagtaatataaaattattacaaaaccagatattatgattgctcatctcgtttttgattcttatgaaggtgattttttttgtgtgtgtgtatagatggttatcaaatttagcattcctgtgggagataatggacaatcagtggaggcctctattcagccaccttactctgcccctagattgttctttacaaatacacacatgcttatatcattctgatgtttaaaaccctttaaagttccccattgttggtgacctgttccctcttacattatcaaacttatctcattactttcactctaactctaaactttagctacattgtccttgtttcaattcctcaaccttgccatgctttctgttgactcagagccttttcacatcctattccatttacctgtgctgctcttaaccctcttttttctttgtagataaaagatgtataccttctgtggagctgatttcatatattactttttaaaggccttttttattgactcatcaaactagatttggttcattatatgcctgcttgtctcctacaccttgtgtgtgtgtctgtgcattccagtttttctcagacagtcctggtttactcctattgtgcagacataactaccaatagcacctctttttgatcttaaaaatgttgaggtttggattgcaaattatggtttccctacttgtatataacctttactgtgcatacacaattgcagttaaacacttacttgtataattaaattgtctatcccatttaaatttaggcttcataggaaagacctgtgtctgttttgtttgctgaagcatcttgatccttagcatcatgcctagtgctttgtaggtgccaaataaatatgtgttgaaacactataaataaataaaatatgccctgaattaaatacagtcctcccttggtatccatgaggagttagttccaggaccgacctcccaacatacaccaaaatataaggatgcacaagttccttatataagatggcatagtatttgcatgtaacctatacacatcctctgatataccttaaatcatctcctataaaattacttatactatctaatacaatgtaaattctatgggaatagttatactgtgttggttttcagttctattacttttatttaaattattttattttgtaagtgtattttttatttcaactttccttttaggttcaggggatacatgtggagatttgttacatgggtatattatgtgacactgacgtttggggtaggaataatcccataacccagatattaagcatagtacccaatatatagtttatcaacccttgccctccttttcctctaacatctagtatcccccaatgtctattgtccccatctttatgtccatgtgtagtcagtggttagctcccacttataagtgacaacatgcagtatttgattccgttcctgtgttaattttcttaggataatggcctccagctgcattcatgttccaaagaacatgatttcatttatttttatgactgtgtagtattccattgtgtatttgtaccatattttctttatccagttaactgctgttgggcgtctaggttgattccatgtctttgctattgtaaataatgctgtgttgaagatatgagtgcatatgtctttttggaagaacaatttattttcctttggttatatacctagtaatgggattgctgggtcaaatggtagttctaagttctttgagaaatcttcaaactgctttccatagtggctgagctaatttacattcccatcaacagggtatattattttggttgaattcatggatggggaccaggggatatggagggccggctatatcaaatgacaaatgagaagcagcagttgaaggagactatactggcacaagggcagagtggatggtacatagtatttcaccaggtattgacagaataattacaagttttaactttggctttaaatagaaaatctattttacgaagttgatctttgtgcttattattgccattttacatctgtttttcttttttattaccttaaatatacacaattttttattgccattttacatctgtttttcttttttattaccttaaatatacacaatttttatttgtcaattatatctta

SEQ ID NO: 445 - DNA, 1672 bp, Homo sapiens
cataacacacataacacaacccattaattttttttttcaaaaagcgaagtttattttactttttcccattatgaaagtcatcaatgctcattatagaaaatttggcaaataacaaaaagtggaaagaaaaaaattccttaataatttagtatatttgatatattcatccttaatccttaatcttttactatgttatatctttaatagttgtgatgatatactttttgtaataaatatatatttgtgttgtggcttttttaaaatttaaaaacataacaaatatttccccaaatttctatattgtttgggtaaacaattttaatagctgcataattgtttatatatagcatagtttatttaactgtttactcgctgttaagtatttaggtagttttcaatatttttctttcttttttttttttttttgtgatggacctggttgagagtgcgagactccgtctaaaaaaaaaaaaaaaagccttctgtctgtaagttttctgactgctgcttttgtctcttaatcacaaaagtaatctgtagaagaacttccaaatatttgatctttctgtttgtttgaatccgcattgaacggaacttagtgtaaatatgtacttggaatactcactacgtgctccaacaaaacagattggaagatgcagagtggattgctctatcagtggctcaatcaaatgaaatgtttactttctgccctttgaaaaaaaaacctgttttttttctctggagcatgattcctttcacacaaagctactgacatcctctcaaattaaaaaggggtagagaaatacgacatttttctgtttattactgtgaaaacgtggcctcaattctgtaccagaatggccatgagctcttgctgccatgccccgtttaggaacactgctctttgggctgagctcattaaacctgaaagactcagacagacagaaatacttttagaacacactgaagaacagtgtgaaatgatgtgtcatagctgtgaatggctggaaaaatcctgatttgctagcattaataactgctgaaaacaaaaagaaataaggaaacaaaaaacaactgctgagatggctgtgtcacattgaaataggcaaggagaatcttctaccatatcttgatgacaaaaaaacagtggaggaaatgcagtgagtcgattatattaatatttcacttcatcataactataatgacagctgacccttactgagtacttattatatactcagtacactcaggcactgttccaatcattttacacatatcaataatgagtagcgactgttattatccccattttacagatatgaaactgaggtatgacgtaattacttaacttgcttgaggtcacacaggttgtaatggaagagcgaggatttataccagatagtcttgatctaaagcttctgttcttaaccatggtgctttactgcttctcacagtatttgcacagataacttaataagcttgtaggaatgtaggaatacaacctttgggaaagtcatcttgcaacacctgttaaaatttaaattacacgtacctttatttttatgtatttatttgtttattttgagacagtctcactctcaccgagggtggagtgcagtggttgagctgagattgccccagtgtactccagcctgggcaatagagtgagact

SEQ ID NO: 446 - DNA, 2912 bp, Homo sapiens
caggtttcttttttctctttctttctttattattattattttttgctatcactgtaccttcagtgactggcttggaataaacacttgataaataaatatatgttgagtgaatgtatgatgtggccccaccctagttcctaaaaccgagaaaaaaaaaacccaaaagccaatttcactcaaaatgctgattgaatgacagaatccattctgtttcattggcatgctattttcagaaagaaagaatggaatgtatgaagccgttgcaaagtgcagcatggattttcattcttcttgaagccttcttccaaagcacagttcctctgttctgaaattattgctgtttgatgatatggatgatgcatgctctaggaattttaccaccatcaccatcctccgatacaagggatgcatagtagataattgatgttgtcatttaagggttacccacgaatctttggaagccatgatgaggatatgccattggctacccttttgatctgactcactgggccctcaacagtgcccccttcttcctcaaccttcattccagtccttctgcctgctattcggagtttgtatatacctctttctttcatgccttgggtttggagacgttctgccttcattccttttgtggattcttgagatgtccagatgtcttcaatattctttttctttcttttctctcaccataacacatccatagcttcacatggtcagcgtgaccactgtgaataaaagacttgactttttagcttctcttttgcatagatgaagtcagatgctggtggtccgttcaatggaaatatgactgaagatgatgcttgcaattttcaaattggaccatgacaagggggtgaggtctcaggacctcctccctcctccatgctggttagaaaatgagctgtcctggactgtgtggatgaaggaggacaaaacagttatgatagtgaagcaaaaacacaggatgcccaagccctgttgccattgagctctgggctgcttgcatcccgacagcctaaggaaaaacagacggctgtctcatttaagcctctgtgatttctggtctttgctgcagcagatgcaactttcttctgagcgatcttctcacccttcatttacacagccagtgaaaaagaatggaagcacaattctgcgtcctattcattggacatgttgccagattcttacaagatccatgagaaacaggggcagaaagtgaaataacgtttggaagcatttgccacttaacagtatacctgtgggagcagcaatatgaaaagtgggtaattggaaggaggaagcgccttgcatggtgattgtattagaagggacttggtggaggcaagcctgctgcagcgatctgttccagccgtggcggtgtcagaatgtggcagttctgacgtggtggccctttgtgtctcaggagcaaagtgacaaaatggcaaggatgggggttgctcatctcaacctcacaaatgagacggcacctggagaaacacatttcatcccagacacctcattagcagagagacacagataaattggacagggtttggagaggaatgaaaaaaattcaaaatataaaaagagcaacactttaaccaaggtactgaaggaattgatttatgaagaaagattaaaggaactaaatatgtactcctggctaagtggtaactaaaagagaacataatagaacatatttgagagggtagaaacaagatacatgaaaagggatggtttaatttagtctcagaggggagtgagtaaaaacaatgaaattcagaaaagagaaatgtactataaaagtccagggaacaatccaggagatgaaatctatttgatcacactaatcttgcagcaggagattcactacttgatggaattaatcctactggtaagaacagaataaatgtttttatgttgagctgcgataactctgtctgctcgccgagggactgtggctgtagatttgctcaaccacccatgacgataagctttggtccacctgcccacaagtcatcaagcagccaaccaaaggagatcagctagttgtcctggagaatgtatattacctgcatcactgttctttctctagggtattagttgctggttgaaatatgtgtgggggtctacacggggtaggagcaggagatagcaagaaggacagctggaagtgaatacttatttgtatacttgaatcgtttgtccttgcatatgcgggtaggtaccaattgtccatcccttcccccatgtattagtctgttttcatgctgctgataaagacataaccgagactgggtaatttgtaaagaaaaagaggtttaatggacccacagttccacgtgactggggaggcctcacaatcatggtaggaggtgaaaggcatgtcttacatggtagcagacaacggagaatgagagccaactgaaaggggaagcccctcataaaaccatcagatctcatgagacttattcactacaatgagaacagtatggggtaaactacccccatgattccattatctcccactgggtccctctacaacatgtgggaattatgggagctacaattcaagatgagatttgagtggggacacagctaaaccgtatcacctcacaaaataaagagcttctgactgatgtctcctacttgtttaaaaaaaggaaagaaaaagatgagtgcttccaagccattcgaaccactgcaagaaggtttcttctattttaaaccccagattgtgtatcacaaggcacggctgaaggaaggcattctgataatgctttcaggaatggtaccaatgagtaattttgttgctactttccattttggggtatgattccagaagcaaataattaatatattaaaattaaacattatttggatttataaatgagtataacacggtgaaa

SEQ ID NO: 447 - DNA, 2800 bp, Homo sapiens
atgcaatgcactttcttcaatatagaatttcaattttctatttcatacagttttcttgaactataatttgtgatagttgttctattccctaacttttgtatattttttccagaaactcctgttagccatattttatatcctctttgtctatcttcaatatttatcaatttctctttgttttttcatatagttcttcattttttaacctttaaaaatgttttccctttccatcttctatttcttttaaggagtttgtgcttgcattttttcttgtttagttttaagaaattatttattttctttctggctctttccttagttctatgaccacattctaagtttttaaaaattctgatttgtgttattttttcaactttttgtacaattttcttattgccttttagctcattgtgaaagagaagattacaattttgatcttctctatggacatggctttctgccatgctttcattgtctgtagggatattatcttgctctttgttatcttttttctaatagctttgtgtaggaattggttgctatcaatttcccacactcattttatgtgaaattggtttgctgaacttttagaaggaggcatggttcagggtagctttttaacttcatacgaccctcacttctgttacctgaaaacaataaaatgtaatgaatacagtgttcaaaaatcaagtggcttgctttctgagatttcttgggtgtgtcctctctcacatgtatttagatcttctctttattattctctcttactaccagcactttttcttcaaagtggagtgctctcttggaaaaaggccctggtagaccaattttgagagttcataggtctttgggcctcctgggtgtgagtcatagccatattcccaggtgtagactatgttgcctctagaattcatagaggcccaccaggcgctgtgcttccttctttgtgttaggggatgcaagatgcagcaatatgcccttactttggaggatatgtaccaacacaatcctgtccactagacccctaaaaaccttcctgatttggccgatctctggatctttgtaggatttatttcccattctctggagtacatgggtctcaccaaggcttccaacttgcacaagccacctcttactcatccttcctgaccagtatgatatcacagatgtaatgaatcaatgtgatgttctacagcactgattactagacaaaaaattagaaataaaaatttctaataaaaaattagaaataacctaaatacccatcaatgcctatgactgagtaaattattacacaatattaattattatacaataatttactcaatcataggcattgatggatatttagcatattcatcaatggatattcagcatatctatatttaggcattgatgatatttaatatatcatagaaacctctgtgaatctatagatgtttccttttttttttttttttttttgagatgaagtctcactgccattgcactccagcctgggtgacagagtgagactctgtctgaaaaaaaaaaaaaattaaagagtgaagaataaaatatggcggtaaaaactggagaagagaactttcagttttaatttccacaagactgtcagaaattctgttgctcaatagtgccttggggataatatgattttgaggaactgtaccatggttttagctcacatgtcacatatctgaaacaatgagcccctgaaattaaggggaaaatgagcattgaggtcacatacacttttccacagaacatggcttcaagattattcattttactgctacttagtatttcatgaagaggttgtatctgtttatttaagttttacctgcctcttgaacatgaaagttacctgcaatttcccctgctttcaatatctctgctataaatatatttgaatatgtaggtttaaaaaaaatctttgggattattccttagataaatcctccctgggtccaaatctgtgatcctctcacaattcatgttacctattatcctccagaatgactgtcacctccaagtgaaattgccaaaagcagttcccacattctcaacttcctcgagtcctatttattcgtgttaagttgaatttattctttcacattaaaggccagagagttctgcctagactacttagaaatggtcagtgtgccacttaacaccattggctttcctagctctcagaccttcaaactcaaactggatctaaaccaccagctctcctggatctccagcttgctgactacagatcctgggacttcttggtctctgtaattgctgagccaattccttatattaaacatatatctcccattggctctgtttctctggagaatcccagctgatacaataagatagaaaactagataaataaagatattataattaaaataatatttcagtttaattattaactatgatatgcataggtatctctgtaaccgatgctctctgaataaaatattcaaataaacatgttacagtgtgcatataaacacatgggtgcatgtaaaaaataactgaaactgaggctgggcgtggtggctcacacctggaatcccaatactttcggaggctgaggcaggcagatcacctgaggtcaggagtttgagaccagcctggccaacatggtaaaaacccatctctactaaaaatacaaaacttagccaggcatgctggcacacgcctgtaatcccagctactcgggaggctgaggcagaattgtttgaacctgggaggcagaggttgcagtaagccgagatcgcagcgctacattccagcctgcgca

SEQ ID NO: 448 - DNA, 1828 bp, Homo sapiens
cacccgcacagcaaggaggctgaaaagaggtgaaaaaactcccttctgaattgaaatcacagacctgtgacctgcatgctctcctaaatgggaaggggtgaggagcgctgtgggaaaggctcctccttggcaaggaaagggatgatttctccggagttcaccggactccccctttccccagcgccaagcatccgggtgagtgttttgcaatggggtctgtcaccatcaggaaccgaggagagtttagactctgcatttactacaccagacccacaaagcaccacctgtgctctcagtctcaggagaaacagcctctttcacttttcaaatcaatcttctgtgcatgtatttactataatgttacagcaccattggttccagctttaaatcacgaagatatgctctacttaacgactttagttgaagcagcacacagccacatgggaaataccatttagaggccaatcatctctttcccagaaaatctctggggacaggggcaggggagggagagagggagctgagaagccttgatgtgcaagttcaatttctacaggtgaacaccagcggcctcggcaatgctaggttgttaggaattcggccttacttcttttcatctctgtggtttactaactggctctcaaaggcgtatgactgcggtgccccactgcacgcacgcagcagagggcatgacagggttcattggagaagctggctcccgcagggctgctgggctgacctctttcagctggagaaagccataagcgctcactaacctgccgtgctcaccatgaacttccacttgaccacataggcatccccctcgtggaactgcccgatgctttgtttggggagcctgctatagtcaaattccaggatgtaccagacatccacggagacactggtgatctcaaactgcctcctgtggtgtccttccaccaggccacagccacagccaatgttcaccctgtccaggatggcacctgctgtcgtctggggcacggacaccatccgtgtcacgttgttccgcttgacatcggccctggagtcttcctataggggaacatgaagacatcggttcagttgcctgtttccgcagctattccttgtcacactcaacgctgcttcagctgttcatgggacagcataaaaatacaccttccacactctgtctcaggatgtttaactgcagtcagggaaaggctgcattttcagaaagactctggcaggtctgaacacccattggccacactggtgcatggggtgcctgaaaacaggtgcaaacccgactcttcacctcccagagtcagattagaattggaggccagtctgctgttctattagtgaatcctgggaaggcctcttagctttaaggggtgtgtgtgtgtgtgtgtgtatgtgtgtccattttttttaattaaaaaagtattttagttgacacattattatggatatgatttgatgtgttgataaatgcatctgtttcatggggaagggggtgtgggaaggagacgggacagggaggatagggagatgttggccaacaggtacaaaatgacaatcagagaggaggaatgagctctggttttctcctgcacagtggggtgactgttttccaatactgtattgcatatttcaaaatagctagaagagaagatttgaatgttctcaccacaaagaaatgatacatctggggtgacagccatgcgaactatcctgatttgatcattttacatcaaggccacttttgttgggacccgaaccaagccctgaggtttgagacttggtcactggaataccttgtgctgggcaagttccccggggttct

SEQ ID NO: 449 - DNA, 1505 bp, Homo sapiens
gctgccatgcaagttagctcttggccttctgtctcttgcaggattcccaggtcagaacacaggaaatacaaggtgctgtacaactcacagctgctgttccgcagccggctctatggggacctggaggccatcctgtaccacgtgcacctcttccagcccacggagctgctgctgcagcaggcggtgttcttcctgcgagacactgagcggaggcgggtcttccaggctctagccaggatccacgacatctgctataacagcaccaccctctgggacgtgacggtgagggacctgctgccctcctctgctatgataaaagacttgagccaagagtttgggatgcccctttcgcaagaagaactcacagatgagaaactgtttgccctaccacctcagcctgcccccaatcttgaggactaccacagtcggaactccaccctcaccttagagatccacgcccaccaggagaagtacctgcagtggcggagtgccatgcttatgaagaacaaagacaaaaagcacagtttcatccagaaaaatatcacagaagcctaccaggtcagcaagaagcctccgaagtccgtggcgaaggtgattaaaatttcagcccctgccaacaaggccgtctacaactatagtacccagaccatgaattctacagagcttgccaagaaggagctgtatcaagagatagccaaggagccaagaaagagattcacgtactcacaggattacctctcagccatggtggagcccctggacttgaaggaagaggagaagaaagcccagaagaaatcccgccaggcctggctcacagccaggggattccaagtgacaggtcttcagagcgacaccgaaagcagctttcaggatctcaagctgccacccatcaaagagctgaatgaggagtggaaggaaaactccctgtttgctaatgtactggagcctgtgttggatcgagacaggtggagctgggacaggcaccacgtggactttgatctgtacaagaaaccaccacctttcctcgagctgctcccttcgcccgcaccaaagcctgtaacagtcaggaagaagaaagggaacagccccatctcctgagcagcacagaccctcccacggccaccgcatggtgaacctgcacagcctcccccacacccgaccacaccctcctcaacaatcaacttcattaaagtgcagcaggacagatggcagcagccaggccctgtgtgaggctgggctgggctcacctcgtggtcgtggttgcggagcccaatgcggatggagcggctggcccgcgacagcacggccgtcatgccatacaggttgatgaggatgttggccacccgcttcagtaccagctgctcctccatgatggtctgggatcacagaggctccaagtggggactcactacctagaccagtcccccacatggtccctccctgggctgcatctttgcctgtcttagtctcctgtgttccttgagaaagtggagtcaataacacct

SEQ ID NO: 450 - DNA, 2421 bp, Homo sapiens
ccatcgctgtcgctggaatcagccgcgcgctgccgaaaagtccttttcggcctgtgagccatggccgaggcccgagcgcccggcgccctagaacccgctgaaccgcaagccgcagcttcagtgcccgccccctagggcgcgctcccgccgcgcctggccgccgagtgcgcgcgcccgtcccgcctgccttctgctcaccgctactctgtctgcgcctgcgtgctttggctccctggatctggtaggccgagttctgttctggggcctgagtttgcaaagcattccctttgccaggttaggttgcgtggactgacaccttagagattttcacggcctatccggtctgagtcccacggagagcagaactcacgtgctgtccttgtgcaaaccccatccagaaatcaagcgttatgctttccgtcctggactggagccagatccgttgtcttttactttctggagagagtgccccctccaaccaagatttaaaacaaacgaaaccccagcagtgtgaggctcggggtaggaggagatgcctttgagaacaaaaccataaagtctgtacataaacaatgcatacagactagcaaaaatcaaatgacttcccccaggcagcagggcttcaagagagcctggttaaatgttttcacccttaaagaaactatctaaaaacaaaaagctattgtctgacgcaaatttgttctatattcatcaagctctgtctactactgtatctgtatggtagaaatactatataatgttgagctactgcacacctgttatcaactccatgtttagtggcgtcatattgagaggtgacagcgggccagctggagttccgggtgggcatgggcttggcgggcctgcactcggagcagccggccggccctgccagccccgggcagtgagggacttggcacccgggccagcggctacagagggtgtactgggtcccctagcagtgccggcccaccggcgctgtgctcgatttctcgccgggccttagctgccttcccgcggtgcagggctcgggacctgcagcccgccatgcctgagcctcccatccactccatgggctcctgtgctgcccgagcatccccgacgagcaccaccccctgctccacggcgcccagtcccatcgaccacccaagggctgaggagtacgagcgcacggcgccggactggcaggcagctccacctgcagccccggtgcgggatccactaggtgaagccagttgggctcctgagtctggtggggacatagagagtctttatgtctagctcagggattgtaaatacaccaatcagcactctgtatctggctcaaggtttgtaaacacaccaatcagcaccctgtgtttagctcaaggtttatgagtgcaccaatcgacactctgtatctagctgctctggtggggccttggagaacctttatgtctagctcagggattgtaaaaacaccaatcggcactctgtatctagctcaaggtttgtaaacccaccaatcagcaccctgtgtttagctcaaggtttgtgagtgcaccaatagacattctatctagctgctctggtgaggccttggagaacctgtgtgtggaaactctgtatctaactaatctgatggggatgtggagaacttttgtatctagctcagggattgtaaacgcaccaatcacggccctgtcaaaacaggccgctgggctctaccaatcagcaggatgtgggtggggccagataagagaataaaagcaggctgccccagccagcagtggcaacccgctccggtccccttccacagtgtggaagctttgttctttcgctctttgcaataaatcttgctactgctgactctttgggtccacgtggcttttatgagctgtaacactcaccgtgaagatctgcagcttcattcctgagcccagcgagaccacaaacccaccagaaggaagaaactccaaacacatctgaacatcagaaggggcagaccccagacgcgccaccttaagagctgtaacgctcaccgcgagggtccgcagcttcattcttgaaatcagtgagaccaagaacccaccaattccggacacaatattggcaccttgaaatcagccataaagggactacttataccatataaattggaagaccttagaagtccaggtgtttctcccagatatccagtcattaaacatttaccattgtatcactgactatatgtcaccaaaggaatcgagaagaggcaaagtttctctgattgacctagccccggaatttttttttcttggctttgtctttttttcttggcttttctcaataaactgaaactccttgatgtaaatttgggaaattatatgatacagtattatcagttattcacaagttattaatgtcttattataaagagaggcttctctttttaaatcc

SEQ ID NO: 451 - DNA, 1808 bp, Homo sapiens
tcttccatttcccctcattcatggtggaaaatgcttttaaaagtattcgatgacccgtatgtgaggaaagaggagataggcccctttttcaccaaatgtgactttaacctccttagtaatccaagaaatgtatcaagtttaagtcacttaacaaaagatgttcttttaaaggtgcattttaaaaaatcacttttaaaactgggtaaactgggtaaaaagaaagggggaaggaaaacaatgtgccaggcatttgtattgacacacattcttgcctgaagaggtgtttagattccagggtattcccttatattctggtataaatgtttgaattcccgtgtattttgtttcaattttttggtttatcttttattctgtctcccttttcttcttttacccgcctttgcccttaatgctgcagatagcctctgtcattctgaattatttttcaattctgttttcttccctaatctttttttctgggctcttcagctgtctcaggtttgacagtcatgggttgtagtatcttgctatgcaggaagaagaggatacgtgagattacttgcatcaactaaaattggtcacttttctttctcaagtttccttcataatacccagttttagtttttttgttttttggggtttttttgttcttttttttttgagacggagagaatcgcttgtcgcagtgagcccagatctcgccattgcactccagcccgggcaacagagcgacactctgtttcaaaaaaaaaaaagctatatatatatttttttctggctaaaaattaatccaagtgtaataggggtgtgtgtgtatataatctattgaatataattcaaatagatttctagtctgaactttgtgtgtgtgtgtaggtgtttgtgtacatatgtaagtatatgcatatatgtatatatgtacacacaaatatgcatatgtgtgcacacatatgtgcgcatatgcgtatgtgtgcacacatatgtgcgcatatgcgtatgtgcgcacacgtgcgcatatgcgtatgtgcacacatgtgcacatgtgtatgcgtgcatatgtgtgcatatgtaagtatgcatgtacacatatgtacatatatgtacgtatgcatgtacatatgtttgtgtgtatacacacaaaaaacccatacatacattcaaaaatgaaaagacccatgtctgattcttcttagtatccctaaacactgattctactcaagaattcctttggagctagtagtccagaaatccagcatgtggagctgaatatgattaccttgggaatgactagtaaaaggaaggcaaggtgcgatgataaaagattcaagctgagataactggggagataaaatctggatcaggagtgcagattcatgaacaccatactgtgcctactgtccatggcttacatggtaattgggatgtattcctagatgtggtgtggtgtcaccagtgtgaccaggcatactagtttacccatcggatatgttgaaagtacctgaccctgtgctggactcaaaagttcagagaatgggaactgtctcattctcactccattgtatccagtgtgctgcttggcacaccagtaggtagtcagtaagtatttattaaatgaatgacatacttaattttgttctctactggtagtttgccctggcgcatttctttttatttctattagccaaggcagggttaggtgttttgttttgttttgtttttccatatttttgaggattaatgataaagcacttatcttgttggaatgagagtctactaacaaatcctgtacatcataataacagaactat

SEQ ID NO: 452 - DNA, 1596 bp, Homo sapiens
ggagcgggagccgagcctcgcggcccggggcacggcgccgcccgcccctccccggcagtgcctcccgaccccctgccctccgctgggacatggcctgaggccccgcccgccgcccgggacccctcccccacgctgcccccggaccctcgccccaggaccgcggggtctcttcccggccccgccgcccgccccggcgagaacaggcccggcgggcaaggcggcggcggaccgagggaggcctggcccggcagcggggagaagggtgcggcgcagcccgagtttcccaccttttctcctggcccagacgcggctggggcggacgggatctctcgcgctctgcctcctcctcttgcttcatggagccatgcgcctgggtggaggctcccgagagaagctggcctgcgggcgggccggacgagctgcgcggacggggcggggcgaaggaggccggcaggcggaggaggaagcggcggggcggaggcggcggcggccgggaagaactagaggtattccccgggcggctggaggactgagtcgagccgggacccgagtcctccggtatcccagcagccaccggaggcagtgagataatggaggaagaatgtagggagacctccagtggaccgcactccttctagcgagccgcggaaaccatagagatcagggctcagccggagggccggcccactgctgtcacgtggcctccatccgtgcgctttattggctggtgctggctttacggggttgaattttggacgctgcctgccattcgtgggaccgagtttcaggcagctggaataaagagagacatcgggggaaagccgcgagaaggccagcgtccctggctgggagcagagctagccgagtagggcgcccggctgtcaaactggccggcgcagtgcacgctgggccgccccggagcggtcgcagagcccgtcgggagtcgtagtccgggacgggcccgcggcattgtccgcgacgcagctcgggatccgggtcggggtgtcggccgggttgctgccgggcaccgtcgagcgttagccacccagcattgagctgccagcggctgttctccctaagcaccctcgctcacgtcgcctcgcctcgcctcgcctgaccggccgcagccttggataccagccttaaatcgagccgactacggcccagccccgcccgcggcgaggtgcgcggggttcggtgccagccgctcgcccctggcggggccagcctcttgatgcaccggggacgggcgcccccatccgactcttcgaggggcggcagggccccatctgtgtctttcgctctcgagcccccagctagagttggcttcagcggaatacctactgtgcgggattattcaacaagccgattgatcacattcttcagctctagcagtgcaaaggcttcactgaaaaagaacattacaaattttttttgaaagaaatttgggggtaaatttaattttaaatactgatttttgataacatttgatcttatgacagcatttgtatttttaattacagataggattacactaaaagtcatttctgacctgctcccttacccttatggctaaaataaactgctaggccctga

SEQ ID NO: 453 - DNA, 2820 bp, Homo sapiens
gcccgcaggcgcactgcgaagcgtctcttacgagtctggcggccgacttggccctacgtggcgcgggtgtggcgccagaattcaaacagctgcagttggaaacgacgctttcgcctgccacgagccaagtccgtccaggacagattgggtccgagagtggacacaagttttgcagcgcagggcggcctcagcttaggaccgattcgagaagctgggctggctggctggctggcgctgggctgtagtgggcccccagcggaggccgccggaggagcgggcgagccctggccgcagcactccgggaaagggcagttggctggcagaaacgcttgggtgaaaaagcggaaagcgccacgtgagaacagccccggcgtgcggccctgcagggcctcagggcgggcatcaaggccccatggggatccattcctcctcacgcttcctcgtgccaggcatccggttacactaaacgtgaccatacaagtctccctcaaacagcgaacgtgaggttcaatactccatttcgcagaggagaaaactgaggcttaaaacagccagtctacaggatatcagaattctaagaaaaacttggaaatcatgttcaggactatacaggactttaaaagctttatcttcccaaactgaaaaccagacctctcaagtctagctgctctcctccaggtgctgcataaatggacctggtctttggctaccttactttggatcacttggttgaaacagttcccttagtcaacttgtccactggacttaagctgtgggacacagaaattttccctggaagaacagaacaatgaagatgaatagttgaaaaatgagagaatcctgcattctaaaggatctagtctggatgccaggagctgtgtataggatgagagcctggtggacctgattgaccgcctgttgttcagttcagttcagagactgaaaatcaggatttgtgctggagagtctttgaactgtgtgtgaaagtatctgaaggtccatgacaatttgaaaggtaaacagtaatggactttcatagatctatagtatttgctcttgaccttggctgaaattgtaaaacaggctgccacatctctgtaagtacttttctcttcttgcatacaaatgaattggttttttataagtgtccattcataactgtagcccttttgcatctgtgtctagtgacttttatgggaaaacttttgggggataattgattataggaatttgtcatttgtgcacaaatgtctgaagggtttaatgtctaatttaacagaaggcatatgaaaaaggtgcaggaatcgaaggcacgagatacacctgaaagtggtggagcaatgaatctgaacaggatgatggattgaaagtgctcaaaagaggcagctaggatcctgtattcatgtcctaccactgcagccagggaaagatcctcccacatctatcagaagactggaggattatgtctggagagattgaattctagggatgctcaggcccaaatgaaggtgggatgacaagccttgataaaatctttcagtagaaatagtagagaattttaagagaattttattgaaatttttgagacatttttgtatgtctgggaaaagaggaaactaacttgttagtgcttttctgatttgaataatatgtgtcggaagaaactggaggtgaaatgtgaatgatccttttctggcttgtctggaggttttgcctcaacagagaaatctaaagggttatgaatttataacattatttctttgctctccaaaaaatggaagctttcataaaaatacatcaaagttttagaaaacagcatatttgtctctggtctttctttttggaatgattgttatttggaagttagaccctccttggtcaatcatctcattttattattattttttcttattttcttgttctagtctctggcagattttctcaactttatttttcaacccttcttttgaattcttcacttcctatattgtattttcaatttccaagagccttttttgtttgtttgttctctgatggttctttgttttttcagcctcttatttttatttcatggatacaatatactctcttcttttacaaggagattaattattatattttgaaactttcttggctattttctctgtttcttggattgtctttctttttaacatttttgtctctcttttcattttgaatcctgtctttcaagagagggctattctttaagatagaagtaataacaaaacagactagaaactgtatttggatggagaatggatataaactggtaggctttactcgagaaggttctggctgggcctttttgttggagagccatcaacttgcagtatctttaggtatttttggagggttggtaactttttccagagaggactccactaatcttttgtcaaaagaatctaagcctgcttgcaggttctgccagctgactggggactagggcttgagaatttcttttagttaatacccttgtttttatcagggaatctcgtattcaccttaatctgtgcctgcatccggcatccgctgaattcagttttccacatagtaatcctcgagactcctggtatgatatgggggtctgtcagctgtctgtgatgggtggaaaaggagccctggaattctatctgcctctttttacacactttcaatcctatttttagatcctactctgctgctttcagaagtatttggtaccttcatccctgaggtgttataccagggtttggcaaactgtaatctgccaaccaaatcatgatttgctttgtcctttcaatctttgcctgtttttataaa

SEQ ID NO: 454 - DNA, 1618 bp, Homo sapiens
gcactgatggcagctccttctgtgacgggctcaggaacacctggttgctgctctccatgagctgttccaactgtgaagcaaaagcttcatgcataaaggcatcccaacgcagtcaatgacgcacaggagaaagaggacagagataagcttcttgcctgttccagacactgaggatcaggcagagagagaggctcctgccaggctgagctgtgctctgaggaacatttcgggagcctccagaactgaagagggggccaactctctggagaaaaatgaatggatgtaggcccaaaggagccgaatgttttccttggctgaatggcaagcaagcaccaagtgtccacagtttccggagtgtggatatgaacagccgtcttgggtcctgaggtggaagccatgtgtggaagatggagggcatcggttagaaggagtctagtccctgatggtcactgagctgcagaaccagcctgggctgcttcctgctggatgtcacttactagagagcgaaattaaatgtgcttcagctactgttactttgggttttctgtcatttgtagctgaaataatcctaatcaatatgagatatattaagtaaacaaaaatgcaaatgaaaacgattatgaattgtatgattccaattttatcttcttttatgtgatttaagtctttaacttttaacccagatgattttattttaaaatgtgtgtttatatacatccatatgtacatagaaaaaagacaggaatgttgaaagctaaagtattagtagagattatttcaggatggtaggttataaggatttcatgtgcttatttatttaccttttgtttgttcatctgtatttgctaatttttctatttttttatttttattttttgagacaggatctcactttgtcactcaggctggagtacagtggcacaatcatggctcaccacagccttgacctcctgggctcaggtgatcctcccacctcagcctcctgaatagctaggacttggtatgtgcccgtgatcccagatacctgggaggctgaggcaggagaattgcttgaaaccaggaggtggaggttacagtgagccaagaatccaccactgcactccagcctgggcgacagagcgagactgtgtctcaaaaacaaaaaaacaaaacaaaacaaaaaaaaccttattctcagaatgaagatctgaaataagaaacaaacaaacaaacaaaaaatattttaaaaaggaaatagaaaacaaataacttattcatgattccatgggaaaaaagactgaaaataagtgttctaaagtaaataagggcatgagaaattgttcaaaatatagtgttaaagagaaaaaaggttattcaacaatatgatgccattgtttttgataaaaagaattaaacaacataaatgtatgctagaaaaagaactagaaggaaataagctgatatattggtggtctggttccagaggagagattttagtgattcttattttcccctttatacttttggtattttctaaatatttctctaataaacctgtgctaatcttatttatttcaaaatgtttctacttagagaaattttaacaagacagaaaagcatgttataaagaataataaaa

SEQ ID NO: 455 - DNA, 2797 bp, Homo sapiens
taattgctattctgtaagacatacagtctgtgtaagatgtatcttatttacagagacatttttgaaaattaaaatattaaatactttttgttatatagagacaatgatctggaagtataaaaagaaaaatattatcttgttgatgtaaatatgatatccttatatatattagaatccaataagatatcatgggcgcaatattagcaaaaaaaagtcagtcttaccctgaaaaaaaaatagatatgaaaatctcagcactacacagaaatgtttagatgtcaatcagttttggtatgcagcattattatagtagtaacaaatttaaatctaaaattttagagtaatactttccttctgatcagttgccctgtggttatttttgttaaggaaactaccattatgacgggaaactcttatttttgctgttatattttgtttccttatttcaccactagaggtgacatcattattttgtaactttagtactaggctttccatttgtggtatggacacgtatgatatggagcaatttttgaattgtctttcacataggggaaattgcatcttatatacccataaattgggttttagtgaaagagccaggatggaacataatttcctaacttccatcctagtttcctttctacttgttcagaatggccagtgaaacacacaaatggcactcagatttgttgatggctcaccaactcaaagaggttccttctctcctttgataagtcccacaaaaacatctactgtaaagacttgaaatacaatgcctttcttttttatgagagccaagtttacccacaactctcaactaccagtactggtctgactgtgcagaaaagggttgcccgcaaaggtcaatgatatcaccctagaacattctagaatgatgaattttgagaatgtgaaaccttagaacctcgtgcggttgtctgccttggaattcctgtgctgtatcctgaaattttagctgaatttggcagagaattcagagaaaatttgcttttctaatccttgctgcctgaaagtgctaattattttccatagctaggatattaccatggagcaatattttctctgcctttgataactcccagcaaattactacattaaccgcctttgaaatccaggagccacaaattttaattcagtggcctgctaaaacatgcactctctggtttaaagtttgccagaggtggtgctgccccatatccactgcgcccggcctcttttctttctttctttttctttctttcttactcttgctctcttgctctctctttctctcactctctttctctctctctctttctctctttccctccctccctctctctctctctctttatttcctaagcaagctaacacaggacatattctctcttttttttttttttttgagatggagtctccgtctgtcacctaggctggagtgcagtggcacaatctcagctcactgcagcctccgcctcccaggttcaaacgattctcatgcctcaggcttccgagtagctgggactacaggtgtgcaccaccatgcccagctaatttttgaatgtttagtagagatgggatttcaccatgttggccaggctggtctcaaactcctgagctcaagtgatccacctgccttggcctcccaaagtactgggactacaggcatgagccactgtgcccagctgacatattctcacttaatagaattatagagaaatttttcatgtttttctttttctctcccactttttcatattcctctttttcatttttgcctttccgtttctgtctatgatgtaggcttctgaggagaaccaagaagcttggctttagtggtagaatgacagaacttagggatcccttgcaggctagaacaaagttctgacccttagaccaaatctttatgttaagagttttccagaattcaaaaaaaaaaaaaaaatcaatcaacacaacacacacacacatacatacacacacactgcctttaaaaggaggagccaatatttgttgtacctgtgaactgaggaattatagataaaccttaggtcaaatcatttcacaattgcattggtggtattgaaaaatgatgagatttctctgacagagagctttgtcctagtttttgttcttcataggtcaaaactggcaatattctcttgtctgcaagataaagtgtttgtgcttctatcaccatatgcatgaacatgtaagaatcagatacaatttctgcttcatcagtttcacatgttcatgttgtcactgaaaaaatgcatctactgtttatagctcccaaggagaccccaaatcctttttttttcttttgagatggagtcttgctcttgttgcccaggctggagagcagtagcgcgatctcagctcactgcaacccccacctcctgggttcaagtgattctcctgcctcagcctccccagtagctgggatttacaggtgcccgctaccatgcctggctaatttttgtatttttagtagagacaggctttcaccatgttggccaggctgatctcaaactcctgacctcaggtgatccacccacctcggcctccgaaagtgctgggattacaggagtgagccaccgcgcctggccccgtattcattttttatactggaaaacattttttttgtaatttttctttgcaaagaaatgagcataaaaatgaatactccaaagaaaaggaattattatggcaaattaaaagggacacggtactatgatttaattactgtcttgtctttttaaaatgtcatctcttgttttacccttttttt

SEQ ID NO: 456 - DNA, 2818 bp, Homo sapiens
gaagattacctttatgaatcatatgtcccaggtctattgacttttcatatacttaagaagaaattttctggctaatttttaactaattgaaatctttcactttatgcatttatttaaagtggcttgagcagataagagctctgtaatttttaaatgttccttcaaatataagttgattttaaaggtatgcattatgatattattttattgtataatgcaatgaaaatcaaaatatattaagaatttcctatgcagcacattttatattaatagtactcatattagaattaaaacaacaaaagcaaatacagaagcatttacttagctcttactgtgatctgggcactctgctgagtacattgcaggtattatcttattcaatttacccaaccatagtagatactaaagagaatgcatggcttgatccaagcccacaaggagtatgccattgaatggaggaatgagtagtgtacagttatgttattcttttctttttttctcctttttttgagacaaagtctcactccagcctgggcgatagagtgagtctcgtcttaaaaacaaaaacaaacagacaaataaaaaacatacgatggagctccaatatgtctggcagcagacttcttagtggaaaccttgtaggccagaagaaagtagcatgacatatttcaagtaccgaaggaaaaaaaaaataccttgtatcctagaatagtatatccagcaaaaatacccttcaaacatgaaagataaataagaactttcttagacaagctaaaggattacatcaatgccagacctgtcctaccagaaatgctaaagggagtccttcaatctgaaagtaaagggcgttaatgagcaagaataaagcattgggaggttcaaaactcactagttacagtaagtacacagaaaaagccagaatattataacactgtaattgtggtgtgtaaactactcacgtcttgagtagaaagactaaagatggactgatcaaaaataataactacaagttttcaacacatagacaatacaataagctataaatataacaacagaaagttaaaaagtggattaacagaaagttaaaaaaatgaagttaatgtatagagtttttttttgttttctctttcttatttgtttatgcaatcagtgttaagttgtcatcagtttaaaataatgggttataagatattatttgcaagcctcatggtagcctcaaataaaaaaatcatataacaaacaaaaaaataaaaataaagaaattaaaaacaccactggagaaaaatcatcttcactaataaaatggcaggaaggcagaaaagaaggaagagaagaccaaaaagcaatcagaaaactaataacaaaatagcaggagtaagcccttacttatcaataataacattgaatgtaagtgaactaaactctccaatcaaaagacatagaatggctgaatggataaaacacaagacccaatgattggttgcctataggaaatacacttcacctataatgacacacataggctgaaaataaagggatggaaaagatgttccatacaaatgaaaaccaaaaaagagcaggagtaaactatacctatatcagataaaatagatttcaaaataaaaactataaaaagagacaaaaggggtcattttagaatgataaaggggtcaattcagcaagaggttataacaattgtaaatatatatgcacccaacactagagcacccagatattataataagcagctaataacaagcaaattaataataaagattggagcagaaataaatgaaatggaaatgaaaaaacaatacaaaagatcaatgaaacaaaaagtggggtttttgaaaagataagtaaaattgacaaactttagtcagactaagaaaaaaagacaagacccaaataaaattagacctgaaaatggaaacattacaaccaatgctgcagaaattcaaaggattattagtggccactatggctaactatatgtcaataagttggaaaacctaagagaaatggataattcctggatacctacaacctaccaatattgaatcatgaagaaattcaaaacttgaacagaccaataacaagtaatgagatcaaagctgtaataaatatatagcaaagaaaagcccagggcctgatagcttcactgctgtattttaccaaacatttaaagaactaataccaatcctacccaaactattctgaaaaacaaaggagaagcaaatagttccaaatcattcaatgaggccagtattacctgacaccaaaaacaaagacacatcaaaaaaagaaaactacagaccaatattgttgatggatgttgatgcaaaaatcgtcaacaaaatactagtaaattgaattcaacaaaacattaaaaagatcatttatcgtgatgaagtgggatttatcccagggatgcaaggatgattccatatatgtaaatcaatcaatgtgatacatcatatcaacagaattaaggacaaaaaccatatgatcatttcaattgatgctgaaaagcatttgataaaattcgacatccctttgtgacaaaagccctcaaaaactgggtataaaagagacatacctcaacaccataaaagccatatatgacaggtccacatgaaatcctttcctctaagatctgaaacacaacaaggacacccacttcataactgttactcaacatagtactggacatcccagctagagcaatcagacaagagaaagaaataaaaggcacctaaactggaaaggaagacatcaaattattcttgtttgtagatgacatgatcttatctttaga

SEQ ID NO: 457 - DNA, 1938 bp, Homo sapiens
gccactggtaacatccagccagtcagcagtgagcttctgggcaattctttcaagagactagattgacacctggagccagactttatcggacttaccaaagtttgaccccaataaacagttcagttttccagagttttctggctgcatgtagttgaaagcaggactctgccaagttctgagtccagctctgagctgtttgtcttccaccacctgctgcagaacacgaggtgaggaagtggaggctcccaaatgttgacttgtcacaggtgacatggtttgtaagtggcagaactggggcgagaagccaagtggttgcctcctgtcccttccttccattgtaccacagcgctggcctctcctctcctcacctgacactccatgggttcaagtcattgctaccggatgccaaactctgcaggtgctgtgctggacaccgggaagccaagtgactggatgagagacactgtcctaagagtacttgccatctagtgcatgacacagacatccagtgaataatagcagcacagtatcatgaaggctatggtagggacaatccaggatcctaccagagcttattcacatgggtggtcagaaaagaacggctgcccatggcattggggcagagaaaaaattgtacaacaagccaaagaatgcctatcctttcatgaagcattttggtggggccattctgtgacctgtctatgcttaatgattctaggaatggattcagttacaggctggactcagggcttctttgcagtaagtgacaggctaccacatacataccacatatgcacactgtggtctgaacaagaacactcgcttaaaaggaaagtcaggaggaaaaggaaagggtgacttttactgctttggctgaaggtactgtatttgccagtagagaacttggggaaatcaaatatttcaattaccctataatcatggaaggtaagactgatgggtgagcttttagctgtcttcacggtgtttacacatctttacatcccatggacatccctttggatctaaatggaagtcctaattccgaggtcttgatttattttagacatgggaagtacttcttaactgaacagtcagggttaagtcagggaagggaaccccttggagttatgggataaaggacacacttattataggaattggacattacacaactttgggaggaactggagaggtgaaggccccggaggaatgggaggatcagagaaggagccgccaagcagtcccttggacaagctaagcacatcctgctgctgtggaggggctgaaaagagaagcctgtggtccctcctccgtggaaatgccatctgagggaggggctggggctgctgtcggtcctggagtcagcagtcaggaagaggactggatgtggagtgcaggacagcaaggacaagctggaacccgctggcctctctgtgtctgtcaccacatctaaccctgatgactttctgagagtgatggccactgcttcacattttccaaacctggtacaagttcttcttctggccaaatttaactcagaactgtacagggaaagggatctcgaaaagatagttccatcttagccaagtgaacagagcacaaaccatcacatttgaagaaaaggctgaaggtagagagagaatactctgccttaattaagaaacaaatacttggctgggtgcgacggctcatgcctgtaatcccagctctttgggaggctgaggccggcggattgtttgggctcataagtttgaaaccagcctgggcaacatggcaaaaccccgtctctacaaaaaaatacaaaaattagctgggtgtggtggcatgtgcctgtagcatcacctactctggaggccgaagtaggaggatggctggagcctgggagacagaagttgcagagagttgacatcacgccactgtaccccagcctgggtaaca

SEQ ID NO: 458 - DNA, 3008 bp, Homo sapiens
tgaatgtgtatgttggctgcaatttagataatgtattaccttcatgacagttttcctttccctagggaaaattgccaggaatcattaactgagtggttcttaatggaaaacctggattggaggatacttatattcccccccaccccccattcagtgtccttatattgggaggagtcagtggaaacctttgtaggggatctttgttgctttggagtttatctgttgctgttactatctggagtcagtgattgtggaaagaacatgtgatcccaggtgtgcagaaagcatcagaaataggctgtttccatttacttccctttgggctatatagagaaaatgaggaaaaagtagggattttgattttctgcagataaaatgagttgaggtttttagtatgactcaatagatcttaaagtgtttatctgcagtttctgtactgcctaagatacattataaaaatctgtatgtaaagtttgttaagaggaaagattatgcttatcttttaggggaagagggaatattgagctacatttatagagtgattttgtttcaggtttttctgaggaattttaaataccctgtgtcctcttaatagtaaaaacagtttgaggtactctaagtttcttacaactatttgtctctgaatagttttctcttaaaatttgacaaatattaaaaacattgtccttccttctttggttacagtggagtctcttctgttttgccaggaaagttaacatattgtttttttcatttttgttcatcatttacaaattagatggtatagattttagcaaatagatctcaagaactggattcaattagcagcttttaaggacataaacattaaagggttagacggaaaagaatcgtttcgaccgaccggataaacgacagggccgtgtttgacgtgtgtccaggaatcggtctggagctggtgcccaacttctcagtccatcttgagaagccaaataaaatacttaacctaggcctcattttgttcaaaaaaaaaaaaaaaaagttgattctttgtgaagaccttaagaaatgtttggcatacctttagactaagtgacagttcatttagatctgggttccctaatagaggaaaaaaaatcacttaaaaaaagacagatcccacagcgaatttatatttggtttacaatacacttgggctataagatgttttaaaattattgttctttgctttaagttaataacatattagtattttaaatttatagagtacctttgatttcaagatgctgtataaacttcagttacttggtaataaccttattgtggaattgcccctttactgaaaggtatcaacatttcttgcgtggtgacacacacacacacacacactctctctcacacactcatacacactctctttctgaagaaaaggaaacatttaaaccatgagatctctgcccacaattgatattagacgtttccacatactattcttaaaaccactatcagtgaactgtttggtgtttgtcaacgctaacaaggtggggctaatttaataatttaatcccaaattttagattcctcaggcaattgggggttagaatccgatttcatccatcaagctacttctgttctttatgacacattaacatatgatcattaaaatgggctgtgtggtaacactattaactttcactattatttctataacaaattgatgacaaaatgtataatttctatatcattatgtttatatgactgtcttatacaggcttttcttccaatgggttggatagaaaaatattcttttttttttttttttgagttggagtcttcctctgtcgctcaggctggagtgcagtggtgcaatctctcggctcactgcaagttccgcctcccaggttcacgccattctcctgcctcagcctcccgagtagctggtactacaggcacccgccaccacgcccggctaactttgtttttgtattttaagtagagacggggtttcaccgtgttagccaggatggtctcaatctcctgacctcatgatcctcccaccttggcctcccaaagtgctgggattacaggcatgagccacggtgcccggcctagaaaaatattctttacagtaaaaactttgggataattttttagaccattattgtttctgaaaaccaaaggctggaaaaagcagcttagtgtaatttctaatattttaaattcttaggtatggagaagtagaataagattactcctccagaacatttatttcagacatttttatacatttaatgtttttgtatgctgtttctcatgttattaatttcaaagcccttggcaataaggaggttaaacttgtctatagtgtaaggaaatttaactttaaaaattttcctttagatatgctagcatcttaatcttggtacactacatattttatcgagtcaaggctttaaaaattatagcttcattctgtttgacaggtttttaagtatgcctatacaccataatgattttatttattttttaagatggaattttgttcttggctgaagtgtaatggcccaatcttggcccactgcaacctccgcctcccaggagaatcactgcaaccttgggaggtgggggttgcagtgagcggagattgcaccactgcactgcagcctgggcaacaagagtgaaactctgtctcaaaaaaaaaaagaaaagaaaaagatattagatattatactaataatcttaacttgcttagaagtatgtgagtacagctgggtgtggtggctcactcctgtaatcccagcacattgggaggccggcgggtggatcacaaggtcaagagatcaaaaccatcctaaccaacatggtgaaaccccgcctgtactaaaaatataaaaattagctgggtgtggtggtgcacacctgtagtcccagccacttgggaggctgaggcaggagaattgcttgaacccaggaggcagaggtcatggtgagccaagatggtgccatcgcactccagcctgagcaatgag

SEQ ID NO: 459 - DNA, 3121 bp, Homo sapiens
ccacggagtcagtgcaggggctatgagaatttgcatttgcatcaaaacggtaaatcaaagtgaaagaagtcattaagtcaggtaacaaatacttatcaagtgcctgttgtttgctggacatagcaggagtgctcccaggcaaggaatagacacctatttttcagacttctgatctctttggggattgccaaaatgacctcaagtagtgaagatatagtggaataatctgtatttctgctgaccttgatgacatttggatctcatttgaggtactaagaagaataagcaattggtttgaaaaaagcccttttaagagcaaaatgaattctgctaaagttgaagcaggaacaaatatcaaatttatggtgaagctcaggtggaagaatgatgaaatcactgatgctttacaagaagtttatggggacaatgccttaaagaaatcagcagtttacaaatggataacttgttttaggaaaggatgagatgctgaagataaatcctgcagtggcaaattcatccacatcaatttgtaaggaaaaaatttatcttcttcatgccctaactaaccaacatttaatagcttggacaatagccaacaccacagacatctcaattagttcagcttacacaatcctgactaaaaaatcaaagttgagcaccttccactcaattggtgccaaaaccatggcacccagatcaactgcagacaagagtggagctttccatgaatattttaaacaactgggatcaagatcctgtagcatttctcaaagaatagtaacaagagatgaaacatagctttaccagtatgatcctgaagacaaagcacaatcaaagcaatggctaccaagaggtggagtggtccagtcaaagcaaaagcagaccagtcaaaagcaagggtcatggcaacagtgttttgggatgcccaaggcactttgtttgctgacttgctggaaggccaaaaaacgatcacatctgcttattatgagagtgttctgagaaagttagccagagctttagtagaaaaatgcctgggaaagctccaccaaagagtccttctctaccatgacaatgctcctgctcattcatctcatcaaacaagaggaaatttgtgacagtttcaatgggaaatcaataggcatccaccttatagtcctgatttggctccttttaacctgtttttgtttcttagtctttttttaaaaaaatctttaaagggcaatcatttttcttcggtaaataatgtagagtgcattgacatggttaaaaagactgcattgacatggttaaattaccagaactctcagtacattaggtatggattaaatggctgatatatcactttctcatcaatttcaagacatttttgttttctcatatgatgcatcatgaagcaaagatgactgaagtgcctctttgtacctgatagtttcccaatgtgtttttataattttaggttctaagcttatttttaatatgtatgtgtttctcctgtagaaaatcttggttgataatttgtccttttgctacttctaggcttcccaggatatcccaatttcagagcactttttatgttaatttctcacttagaggttggcaaacctcacatatattacctatttcaattataagcccccaaatttatgtgaggggaggcatggttacaaactcatagggaagacattttcctctttacttagagcccaaaccaagacattcatttatgcaataaaagcttactaagttactgtgctaaagactagggatgtcataagaaacaggcaaaatctctgctctcatgaaagttatattttggaagaatatccttgtgctgatggggaaattttctagtcctccagcaggaatctcagttttcactccctgactccattaggctcatggcctcatttcatattcttttatggttattataacccatttccatgattaccaaggctggcaatattctttcctccaccatcttcaaattgaccttaccattttattttgccttcttcatttttggcaagtaggagcttggcttactttattgtaagctcagctatacatttaaaagtaaatatacaatttcttttcatcatttctatgggtttggtggcacagggagttacaatatctggtctaccacattgcaggaactagatttccccagatgactaactcttcagtcaaaagtcttcttttttccccgcctcaaacactttagatttacttatgccactgtggaaaattatttccactgtccttgaagtcccctttcccaggttttccctctgctgtttttttctaatctttcacggtgtgctcaaatgttacctcttcaaagaggctttcctggaaaacctcaccttgagcactctatctcactcactaatttctttgttttttttcttatttataactttatcagtattttgaattattatacttatttatttacttacttgttgatcatgcctctattccactgtaatgcaacttccatgacagcatgatcttttttgtcttgtttatcattatttctctagggtcagtctaccactacttttattgtcttggcagaattaggtgtctcctgaggtgatgcaaaaagaaaagacattatcagtgatggcttctaaccaaggattcaaggcttctgatttaaattctatattatagccatcacaggggtgaaggcacaaagtaaatgacaccagaaagaggatttaacacaaaatccgaaacttgggatatttcacaggataaattgctgagcctcttcagcaaataagtgtctagcaaatcctgtattagtatctttagtgatcatacctgaatttgctctccaaaggcaccatatctatacattcttagtatcctatctgttttaagcagataataaagtcagctaaaccttcccttactgttacacactgagtaagtgcaatttggatggtttgaagatattaaatctttcagcccagatttaagaatacctgtttgagagaaattacaagagcttttaatgcttgctacagaaaggtaatgtcttatcttttctgtttagtattttgcattccaaaaaatgagaaaagttattccataagttgataattga

SEQ ID NO: 460 - DNA, 1611 bp, Homo sapiens
gtttttctttttgattatagccatcctagtaggtgtaaagtggtatctcattgtgattttattttgcatttccctaaatgattaatgatattgagcatcttttcatgtgcttcttggccactagtatatcttctttgaagaaatgtctattcaagtctttggcagtttctaaatgagttattgtctttttgttgttaggttgtaagagttataatctggataacagatccttattagatatgtaatttgcacatattttctcccattttgtgggttgtcttttgtcttttttattttttattgcttgtgttttggtatcattattgataattcattgttaaattaaaggtcatgaagagttactcttactttttcttctataatttttagttttagctcttgtattcatgtctgtaatcaattgaagttaatttttaatatagtatgtgttaggagtccagcttcattgttttgcatgtatctatgcaattgacccaaagtgatttgttgaaaaatattattttctgatggtatggtcctgacacccttgtagaaattcaattgaagatatatgtatgagtttacttctgcagtctcaattctattctattggtctaaatgtttatctttatgccagtaccagcctgttttggttattttagttttgtaataaaatagacaaacatttggccgattaatcaagacaaaaagaaaagtatatagaaataatattatgaaagaaaagggggagcatattgaaaaacaccataaagattaaaaagattaagggacagtgttaacaatttatgccaatagggtttgaaagtttagagaaaatagacaaattcctagcaaaatataaattatcaaaacctactcaagaaaaaatagaaatcttatatagtcacaaggtattaaatattcaattcttcctagagacccaaatatatgctttctacccgtaactcactgtaaataaaaagaaagggataatatattgtgcaaactctagtcaaaaaaaagttggaatgacaatattaatataaacaaaagtggacttcaaaagaagcaatattatcagagagaaagagggcactgcttggtgaattgccccctgataaagacataacaataccggaagtaaaaattgacataactgcaaggggaaattgacaaatccacaattatatttgggatttaaatgctttactcttaataattaataaaagttagctaaaaaattaattttagctaaataaattagctaaaaagttaacaaggatagatctgaacaacactaggataaacttgtcctaatggacatttatagaccactataaccaaaagtggtagaattcacaatttttgcaagtacacatggaatattcaccaaaataaaccatatcctgatttataaaataaactaacaaactggcttctctcacttagcaatatgaatttaaatttcccccatatctcttcatggctttatacctcatttccttttattgctgaataatgtttcattttatggatgggccataaaatgtttatctatgcacctatagagggacatcttggttgcttccaagttttggccaatattaataa

SEQ ID NO: 461 - DNA, 2449 bp, Homo sapiens
gatctcttgtttttattttacttgccctctcaacactgttttatcaaagccaaccactctcacttagaaacactttcttctcttgttttctgacaccacacactctgggtccttctgctacctcactcattatcctttcttgggcttcttaattggcttctcaatctataattatcaaagtgtccagggttctgttctgagtcaccttctctatccaccctccctatgtgagtttacctagtcctgtgctttagataccatttataaatagatgactccaaaatatgtctctctgacaatgacctctctgtgtaattcctggctcctgtaaataactgcctacttgatgtctccatatgaatgtcagactggcatctcaaacttaatgtgtccccccaaaatcttgattttcacccccaaacctgcacctctctcagtcttgtgcattttagtaaatgtcactggtactcattctgttgttcaagctaaaattctacgagctgtttttgatttatctcatccctttacctttaaatccttcagcaaagcctgttgatgctatcaaaatgtatcctgagtctttgacttctttccacctccactaaaccatctagtccagtagtagatgatgctgctggactacttgagagagagcttcctaactgaccgtcctgcttccattcttgccctactgtagtccataagtgagaagccacctttgattaagccattttataggtgggtgttctgtaacttgtagctgaacccattcctaacaggcaaataatttggtaccagaagcaggatgctaaaaacaagacactaaaatatggaattggctaactggaggttaaggtagccagtgttaaaagactgtatgtcaaactggaaagttggtgacccttataagccgtagtgaaactgtcacttgctggaccttggatataggccacatgccaaccaatgctatatttttggagaaatctcacgagcaatatggaacattagtgagtgttgccttttttagtagctttcagcagactccttcaagagaagggcaactctacgttggaaataactgatttgaaagcagagattaaagaaagtaacgactaggtaagagaggatctctttacctgcaaccaggagtctcataacttggaaccttgctgaaataccccatttactttgtgtactcaggttgaaactagtacaagtagagacagagaggtggctttatcaggagataggacggccatcccaagataccagaatggcctctaggtggcatctctttcctcagctgttttgaattgtctcaagacaatgttttacctatgtaggggattccagagtggatgagaataggggtgaaaataagatttcaatttacaccttttagtattgctttgatgttgtaaactacatattacttatttggaaaaataaatagaaaaatattgaataaaaacaatttaaagactctagataggctgggtgtggtggctcacgcctgtaatcccagcactttgggaggccgaggcggcaaatcacctcaggtcaggagttcgagaccagcctggccagcatggtgaaaccctgtctctactaaaaatacaaaattagctgggtgtggtgacgcatgcttgtaatcccagatactcgggaggctgaagcaggagaattgcttgaacccgggcagcagaggttgaggtgagctgagatcgcaccattgctcttcagcctgggcagcaagagctaaactctgtctctaaataaataaataagactttaggtaagttatttgtgattactatacatgaaattgatcagaagtctaacaaatgattgaggtaatcctgtgtccctaaaatctcaagcctggttaacaacagtctcttgctgttgggccctgtggtaatctctgaaccctggaattccttctaacagggagtaggatgcaaaaggtgtgcgttctgcacaagtagaggaaaaaacaagggtgcggcttccagattctccaactgagaacttaatacactgccaccaaagaaactgcagtttcttttgtttagagaaaaggtcttgctctatcacccttgcattggatgagggtgacctcaatgggttgcatgttctaggctcccctgtcagctgggatggggctttgtatttctctctcccttattcatatttctgataagggtgaactgtggtgttaaaagcttggaattttggcatgtgttgtgttgttctgaattcagatatactataaactggggtggtgaatatgatggccatctgcatttgatgaccttgtttcttccgtccctgactttatcaaagcacacatcactcctaatgaaaagtctgactgttgatttcttcaaattcatgttgggggaaggtatatatattaattatgtatacattaatacataatgtatgt

SEQ ID NO: 462 - DNA, 2310 bp, Homo sapiens
cacttctaaggggattttcacaatcggttaagcccccgttaatcctcacatggttattgcggcgacccccatagcccacggtcccaaacgtgtgcatctgaatcatccgaagctctttctaaaggcaggctcccctggccgcctctcctaatccgactcggtacttcacggggcgtgcctagaatctgcattttagcaagggcctcgcgtggacagaattgcacgcttgagtttgagaagtactgcttggcctctgccccacttacccttaccttcttgagatgaggtaaagcacttggtaggcactaggcgggtaggtttatctttgctcccaagtctcttccgagaacttgggaaggctgcctagcttagtgagggaatccattctggcttcagaccagtctgggtttgaaacctacacatcccactaacttaggcagggtaccgaaactccctgagcttcatgtcctcatcagtaaaacgaggttaatcacacctacaccttagcgcgatggcttaaaaaaaaaaaaaaaaaaaacaaacaaaaaaaaacatagtccatcaagagaaatacattttgcatacagaccagtacactgctatttgtatgagagtgtcataaagtttccgaaaacaatgcttactacttgtgacgcactgatagtttctatgcttatcctatcctatcttattctccttcttgttgatcatgactggcagtttgaaaaacactgctagaaaaggttggtgtgataatacaaaatgcagagcagaataccgggtacacagtaattgctgaataaatgttaatagcctcttccagtcttctcctctcagtgttagcctttgaaaaacaagactttagacaataatactcctctagtaaagatccttcatgggatccgtatctcctagataaagcccaaactcctttctaggcaaagtcctcccactcttttttcttcttaacctcagcatccaccagaggacacactcttactggtgtatctttcatgctttatctcctccctctacccccgttcccttcctacaaatcctacccttttccctccaggaaagattccaggttcctaaacacttacgcttttttccactagttgacttcctatgtgacccttcccttctggtaaacccctatgcatccatggccggatgtgttggctcacacctgtattcctagtgctttgggaagccagaggcaggcggatcacttgagcccaggatttcgagacccagcttgggcaacatggcaaaaccccatctctacaaaaaaaattagctgcatgtggtggtgcgcacttttagttctagctacctgggaagctgaggtgggtggatcacttgagcctgggagtttgaacttaaaatgagctatgatcacaccactgcactccagcctgggtgacagagcgagaccctgtctctcagaaaaataaataaactcctatgcatccttcaagaccctgctcaaagtctgcatacctcactcctgcccagccatagcttcttcctcctctgtgctgccctggcacatgcaaaagagcttctctctcacacactatttccacacttttattctcacttcttactctcctgctagactgagctgtggtagaaggattgaggccagcacagaggaggatcagcaggaaatgtaaggtgagttaaagaatgaatatttgcatacaatgacacctgcatgacttgtttgtatagtccatacattctgttcccaggccatttttcaagtttagattgcttcactgtcaaaccaaggactccacccgggttccatgactcacctgtattagggtgcaggggttttggggggaggtaggtggggttgaatatcctgctgggccactgtcaaacctcttctgggagaaaaacggggtagctatgcttctcctatcctggaggccccatcatgaagaccgcagaggtgaaaaaaaaaaagtgtgcgcacacggggcggaggacagggtgtgaatacttggaagaattctgcagtagttttgaattatttgattggttttgcccttttaaaggtcaccttatccttgttttgtttgtttctaggctgtctgactcttcttactagagacacagccaaagaagggaaccctttgtttttttttgttgtttttttttgtttctttctttcttttttttttttttccccgagatggagtcttgctctgtcacccaggttgcagtgagccgagattgcgccactgcactccagcctgggcgacagagtgagactctgtctcaaaataaataaataaa

SEQ ID NO: 463 - DNA, 2306 bp, Homo sapiens
atgggagccgcagagaagcacctagaaccctggggcctgagggccggctggaggcaacacagggctggtaagaacgaatatgacctcgtgttaaactggaaacgagaaggagggcagaatatgggtagacaagtcagaacagctgctcccatagccgccagaagccgtagactcttctgaactgtgggacccaagactgccgggtacgcagcgggcacaggacggcaggggcacctgcgctgccgcctaccgccctggagggcgcttcaggcccagatcccagggcagcagagggtgcgacaaggcgtcccagcagccgcggccctgcagatccttggggcacctagccggcatcccctcccctgacccgacgccttctctttgtcctgaaggatggactgcaaagctatgtccggcggtgtggacgaaactcagtaaaaggggtttcggcaaggggagactcacccgccatctccgtaggaaggtggctggggctgtgtccgcgcagcccagtcccggcctcctcctcctcccagagggcttccaggtctgaccgagggtcgctgggactcgagaactggaggctctgactggaacactcctgggcgcactctggctcagcctggacccgggcaggcccgttttcaccggggtgtctacactagggtctcccaaggcagcgaggcgtccgggcgggcgccagggtggcagggccgagtctccggaggtggccgctctgaaggccgaatctggccagaaaatctggccagaaaaagggaccactgcgtccttgcggcctcctttccacgttaaccgggggcagagaacacccaggccctgctgtgggtgcctaacgcgctggggcgcataggccaagccagtccaaccagggtgtccagtcaggggcgcgctgaggcagaaggctttagagatcgagggacatgggggacccctgacgccaggtctccttctcgcttccgggtccgcactctgggtccgcagtccctcaacccaccaggaggccagagatgcggctgcggtggatccagggagctggggcccagatccaccaagatcagggaaggttcctgagggcccgctgtccctcagggcctgaagaaaatccattctccgcgtgcttgcgttggagaacacagtggcccccaccgcccgccctgagacctccacagggtcctgatccagaacttgacgaccggacgccatctcatctctccccacagaacccctgtcagcctgccgggggacgcggtgcccgccctccggcatccgggaaacacagcgtcccagccggcgcctgcgcaggtgccacacttcctggtcctctctggctgcgtctgagggccttagctctggccaggggcgctgcaacgggctcagccttctaggggtggcgggtgaaggcctacacctgccccaggacggccaggtccaccagcacctgcaacagacggagaactgaaggagcatcttgaagctgtggctgtcttgaaggtgtcgccgcttgtcctcacgagcaccattttgattctcgtgccctgtgtgcatgggtggccagcgttgctggtagggctcctgcacgctcagagtggccttggccaggaagtccagggtttccgcaggcagagtgtatgaacaaggtgtttatggattttgtatttgcccatgtttaatgagctttacaacacttccatcagagaacgctggagtgcagtggtgcgatctcggctcactgcaacctcggactccctggttcaagtgtttctcctcctcagcctcccgagtagctgggattacaggcacgtgccaccacaccgagctaatttattgtatttgtagtagggacagagtttcaccatgttagccaggatggtctcaatctcctgacctcatgatccgcccgcctctgcctcccaaagtgctgggattacaggcttgagccaccgcgcccggcctatatactgtattcttacaataaagtaagacagagaaaagaaaatgttattaagaaagtcataaagaagagaaaacatatttactaatcattaagtggaagtagatcatcacaaaagtcttcatcctcattgtcctcacattcagtagactgaggaggaggagaagcagaagagattggtcgctgtctcatggtggcaaagacagaaaaggtggaggctgtgaaaagggaggtaagaggcagacacaccggtgtaaattttattgaaaaacgtccacatatgaaaaccgcgcagtttaaaatcttgttgttcaagactctagtatattaaata

SEQ ID NO: 464 - DNA, 2417 bp, Homo sapiens
tctcagaggtgcaattaagggaccttatgcaaactgttgttttctataacccatggttcccagaagaaggcttgccaaacctagagctctgggaacaagtggggagaaatcttaacatcatgcacaagggcaacgggtcccagtaacatctttaacgttatgggccttagtcagggctgctttgtccccactctacacagaagagcctaaaaagggaagggaggaagaaccatcatctaccttaccacctcctccttctccctcagccccgccgttaccgagtaaaggtgccacagaggagacaaatattttccctgagccctctctcccaatatattggaaaaaatacaagggatacactactgttatgggaccctgtcttagtcaagtggaattagaagggagctcttgacctgcccagtgatgcaagatcaacaagacaatcagtaagaaacgtacaaacattaagaagagcctattcagaaaagacagtcttccagttaccttataaaggcaaggaagaactggtagtttgaaataaaataatttcaaaaagtgctgtaccccctgagtattctgctgtaataaggtaagcacctttctcctttgttgatgcaatcatttacatcttcaccccgcaaacacctgtcacatccttttaaatatttgtttttccttttcctaggatgaaatcccataagtcaattttttctcgggattgtgaagttcactgcacacaaccagccttattttacatctctttcttttatttcaaccatctggggactgggcagaagtcatggtcagaggggcacatatgtattgctttgatgatgggggcagagaacaaacaggaagaactgggaaatgaaaattgctttacttcttatattctgactagaaaggcaacccaaatgggaatcataacagggatagttcagtggcaaaaagacacccttaaaaatagaagctagaagttaaaggaggggtgacatcacacttttagagttgacatcagaggaagttgcatctcttggtgccatgttatacatgcaccgtcggggcagcagaactgattagtgtgcccagttgcacaaaaggactgtaatgtgcatgggccccagtgttagccatgtgactctgttaatggatccagcttgatctttacgctcttcaactaagctagtgcccaagcagttcttgtatttcagcttagagggggttctggaaaagctcttggtgatagcatttttggagaacggagagaaaaaaacatttctattaagtctgcctcaaaaagtaggaatttcaggtcaatgttaaatgcctggcatgagttttatatatatttttaatatttcaggcttattttaccaaaagaaagctggtgtaacacgttcacatcagacaaaataggtatcaaagcaaaaggcattagagatgaagaggatcacagcacagtgggaaaaggttcagttcagatataggtacatattatataaagcaaaaatacacagaaacaaaaggaaaataattcgatatttaaagatattaggttaagtgaaatttttataagtcttgaaaacaaatcagaaacaggtgagacacaggaaaatcatttttagcaacaaagtactaagagggtgcatggacaaggaaaacttttccctccctaagcccatacctctagttgataatctgttcccatccccaggtcccagcaaacattctaagacccaagaacaggtttactgggaggaattcgaggcactgcagttcatgtgctgagttaaataagctctggaaatagaagctgaagaatcacaatacccaatctaatttaaccaaagtctaattagagaatttaccagaaattgagagacttgctagacataatttcctgttaggaagtagtatttttctcctttcaaatttttaaatgtttggtataatcagcaaaatagaagtaaattatcaatttaagcttaacagagatattttgttaagtaatatcacagtaccttataaataaaaaatattaattatttccagttagtcacacgtattattactaataatgagtaaatacaaaaatatacactcaaacagaaagagccaaactggctcccataataatagtaggtattttcctaagccttgtacacagtaataaataaataaatctgccacatctttaccattctttaaatgacaagctgggttttgatgaattccactttttcagtcaaaaatcatattcatgatttaaattgtgctgtatgaaattgtcccttttatttctgtatattctcagatgagatgcatacatattggttcaagacctcttgttgcctctttttaacattgtctttgtatcactcgcagcccccaaacaacaggttaaaaccagcccaact

SEQ ID NO: 465 - DNA, 2189 bp, Homo sapiens
tggcggccacctccagtgatgagcacctccttagcggttggggccggggctgtggtcacggctgagagcctggaacccagccgcccttcagtggaggaaggccaggggaagggtaccgcggcagagggggtgtgtgtgtgtgtgtgtgtgtgtgtgtgtgtgtgcgcgtgcgtgcgcgcgtgtgtgtgttttgttttttgaggttttgttttttttttttttttgtgggaaaaacagaaatctgagggaaaagaacacagttaaaaattgctgtaaatgctggagtaactcccagggagttaactgttctgaaacaaccacttctactaactccgggttaaacaaagctattttgttcaaatcctctttgaaaatcctccagaagtatcacagttctgcccaaagataagaaagtgtaagacacgtcgcttgttttacacctggtagctcatggctcctccatgatttaaaacgtcgggtttgagtttggtcagtggtggtcctagctgtgtgcgctgattaaaggtagccacgaaacgctggggaaaaaagaaaaaaaaaaaacaagagaccaaactttcttgtgatgtttctatttctgcctgaatttgtaaatgttaattgtaagttattttgtttgacttttaatcctcctccaagttctttaaaaatatgacatgcttaagatgagcagctgcctctgtgccttttctgtcgcctgggagtgtttatcgactgcttgcacgttgccagactctcttttgagagcggtttgtcacctcggataacagtcgggacggccctaggatgccctcactggatgtcaccatcctattcatatcttcattttttactgttgaggaaacaaggcccagagaggttaagttactctaagggcacacaagaagaggcgggattccatccggggagtctgcaccagcgcctgactgctgctctgcaccgccgtggtgagaaggatgccttgcaaagaaccatgtgaccaccaacacatcagaggacaggagagtttcatcacaggatccttgatgctactctttatagccaagctgacttcctccttccccctccctgaggctggcaaccacagtatggttcctattttctataattttgtcgctttaagaatgctctataaatggaatcatacactatgcaacctttcgagactggctttttccgctaaacgcaattcccttgagacccatccgggttggcaagcatgtccataggtgtttctttttaattacttagtggtgttccgtcatctggatgaaccaaggtgtgttcaaccgttcacccaggaagggcacttggattatttccaggttttggttattgcaaatagagctgctgtgaacattcttgtacaggtttttgtgtgaacatagttttcatttctctgggataaatgcccaggagtgcaattgctgagtcatatggttagcacatgtctaattttatgagaaactgctaactcttttccagaatggctacaccattttgcattcccaccggcggtgtgtgagtgattcaggaccttcccagcctgggcagcatttggtattgccactattttttttttttattattttagctcttctaataagtgtgtagtgatatctcctgatggtttaatttgcattgtcctgatggctaatgacatcaagacctttttatgtacttatggccatctgtgtatcctcctcagtgaaaagtctgttcatgtcttttgcctactttttaattggattgtttggttttaacaactgagttttcctaattctttatattttctagatgctggccttaccactcggtgattattttctaccactttatcttgttttttcaccctcttcacagagtcctttgcagaaagttttacattttgttgaattcaagtttattaattttaccttttttggattttgatttttggtgtgaagtctaagtcttcacctagcctgaggttcccgaagattttctccttttttccctacaaattttatagtttcacattttatacttaagcctattttccattttgagttaatttttgtatgaggtgtgaggtttaggttgaggcttatttgttttggtttggtttgggttttgccaacgaatgtccaatagctccgggactattagttgaaaaggtgatccttctt

SEQ ID NO: 466 - DNA, 2309 bp, Homo sapiens
gctgaccttgagagagcggaggaggcgcaggcgcagcacaggtggcaattgaagccggaagaacatctaccaagagcagagaacccaggaagaaaattctgcctctttaatacgttccaatatggacgttttccatatagatacctatctatatagatagatgctctgggatctgacggtcctggacacctgtatggctgtgtgctgtggtctttgcctagcctgcggttcacttttgctctggccaccacctcccctcatgtacaaaccgcgtctctgctctgccagtcttggcccccgtcaggcagcggttcactccctcaccagggaagtctagatagatagatatagatagatatcgatcgatagatatagatatatatatagatagatagataggtagatatatggatagatatagatatttggatagatatgtagacgtctatctataaatatatcgatatatcggggttctcagccatcccaaagaagcttgcacagggccagttgctttatgtcaggaaaagggacacagactgagtgtatgagggttttttttttctatagatgtatagatatttatatcagatgcaaataagtgaaactctcatacaacttttaattttgtgttttttccaaactctattccattcaactttttctgcctttttatttgattataatgagacatcgtactggaggcaggcttgaaatggatatacagagcactttcaacccagaaatttgtctcacgacttggggtccttacttaagttgtagctgttagtaatttcatccatgaattcactttgtgccatagtgtgatgttttgttatgaagttggaattgggaagggaaactaggaagttttgggagaagttaagaaggttgagtaggcaggcgttgttatagatattaaatttcaccctgcttatcaaagtacctggagcgtatggtatccaagctgtaaatggcccaaaggaatcggggttctcagccatccaaaagaagcatgcacagggccagttattttatgtcaggaaaggggacacagactgagtgtatgagggtttttttttttgttttttttttttagtttaaacttgattttgtgaggggacgaacagcgtgacaattaaaagaagatctgggaatcgtaagtactgacagaatacctggagggaaggtgggactggagaatagtttgtgaaacagatcctgctgaggatgaatccagaacttcctgaagggttccgcttctgctgtgtagggacaaggttgtaaatagacaagccacaggcacctgcaaatggtaagtgtggtagagaggggctggaattgcctgttggtgtatctgtgggaagcagctgctatccttgaggaagaaaagatggagaaggtaagtggctatcaacctgtcagagacaacacattctgtgaagttgtactctttcatttctgacactgtggttcttaatatctgtcctaaacctggcagtcattgaggttcgcaaagtatctccttctatgtgacagtaaaactggaattactatcagaatggaatccattcctgctagagtaaaaccatgaaaagaaatcgcagttttgattatctgcttatcatttttaatagatcacagagtgcagtgttccttatttgttgtatggcaagttgatgaataactcttgattaatatcttttaatgttctgtttcacacagttctctcaattacatgtgaaaaatctgttatataagtaatgctatttaatgattaaaatggatatatgttttaaggtagtgccacccagtcttttttttttggtagaataatattattatctattggttgcctgccttgaagtacagtaatttctctgtttccattatttaaaataggtgagtgtctttgacaaagatacaaagaaaggggacagttatgaaagggaagaaaagatagatggagatgaaaagaaattcagtagtttcagcaagggaattaggagcataatctttgagaaaagtcttggttgcttaccattcgggctttgtgatgagttgaaatgtcctgaatgagaactgttctcaaatgctttcaagaccaacaaaaagtacgatacccacactgacctggaactctttaaaccattaagatatatcactttttcgcagttcagtccatattttttgcaaaatgttctgcttagtttttctgctaagaaattactgtttctgaacaaggttaagtgtcgtatgctttcattgtagttttttttcccctaacatatactgttttcctaattaga

SEQ ID NO: 467 - DNA, 2362 bp, Homo sapiens
gaggtttgtcactagaatgtgaagacagccacacagatattgcacagactatttacagatcgtttggtttacattgagagtcattgctctacttttgtgcggtaggaaaatgagatttcagcaattcctttttgcattttttatttttattatgagtcttctccttatcagcggacagagaccagttaatttgaccatgagaagaaaactgcgcaaacacaattgccttcagaggagatgtatgcctctccattcacgagtaccctttccctgagatctctctagctaactttactggatctatcagaagaagaagaggagtgaaggaaagacacccagccacacaaaagaacttcatgatgccaacagcgtgattgcttagaagttcctacacaaaaaaaggatcatttgaaagcacctggaatggtttattagcttcacaggattttattcttcttggcttctatttggagggaaaataacataaattcaaaaggattccaatctgaagcccaaatcgtttgcctacataacaaaaatatctcatcttttcctgcacattattattcttttatgggttaaaaagaaaaataccttttagtgttttagaactctctcatggtaaaaagtgcaagaatttaaaatgttgctttcatattcctataattctccaaaagtattaaattcgtatatgtttgagtgattttctaaaaactgctcaacctgaaatcaattgcattgaccatttggcttcgcacaatagggagaaaataattggttcattgattatatagagagaaagactaagaaaagctattaattgctaccaattttatgataagctttaaggtttatgaaagtatgtttttttatttaatgagtaatgtccatttgaagttgaaagaaaacatgaaatcctaattgtagttcattttatgttcaaatgaaaccattgtttttgtttttgttttgaaacagagtctcactctgttgcccaaggtggagagaagtggcacgcttttgtctcactgcaacctccacctcccgagttcaagtgattctcgtgcctcaacctcccaattataggctgggattacaggtgtgcaccactacacccagctaattcttgtattttttgtagagatgaggttttaccctgttgcccaggctggtcttgaactcaggctggaaccattcattttttaacctttctcatcatgtaattataggaacccaacgtttgatttcctttgaagttttgttatgtcctttattattttgtatggataatttctttaaaagtcttacttaaagttgacatctaaaatacagttatgccaatgaagtcccactcagggtgatatctgtatctaaaagatgagtgctcatcatcctattaggctttgtcttggtggtgttcatcctgagatgctgagacatggaaataaaaaatcagaaggaatttagggatatgattactcaaaaaagaaactatcctgtctaaatttgaattgtgttgataactaggtgttccccagatgctaagatgttcttaatttgtatttattgaaggattgttagcttagtgccacaaaatttttcttactttatgttaattccagataagaaatttacaagtttatatctttttttttcttttttttaagatgagatctggctctatcacccaggctaaagtgcagtggcatgatctaggctaactccctggctcaagcgatccttccacctcagcctcccaagtacctgggactacaggcactcacggccacacctgactaatttttgtatttttttgtagagatggagtatcgccatgttgcccaggttggtctcaaactcaggctggtgagctcaagtgatccgcctccttggcctcccaaaatactgggattacaggcatgtgccaccatgctgggccacaagttcatatctggagtagaagttttactttgtaaatattataaagtagaagaaaccataaaccattttgctaaaatgaaaggttggggttaatataaatgtaattttaaatagaaaatctgacaacactgtcgagtttgtcttcctgtcaaagcttattaaaagtgtctttgcggatgaatggtactttccacaagtgcatttgagtagaagcataacctattctcagttatatttatgtttaaaacatgtactggtttgtatattttgtactgaaaaagaaaacactttatagtcaagatacatctcattcaatacaagtctaaactctttcaaatacaaattcgcatattcacagaaaaagttacaaatcagttttactattgtaaagtaatgaaatggttatacatttcttaattgt

SEQ ID NO: 468 - DNA, 2117 bp, Homo sapiens
gggcctcacgggagctggatgctcggagcagcaaggcggccctgggggccccttccggtcccggggctgacgcctggcggcgtctccgggaacccgcgccctttcaagcgccgctggctcctgtttcacagatggagaccctgaagccccaacaagcagcctccccagcacctcccagtgactgaaaaagctggcactgggcgcccgtctcactctccacccagctcccaggctgccccacgggaccagcacctttggggagagacgagacctgaacaaacgctggggctggatccccagggtgggtcagaacagccgttgccctgaaagctggcccggacagatgcgtgcggggcgtgccaccggagctccttccagcttgcgtctggggccaccatgtcccttcctcagggcactggatccatctcttcaagctcagtggtccaggccatggtggcagaatcaccccccagggtgtgacgcctcgtccctgtcaattccacacagctgcacccggaagagctgtggccgggcgccgctctgacctgtgtgcggtcccctgaatgggtctcaggtgtcttcagaatggtgaggccgcctcatttcctcaccacacaaccctcctgaggacgttgcctgcaaatgggcggaattcctaatttctaccattcagttaaacgagggatccttaagccccaaagctgggaaagctgtccgttaatttagtgcgcgtgggccgtctcagaacagcagggttcccaggagtccagcgggacgggggaggggatgagtggccgtgtccccctggcagagaaggccttgtctgaaggctacgcccgcctccggtacagggacacctccttgctcatctggcagcagcaacagcagaagttggagtcggtgccacctgggacgtacctgagcaggagccgaagcatgtggtactcacagtatggaaatgaggccatcttggtccgagacaaaaacaagctcgaggtctctagggacacagggcagtccaagttttgcacaattatgtaattccgatgtgagcacctgaacccaggaccacactttgaggaaaacagacctgagtttcattcagttgtcttgttgatttccaatccttgctggaagattttgaataatgaacgtgaagatcaaactgtggtgtaagaacccaagaagcgcagaattggcccccgaacccctcagcacagccgcggtgtgactggtgcacaggacacttcccctctagagtcccctccgtggtcagctgttgtgacatttgaattctccaggtgctgctagggacagcataggcccgggcactgctagatgcttggtttaaaccaggccaagagtaaccctcccatccccattacaaaccaccttttcaagttttttaggttttagttttttgtttttagcataaatggcggagtagcagcagcatctcaccaggagctttgtcgcattacattttgtattttgcttttttgagacggggtctcactctgttacccaggctggaatacagtggcacaatcacagctcactgtaggctctatctcctgggctcaggtgatcctcccaaactcagcctcccaggtagctgtggctccaggcgaactactgtatatagagacagggtttccctatgttgcccaggctggtctggtcttatagtcctgggctcaagcctcccagagtgctgggattacaggcatgggtcaccgcaccaagcccgcatttatattttgagttaccaaaatgatcatcatctgtgaagcatcatggtagcttccaagcatttgcggcccactggccagtgcatccaaaacacattcaggaaacccgagagttgtaagttgcacatgtgtggcctacagtgacttccatgtagtaacagggctgagcaaggcaggctctccaccagacttaaagcattcaaccactctgcccccaggccgacactgacatgtgttcactgtcgctgccacagctggccacctgtgttcagtctccatgaaggattcaccttctgctctcactgacatctccttggctacacaaatgtgcaggtgtctcttgtcaaagctaatttttcttttcta

SEQ ID NO: 469 - DNA, 2586 bp, Homo sapiens
catttggaggagggaatgcgactctgagcgggccaagccctagtgggtcagagtaggattctgatggaatttgcggtctcaccgcctactagggaggaaaagcagttttcaggagtcccgcaccgccgccagccgtgggctgggactacaagtcacggtctgtggagtccaggagtgggggcgggcgggaggcaggctgggagctgtagtagtgtcaccgccacgttcgcagcaggtagatgctcatcttgctaacggattggtgagagtggacagcaattaggctttggcaccttggagcgtggctccagctaagggaagcgaaacgcttcctgggtgagaggacttttattctttgctcatcctcttaaacctggcgagtgagcgcctgaggttcggagtgccttcttcacagacgacgcgaatgtggggtcttcgtgtttgccgaactgaagggaatttgccttcctgcctttaggttcgcagcagttcataactgaataagagactttcctccatagaatggactttataaatctattaatttttttttttttttttttttagagctaaggtcttgctctatctcccatgccaaagttgcagtgagctgtgatcacaccattgcaccccaacctgggcgacgggagtgaggccctgtctcaaaaaaaaaaaaaagaaaagaaaataggtgtaacaggatatagtttatttacttcttcaaaaaaaggaaaatatttctgttaaaataatccaactaatacatccactccagaaatattgcacctggctcagtagttgaaaatttgttaaaaggtatttactgttaatagctagttacgattagtcttagttttcaattagcaagcattttatggttttaaaattgtttttaatttccatttaatatgctctaccagttactttttaaaagtatacttttaaaatagtttattttaaactttagggcatatccttttttttttttttttcaaagaaattaactatgtaatttatcaaggcaatcatcaactgccctttccttgtcaatttttaaatagttccattatttcaaaattctggtgaaatatttatttatatctttcttcagtagtaagttgtctcctagagatatttcatttctttttctagctgaatgtttggtatttaatatttcttcagtagatttcaatttgctatctttctgtagttctcactgagtgatctttctgttgtgttacatgaatactatttttaagtatattcataatatttcacaaatccagtatcatgtgtcatacagatgagtctaggcagtggttttatttctaatgagcaatttgtgagacatactattaatcatgcaatacctttaacagttgaactttttaattgtgggtttgtgtgtgcgagggagatatttatgatttctactaaagccctcaaagctgctcagacagcccttttgtccatttttggtcagctcccatggctttatcactcgcttcaagccctacaccctgcttcttcattcttagaaaaatggagttatcaagttttaactcagtttctcctttccaagtgaaattgggaaatcacaaatttacccctataacttactcatttctctccactattaggattctgttgaccctctctacccttttatgtctcttcctactcttgctgtacaaatatctcctctcctttattgtcagtcttttccccttcagtgactctccttatcctacaaaatgctcaagactcccacaaactactgcccatctcatgcaacccacaacagtctgactactggcacatatctcctaaaggacttcctacttgccaaatctcatgacctcttagtgtacatccttcttaacccttgtgcagcatttgacccttttcagtgaataccattccttcttgaaattctccctttccttagtttgttttaattgaagtgaaatttatataatatgtaattaatcattttaaagttaacaattcagtggcatttagtacattcacaatattgtgcaactaccacatctatctagttctaagacattttcgtgtccccaaaagaaagccctattcccattaaacagttactccctactccttcctcccctaacatcctggcaatcaccaatctgcattctgtctatatggatttacctattctgtatatttcatataaaaatggaatcatatataatagtatgtggcctcttgtgtctggcttctttcacttgcataatgttttgtttcaaatcttaatattttcatatttgcctcagattttcagtaagaaaacagtatggatagagttgagatgcctatagagtccttcctttctctccccagaaataaccattatcttgtatttagaatttttattgttccagtgcatgtataactttatgctgagtagtatcttgcctttaaaaaaattctggtaaaatcacataacaaaatttactatcttaaccatttttaagtgttcattagtgttaaatatatttgcattgttgtgcaacagatctccagaaacttttcatcttgcaaaactgaatatgtata

SEQ ID NO: 470 - DNA, 2903 bp, Homo sapiens
tggtgatacaatgtgggactgattagtgaggacagagacaagaggctatgcaagcagttaggaggctgctgcattattactttggctgagaaaggtgaggaggctgcagaagaaaagttggaagctagaagaagttggtttatgaggtttaacgaaagaagacgtctccataacctaaaagggcaaggtgaagcagcaagtgctgatggagaagttgtagcaggtaatccagaagatctaactaaggggcacacccagctccaagatcaccttcccaggcctctcttgctatgacagatgccatgtgacacagttctagccaatgagaagtaaccagaacaccctgggtgcagtggcttatgctgtaatcccagcactttggaaggctaacgtgggaggattgctcgagaccagcctgggcaacatagtgagagcctgtttctacaaaacatacaaaaattagctaggcgtgggggtgcatgactgtagtcccagctactagggaggcagagatgtgaggattgcttgagtccagaaggtggaggctgcaatgaggtgggatcatgccactgcactccagcctgggagacagagttttgcttgagaccttgtctcaaaacaaaataaaacaaaccactcctacttactgaacatacattatatatgtgtcactcaatcatactttatcacatttaatatctgcccccattttttaaaagtagctatttactgataaagacactgaggatcacagtgcctgtaaattagagctgagatgaaattaaggtgatatttagttccaagctccttgctatttccaacattactgatcacctctcaactgccaaactcttaataagtgaaaaaagctactttcattatcattgaagttgccaattaagcgctcaacaaataagtagctctaaacctgcaataccaaatctcttcaggtgtttccagatataaatgatggacacacaagtctctaccaccattacagataacaaaaggcatagatatgcagaataagtatctttagcagcctatgggtaaaccctgagaggaaaatttgttttcctttactaaaagagagggagaaggcagttgagagaagaccttctgaataggaggagctgtatgattatccttctgttgtttcgttgtgaaaatatgttggtactctgatcacttcggtcacttcctaggggagctttttcttagttttcaaagaaacatgaaaaaacatattgatttaaagggcagaaggttattatatggaaccacagagtgtgacatttattcctgttcctgcaaatttctttatcaaagaaatatactttgagataaatgttataataataagtcctagctttcttaatatttctaaaaacaaagatgaccaacttacaccaaagtaccaaagtagaattaaggttataactaccacggtaagttactgccattaaaaagtactgctgttctaagtttctggctggataacagactaccataaaggattataaagatgaaatggcttattttttgttctaacatggaaaatatttcaggtaatttttattataaatagaatatgacaagaagttcaacaaagaggtaggttttaagcattcctgaaccctctgtaacatctaagcttggtgaatctttaacagtccagaagcttagtttctatattgccatacctaaaaatgtaaatatcagtaatttctttgtctttgattttttttatatccattagtagaccctttgacctcaagtgtaagaggattcttggtagattaaaaacataactactgatattaatagttttcttcagatattaaaaaacacattgaaacagattaacaaagggatatcttttataattttcagcatgactccatattgtcaaggtgactgaaattcagtagtgtacaataacgttgcttattgttattgttactgatattatttttccggtgaagtgcagccgtggatcttctgatgcacagtgatggtaccttgctctatttccatatatcacatttatccaattcacccagtcatctgtcttactcttttcttatcaccaacataacaattttagccttcataatggctggaaattggcaaacagcaatgagggactaccacatatcaaaacctatcacaaccacctttggtgtattctgattcctgactgagaaacagaaacaatcacaatccaatgtttcagaaataatgcattaatatgagcagtgggcagaaatctattttcacttttttctgatacatttttcctcatatttcatagttagaaagaaatcaagaacattacaaaaagatgtttttgctttcacagtgacaccttaaattttttttcagtttggttttagtctattaattgatggtggatgtaaatattttgccatcagcaactgtagtatgaaaatgcaattaaatataccttccaagaaaattctattgaatataaaatttgaaccatttttaatatattttttctaattttcaagaagacattggagattaataatatatgccctcttatttttataaagcaattaaaacaaatttaaatttgggtaaacctttatttatgtattttctgaaacagttaaacacattacatatctagctctgaaaagtatagctgagttattgggtgtttttgtaaatgaagacaaaaagaggaaggaagatttgaatcaagtgtgtatagcattttatatcctgattttcttataaatattttgcatggtcactaaatattctttgaagatgtaatttataatggttacagggcattgcatttactgtatgtgttgtaatttattcagttcatcttttattattgagttttgttattttttctattataaata

SEQ ID NO: 471 - DNA, 1706 bp, Homo sapiens
gggctgctttgcttctaacgtaagtcaaatagcccagcatggaaaatacctaggaaaaaagtgggatatgcaaagcaaacctcaaaaaaaaaaaaaaaaaaagacaaaagctggtgggctggccacggtgtcacaaatgaagtctccagtggcttcatcataacaaatcccatctgtaggaggtaagccaacaaggtgtactgcacaagaagaaaagaatgcagagctttcagtctggccaagaagacaggctaggttcaagcagtactcgaagattgcttgttaaccccagcatagagggtaagagtatacccttggggtcagactccctggtttcaactcctgggtaacttagaaagtgttacctaaattttcaacgtgttggtagactttttccatctgtaaaataggaagcataagaattttagatggcatccctacccaatctacataattttggcgaacatcattcctcctactctctctacttgctaaggaatgacatagattgccagaaaacaactaccagaagctaaatgagagacatggaacagttccttccctcacagccctcagaagaaaccaactctactgatgtcttaatttcacacttccagcttccagaactgtgagacaatacctttctgtggtttaagccacctagtctgtattactttgttatagcagtcttagcaaactagtatggaagggagagcacagataaaagatgtgttatcaatcaagtcaccagtgtgagcaaatagatcctaatcccactggagaactctggcagacagtgtagaatgtacaactcaaaattatcctgccagacagaccccaacagtaatgggtaaagatttgaatagatgtttttcccaagaagatatacaaatggccagtaagtacaagaaaagatgttcaacatcattcatcattagggaaacacaaatcaaaaccacaatgagatatgtcacacccaccaggataaccacaattaaaaagacgataataacaaatgttagtgagaatgtgaaggaattagaactctcattcattgctggtatgaatgaaaactggcataactgccttggaaaacagttttccagttcttaaaaagttaaacatagagttatcctatgacccagaaactccactcccagttgttacatacccaagaagattgaaaaaaatattcgcatgaaactcgtacatgaatattcacagcagcattattcagaatagccaaaagcggaaaaaaatccaagtgtccatcaactgatgagtggttaaacaaaatgtggtatatccatgcaatgaaatagatttcagcaataaaaagaaatagattattgatatatggtacaacatgaataaatcttgaaaacattatgttaagtgacagaagccagtcataaaagcccacatattatatgactccatacaatatgttttatataaaatttatataaaatttccagaataggcaaatccatagagatggaaaatagattaatgtttgctagcagctggtgggagggtggaatggggaattactgctaataagtacagaatttttgtttggagtgatgaaaatgaactgaaattaaacagtggtgatggttgcgcagctatttttagttaatatactaaaaaccactgaattgtacaccttaaaagtatgagctttatggtatgtgaattatatc

SEQ ID NO: 472 - DNA, 2286 bp, Homo sapiens
cctggcctttgacgtgtctgttctctcctctctcactccaccaccactagcttccagggatctccagcctgtatagaaacttctcactacgctctgctctcgcccttttagcagtctccttatgctgctgactccacctcccaggcaaatcattctaatttaattctttacaagtattctccctcctggagtgctgggctccaccacgggggcaatcaaggcacatcacccacagctttccagacacccacagccttcctgtcctgctgtgagcgtggctccttcagccttcttccttcttcgcctgtggggagtcagacattcgccaacctcgtgctcaatccctgcctctgtccaggcctgctaagcccataagacatatctgcatgtgtcagaatatgtgcaagctttcgacaggatccccgtttctccccatggagcttttggcaaatgaagggtgtggttcagcagctggtctgatgtcccttaagctcccccacaccagcacaccaggactgtctctgtctccacattccagcatatctgggtctcagctttgacacaatcagacactgttgctgaagtttttatgtttatatgacagaagaaactgtcacccctcccctgccccccgcccccacccacctgttataagaaagagctccaaggtcttgagaccacacgcaggggatgggaaagctgacattaaaggggaatgaggtctttctgtgattccagcagcctcaggaggacaacgtcagaaaccaaaagctcctctgcccagggatggggacccgccgacttcaagggcagaacaggtgcctggctagtgggggagttccccaatgtggatttaaactgtagtgtcttcagagaacataaccaacatttgtctttaggagatgtgtttgtttgagtagatcagctatgaaaatacccagccggcctgcagccctcccggcagatgctgaaaaggaaacccgactgggagatgccgcccacccttatccccagagagctgcgttgtttcctcttgccttggcctttggatccggccatagcaagcatccctgggcacctggctgaagctggaaactcgggataagaatggaagccctggaagggaagaagaaccgaggagtgagcttgccctgaggacagcacattgcactctcacctcctttgcagttaagtgtgaaatagtgccaatagagtgtaaaagaaaagaaagaagatgattcacctcccaaagattccccactgctgtgtcccttctatcaacttgatacaaaaaaacacaatggccccgggaaccacgtgttgaagatgagagagctaaaagatggaaagagactggagccctgagtcaccacctggaggaaaactacccacccatcaggaatttctactacacatttgggagtttttggggttacagcagctaatttaacgaaaacacccagagacaagagctattactgcacaaattggttaagttacctttttgtatgtttgggtcctctctattcgatgagactgcgcgttcctttgaagcaaatctgtttaaaattcttatatccccagagggcctagcagagagtatgcacagagtgagccttcagtaaatgctgagtaaataagctcctaatggatgaaactgtgtcttcgaagtttgttctgaccagtacttagtataggattttgtgctgttggtcacttttcataaaggttcttgctattaaggaacttgacatccaatgtactcatagaaagtagaaattcaggggaaacaaatagaagagatatgtccaaaaatccatttaaaatttcattgacagatatttatctctttaatcctggaagttgtttcatgtaagggaaggttcagtagaatgaaaacaggactgtaagctgggaggagatttggggtctcttggccaactctgccacaaattgtgttaccttcaacttccctgcctgtaaaatgaagtcagtggtccctaaaatgaaattggactaatggcaaattagctagcatttgattatacatcgatagtttccccatgtgattttaacgcacaaatcagattttggaaccattaggtaggtgatcttatcttatgagtttatctttttcaacatggaaaaataaatttgttttgctgaccgaaggctgtgtaacttagaactggaaagagacatgtaattgaataacccatctctatggggcaaaaaaatgaaacacctttagtttgttcaaagatgaaatggaaaaataaacatgtt

SEQ ID NO: 473 - DNA, 2167 bp, Homo sapiens
ccggccacttgagctgctcttacactgtatacctgtgtctgagtactcagttcatccaccagtcagggtctgtgggacagactaggcaggtggtgccccgtgtgaggagcagcgcagtggatcatgatggaaccctcgaaaatgaacgtgaagtgactgcgcagtaagtaatcggtgcccgctggggatttccaagtttgaggggattttccagctagggtttcatcatgggacaacagttatcccctcaacagcaacagtgtattgaaacaattgcctaaagctagcagagcctcagtctcagagatccaattaaaggacctgatgcaaactgttgttttccataacccatggtttccagaagaaggtacactagacctagagctctgggaacaagtgggaagaaatcttaaacgacgtgcgcaagggcagtaggtcccagtaacatcttaatgttatggtcctaatagggttgctttggccctgctcttcatagaagagcccaaaaagataagggaagaggaaccactacccactttaatgcctcagcctccctcagccttgccgtcactggctaaagataccaaagaggagacggaggttttcctccccctccccccaatgaactgggaagaagacaagcgatatactacagttatgggaccctgccttaggcaagcagcattggaaggggaactcttggcctgcccagtgatgcaagatcaacaaggcaattgggtgcatgagcccattacattgaacacttctaaggaaataagaaaaagcattagagaaaatggggccactagcccattcacaagaggattaattgaggcaatagcagataactaccatatgactccatgggactggtcagtgctagctaaaacaactttaggaggcgagtcaataccccctctggagggcagaatatgatgaattgtgtgaacaacaggctaaccagaatcaattgcctggacaaaacatcggctgctatgctccaggggaggggtccctatgttaatgtacaacaacaattaaattttgtcccccaagcctatgcacaagtgtccttatgcatcttcagggcttaggaccaaattcccgaaggcagagttcaacagggatcttttgtaaatgttcaacaagggccacaggagccatttattgaatttatccatcagttaacccaggcaattaagagcacacatggaacatcgaccattccacgggtatctcgtataaccctcaaggacaagccatagtggaacgtgcccattccacgcttaaaaatatgctttaaaaaaagggggaatatgaataaggaccctacaacactactagcacaagtcttattcacccttaatttcttaaatttagataataaatttcaatcagccatagaaaagcactttgctaaaacctcccaagatacaaagccttcagtgttatggaaagatgtaaatagtaatctatggtgcagtccacatgatttgctaacatggggaagaggatatgcttgtgttcacatcccctcaggtcctcttgggattccagtgcaatgcatcaaaccataccatggcatggctgggacccaatgcagtactggaaatgaagaatgtgagcctgtaggacccgcagccccggacaatgcagcttcctcggacaacacaggtcccggatggggaatgtgaaatagacaactgggggatgctaaataagacaactcaggaggctaagtgaatcacagacaccattcactccagataatctgttccttgctatgctgtctgttgtacattgcaactcttgtaaggatgcaaagctggaacacaagctgtaaccgctgcacctgtcaaacctgtcgctgcacacatctgtacgcttcaatcaactaaacctgatgcaaaaaacagaaaagggagagatgtaggggatcactcagagtggtgggaaaaactatagggaaaggacacaaaccttctgaaaggttggaagggtctgcagagcccctggggagaatagctggaagcagctgttctataaccctgagacagagggcaaggagtagatacaagggagtgtggggggaattgatcttaaacaatcttgttatgttgaccaggaactgacctttgatcatccctgcatgtgaagttccctgaaa

SEQ ID NO: 474 - DNA, 2532 bp, Homo sapiens
tcatttttcaggcctgagttgatcgttaatcatcttaattatgttcattctgaagccaacaggagaaccaagaccaaaactttattgtctctgctttcatttcttgatgaaacctctggactaagcacacatcttccttgtttatctctctcaaaggagtgtggagtgcttcatctggacatccacgggaagaaggaagacatgagggaatgctggaagaggagacaggccccagatttgggcaggaagtaaacagttttcaggctgaggccaatctgagcaggaacattccaatatttcttcagctacgttgtcccagcacttcactggttaaccttttatgtccaccatttgtggatttcacagctacttgtcaatggtgaatattgatcatcatcattatctactgagctgctaccatatcccagctactccttgcatgttgttcattattttctcaacactcagcatatttgcaatatgttatgtaatatcacagacaaggaaactgaacgcagaaatgttttatttcttgccaaacatcacatgaggatgaacaatgaaaccgatttgaaaccaggattgtctgattccaacatctctgggtcctttttcactctgatatgctgcaattaaaaagccatttctaagactgtacgttccaagattgtttctatagttggttgtgagagaagtttctctgaacttgcagagcacgggaaaccaggaggaggaggctcagggtgctctccacacgtgtgaaaccggctgtgggtgttgctttcctgtgactgccatttgccactgatgatcttctcttcctctggaagagtaagagggggaaggacacagtttcagtgcttcccatttataattaattaattaattaagggcaggtgcaggggctcacacttgtaaccccagcactttaggagattgagcaaggaggatcccctgagcctgggagtttgagatcagcctgggaagcagagtgagaccatgacttaaataaaaatttttttgtgttttgttctaatttttacttttttgagacagggtctcgctctgttgcccaggctggagtgcagtagcgtgatctcagctcactgcaacctctgcctctgggttcaaacaattctcatgcctcagcctcccaagtagctgggattacaggtgcctgccaccatgcccaactaatttttttgtacttttagtagagacgggttttcaccatgttggttggccaggctggtctcgaactcctgacctcaagtgatccgcccacctcagcctcccaaagtgctgggattacaggcatgagccaccacgcccgaccaaaaaaaaatttttttttttcaattagccagacattgtggcatgtgcctgtagtcgcagctaccatgggggctgaggtgggaggatagcttgagcccaggagtttgaaaccagcctgggcagcaaagtgagaccctgcctcaaaaataaaaaataaaaaaaaagctatttcttacagatattcattgagtggaaagcaatttaattttacgtataataagttatttcaaaaagaattgtagttatgtgtggcaatttttcataaatacctgatacaaatcaagatctaccatggtaagtgtgctccagagatacccttcttgaccaaaacaaagcagggcagggattgggaggccttttgggctgggctccaggacacaatgactagaagatgatgcaaagtgcatgacagagtagagagctacacagtttacacatattagactctgcacatattagagtctacacatattagactctgcaaaagtagttcttaaatgttagctcacatgcaccccagtcacgttactgagcagattcctgggctccagtcctgggtgattcttgcaggtggtccattgaccagattaggagaatcatcatccaaggtctcaaagccaaatttaggacgcagagagaaagaatcctaagtggttgaatgggaaactgattctcctcttgagcacaccttagattcaaataattgattggagaagatcattccatacaaaaatcttctgacctgcagagtttctacaaaaagtcttgagaaagaaaaagaaaaacatatatttttggaaaagattgaatttaattataattttgcaaattatactgtgggaaaggtcaacaaaaacaagcgtattgcggatgatcttataaaaagaaccttttgtaaagtaaacaattaccttctgatttgtctactctgaacccttgatgtttatttaaattggatgttctcatagcaggacacacctgatacctggcttgtctaaacacttagaaagtcaaattgtctagttatcaaagcccaggcttattttctaaataagtatggatggtctttcaggagtcatttcacagtgatgtgcagagagaaaaacaatacaaacaaacaaacaaacaaacaaaaaacaaatcattgttcctaagtgtgttttccagcctatgtctaactttctagcagataatggc

SEQ ID NO: 475 - DNA, 1834 bp, Homo sapiens
gtgatggttgtcgctggcaagatgcagcaaacagcctttctccagaccctggagagtctggagactgccggacacagagatctggagctgagacacagtttcctctatatgctggaataccaaattcctttgctggagtgagatttgctttgtaaactaaattgccatgtgacgtttttctccagaaaaacaacaacttcatctgcaggtcctgccagagcaagctttgcagctgcagatgctgctcacacaccccgaggaagaaaaagaatgcctcctgccagcaatctgtgagcaatcttgaatcctgccaccttgctcccagccactgagtccaactatgaactggagcataattgtttagaaattcttgatgcagtctattccagcagaccagatctgtcagatcatctgatggccactccggactgggagctatacactgactgaagcagtttcacagaaggaggacaacgccaggccaggtatgctccagtgaccctagacaaagtagtagaagcctatgtccccgctgctggtacttcagcacaaaaagctgaatggattgctctcatcagggccttaaaacagtctcaagagaaatgggtaaacatttacacctactccaagtatgccttcttggtagtgtatgcccgtggggtcattcagaaagaaaggggattcttaacctcaagaaataaagacattaaacacctgctggaaattttagctttgtcagaggcagttgttctgccaactcaagttgctattatgcactgccaaggccaccaaaaggatgactccccaataaccaaagggaatcaagtggctgataaagctgcaaagcaggccactcaagaaacatatttacttgggaccttaatactccatttaaacttgtcagaatttaaactccactacactgaatgaaatgaagaatgcacacatgagtggacattgaccaacacagacccttgttcctcatggaaagctaacactcatgggatattactctctgaggccctggtatacccagtcctgaaagacctgcatgaggggacacactatggcagagatggactggctcacctggtgtagccatatctcaaagggccacaccttcaaaagatcattcaaaagattatacaagcatgttatctatataccccaaatgattcaagaactgcatgcaactcttcccagaggaggatacaatacaaaggaatatgaccattcgaggactggcaggttgatttcacccagatgccaaaaacactgggatttataagtacctactagtgtttgtagacacattttctggaaaggttgaagcttacccgagcaagagattgaagcctatccgaacaagtctgagaaagctgcagaagtagctaaggctctgctaaaagaaatcattcctcgatttggacttccttactccataccaagcaacaataatccttctttaatttctgagatcacacaaaggataagccaagcattacagatcaaatggaaatgacactcttgttggaggccacaatcaactggaaagactgaaaagatgatccacatgctaaaaaacaaaaacaaaaacaaaaacaaaacaaaacaaacaaacaaaaaaaccaaaaaaaacccctggcaaaactctatcgggaaacagatttaaaatggtaaagggttttacctattgccctgctatggattggggtagcaccctgaagtgggcttaaattaagcctttttgaaattgtgtgtgggagacccttctgaagttcttggccagggaccccacccttggacttaataaattaatcaagaattaaacaat

SEQ ID NO: 476 - DNA, 2189 bp, Homo sapiens
gactggccgcccgtgcatccagccaagctcctggcggacggatatcggaggcccaggcattcgagggacgtgcagttgcatcacggagagcctttgggaaccgtctggggtcttggcggagacatttgtgtttcgggggcagcgccccttcctacatccaggaggccgcgttgcccgggttaccgaaggccgccgcaccgacgccccgcccctgaccgccgatgcccctcccacgccggctgcggtcgccctcggcgctgcctgagagagtttgcgggatttcaagggtgacttgatttcgcctgatcctggtcggcagggtggggtttcttggcattcctcaggccgggggatgcgacgtctgggtgtggaggtgatgagctaggggttctgtggcactccggctctgctcagggcgacctgcgtaggggcctctggggcaggcccgctttctgtcaaaatggcagcgcccagcgtgcgcgggtatctctgaagttggggtttaagattcttgcccgtgagagcgaacgagcctgccgtacttctgattttgaaacacctgggtctgggcttcccgggcgagcggtgggtgagtgccggcctcattgcagcctggggtacgacgtgggcggttggtgttgtagggggtttacattggccgtggattttttcctctttccggatgtgtttttgcttttgagtgaagagttcccgtggttttgtaacctaataaatgcataaaatgcagcttgggttgtagactttggagaataattagagacctttagggctttaggctttctccaagaagcggagcaagttttcaaccatggctttcttcagaagactgcagcttttttgaatcatcttattaaagtggtggataaagttgcttgttctgatctttgagcatggcactggacgcaggaagcttcagccagtcctagttactgtgtttgcaaagcacttttcttgtttttggaatcttgctttatgtaatcaaccttaggaagaaccacacctttctgcactcaagcacagcgagaagtacgttatgcgtgaaaacaaaacaactttgcattaaaatccatctttctccgaaccacttggtcacggttgtgttctcatctgctttagtgtatttggttttgaaacgatatacctgctgtatgtctaaacaactttatattttcaggaacttttctgtttccttttcttttttttgagatggagtctcgctctgtcgcccaggctggagtgcagtggcgggatctcgggctcactgcaacctatgcctcccaggttcaagccattctcctgcctcagcctcctgagtggctgggattacaggcgcgcgctacctcgcccagctaatttttgtatttttagtagagacagggtttcaccatgttggtcaggctggtctcgaactcctgacctcaggcaatccgcccgcctcggcctcccaaaatgctgggattacaggtgtgagtcatcacgcccagcctctattttcaggaactttaaaggcagaaaaaacacttctctgaccttaccattctttagtgtacttgcaacctggtgtccttgcatccagatattcttactaatcctgatgcaatgacattttttaaaaggcactgaagagaaaatacttttattaagatatctttaagtctcctccctgatttcttgaaaatctcatgtctgttattgtttattattattttttaaacaggatagcaaaacttctattacttcctggttggtcaggtcattgagcaaatctagcctctgctttattgaaaggacttttttgagttttccctttgctatttcttaaaggatccaagaccaaggcaattgcacattgttatagagtaagattcagagctgatttctcttgatacactattgtgtgccagtgatatgaaatgatatgcggcacctcaaagctacaacattaatgcaagttactgatagagaatgctatcagagtgaaaaggaaaaacttgaagattattttggcttttttaaaaatagcaaagattacattattttattatattttgcttttgagagtaaagttggaataattactatatacaattgtttctacctaattatcaaggaatttaatcattaacgtacataattaatgtatctaaattactcaactttgttgtaat

SEQ ID NO: 477 - DNA, 2021 bp, Homo sapiens
gccggcgggatggagacagggaccggctgctggggtttgggaggggcagagattgaggagccagttgctgtgccctgttctggcaaaagaacaagatgcgttgcaagccagaatgatgaattccacttttcacgtcgcctccgtggcattctgcctaaatgatgggcatggaaatggtgctgagggatgtgggctgaatcgagaacacagggacttttgtcttcagggtgagaaatgttccggtccttaaggaaacatcgtgcagcagttgcatgggtcttccaggcgttcctcccaaaacattattttttttctttcactattgagttttaaaacttagcgttctgtcgctagcattgcatcttagttacacatcctctcccacccataacgtcaaatttaggagacgatacatccccaagctttcctgcattattttttgcagtaactggttattcagtttactttctttaaactatgtccttctcaaactagtctcacctgttcgttactgtcgcacatttcctgcgaataacacgtttctgtagttctgttgttagagcctggcatacctctccccgttcctctttcccacaaggtcatcatgcagaggggattttggtctgagttaaaagccaggtattctttcaccatcttttctgtcgatagtcaaactactacaaagactactcaaatttagtagtttcttatttccctatagcttcgttatattagaatggataacttatatttgcattaccttgctgtaatgaacacacaccaagttttcatttaaaattatcaaggacaaatgtcctctgaatttaaaaatgtgtatgtagtaaaatataactcagcatttcttgattacatatcattaaaatatgaatggtactatctgagccaccagaatcagcagtgaaggtattatattaataaaaaggactaagaatcaggactgttttaagggaactggaaccgaagaggatctactatttcatcctaggctagttaacatgaaatagtgaattaaaattgaaagagcttacatggatgcttcatatctgagctactttggcagggatgtttcaagaggatgtaggtaagtatggtgttcacctagctttccagaagtctcatattttgtgtaaaattggtatgcttattcatggttactttgggttagtagaagctatacttcttttccaaggaaagacagactgtcttaagattctaactgtgcatcttggtgataagaagttcttagggttgttaaatagagccaggaagcgtgaagctttctctctagatcatgacaactcatctagtaccttgcattacaaaggagctctaagtccatttactttgcaaggaatgaagttccagtgattaatgtttttgagaaatttgtattgggattgtggggtccagaaggttgccttaagtaactgttttcatgactcctaggtccctagatgttgagaagctctggattgaagctgtcacaaaaatagtgagtgggctccgtagtcactccatttagcataatacagagtaggacagctttgtgacatttctcaggctatttcccctgacctctcatcaatgctgacgtttttgataagaaggggtatagagactgcagcccttggggtggttacagttcatttatttacaggccttatattgacaggcctcagatttcatgctcagtaccataaaggttgatttttaaccaccaagaggcaaaagaaacatgacaggtcggagcatattttctcacctattctggcttaaatgatggtacttacctgatttgtggttgtaggtgaaatgttgggtgaaagattagctgctataaaaaggatgtcaggacgtgtagagaatttagaggacttgggtgggagatggatgtctattttctaaccccttagtcatgttcattccgatgccagcataaactctattttgagatgattatagaaataacttatatcttcatttgtttgtgggaaataaaa

SEQ ID NO: 478 - DNA, 3169 bp, Homo sapiens
cgtccccgccgccccgccgcccttcctggccgccgggctgcggtccagcccccggcctcgctatcctagttctctgcttggggtcctgcttctgctcttgccgccgcctctcccgcttcttccgggggggacatgccgcagaatctagactgctcagtgctccgacgtctgctctctgacaaccccgccgacgccgccattcgctgcctcagcaaaggggtgctcaaaaacggaagtgatcattttaccaaataggcattttccagcttaaaaaagatgtgcacattatttttcttggtttactcttcctataatcgataccgtttagtgtagaaaagccttctccttgctccattgttttgaaagaattaatatagactgcctctcatcaaatttttccacgattttaaattaagttaatctcagactgttaacttttcttttcagctggacacatttagagactcagtgtatctttctcttcactccggaaagagacagccactaagagattagctgatttgagtatccctctgtggtcgattataccattgcaaatcctgctctgctttaggttggcagtttattatttaatccagtaattattcaaaattcacttggacagatttgtaaggtagcagcagatattacttaaaaatcttttcctttcctgcttttacaaacatgtagttccttcctgaaaggtaatgttagttttgtggggggtttggtttttttttggtactgcttctctactgctactaggtgggaagctagttcttatttttagggccttgaagtgacacatacacaaaattataatctgctaatgaagaaataatttattcaggtcaataatataagtgcaagttaatcactgttccagtactatacgatgtatttttcatgagtgatgaacaggcctgcaattgtttttctttacaagaatcacagcaaacaaggttatgttctattgcctgttatctgagctctgtttttcagacatattctcatattcaggaataaactaaatctgagttttatgacttgtatggatttaagaaatgtagggaggagtttcattctaatcaaattcttcctgacacaatggttcaccctgatttaccagtttttgactacattgctgagcagagagaaaattgcagagttgacttgcagtattttgactgattcatgtcctagtgcttcggggaaattgcttaaggaagttggcgttgctcaaaagtatagttgagcaaaatataatccttccatattcgtgcttgggaaaggccactgtattctcagtcctgaagaagaggaatgtctagttaggttgggaccaccctgacagccaccagaaagtgcttatttactgccagtacaatagtttgacttaaaagctgcaagcgtcttgggtaccttgctctgctctagccacaaatattttcccacttttcttcatctattgtgtctccatttagtgaacatttgccagagctgtcatgagaataataccttagatttgtatggtagtttacagtttacaaataatatataatagacttattttgagtaccaacatcctgtgtgacgagctgaaaaaaatagcaggttttgttttttaactttaagaggtttggaaattgaaccacagagttgggaagtcagcaggtttcaaggctaagacaagtgtctgtaagaaacaaaccatttgaaaacaatgtatgcagataagagggtttattataaggatattggaggatcttatagatcttgacaacttggttgtcacctctgcttttctcttctttttctccaaataagatctctctgcttgtgcagtcaaaatggctgaatatggctaccctacactttccaaattgacagggttgtagctgcatgcagagattacatagctcttctaagtccaagtttaaagttcctaagagaaagcacgtgaatgaccctgcttggcttgtgtcaaatcattcttggccaaggtggctggtttacccaatatacactgcccatgagtgaaaggcaattatcacaggaggggagatggatcttgggtgtgggaaacaggtgtagaattcctactttttacagtttggtagaggtaatgtttagaacctaagatggtggtcacaaaacacaaatgcttatggggtcagttaaataacataaatgaatgaagctaataaatggagactgtggcaatgagagggagcacaggatccatctaaaatgagctactactaggccaagtgggatggctgacacctgtagtcccagctctcaggaggctgatgtgggaggatcacttgagtccaggaggtagaggctgcaatgagccatgatcaccccactacactccagcctgggcaacagaccctgtctcaaaggggagaaaaaaaaaaaaaaaaaaaaaaaggagctactactactcaattcaattctgactgactgatgctctgtgggaattcaagcgcaaggtgaccaatcttatgttttgcaagtgatgtcggaaactgggattttatgtaaaatctctatttcaattatgtaggaaaacaaaacaaaaagagaactataactgcaagtcaggtttgacctatgaaatggtagtttaggacttctgacctagatcatctgtgtggtcaattgccaaataatgacattttaaaatcacagcgtccataaagtctttttaccatagaagagactatgatgcgtgccacattaatgtgagcagtatacatgtttgtgtatagtgcatatacacaggaaactaaaaaaatcattcatccatccatcttgtttcttctctgaattgaaacctttttgatagattcatttcttctttacttggtgaggtaattataaagagctgcactatttgcgttagattttcctagagaagaaaccctgctgtaaaatttttaaaaacacatagaagttgtatgaccatgaacaacattatttaacctaagtctccattttctctcttgttatatgtcatacccatgtctcatggttgtcatgaaaatcaagtgaaatatgactttatgaagctctaacatgtatgaatgctcaatgtatgttaggcttcatattttgttagaattaagtctaacacaatacattacaatacagtatgattcaa

SEQ ID NO: 479 - DNA, 1842 bp, Homo sapiens
gcgcgtgcgcggcgtcggagcaagcggccccttccttcggccccgcccagccccgccggccgcccgctccctggggaaccgagggaggaatgggcggggtcctcaggtgcccggcgacgtcatccagcccccaaggctccaggacgcaaagggtcccctgatctggcagtccctggacctaccctggagagcgtggggggacaaaggccggacgcgccgcccgctgccgcctccaggaagccctcccttgccaggacagtggagttggccgggtgtggacagcaggaagggccggcgggaagggggggtggaaagagccactcctggttaataactctgcggggttaacctagggccggccatactcaagtacggacttgagtaaaattcagcaggaaaaggagcagggtgggcgccgctagagggaggcttcaggactgagggtcagaggcccaaggcctggcagggcccttccctggtcagctccaggcttggcctgatgcccggtgggccacgggcctcttccagcctcaggctgaccccagagaaagaggacagcccacaagcccgggaggccggcttgctgcctggtcattgctgcagagcctggagggggcctgcgctcagtccccgctgaccgtcccctcccttgaccctgccatagccatctgtgtaacccaggggaaattattccaaagggacttccacttctgataatctatgattctattttctaaaaaacaaaactctaaaatgcttaatgtcaaacatctgtcatcagctgaacaggaaaaggaacagtaagtacaaagcaaggcaacatcagaaaagggagccctgaggaatttctccctccttccctgagccgggtgaggccgtgactgcagcctgactcacaggttttatttcacttattaacctaaagatggtgaggatctgctggttctcccccaggcctggaggctgcctaccatcggtggctctcatcggtgagctgaggccacagttgttatagtctctgacttcaaaagaaagaccgatttcatgaacaattgcctgtctctctcgacggtggcctcggttttttgttgttgtttttctgtttgtttttgagacagagttttgcttccagcctgggcgacaaagtgagactccatctcaaaaaaacaaaacaaaacaaaacaaaacaaagagttactattcagaatatattaagaattcctacaactcaacaacaaaaaaaaatccaacccaattaaaaaatgggcaacagacccaaatagacatttcttagaagacacacaaatggccaataagctcatgaaaacatgtttaacatcactaatcattagggaaatgcaaatcaaagctaaaatgagataccacgtcacactcattaggtgttattttttaaaaataacacaacaacaaaaaataagaaatgtgggcgaggatgtgaagaaattgaaacccttgtggactgttgttggggatgtaaaatggcgcagctgctggggaaaacagtatgggagttcctcacaaaactaaacagaattgccaaacgacccagcgatcctgcctctgacatatactggaaagaactgaaataaggcgctggaacagacacttgtaacccatgttcacagctgagccaaaaggtgggagtaacccaaatgtccatcagtggatgaatactggcaataaaagctgtgtctggaggccactccgtttcacacagctctccacacgggtgagggcacagccttctcgtctctttcctatgctgcatcttcagagctagctatgatatgatatgatgtgatatgtacatatcgaatatac

SEQ ID NO: 480 - DNA, 2701 bp, Homo sapiens
tatggatgtctctgcaaaattctctcttttttgacacttttatctagaccggaagagttcaatctcaaaatacctctccctgcaccctggttttccatcaccccaaactccatttctttcctcccagtcacaccaaatttctgaagtagcccacccgtgctagccgtcccttgctcctacacactccttagtgccccctcatggggcttctgcccctaagtccctggactgaccacccagtccgaaggggtttctggtcctcaccaatctggtggctcctggctctttctgagacactgagatccccctgcccccacctcattcttgctgcttcctagacgcaggtggtcctcaggcttctccaggctctaaccttctcttggcattggcttttccaggctgtacattctgtactggtgccttctgcatcctcatctcatgctcggtctctctcttcagcctcaggcccacctgtcttctcgagagacattgcattttgcgtgtcccacaaatatctcaaacatgtccccaaacgaactcatccattctgaagctgcctggcttaacgaatggtgccattgtctccccagaagcctagaaagaaatgtgggagcctcagtccatgaccatgcccaatgctccccaccgaccctcgtctgagctctggatgtttctgtccgaggttgccaccccagcagcctagaaagaaacatgggatcctctctccatgactatgaccatgcccaaggctccctgctgtccctggcccaagctctggatgtctctgtctgagactgcttactcccagctggtctccctgtgttcaccatgtccacctttaacccattgtccacaccgaagatccaaccgtaaacctgatccaatcctgtttaaactcttgcgtgtttcccattgtccttgctggaatcccgagtcctaaaggcagggctcaggcccctcttggtctggttcctgtttgtgtttctatccttctggtccttcctctcctcccagacctcagttgcctcctccacactcttcatattctaatcgtacaaatactttgtttttggctttctgattcctcaaggtgttctgtgtgtttttccttcttcctcctcactttctctggctattttctgcccagccctttggctcttggctggtacagctctccctcagtaaccttctttcccatcctagccaaggcagggctccaggttcacacttctgtagcaccctcactttctatagcatagaccccgtgacactgtgttgaagctgattttacattatatatagccctgctggtctatagctttgagaagatttggatcctcagggcttggtcagagtatgagccatagtaaactctcaatgaatgctagttgaatgagcaaataagtgtattcagaggtactggagtgccaagctttgtatatatgcacgcactcagatatacatatggatgctcctggactctacttacacatatatgtatatgtgtatatacatgtatataaatatatatgttaaatatgtatatatggtccatgtatatgtgtgtacatatatatgtatatacatatttaaaccacattattgaggtatgattgatatgtcaaaagctgcacatatttaatgtatacaacttgatgagtttggggctatgtatacacctgtgaaaccatcactgccatcaaggtcataaacattcaatgcttttaagagcagggcttcccagaattaggaatttcatagaccattaaaattgcaggaaaaatgtaaactacattcaggtcgccatctttacattttctaagtgaagatgttgattaaaataatgtttgtttttttctcaccacaatcccacaaaagacatagcatttaaaaccaaaatggggctaggtgtggtggctcacgcctgtaatcccagcactttgggaggccaaggtgggtggatcacgaggtcagggagatcgagaccatcctggctaacatggtgaaaccccgtctctactaaaaatacaaaaaaaaattagctgagcatgatggtgggtgcctgtagtttcagctactcaggaggctgaggcagaagaatggcatgaacccagtacgcagagctggcagtgagccaagatagcaccactgcactgcagcctgggcaacagagtgagactccatcaaaacaaaacaaaacagcaaaatgcctactgaaaatctccttgcagtaccggcctacaaatgtaccgttaggaagctgttctcttcaagagcaggcttggaagctagagaggactgagattgcagctgtggtctgaagggctgtgtgtgaacgggggagccagagagacttggatctatcagtcagggttttctagagaagcagaaccaataagaaggaaagaacaagagttatgatgaggaattggctcacatgattatggaggctgatgagtcccaagatccgtgggatgagttggggagctgggggcccgtgagagccaatggtgcagtgcagcaaggtgctacggaccccggcagacaggtgtggatgatattctttgccaactgcctttggacaacctcagatttcaccacggtcaacaaatagagttacctctgacttcagtttggaaatttttttttttttgcagggaattctttgtagattttcaaactgctgttgattagtttgtca

SEQ ID NO: 481 - DNA, 1802 bp, Homo sapiens
agccctcaaaggaactgagaaaatttctccccactttgttctgaggggtctcagctactctggtatttaaaataaatgggttttgaaaaataggttactgccctttagttgatgactaaaacagaagccaagaagtgtgcaaattgcaaactgacatgcatgagccaaacatattctctgaaatgacaatgttcaagacgcagctaaagtcaccactctggcggtaagcgtgttacagagaactagatttctttccggcatccgcaacttggctggagtgaccaaggaggagttgaagagcgctggagatgcgagggtctcgctccatcgcccaggctggagtacagtggcatgatcactgctcactgcagcctcaccctccaggactcaagcaatcctccccactcagcctctcaggagctaaggactggctcaattgactataaagaatcgagaatgtcagctgaccaggcaaccaggagacgctttcctgacttccactatgcacgtgggctgcataattgtgtctgtgaagtaatgaagaacgtgcttgctctgtaacatccaaacgcgtggccaccattcacagatagtgtcctttgggaaaggtgtgggtatagatggggaatggtcagtcctatgaatatggggctataagacagcaaggctagaaagtatctgtgctttcattttttaattttatctattttttttttttttttgcactaatggtttgcattcacattgaggaactgagactgttttcagacttttggcctactgatgaccttatgttcataaaaggatagaagtataataagttgtttaatataaaacatactgaaattttattttctgtctccttggatataaagtttccacttatcttgggtttaagtacctactcctccacagagagggaccttgtgcatgtagattgatattctcaggatgaaagcagtacctactacaactaggaactcacagaacaggaagacgatgcttgttcctgatggcatgtaaatctagattcacttctagctggaaggaggcaggccagaaattcagagttgtaggtccttggcctgggtggatgtgctacaacatgaaccataaacctccatcacttatcttggcggatagaaaattggaactttggaggaaagtttcgcatcaagcactcagacccaataaatactcccactccttgcagagctaccgaacattctgcactccatttatcaggctacccaaggatgcttggaatgtgctggccggagccaccatcagcacttgctgatgactctttgtggtaccgccactcactttctgtactgtgttctttcttcttccttctcttttgttcttctgcccagcgacacacttgtgcagactgacttggtgccttggtgcctgttatggactgaatgctcgtgtcctcccacattcatatgtcaaagcacaatccccaatgtgatggtatttggagatggggcctctggggggtgatcaggtttagctgttgtaaaggtgagccctccgcgtctggattagtgcccttataaaaagagacacaagcgagctttctcattctccttccaccatgttaagatacaatgagaagacggccatctggaaaacaggaagcaggccttcacctgctgtaggcaatcataacacaatgagaagtattgtgttggcactatctggtccaggagcagctggacctcccagaaagacatggggtcagcctccacctggatcctgaacttcccagcctccataagcatgagaaataaatg

SEQ ID NO: 482 - DNA, 2315 bp, Homo sapiens
gcttctcaatgttttgtcacttttgaggtgccacggtgtctttctttggccaccatttcctggtttcttccttgtctgcagatcttactagagttaggcaaaatcaatttcaggttcagctctagtggactgggaacaatctccagcagggaactccctggacagtctggggacagctgggacgattcctgaagcaacttagataggaaaaatcacgatgagctttgatttagggaagaacattgttgggcaccctgagtccacctgccaccagccgcggagggcatgttataaacacaccagacacgccaaggatgggaacattggacctaatttagcaacaacaaatcctttccctcatgcatacttctctaattacaaatgagattgtggtaggcttagcatgggtaaataaaattgattctgtccacaaaattggagatgataaatgtgttcaagaaagggcttcaaatgcctagctgctgattagaaaaggcaaaataattgtgagatggtgactaatagatggaaggaattgtggtagaattggtctccttgcagaaatatcactggtctgatcttttcaagataattaccatcaaatgactgtgtgtttcatgctctacccttcagttggccgaggcattgcaggaatccccacattgactcggctgtaggtggctctggctttctctctgtttgaagcctgtaacggagcccctctgttttcttttttcttttttttaattatactttaagttctagggtacatgtgcacaacgtgcaggtttgttacataggtatatatgtgtcatgttggtgtgctgcacccattaactcgttatttacagtaggtatttctcctaatgctatccctcccccaggcccccacccgcaacaggccccagtgtgtgatgttccccgccctgtgtccaggtgttctctttgttcaattcccacctatgagtgagaacatgaagtatttggttttctgtccttgtgatagtttgctgagaatgatggtttccagctttatccatgtccctgcaaaggacatgaactcatccttttttatggctgcatagtattccgtggtgtatatgtaccaattttcttaatccagtctatcattggtgaacattttggttggttccaagtctttgctattgtgaacagtgccacaataaacatacatgtgcatgtgtatttatactagcatgatttataatcctttgggtatatagcacccaataatgggatcactgggtcaaatggtatttctagtcctagatccttgaggaattgccacactgtcttccacaatggttgaactaatttacactcccaccaacagtgtaaaagcgttcctatttctccacatcctctccagcatctgttgtttcctgactttttaatgatcgccactctaactggcataagatggtatctcattgtggttttgatttgcatttctctgatgaccagtgatgatgagcattttttcatgtgtctattggctgcataaaaatgtcctattttgagaagtgtctgttcatatcctttgcccaatttttgatggggttgtttgattttttcttgtaaatttgtttaagttctttgtagattctgcatattagccctttgtcagatgggtagattgcaaaaattttctcccattctgtaggttacctgtttactatgatggtagtttcttttgctgtgcagaagctctttagtttaattagatcccatttgtctattttcgcttttgttgccattgctttaggtgttttagtcatgaagtccttgcccacacctatgtcctgaatggtattgcctaagttttcttctagggtgtttatggatttagtctaacatttgtggatgaatgagtcttcgtgtagcattttaaggaggggagatgtacagactcattacagatgaacctgtttgctaagcacagtacttaaactgccattttgatagattaacactagtagttagaggacatcttcaaatttgcaggaaacaaggtttgaatagaagatttgtaagtcactgttctcagtggctcatgcctataacctcagcactttgggaggctgaggcagtcagattgctttaggccaggagtttgagaccaggctggccaacatggtgagaccccatatctacaaaaaatttgaaaattagctgggtgtagtggtgtgtgcctgtggtcccatctgcttgggaggctgaggtgggagaatagcttgagctcaggaggttgaggctacagtgagctatggtcctgcctctgcactttagcctcg

SEQ ID NO: 483 - DNA, 2185 bp, Homo sapiens
gcctgaatatgctgttgttgtcactgaaatctggtcgtggtagaagaagtgagtctgcaggatgctgtgttcgtgtataatatatcacagtggttgctggagaagggaacttcccagagggatcgctgactttaagctgcaacatagctcaacggtcagaggtgatgtttccacccaaaatctaactgtgattactgcaggctacaaacttatttgcctcatggaatagtgaaagaaaccaaaatgttcaccccaaaatattgaggattgctaatgacactgaaaactcagggaaacactctgcctccacttctatttgcctgatcacaggacataaatccttccttactgaaaacagcacttgtgtgttgtcccagagaaggcaccagcaggcaccaacaggatctggaaactgattttaccatctttccacattctcccaccttttaaaagaccaaaattgctctctcctttgccttgccactaagtaggatttctggctctttgtgaaaatactatttaagcaaaccctctaagctaccaccttgagagagaaatacttttgagtagaggcctttcccacacaattggtacagtgtacattaatgtatttctgcttgtttttgttttgttaatctgacttttggtttcaagagagtatctcaactgagaacctaaaaaggaaaaaaatattatattttatcttctacaataggtatgtattttatccctatcatacagatgagaaaattgaggcacacagggttttttttgtaagaaagttgattaaaatgacataactaggaatcccgagaggacccacacacccttggaaaaaagcagactgctctgcaggacccgggagacactccaaatactgtgctggtatccacggctgagagacccatagatggttcacatcacaggactctgtgcagacagcccccagtaccagcttgaagccagtagatttgctgggtggctagaccaagaagagagataacaatcactgcagattggctcacaggaagccacatcctcaggaaaagggggagagtactacatcaagggaacaccccatggatcaaaagaacctgaacaacagccttcagccctagaccttccctctgacagagactacccaaaagacagggaaccagaaaatcaactctggtaatatgacaaaacaaagctctttaacaccccccaaaaaatcacactagttcaccagcagtggatccaaactaagaagaaatctctgatttatttgaaaaataattcaggaggttagttaccaagctaatcagggaggcatcagagaaaggcaaagcccaatgcaaggaaatcctaaaaaactatacaagaagcaatgaaagaaatattcaaggaaatatatagcataaagaaaaactaaccaaatcttcaggaaatattggatacacttacagaagtgcaaaatgctctggaaagtctcaacaatagagttgaacaagtagaagaaagaaattcaaagctcaaagacaaggtcttcaaattaacccaatccaacaaagacaaagaaaaaagaataagaaaatatgaacaaagcctctaagaagtctgggattatgttaaatgacaaaacctaagaataattggtgttcctgaggaagaagagaaatccaaagtttgaaaaatatattttagggaataatcgaggaaaacttccctggccttgctagagacacagacatccaaatacaagaagcacaaagaacacctgggaaattcatcacaaaaagatcatcacctaggcacattatcatcagggtatctaaagttaagacgaaggaaagaatcttaagagctgtgagaaaaagcaccaggtaacctataaaagaaaacctatcagattaacagcagatttctcaacagaaaccctacaggctagaaggaattggggccctatctttagcctcctcaaacaaaacaattatcagccaagaattttgtatccagcgaaactaagcatcatatatgaaggaaagatacagttgtttttcagactaacaaatgagagattttgccagtaccgagccaccagtacaagaaatgctaaaaggagctctaaatcttgaaacaaatcctggaaacatatcaaaataaaacctctttaaagcataaatcacacaggacctataaaaca

SEQ ID NO: 484 - DNA, 1599 bp, Homo sapiens
ggcaacaaaaaccgggagctgagcgtgaaggcctgggcgagcggaagggtgggagagaggggctaggccggggagggaagcgctggggccggggccggctttccaggagccggcgggagcggggtgcgtaagaggcgggcgaggaagaaccggcgggcaacgggaagggcagaggtggatctctccctcagcgacccctctcgccgagtgaagggtcgagggtttggggcacaaagggcgcagaacgttttttcctgtgagaggaacattaagactcctgggagggctggggctaacgcctccgagagccccacctggtcagcctagggttagacgtgtagccagggtgctgggagggggccgctgctgggccccagatagaggggtagaggccgctcaatcacggagccaggccccacctgctgtagaaaccgccaccgagagccggccgggcttgctgcacgctctggattccgcctgcctataggcgaggcacaagggactgcgagaccaggcctgaaggcagagaggtcagcaggctcgggggcctctgctccagcggacaacaagcgcaccgggcgggaacggccgcctccccaggcgctgcagcctagctctcatgtgacccacggaacttagcccctctgttctgtttgcatacctgcaaattgtgggcattcacgccgggactgccaactcaccaggttatttatagtgacaagagagtgagacagtgggcttgaaagtgttttccagaccaaggctctgtcccacatataggtggattaactgtctggcaaaggcaggtctcttcttcggcctttctgacatggtgccagccctactggagtcagcaggagtccagcacaacagatggtggtcagtggtgacttcggcacaggcagttcgtgaggagtggttggtggccatggagtcctgactggagtaggttaaaaagaaggaggaaaatcacaagaaagagagactggaggtggaggttggggagaaaagagataatggtatggtgagatgtctactgaactgctcctgccaaggcccctccccatgtactctcaacgaaagttgcagccacccattctcattttgtggatataattccctgctttagttttctgagcacaagaaatcaactttggctgcacattggaatatctgagaagcttaaaaatactgatgtttggaccaggtgcagtggctcacgcctgtaatcccagcactttgggaggcagaggcagccacctctcaacaacccatgaagtgtctggagctccaccctctgctcaagaggaacggcacatgacccaggcctatgccaatcagcatttcacattcctcatggttggttcaaagctttgtatctgggtacacctgtttcctcctgagacatgaaggtccttgaggacatttgcttcctggagagaaaattgtcaccaaaattaaaaatacaatctagaggtagctttctgaaatcattttaaacataaagagatatgacttaaaaatttttcatctgaactgtcaatttccataaatagcttaatatagtgaaaaattgagaggttcttgaagccactaagt

SEQ ID NO: 485 - protein, 6 aa, Artificial Sequence
  Description of Artificial Sequence Synthetic 6xHis tag
HHHHHH

SEQ ID NO: 486 - protein, 10 aa, Artificial Sequence
  Description of Artificial Sequence Synthetic peptide tag
  MOD_RES (1)..(10): This region may encompass 3-10 His amino acids
HHHHHHHHHH